(19) United States
(12) Patent Application Publication
(10) Pub. No.: US 2024/0092922 A1
(43) Pub. Date: Mar. 21, 2024

(54) ANTI-TNFRSF9 ANTIBODIES AND USES THEREOF

(71) Applicant: Eucure (Beijing) Biopharma Co., Ltd, Beijing (CN)

(72) Inventors: Yi Yang, Beijing (CN); Jingshu Xie, Beijing (CN); Chunyan Dong, Beijing (CN); Fang Yang, Beijing (CN); Chengyuan Lu, Beijing (CN); Yuelei Shen, Beijing (CN); Jian Ni, Beijing (CN); Yanan Guo, Beijing (CN); Yunyun Chen, Beijing (CN)

(21) Appl. No.: 18/465,115

(22) Filed: Sep. 11, 2023

Related U.S. Application Data
(62) Division of application No. 17/677,339, filed on Feb. 22, 2022.
Application No. 17/677,339 is a division of application No. 17/200,021, filed on Mar. 12, 2021, now Pat. No. 11,292,849.
Application No. 17/200,021 is a continuation of application No. PCT/CN2019/105315, filed on Sep. 11, 2019.

(30) Foreign Application Priority Data
Sep. 12, 2018 (WO) PCT/CN2018/105162

(51) Int. Cl. C07K 16/28; A61K 47/68

(52) U.S. Cl. CPC C07K 16/2878; A61K 47/6849; C07K 16/2818

(57) ABSTRACT

This disclosure relates to anti-TNFRSF9 (tumor necrosis factor receptor superfamily member 9) antibodies, antigen-binding fragments, and the uses thereof.

### CLAIM OF PRIORITY

This application is a divisional of U.S. application Ser. No. 17/677,339, filed on Feb. 22, 2022, which is a divisional of U.S. application Ser. No. 17/200,021, filed on Mar. 12, 2021 (now U.S. Pat. No. 11,292,849), which is a continuation of International Application No. PCT/CN2019/105315, filed on Sep. 11, 2019, which claims the benefit of International Application No. PCT/CN2018/105162, filed on Sep. 12, 2018 under 35 U.S.C. § 365(b). The entire contents of the foregoing applications are incorporated herein by reference.

### SEQUENCE LISTING

This application contains a Sequence Listing that has been submitted electronically as an XML file named “45124-0021003_REVSL_SL26.XML.” The XML file, created on Oct. 20, 2023, is 499,269 bytes in size. The material in the XML file is hereby incorporated by reference in its entirety.

### TECHNICAL FIELD

This disclosure relates to anti-TNFRSF9 (tumor necrosis factor receptor superfamily member 9) antibodies, antigen-binding fragments, and the uses thereof.

### BACKGROUND

Cancer is currently one of the diseases that have the highest human mortality. According to the World Health Organization statistical data, in 2012, the number of global cancer incidence and death cases reached 14 million and 8.2 million, respectively. In China, the newly diagnosed cancer cases are 3.07 million, and the death toll is 2.2 million.

Recent clinical and commercial success of anticancer antibodies has created great interest in antibody-based therapeutics. There is a need to develop anti-cancer antibodies for use in various antibody-based therapeutics to treat cancers.

### SUMMARY

This disclosure relates to anti-TNFRSF9 (tumor necrosis factor receptor superfamily member 9; also known as “4-1BB” or “CD137”) antibodies, antigen-binding fragment thereof, and the uses thereof.

In one aspect, the disclosure relates to an antibody or antigen-binding fragment thereof that binds to 4-1BB (TNF Receptor Superfamily Member 9) comprising: a heavy chain variable region (VH) comprising complementarity determining regions (CDRs) 1, 2, and 3, wherein the VH CDR1 region comprises an amino acid sequence that is at least 80%, 85%, 90%, 95%, or 100% identical to a selected VH CDR1 amino acid sequence, the VH CDR2 region comprises an amino acid sequence that is at least 80%, 85%, 90%, 95%, or 100% identical to a selected VH CDR2 amino acid sequence, and the VH CDR3 region comprises an amino acid sequence that is at least 80%, 85%, 90%, 95%, or 100% identical to a selected VH CDR3 amino acid sequence; and a light chain variable region (VL) comprising CDRs 1, 2, and 3, wherein the VL CDR1 region comprises an amino acid sequence that is at least 80%, 85%, 90%, 95%, or 100% identical to a selected VL CDR1 amino acid sequence, the VL CDR2 region comprises an amino acid sequence that is at least 80%, 85%, 90%, 95%, or 100% identical to a selected VL CDR2 amino acid sequence, and the VL CDR3 region comprises an amino acid sequence that is at least 80%, 85%, 90%, 95%, or 100% identical to a selected VL CDR3 amino acid sequence, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively;
(2) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(3) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 16, 17, 18, respectively;
(4) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(5) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 28, 29, 30, respectively;
(6) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 34, 35, 36, respectively;
(7) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 40, 41, 42, respectively;
(8) the selected VH CDRs 1, 2, 3 amino acid sequences are set forth in SEQ ID NOs: 43, 44, 45, respectively, and the selected VL CDRs 1, 2, 3 amino acid sequences are set forth in SEQ ID NOs: 46, 47, 48, respectively;
(9) the selected VH CDRs 1, 2, 3 amino acid sequences are set forth in SEQ ID NOs: 49, 50, 51, respectively, and the selected VL CDRs 1, 2, 3 amino acid sequences are set forth in SEQ ID NOs: 52, 53, 54, respectively;
(10) the selected VH CDRs 1, 2, 3 amino acid sequences are set forth in SEQ ID NOs: 55, 56, 57, respectively, and the selected VL CDRs 1, 2, 3 amino acid sequences are set forth in SEQ ID NOs: 58, 59, 60, respectively;
(11) the selected VH CDRs 1, 2, 3 amino acid sequences are set forth in SEQ ID NOs: 61, 62, 63, respectively, and the selected VL CDRs 1, 2, 3 amino acid sequences are set forth in SEQ ID NOs: 64, 65, 66, respectively;
(12) the selected VH CDRs 1, 2, 3 amino acid sequences are set forth in SEQ ID NOs: 67, 68, 69, respectively, and the selected VL CDRs 1, 2, 3 amino acid sequences are set forth in SEQ ID NOs: 70, 71, 72, respectively;
(13) the selected VH CDRs 1, 2, 3 amino acid sequences are set forth in SEQ ID NOs: 73, 74, 75, respectively, and the selected VL CDRs 1, 2, 3 amino acid sequences are set forth in SEQ ID NOs: 76, 77, 78, respectively;
(14) the selected VH CDRs 1, 2, 3 amino acid sequences are set forth in SEQ ID NOs: 79, 80, 81, respectively, and the selected VL CDRs 1, 2, 3 amino acid sequences are set forth in SEQ ID NOs: 82, 83, 84, respectively;
(15) the selected VH CDRs 1, 2, 3 amino acid sequences are set forth in SEQ ID NOs: 85, 86, 87, respectively, and the selected VL CDRs 1, 2, 3 amino acid sequences are set forth in SEQ ID NOs: 88, 89, 90, respectively;
(16) the selected VH CDRs 1, 2, 3 amino acid sequences are set forth in SEQ ID NOs: 91, 92, 93, respectively, and the selected VL CDRs 1, 2, 3 amino acid sequences are set forth in SEQ ID NOs: 94, 95, 96, respectively;
(17) the selected VH CDRs 1, 2, 3 amino acid sequences are set forth in SEQ ID NOs: 97, 98, 99, respectively, and the selected VL CDRs 1, 2, 3 amino acid sequences are set forth in SEQ ID NOs: 100, 101, 102, respectively;
(18) the selected VH CDRs 1, 2, 3 amino acid sequences are set forth in SEQ ID NOs: 103, 104, 105, respectively, and the selected VL CDRs 1, 2, 3 amino acid sequences are set forth in SEQ ID NOs: 106, 107, 108, respectively.

In some embodiments, the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 4, 5, and 6, respectively.

In some embodiments, the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 10, 11, and 12, respectively.

In some embodiments, the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively.

In some embodiments, the antibody or antigen-binding fragment specifically binds to human 4-1BB. In some embodiments, the antibody or antigen-binding fragment is a humanized antibody or antigen-binding fragment thereof. In some embodiments, the antibody or antigen-binding fragment is a single-chain variable fragment (scFv).

In one aspect, the disclosure also relates to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 225, 226, 227, 228, or 244 binds to 4-1BB;
(2) an immunoglobulin light chain or a fragment thereof comprising a VL comprising CDRs 1, 2, and 3 comprising the amino acid sequences set forth in SEQ ID NOs: 4, 5, and 6, respectively, and wherein the VL, when paired with a VH comprising the amino acid sequence set forth in SEQ ID NO: 221, 222, 223, 224, or 243 binds to 4-1BB;
(3) an immunoglobulin heavy chain or a fragment thereof comprising a heavy chain variable region (VH) comprising CDRs 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 232, 233, 234, 235, or 246 binds to 4-1BB;
(4) an immunoglobulin light chain or a fragment thereof comprising a VL comprising CDRs 1, 2, and 3 comprising the amino acid sequences set forth in SEQ ID NOs: 10, 11, and 12, respectively, and wherein the VL, when paired with a VH comprising the amino acid sequence set forth in SEQ ID NO: 229, 230, 231, or 245 binds to 4-1BB;
(5) an immunoglobulin heavy chain or a fragment thereof comprising a heavy chain variable region (VH) comprising CDRs 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 239, 240, 241, 242, or 248 binds to 4-1BB;
(6) an immunoglobulin light chain or a fragment thereof comprising a VL comprising CDRs 1, 2, and 3 comprising the amino acid sequences set forth in SEQ ID NOs: 16, 17, and 18, respectively, and wherein the VL, when paired with a VH comprising the amino acid sequence set forth in SEQ ID NO: 236, 237, 238, or 247 binds to 4-1BB;
(7) an immunoglobulin heavy chain or a fragment thereof comprising a VH comprising CDRs 1, 2, and 3 comprising the amino acid sequences set forth in SEQ ID NOs: 19, 20, 21, respectively, and wherein the VH, when paired with a VL comprising the amino acid sequence set forth in SEQ ID NO: 250 binds to 4-1BB;
(8) an immunoglobulin light chain or a fragment thereof comprising a VL comprising CDRs 1, 2, and 3 comprising the amino acid sequences set forth in SEQ ID NOs: 22, 23, 24, respectively, and wherein the VL, when paired with a VH comprising the amino acid sequence set forth in SEQ ID NO: 249 binds to 4-1BB;
(9) an immunoglobulin heavy chain or a fragment thereof comprising a VH comprising CDRs 1, 2, and 3 comprising the amino acid sequences set forth in SEQ ID NOs: 25, 26, 27, respectively, and wherein the VH, when paired with a VL comprising the amino acid sequence set forth in SEQ ID NO: 252 binds to 4-1BB;
(10) an immunoglobulin light chain or a fragment thereof comprising a VL comprising CDRs 1, 2, and 3 comprising the amino acid sequences set forth in SEQ ID NOs: 28, 29, 30, respectively, and wherein the VL, when paired with a VH comprising the amino acid sequence set forth in SEQ ID NO: 251 binds to 4-1BB;
(11) an immunoglobulin heavy chain or a fragment thereof comprising a VH comprising CDRs 1, 2, and 3 comprising the amino acid sequences set forth in SEQ ID NOs: 31, 32, 33, respectively, and wherein the VH, when paired with a VL comprising the amino acid sequence set forth in SEQ ID NO: 254 binds to 4-1BB;
(12) an immunoglobulin light chain or a fragment thereof comprising a VL comprising CDRs 1, 2, and 3 comprising the amino acid sequences set forth in SEQ ID NOs: 34, 35, 36, respectively, and wherein the VL, when paired with a VH comprising the amino acid sequence set forth in SEQ ID NO: 253 binds to 4-1BB;
(13) an immunoglobulin heavy chain or a fragment thereof comprising a VH comprising CDRs 1, 2, and 3 comprising the amino acid sequences set forth in SEQ ID NOs: 37, 38, 39, respectively, and wherein the VH, when paired with a VL comprising the amino acid sequence set forth in SEQ ID NO: 256 binds to 4-1BB;
(14) an immunoglobulin light chain or a fragment thereof comprising a VL comprising CDRs 1, 2, and 3 comprising the amino acid sequences set forth in SEQ ID NOs: 40, 41, 42, respectively, and wherein the VL, when paired with a VH comprising the amino acid sequence set forth in SEQ ID NO: 255 binds to 4-1BB;
(15) an immunoglobulin heavy chain or a fragment thereof comprising a VH comprising CDRs 1, 2, and 3 comprising the amino acid sequences set forth in SEQ ID NOs: 43, 44, 45, respectively, and wherein the VH, when paired with a VL comprising the amino acid sequence set forth in SEQ ID NO: 258 binds to 4-1BB;
(16) an immunoglobulin light chain or a fragment thereof comprising a VL comprising CDRs 1, 2, and 3 comprising the amino acid sequences set forth in SEQ ID NOs: 46, 47, 48, respectively, and wherein the VL, when paired with a VH comprising the amino acid sequence set forth in SEQ ID NO: 257 binds to 4-1BB;
(17) an immunoglobulin heavy chain or a fragment thereof comprising a VH comprising CDRs 1, 2, and 3 comprising the amino acid sequences set forth in SEQ ID NOs: 49, 50, 51, respectively, and wherein the VH, when paired with a VL comprising the amino acid sequence set forth in SEQ ID NO: 260 binds to 4-1BB;
(18) an immunoglobulin light chain or a fragment thereof comprising a VL comprising CDRs 1, 2, and 3 comprising the amino acid sequences set forth in SEQ ID NOs: 52, 53, 54, respectively, and wherein the VL, when paired with a VH comprising the amino acid sequence set forth in SEQ ID NO: 259 binds to 4-1BB;
(19) an immunoglobulin heavy chain or a fragment thereof comprising a VH comprising CDRs 1, 2, and 3 comprising the amino acid sequences set forth in SEQ ID NOs: 55, 56, 57, respectively, and wherein the VH, when paired with a VL comprising the amino acid sequence set forth in SEQ ID NO: 262 binds to 4-1BB;
(20) an immunoglobulin light chain or a fragment thereof comprising a VL comprising CDRs 1, 2, and 3 comprising the amino acid sequences set forth in SEQ ID NOs: 58, 59, 60, respectively, and wherein the VL, when paired with a VH comprising the amino acid sequence set forth in SEQ ID NO: 261 binds to 4-1BB;
(21) an immunoglobulin heavy chain or a fragment thereof comprising a VH comprising CDRs 1, 2, and 3 comprising the amino acid sequences set forth in SEQ ID NOs: 61, 62, 63, respectively, and wherein the VH, when paired with a VL comprising the amino acid sequence set forth in SEQ ID NO: 264 binds to 4-1BB;
(22) an immunoglobulin light chain or a fragment thereof comprising a VL comprising CDRs 1, 2, and 3 comprising the amino acid sequences set forth in SEQ ID NOs: 64, 65, 66, respectively, and wherein the VL, when paired with a VH comprising the amino acid sequence set forth in SEQ ID NO: 263 binds to 4-1BB;
(23) an immunoglobulin heavy chain or a fragment thereof comprising a VH comprising CDRs 1, 2, and 3 comprising the amino acid sequences set forth in SEQ ID NOs: 67, 68, 69, respectively, and wherein the VH, when paired with a VL comprising the amino acid sequence set forth in SEQ ID NO: 266 binds to 4-1BB;
(24) an immunoglobulin light chain or a fragment thereof comprising a VL comprising CDRs 1, 2, and 3 comprising the amino acid sequences set forth in SEQ ID NOs: 70, 71, 72, respectively, and wherein the VL, when paired with a VH comprising the amino acid sequence set forth in SEQ ID NO: 265 binds to 4-1BB;
(25) an immunoglobulin heavy chain or a fragment thereof comprising a VH comprising CDRs 1, 2, and 3 comprising the amino acid sequences set forth in SEQ ID NOs: 73, 74, 75, respectively, and wherein the VH, when paired with a VL comprising the amino acid sequence set forth in SEQ ID NO: 268 binds to 4-1BB;
(26) an immunoglobulin light chain or a fragment thereof comprising a VL comprising CDRs 1, 2, and 3 comprising the amino acid sequences set forth in SEQ ID NOs: 76, 77, 78, respectively, and wherein the VL, when paired with a VH comprising the amino acid sequence set forth in SEQ ID NO: 267 binds to 4-1BB;
(27) an immunoglobulin heavy chain or a fragment thereof comprising a VH comprising CDRs 1, 2, and 3 comprising the amino acid sequences set forth in SEQ ID NOs: 79, 80, 81, respectively, and wherein the VH, when paired with a VL comprising the amino acid sequence set forth in SEQ ID NO: 270 binds to 4-1BB;
(28) an immunoglobulin light chain or a fragment thereof comprising a VL comprising CDRs 1, 2, and 3 comprising the amino acid sequences set forth in SEQ ID NOs: 82, 83, 84, respectively, and wherein the VL, when paired with a VH comprising the amino acid sequence set forth in SEQ ID NO: 269 binds to 4-1BB;
(29) an immunoglobulin heavy chain or a fragment thereof comprising a VH comprising CDRs 1, 2, and 3 comprising the amino acid sequences set forth in SEQ ID NOs: 85, 86, 87, respectively, and wherein the VH, when paired with a VL comprising the amino acid sequence set forth in SEQ ID NO: 272 binds to 4-1BB;
(30) an immunoglobulin light chain or a fragment thereof comprising a VL comprising CDRs 1, 2, and 3 comprising the amino acid sequences set forth in SEQ ID NOs: 88, 89, 90, respectively, and wherein the VL, when paired with a VH comprising the amino acid sequence set forth in SEQ ID NO: 271 binds to 4-1BB;
(31) an immunoglobulin heavy chain or a fragment thereof comprising a VH comprising CDRs 1, 2, and 3 comprising the amino acid sequences set forth in SEQ ID NOs: 91, 92, 93, respectively, and wherein the VH, when paired with a VL comprising the amino acid sequence set forth in SEQ ID NO: 274 binds to 4-1BB;
(32) an immunoglobulin light chain or a fragment thereof comprising a VL comprising CDRs 1, 2, and 3 comprising the amino acid sequences set forth in SEQ ID NOs: 94, 95, 96, respectively, and wherein the VL, when paired with a VH comprising the amino acid sequence set forth in SEQ ID NO: 273 binds to 4-1BB;
(33) an immunoglobulin heavy chain or a fragment thereof comprising a VH comprising CDRs 1, 2, and 3 comprising the amino acid sequences set forth in SEQ ID NOs: 97, 98, 99, respectively, and wherein the VH, when paired with a VL comprising the amino acid sequence set forth in SEQ ID NO: 276 binds to 4-1BB;
(34) an immunoglobulin light chain or a fragment thereof comprising a VL comprising CDRs 1, 2, and 3 comprising the amino acid sequences set forth in SEQ ID NOs: 100, 101, 102, respectively, and wherein the VL, when paired with a VH comprising the amino acid sequence set forth in SEQ ID NO: 275 binds to 4-1BB;
(35) an immunoglobulin heavy chain or a fragment thereof comprising a VH comprising CDRs 1, 2, and 3 comprising the amino acid sequences set forth in SEQ ID NOs: 103, 104, 105, respectively, and wherein the VH, when paired with a VL comprising the amino acid sequence set forth in SEQ ID NO: 278 binds to 4-1BB;
(36) an immunoglobulin light chain or a fragment thereof comprising a VL comprising CDRs 1, 2, and 3 comprising the amino acid sequences set forth in SEQ ID NOs: 106, 107, 108, respectively, and wherein the VL, when paired with a VH comprising the amino acid sequence set forth in SEQ ID NO: 277 binds to 4-1BB.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

In some embodiments, the VH when paired with a VL specifically binds to human 4-1BB, or the VL when paired with a VH specifically binds to human 4-1BB.

In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

In some embodiments, the nucleic acid encodes a single-chain variable fragment (scFv). In some embodiments, the nucleic acid is cDNA.

In one aspect, the disclosure relates to a vector comprising one or more of the nucleic acids as described herein. In one aspect, the disclosure also relates to a vector comprising two of the nucleic acids as described herein. In some embodiments, the vector encodes the VL region and the VH region that together bind to 4-1BB.

In one aspect, the disclosure relates to a pair of vectors, wherein each vector comprises one of the nucleic acids as described herein, wherein together the pair of vectors encodes the VL region and the VH region that together bind to 4-1BB.

In one aspect, the disclosure relates to a cell comprising the vector or the pair of vectors as described herein. In some embodiments, the cell is a CHO cell.

In one aspect, the disclosure relates to a cell comprising one or more of the nucleic acids as described herein. In one aspect, the disclosure relates to a cell comprising two of the nucleic acids as described herein. In some embodiments, the two nucleic acids together encode the VL region and the VH region that together bind to 4-1BB.

In one aspect, the disclosure relates to a method of producing an antibody or an antigen-binding fragment thereof, the method comprising
(a) culturing the cell as described herein under conditions sufficient for the cell to produce the antibody or the antigen-binding fragment; and
(b) collecting the antibody or the antigen-binding fragment produced by the cell.

In one aspect, the disclosure relates to an antibody or antigen-binding fragment thereof that binds to 4-1BB comprising a heavy chain variable region (VH) comprising an amino acid sequence that is at least 80%, 85%, 90%, 95%, or 100% identical to a selected VH sequence, and a light chain variable region (VL) comprising an amino acid sequence that is at least 80%, 85%, 90%, 95%, or 100% identical to a selected VL sequence, wherein the selected VH sequence and the selected VL sequence are one of the following:
(1) the selected VH sequence is SEQ ID NOs: 221, 222, 223, 224, or 243, and the selected VL sequence is SEQ ID NOs: 225, 226, 227, 228, or 244;
(2) the selected VH sequence is SEQ ID NOs: 229, 230, 231, or 245, and the selected VL sequence is SEQ ID NOs: 232, 233, 234, 235, or 246;
(3) the selected VH sequence is SEQ ID NO: 236, 237, 238, or 247, and the selected VL sequence is SEQ ID NO: 239, 240, 241, 242, or 248;
(4) the selected VH sequence is SEQ ID NO: 249, and the selected VL sequence is SEQ ID NO: 250;
(5) the selected VH sequence is SEQ ID NO: 251, and the selected VL sequence is SEQ ID NO: 252;
(6) the selected VH sequence is SEQ ID NO: 253, and the selected VL sequence is SEQ ID NO: 254;
(7) the selected VH sequence is SEQ ID NO: 255, and the selected VL sequence is SEQ ID NO: 256;
(8) the selected VH sequence is SEQ ID NO: 257, and the selected VL sequence is SEQ ID NO: 258;
(9) the selected VH sequence is SEQ ID NO: 259, and the selected VL sequence is SEQ ID NO: 260;
(10) the selected VH sequence is SEQ ID NO: 261, and the selected VL sequence is SEQ ID NO: 262;
(11) the selected VH sequence is SEQ ID NO: 263, and the selected VL sequence is SEQ ID NO: 264;
(12) the selected VH sequence is SEQ ID NO: 265, and the selected VL sequence is SEQ ID NO: 266;
(13) the selected VH sequence is SEQ ID NO: 267, and the selected VL sequence is SEQ ID NO: 268;
(14) the selected VH sequence is SEQ ID NO: 269, and the selected VL sequence is SEQ ID NO: 270;
(15) the selected VH sequence is SEQ ID NO: 271, and the selected VL sequence is SEQ ID NO: 272;
(16) the selected VH sequence is SEQ ID NO: 273, and the selected VL sequence is SEQ ID NO: 274;
(17) the selected VH sequence is SEQ ID NO: 275, and the selected VL sequence is SEQ ID NO: 276;
(18) the selected VH sequence is SEQ ID NO: 277, and the selected VL sequence is SEQ ID NO: 278.

In some embodiments, the antibody or antigen-binding fragment specifically binds to human 4-1BB.

In some embodiments, the antibody or antigen-binding fragment is a humanized antibody or antigen-binding fragment thereof.

In some embodiments, the antibody or antigen-binding fragment is a single-chain variable fragment (scFv).

In one aspect, the disclosure relates to an antibody-drug conjugate comprising the antibody or antigen-binding fragment thereof as described herein, covalently bound to a therapeutic agent. In some embodiments, the therapeutic agent is a cytotoxic or cytostatic agent.

In one aspect, the disclosure relates to a method of treating a subject having cancer, the method comprising administering a therapeutically effective amount of a composition comprising the antibody or antigen-binding fragment thereof as described herein, or the antibody-drug conjugate as described herein, to the subject.

In some embodiments, the subject has a solid tumor. In some embodiments, the cancer is breast cancer, oropharyngeal cancer, ovarian cancer, B cell lymphoma, or Non-Hodgkin's lymphoma. In some embodiments, the cancer is non-small cell lung cancer (NSCLC), melanoma, B-cell non-Hodgkin lymphoma, colorectal cancer, or multiple myeloma.

In one aspect, the disclosure relates to a method of decreasing the rate of tumor growth, the method comprising administering to a subject in need thereof an effective amount of a composition comprising an antibody or antigen-binding fragment thereof as described herein, or the antibody-drug conjugate as described herein.

In one aspect, the disclosure relates to a method of killing a tumor cell, the method comprising administering to a subject in need thereof an effective amount of a composition comprising the antibody or antigen-binding fragment thereof as described herein, or the antibody-drug conjugate as described herein.

In one aspect, the disclosure relates to a pharmaceutical composition comprising the antibody or antigen-binding fragment thereof as described herein, and a pharmaceutically acceptable carrier.

In one aspect, the disclosure relates to a pharmaceutical composition comprising the antibody drug conjugate as described herein, and a pharmaceutically acceptable carrier.

In some embodiments, the antibody is an IgG1 antibody. In some embodiments, the antibody is a human IgG1 antibody.

In one aspect, the disclosure relates to an IgG1 antibody or antigen-binding fragment thereof that binds to 4-1BB comprising: a heavy chain variable region (VH) comprising complementarity determining regions (CDRs) 1, 2, and 3, wherein the VH CDR1 region comprises an amino acid sequence that is at least 80%, 85%, 90%, 95%, or 100% identical to a selected VH CDR1 amino acid sequence, the VH CDR2 region comprises an amino acid sequence that is at least 80%, 85%, 90%, 95%, or 100% identical to a selected VH CDR2 amino acid sequence, and the VH CDR3 region comprises an amino acid sequence that is at least 80%, 85%, 90%, 95%, or 100% identical to a selected VH CDR3 amino acid sequence; and a light chain variable region (VL) comprising CDRs 1, 2, and 3, wherein the VL CDR1 region comprises an amino acid sequence that is at least 80%, 85%, 90%, 95%, or 100% identical to a selected VL CDR1 amino acid sequence, the VL CDR2 region comprises an amino acid sequence that is at least 80%, 85%, 90%, 95%, or 100% identical to a selected VL CDR2 amino acid sequence, and the VL CDR3 region comprises an amino acid sequence that is at least 80%, 85%, 90%, 95%, or 100% identical to a selected VL CDR3 amino acid sequence, wherein the selected VH CDRs 1, 2, and 3 amino acid sequences and the selected VL CDRs, 1, 2, and 3 amino acid sequences are selected from one of the antibodies as set forth in Table 3. In some embodiments, the antibody is a human IgG1 antibody.

In one aspect, the disclosure relates to an IgG1 antibody or antigen-binding fragment thereof that binds to 4-1BB comprising: a heavy chain variable region (VH) comprising an amino acid sequence that is at least 80%, 85%, 90%, 95%, or 100% identical to a selected VH sequence, and a light chain variable region (VL) comprising an amino acid sequence that is at least 80%, 85%, 90%, 95%, or 100% identical to a selected VL sequence, wherein the selected VH sequence and the selected VL sequence are selected from one of the antibodies as set forth in Table 3. In some embodiments, the antibody is a human IgG1 antibody.

In one aspect, the disclosure relates to a method of treating a subject having cancer, the method comprising administering to the subject a therapeutically effective amount of an anti-4-1BB IgG1 antibody or antigen-binding fragment thereof and a therapeutically effective amount of an anti-PD-1 antibody or antigen-binding fragment thereof.

In some embodiments, the anti-PD-1 antibody is an anti-PD-1 IgG4 antibody. In some embodiments, the anti-PD-1 antibody is pembrolizumab.

In one aspect, the disclosure relates to a method of treating a subject having cancer, the method comprising administering to the subject a therapeutically effective amount of an anti-4-1BB IgG1 antibody or antigen-binding fragment thereof and a therapeutically effective amount of an anti-CTLA4 antibody or antigen-binding fragment thereof.

In some embodiments, the anti-CTLA4 antibody is an anti-CTLA IgG1 antibody or an anti-CTLA IgG2 antibody. In some embodiments, the anti-CTLA4 antibody is ipilimumab or tremelimumab.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)2, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains. In some embodiments, the multimeric antibody is able to crosslink one target molecule (e.g., 4-1BB) to at least one second target molecule (e.g., HER2) on the surface of a mammalian cell (e.g., a human T-cell, a human tumor cell).

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., 4-1BB) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a 4-1BB molecule may be referred to as a 4-1BB-specific antibody or an anti-4-1BB antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a flow chart showing the first part of an exemplary protocol of making anti-h4-1BB antibodies.

FIG. 2 is a flow chart showing the second part of an exemplary protocol of making anti-h4-1BB antibodies.

FIG. 3 is a set of flow cytometry results showing that the binding activity of anti-h4-1BB antibodies with human 4-1BB.

FIG. 4 is a set of graphs showing flow cytometry results of analyzing the anti-h4-1BB antibodies' cross-reactivity with monkey 4-1BB (CHO-r4-1BB), mouse 4-1BB (CHO-m4-1BB), and human-mouse chimeric 4-1BB (CHO-c4-1BB). NC stands for negative control.

FIG. 5 is a graph showing the results of surface plasma resonance (SPR) using the chimeric anti-h4-1BB antibody 16-1C4-mHvKv-IgG4 and human 4-1BB.

FIG. 6 is a graph showing the results of surface plasma resonance (SPR) using the chimeric anti-h4-1BB antibody 29-6A5-mHvKv-IgG2 and human 4-1BB.

FIG. 7 is a graph showing body weight over time of humanized 4-1BB mice (B-h4-1BB) with MC-38 tumor cells treated with mouse anti-h4-1BB antibodies 1C4 and 5F9, and urelumab.

FIG. 8 is a graph showing percentage change of body weight over time of humanized 4-1BB mice (B-h4-1BB) with MC-38 tumor cells treated with mouse anti-h4-1BB antibodies 1C4 and 5F9, and urelumab.

FIG. 9 is a graph showing tumor size over time in humanized 4-1BB mice (B-h4-1BB) with MC-38 tumor cells treated with mouse anti-h4-1BB antibodies 1C4 and 5F9, and urelumab.

FIG. 10 is a graph showing body weight over time of humanized 4-1BB mice (B-h4-1BB) with MC-38 tumor cells treated with mouse anti-h4-1BB antibodies 29-6A5, 29-4A10, 29-5F10, 45-8F1, 45-4B9, and urelumab.

FIG. 11 is a graph showing percentage change of body weight over time of humanized 4-1BB mice (B-h4-1BB) with MC-38 tumor cells treated with mouse anti-h4-1BB antibodies 29-6A5, 29-4A10, 29-5F10, 45-8F1, 45-4B9, and urelumab.

FIG. 12 is a graph showing tumor size over time in humanized 4-1BB mice (B-h4-1BB) with MC-38 tumor cells treated with mouse anti-h4-1BB antibodies 29-6A5, 29-4A10, 29-5F10, 45-8F1, 45-4B9, and urelumab.

FIG. 13 is a graph showing body weight over time of humanized 4-1BB mice (B-h4-1BB) with MC-38 tumor cells treated with urelumab and mouse anti-h4-1BB antibodies 45-8E2, 45-7E9, 45-7G9, 45-2B3, and 45-2C11.

FIG. 14 is a graph showing percentage change of body weight over time of humanized 4-1BB mice (B-h4-1BB) with MC-38 tumor cells treated with urelumab and mouse anti-h4-1BB antibodies 45-8E2, 45-7E9, 45-7G9, 45-2B3, and 45-2C11.

FIG. 15 is a graph showing tumor size over time in humanized 4-1BB mice (B-h4-1BB) with MC-38 tumor cells treated with urelumab and mouse anti-h4-1BB antibodies 45-8E2, 45-7E9, 45-7G9, 45-2B3, and 45-2C11.

FIG. 16 is a graph showing body weight over time of humanized 4-1BB mice (B-h4-1BB) with MC-38 tumor cells treated with urelumab and mouse anti-h4-1BB antibodies 16-1C4, 55-8F6, and 54-8B11.

FIG. 17 is a graph showing percentage change of body weight over time of humanized 4-1BB mice (B-h4-1BB) with MC-38 tumor cells treated with urelumab and mouse anti-h4-1BB antibodies 16-1C4, 55-8F6, and 54-8B11.

FIG. 18 is a graph showing tumor size over time in humanized 4-1BB mice (B-h4-1BB) with MC-38 tumor cells treated with urelumab and mouse anti-h4-1BB antibodies 16-1C4, 55-8F6, and 54-8B11.

FIG. 19 is a graph showing body weight over time of humanized 4-1BB mice (B-h4-1BB) with MC-38 tumor cells treated with urelumab and mouse anti-h4-1BB antibodies 54-1A11, 55-1E3, 55-8H5, and 56-2A6.

FIG. 20 is a graph showing percentage change of body weight over time of humanized 4-1BB mice (B-h4-1BB) with MC-38 tumor cells treated with urelumab and mouse anti-h4-1BB antibodies 54-1A11, 55-1E3, 55-8H5, and 56-2A6.

FIG. 21 is a graph showing tumor size over time in humanized 4-1BB mice (B-h4-1BB) with MC-38 tumor cells treated with urelumab and mouse anti-h4-1BB antibodies 54-1A11, 55-1E3, 55-8H5, and 56-2A6.

FIG. 22 is a graph showing body weight over time of humanized 4-1BB mice (B-h4-1BB) with MC-38 tumor cells treated with urelumab and mouse anti-h4-1BB antibodies 58-4B8, 69-3C2, and 69-4B11.

FIG. 23 is a graph showing percentage change of body weight over time of humanized 4-1BB mice (B-h4-1BB) with MC-38 tumor cells treated with urelumab and mouse anti-h4-1BB antibodies 58-4B8, 69-3C2, and 69-4B11.

FIG. 24 is a graph showing tumor size over time in humanized 4-1BB mice (B-h4-1BB) with MC-38 tumor cells treated with urelumab and mouse anti-h4-1BB antibodies 58-4B8, 69-3C2, and 69-4B11.

FIG. 25 is a graph showing body weight over time of humanized 4-1BB mice (B-h4-1BB) with MC-38 tumor cells treated with anti-h4-1BB antibodies 30-5F9-mHvKv-IgG2, 30-5F9-mHvKv-IgG4, 16-1C4-mHvKv-IgG2, 16-1C4-mHvKv-IgG4, 16-1C4-mHvKv-IgG1, 30-5F9, 16-1C4, utomilumab, and urelumab

FIG. 26 is a graph showing percentage change of body weight over time of humanized 4-1BB mice (B-h4-1BB) with MC-38 tumor cells treated with anti-h4-1BB antibodies 30-5F9-mHvKv-IgG2, 30-5F9-mHvKv-IgG4, 16-1C4-mHvKv-IgG2, 16-1C4-mHvKv-IgG4, 16-1C4-mHvKv-IgG1, 30-5F9, 16-1C4, utomilumab, and urelumab

FIG. 27 is a graph showing tumor size over time in humanized 4-1BB mice (B-h4-1BB) with MC-38 tumor cells treated with anti-h4-1BB antibodies 30-5F9-mHvKv-IgG2, 30-5F9-mHvKv-IgG4, 16-1C4-mHvKv-IgG2, 16-1C4-mHvKv-IgG4, 16-1C4-mHvKv-IgG1, 30-5F9, 16-1C4, utomilumab, and urelumab

FIG. 28 is a graph showing body weight over time of humanized 4-1BB mice (B-h4-1BB) with MC-38 tumor cells treated with anti-h4-1BB antibodies 16-1C4-mHvKv-IgG2, 29-6A5-mHvKv-IgG2, 30-5F9-mHvKv-IgG2, 45-2B3-mHvKv-IgG2, 45-7E9-mHvKv-IgG2, 45-7G9-mHvKv-IgG2, 45-8E2-mHvKv-IgG2, 45-8F1-mHvKv-IgG2, and urelumab.

FIG. 29 is a graph showing percentage change of body weight over time of humanized 4-1BB mice (B-h4-1BB) with MC-38 tumor cells treated with anti-h4-1BB antibodies 16-1C4-mHvKv-IgG2, 29-6A5-mHvKv-IgG2, 30-5F9-mHvKv-IgG2, 45-2B3-mHvKv-IgG2, 45-7E9-mHvKv-IgG2, 45-7G9-mHvKv-IgG2, 45-8E2-mHvKv-IgG2, 45-8F1-mHvKv-IgG2, and urelumab.

FIG. 30 is a graph showing tumor size over time in humanized 4-1BB mice (B-h4-1BB) with MC-38 tumor cells treated with anti-h4-1BB antibodies 16-1C4-mHvKv-IgG2, 29-6A5-mHvKv-IgG2, 30-5F9-mHvKv-IgG2, 45-2B3-mHvKv-IgG2, 45-7E9-mHvKv-IgG2, 45-7G9-mHvKv-IgG2, 45-8E2-mHvKv-IgG2, 45-8F1-mHvKv-IgG2, and urelumab.

FIG. 31 is a graph showing body weight over time of humanized 4-1BB mice (B-h4-1BB) with MC-38 tumor cells treated with anti-h4-1BB antibodies 6A5-H1K2-IgG2, 6A5-H1K3-IgG2, 6A5-H2K2-IgG2, 6A5, and urelumab.

FIG. 32 is a graph showing percentage change of body weight over time of humanized 4-1BB mice (B-h4-1BB) with MC-38 tumor cells treated with anti-h4-1BB antibodies 6A5-H1K2-IgG2, 6A5-H1K3-IgG2, 6A5-H2K2-IgG2, 6A5, and urelumab.

FIG. 33 is a graph showing tumor size over time in humanized 4-1BB mice (B-h4-1BB) with MC-38 tumor cells treated with anti-h4-1BB antibodies 6A5-H1K2-IgG2, 6A5-H1K3-IgG2, 6A5-H2K2-IgG2, 6A5, and urelumab.

FIG. 34 is a graph showing body weight over time of humanized 4-1BB mice (B-h4-1BB) with MC-38 tumor cells treated with anti-h4-1BB antibodies 1C4-H1K1-IgG4, 1C4-H1K2-IgG4, 5F9-H1K1-IgG4, 5F9-H1K2-IgG4, 16-1C4, 30-5F9, and urelumab.

FIG. 35 is a graph showing percentage change of body weight over time of humanized 4-1BB mice (B-h4-1BB) with MC-38 tumor cells treated with anti-h4-1BB antibodies 1C4-H1K1-IgG4, 1C4-H1K2-IgG4, 5F9-H1K1-IgG4, 5F9-H1K2-IgG4, 16-1C4, 30-5F9, and urelumab.

FIG. 36 is a graph showing tumor size over time in humanized 4-1BB mice (B-h4-1BB) with MC-38 tumor cells treated with anti-h4-1BB antibodies 1C4-H1K1-IgG4, 1C4-H1K2-IgG4, 5F9-H1K1-IgG4, -5F9-H1K2-IgG4, 16-1C4, 30-5F9, and urelumab.

FIG. 37 is a graph showing body weight over time of humanized 4-1BB mice (B-h4-1BB) with MC-38 tumor cells treated with anti-h4-1BB antibodies 16-1C4-mHvKv-IgG1 (G2), 16-1C4-mHvKv-IgG2 (G3), 16-1C4-mHvKv-IgG4 (G4), 16-1C4 (G5), and urelumab (G6).

FIG. 38 is a graph showing percentage change of body weight over time of humanized 4-1BB mice (B-h4-1BB) with MC-38 tumor cells treated with anti-h4-1BB antibodies 16-1C4-mHvKv-IgG1 (G2), 16-1C4-mHvKv-IgG2 (G3), 16-1C4-mHvKv-IgG4 (G4), 16-1C4 (G5), and urelumab (G6).

FIG. 39 is a graph showing tumor size over time in humanized 4-1BB mice (B-h4-1BB) with MC-38 tumor cells treated with anti-h4-1BB antibodies 16-1C4-mHvKv-IgG1 (G2), 16-1C4-mHvKv-IgG2 (G3), 16-1C4-mHvKv-IgG4 (G4), 16-1C4 (G5), and urelumab (G6).

FIG. 40 is a graph showing body weight over time of humanized 4-1BB mice (B-h4-1BB) with MC-38 tumor cells treated with anti-h4-1BB antibodies 54-8B11-mHvKv-IgG2 (G2), 55-8F6-mHvKv-IgG2 (G3), 56-2A6-mHvKv-IgG2 (G4), 69-3C2-mHvKv-IgG2 (G5), 61-6A7-mHvKv-IgG2 (G6), 70-6F10-mHvKv-IgG2 (G7), 70-3F9-mHvKv-IgG2 (G8), 45-4B9-mHvkv-IgG2 (G9), and also urelumab (G10).

FIG. 41 is a graph showing percentage change of body weight over time of humanized 4-1BB mice (B-h4-1BB) with MC-38 tumor cells treated with anti-h4-1BB antibodies 54-8B11-mHvKv-IgG2 (G2), 55-8F6-mHvKv-IgG2 (G3), 56-2A6-mHvKv-IgG2 (G4), 69-3C2-mHvKv-IgG2 (G5), 61-6A7-mHvKv-IgG2 (G6), 70-6F10-mHvKv-IgG2 (G7), 70-3F9-mHvKv-IgG2 (G8), 45-4B9-mHvkv-IgG2 (G9), and also urelumab (G10).

FIG. 42 is a graph showing tumor size over time in humanized 4-1BB mice (B-h4-1BB) with MC-38 tumor cells treated with anti-h4-1BB antibodies 54-8B11-mHvKv-IgG2 (G2), 55-8F6-mHvKv-IgG2 (G3), 56-2A6-mHvKv-IgG2 (G4), 69-3C2-mHvKv-IgG2 (G5), 61-6A7-mHvKv-IgG2 (G6), 70-6F10-mHvKv-IgG2 (G7), 70-3F9-mHvKv-IgG2 (G8), 45-4B9-mHvkv-IgG2 (G9), and also urelumab (G10).

FIG. 43 is a graph showing body weight over time of humanized 4-1BB mice (B-h4-1BB) with MC-38 tumor cells treated with anti-h4-1BB antibodies 6A5-H1K2-IgG2, 6A5-H1K3-IgG2, 1C4-H1K1-IgG2, 1C4-H1K2-IgG2, 5F9-H1K1-IgG2, 5F9-H1K2-IgG2, 16-1C4-mHvKv-IgG2, 29-6A5-mHvKv-IgG2, 30-5F9-mHvKv-IgG2, and urelumab.

FIG. 44 is a graph showing percentage change of body weight over time of humanized 4-1BB mice (B-h4-1BB) with MC-38 tumor cells treated with anti-h4-1BB antibodies 6A5-H1K2-IgG2, 6A5-H1K3-IgG2, 1C4-H1K1-IgG2, 1C4-H1K2-IgG2, 5F9-H1K1-IgG2, 5F9-H1K2-IgG2, 16-1C4-mHvKv-IgG2, 29-6A5-mHvKv-IgG2, 30-5F9-mHvKv-IgG2, and urelumab.

FIG. 45 is a graph showing tumor size over time in humanized 4-1BB mice (B-h4-1BB) with MC-38 tumor cells treated with anti-h4-1BB antibodies 6A5-H1K2-IgG2, 6A5-H1K3-IgG2, 1C4-H1K1-IgG2, 1C4-H1K2-IgG2, 5F9-H1K1-IgG2, 5F9-H1K2-IgG2, 16-1C4-mHvKv-IgG2, 29-6A5-mHvKv-IgG2, 30-5F9-mHvKv-IgG2, and urelumab.

FIG. 46 is a graph showing body weight over time of humanized 4-1BB mice (B-h4-1BB) with MC-38 tumor cells treated with anti-h4-1BB antibodies urelumab-IgG1, urelumab-IgG2, urelumab-IgG4, urelumab-IgG1-N297A, urelumab-IgG1-FC-SI, and urelumab-IgG1-FC-V11.

FIG. 47 is a graph showing percentage change of body weight over time of humanized 4-1BB mice (B-h4-1BB) with MC-38 tumor cells treated with anti-h4-1BB antibodies urelumab-IgG1, urelumab-IgG2, urelumab-IgG4, urelumab-IgG1-N297A, urelumab-IgG1-FC-SI, and urelumab-IgG1-FC-V11.

FIG. 48 is a graph showing tumor size over time in humanized 4-1BB mice (B-h4-1BB) with MC-38 tumor cells treated with anti-h4-1BB antibodies urelumab-IgG1, urelumab-IgG2, urelumab-IgG4, urelumab-IgG1-N297A, urelumab-IgG1-FC-SI, and urelumab-IgG1-FC-V11.

FIG. 49 is a graph showing body weight over time of humanized 4-1BB mice (B-h4-1BB) with MC-38 tumor cells treated with anti-h4-1BB antibodies 16-1C4-mHvKv-IgG1, 16-1C4-mHvKv-IgG2, 16-1C4-mHvKv-IgG4, 16-1C4-mHvKv-IgG1-FC-V11, 16-1C4-mHvKv-IgG1-FC-SI, 16-1C4, and urelumab.

FIG. 50 is a graph showing percentage change of body weight over time of humanized 4-1BB mice (B-h4-1BB) with MC-38 tumor cells treated with anti-h4-1BB antibodies 16-1C4-mHvKv-IgG1, 16-1C4-mHvKv-IgG2, 16-1C4-mHvKv-IgG4, 16-1C4-mHvKv-IgG1-FC-V11, 16-1C4-mHvKv-IgG1-FC-SI, 16-1C4, and urelumab.

FIG. 51 is a graph showing tumor size over time in humanized 4-1BB mice (B-h4-1BB) with MC-38 tumor cells treated with anti-h4-1BB antibodies 16-1C4-mHvKv-IgG1, 16-1C4-mHvKv-IgG2, 16-1C4-mHvKv-IgG4, 16-1C4-mHvKv-IgG1-FC-V11, 16-1C4-mHvKv-IgG1-FC-SI, 16-1C4, and urelumab.

FIG. 52 lists CDR sequences of several anti-h4-1BB antibodies and CDR sequences of the humanized anti-h4-1BB antibodies thereof as defined by Kabat numbering.

FIG. 53 lists CDR sequences of several anti-h4-1BB antibodies and CDR sequences of humanized anti-h4-1BB antibodies thereof as defined by Chothia numbering.

FIG. 54 lists amino acid sequences of human 4-1BB (“h4-1BB”), mouse 4-1BB (“m4-1BB”), monkey 4-1BB (“rm4-1BB” or “r4-1BB”), and chimeric 4-1BB (“chi4-1BB” or “c4-1BB”).

FIG. 55 lists amino acid sequences of heavy chain variable regions and light chain variable regions of humanized antibodies based on 1C4.

FIG. 56 lists amino acid sequences of heavy chain variable regions and light chain variable regions of humanized antibodies based on 6A5.

FIG. 57 lists amino acid sequences of heavy chain variable regions and light chain variable regions of humanized antibodies based on 5F9.

FIG. 58 lists the amino acid sequence of the heavy chain variable regions and light chain variable regions of several mouse anti-h4-1BB antibodies.

FIG. 59 is a graph showing body weight over time of humanized 4-1BB mice (B-h4-1BB) with MC-38 tumor cells treated with anti-h4-1BB antibodies Urelumab-mIgG1 (G2), Urelumab-mIgG2A (G3), 16-1C4-mHvKv-mIgG1 (G4), 16-1C4-mHvKv-mIgG2A (G5), Urelumab (G6), Urelumab-IgG1 (G7), 16-1C4-mHvKv-IgG1 (G8) and 16-1C4-mHvKv-IgG4 (G9).

FIG. 60 is a graph showing percentage change of body weight over time of humanized 4-1BB mice (B-h4-1BB) with MC-38 tumor cells treated with anti-h4-1BB antibodies Urelumab-mIgG1 (G2), Urelumab-mIgG2A (G3), 16-1C4-mHvKv-mIgG1 (G4), 16-1C4-mHvKv-mIgG2A (G5), Urelumab (G6), Urelumab-IgG1 (G7), 16-1C4-mHvKv-IgG1 (G8) and 16-1C4-mHvKv-IgG4 (G9).

FIG. 61 is a graph showing tumor size over time in humanized 4-1BB mice (B-h4-1BB) with MC-38 tumor cells treated with anti-h4-1BB antibodies Urelumab-mIgG1 (G2), Urelumab-mIgG2A (G3), 16-1C4-mHvKv-mIgG1 (G4), 16-1C4-mHvKv-mIgG2A (G5), Urelumab (G6), Urelumab-IgG1 (G7), 16-1C4-mHvKv-IgG1 (G8) and 16-1C4-mHvKv-IgG4 (G9).

FIG. 62 is a graph showing body weight over time of humanized 4-1BB mice (B-h4-1BB) with MC-38 tumor cells treated with anti-h4-1BB antibodies urelumab, urelumab-IgG1, 1C4-mHvKv-IgG1, 1C4-mHvKv-IgG4, 1C4-H1K1-IgG1, 1C4-H1K1-IgG4, 1C4-H1K2-IgG1, 1C4-H1K2-IgG4, 6A5-H1K2-IgG1, 5F9-H1K1-IgG1 and 1C4-mHvKv-IgG1-FC-V11.

FIG. 63 is a graph showing percentage change of body weight over time of humanized 4-1BB mice (B-h4-1BB) with MC-38 tumor cells treated with anti-h4-1BB antibodies urelumab, urelumab-IgG1, 1C4-mHvKv-IgG1, 1C4-mHvKv-IgG4, 1C4-H1K1-IgG1, 1C4-H1K1-IgG4, 1C4-H1K2-IgG1, 1C4-H1K2-IgG4, 6A5-H1K2-IgG1, 5F9-H1K1-IgG1 and 1C4-mHvKv-IgG1-FC-V11.

FIG. 64 is a graph showing tumor size over time in humanized 4-1BB mice (B-h4-1BB) with MC-38 tumor cells treated with anti-h4-1BB antibodies urelumab, urelumab-IgG1, 1C4-mHvKv-IgG1, 1C4-mHvKv-IgG4, 1C4-H1K1-IgG1, 1C4-H1K1-IgG4, 1C4-H1K2-IgG1, 1C4-H1K2-IgG4, 6A5-H1K2-IgG1, 5F9-H1K1-IgG1 and 1C4-mHvKv-IgG1-FC-V11.

FIG. 65 is a graph showing body weight over time of humanized 4-1BB mice (B-h4-1BB) with MC-38 tumor cells treated with anti-h4-1BB antibodies 1C4-IgG1-H1K1, 1C4-IgG4-H1K1, urelumab-IgG1, urelumab-IgG4, Keytruda®-IgG4, 1C4-IgG1-H1K1+Keytruda®-IgG4, 1C4-IgG4-H1K1+Keytruda®-IgG4, urelumab-IgG1+Keytruda®-IgG4 and urelumab-IgG4+Keytruda®-IgG4.

FIG. 66 is a graph showing percentage change of body weight over time of humanized 4-1BB mice (B-h4-1BB) with MC-38 tumor cells treated with anti-h4-1BB antibodies 1C4-IgG1-H1K1, 1C4-IgG4-H1K1, urelumab-IgG1, urelumab-IgG4, Keytruda®-IgG4, 1C4-IgG1-H1K1+Keytruda®-IgG4, 1C4-IgG4-H1K1+Keytruda®-IgG4, urelumab-IgG1+Keytruda®-IgG4 and urelumab-IgG4+Keytruda®-IgG4.

FIG. 67 is a graph showing tumor size over time in humanized 4-1BB mice (B-h4-1BB) with MC-38 tumor cells treated with anti-h4-1BB antibodies 1C4-IgG1-H1K1, 1C4-IgG4-H1K1, urelumab-IgG1, urelumab-IgG4, Keytruda®-IgG4, 1C4-IgG1-H1K1+Keytruda®-IgG4, 1C4-IgG4-H1K1+Keytruda®-IgG4, urelumab-IgG1+Keytruda®-IgG4 and urelumab-IgG4+Keytruda®-IgG4.

FIG. 68 is a graph showing body weight over time of humanized 4-1BB mice (B-h4-1BB) with B16-F10 cancer cells treated with different anti-h4-1BB antibodies.

FIG. 69 is a graph showing percentage change of body weight over time of humanized 4-1BB mice (B-h4-1BB) with B16-F10 cancer cells treated with different anti-h4-1BB antibodies.

FIG. 70 is a graph showing tumor size over time in humanized 4-1BB mice (B-h4-1BB) with B16-F10 cancer cells treated with different anti-h4-1BB antibodies.

FIG. 71 is a graph showing body weight over time of humanized 4-1BB mice (B-h4-1BB) with EL4 cancer cells treated with different anti-h4-1BB antibodies.

FIG. 72 is a graph showing percentage change of body weight over time of humanized 4-1BB mice (B-h4-1BB) with EL4 cancer cells treated with different anti-h4-1BB antibodies.

FIG. 73 is a graph showing tumor size over time in humanized 4-1BB mice (B-h4-1BB) with EL4 cancer cells treated with different anti-h4-1BB antibodies.

FIGS. 74-75 are diagrams showing two fluorescence activated cell sorting (FACS) procedures for analyzing spleen samples.

FIGS. 76-77 are diagrams showing two FACS procedures for analyzing tumor samples

FIG. 78A is a graph showing percentage of CD45+ cells in tumor cells.

FIG. 78B is a graph showing counts of CD45+ cells in tumor cells.

FIG. 78C is a graph showing percentage of CD11b+ cells in tumor cells.

FIG. 78D is a graph showing percentage of NK cells in CD45+ cells in tumor sample.

FIG. 79A is a graph showing percentage of CD3+ cells in CD45+ cells in tumor sample.

FIG. 79B is a graph showing percentage of CD8+ cells in CD3+ cells in tumor sample.

FIG. 79C is a graph showing percentage of CD4+FoxP3− cells in CD3+ cells in tumor sample.

FIG. 79D is a graph showing percentage of Treg cells in CD3+ cells in tumor sample.

FIG. 80A is a graph showing percentage of hCD137+ cells in NK cells in tumor sample.

FIG. 80B is a graph showing percentage of hCD137+ cells in CD3+ cells in tumor sample.

FIG. 80C is a graph showing percentage of hCD137+ cells in CD8+ cells in tumor sample.

FIG. 80D is a graph showing percentage of hCD137+ cells in CD4+FoxP3− cells in tumor sample.

FIG. 80E is a graph showing percentage of hCD137+ cells in Treg cells in tumor sample.

FIG. 81A is a graph showing counts of hCD137+ cells in NK cells in tumor sample.

FIG. 81B is a graph showing counts of hCD137+ cells in CD3+ cells in tumor sample.

FIG. 81C is a graph showing counts of hCD137+ cells in CD8+ cells in tumor sample.

FIG. 81D is a graph showing counts of hCD137+ cells in CD4+FoxP3− cells in tumor sample.

FIG. 81E is a graph showing counts of hCD137+ cells in Treg cells in tumor sample.

FIG. 82A is a graph showing percentage of CD8+/Treg cells in CD3+ cells in tumor sample.

FIG. 82B is a graph showing percentage of Ki67+ cells in CD8+ cells in tumor sample.

FIG. 82C is a graph showing percentage of Ki67+ cells in CD4+FoxP3− cells in tumor sample.

FIG. 82D is a graph showing percentage of Ki67+ cells in Treg cells in tumor sample.

FIG. 83A is a graph showing counts of Eomes+ cells in CD8+ cells in tumor sample.

FIG. 83B is a graph showing counts of Eomes+ cells in CD4+FoxP3+ cells in tumor sample.

FIG. 83C is a graph showing counts of T-bet+ cells in CD8+ cells in tumor sample.

FIG. 83D is a graph showing counts of T-bet+ cells in CD4+FoxP3− cells in tumor sample.

### STATEMENT REGARDING SEQUENCE LISTING

The Sequence Listing associated with this application is provided in text form in lieu of a paper copy, and is hereby incorporated by reference into the specification. The name of the text file containing the Sequence Listing is 45124-0021002_SL. The text file is 212,002 bytes, and was created and submitted electronically via EFS-Web on Feb. 22, 2022.

### DETAILED DESCRIPTION

The present disclosure provides examples of antibodies, antigen-binding fragments thereof, that bind to TNFRSF9 (tumor necrosis factor receptor superfamily member 9; also known as “4-1BB” or “CD137”).

### 4-1BB and Cancer

The immune system can differentiate between normal cells in the body and those it sees as “foreign,” which allows the immune system to attack the foreign cells while leaving the normal cells alone. This mechanism sometimes involves proteins called immune checkpoints. Immune checkpoints are molecules in the immune system that either turn up a signal (co-stimulatory molecules) or turn down a signal.

Checkpoint inhibitors can prevent the immune system from attacking normal tissue and thereby preventing autoimmune diseases. Many tumor cells also express checkpoint inhibitors. These tumor cells escape immune surveillance by co-opting certain immune-checkpoint pathways, particularly in T cells that are specific for tumor antigens (Creelan, Benjamin C. “Update on immune checkpoint inhibitors in lung cancer.” Cancer Control 21.1 (2014): 80-89). Because many immune checkpoints are initiated by ligand-receptor interactions, they can be readily blocked by antibodies against the ligands and/or their receptors.

4-1BB (“TNFRSF9” or “CD137”) is a member of the tumor necrosis factor (TNF) receptor family. It has three N-glycosylation sites and one potential O-glycosylation site. It is a type I transmembrane protein, and is mainly expressed on the surface of T cells, natural killer (NK) cells, neutrophils, and dendritic cells (DC) cells. The human CD137 gene is located on 1P36 region (chromosome 1) with NCBI gene ID 3604, and encodes 255 amino acids. The human CD137 protein molecule has two forms: membrane-bound and soluble, encoded by the 2.8 kb and 1.4 kb mRNAs, respectively. The soluble form does not have the transmembrane domain. The ligand for CD137 is CD137L (4-1BBL). CD137L belongs to the TNF superfamily and is expressed on the surface of antigen presenting cells, including e.g., dendritic cells, B cells, and macrophages. The synergistic stimulatory signal produced by the interaction of CD137 and its receptor CD137L induces activation and proliferation of T cells and NK cells, and the production of cytokines.

The mouse CD137 gene is located on 4E2 region (chromosome 4) with the NCBI gene ID 21942. Human CD137 protein is about 58% identical to mouse CD137 protein. Human CD137 contains notable differences in its cytoplasmic tail from mouse CD137. In particular, the single tyrosine residue in the cytoplasmic domain of CD137 is found at position 220 of human CD137 and at position 254 of mouse CD137. Human CD137 also diverges from mouse CD137 at the putative Lck binding site, with mouse CD137 expressing the CXCP Lck binding motif, whereas in human CD137 this sequence is altered to CXFP. Both human and mouse CD137 have in common two sites for binding TNFR-associated factor 2, an adaptor protein that is essential for mediating downstream signaling events leading to cytokine (e.g., IL-2) production in response to CD137L signaling.

The abnormal expression of CD137 and its ligand in tumor tissue indicates that CD137 and its ligand may have co-stimulatory signal disruption or inactivation during tumorigenesis. Particularly, CD137 expression in tumor vessel walls is correlated with tumor malignancy, and evidence shows that agonist anti-CD137 antibody can act on tumor endothelial cells to enhance recruitment of activated T lymphocytes, which suggests an additional mechanism of action that can explain the immunotherapeutic effects of agonist CD137 antibodies.

A large number of studies have shown that CD137 is one of the potential targets for antitumor biological therapy. Anti-CD137 antibody can kill tumor cells or inhibit tumor growth probably by inducing activation and proliferation of T cells and NK cells, increasing the production of cytokines, upregulating the immune response, and/or recruiting activated T lymphocytes to the tumor. To date, two antibodies to the CD137 pathway (Urelumab (BMS-663513) from Bristol-Myers Squibb and Utomilumab (PF-05082566) from Pfizer) have been tested in clinical trials for treating melanoma, lymphoma, non-Hodgkin lymphoma and some advanced solid tumor. Some clinical trials already have promising preliminary clinical results. For example, PF-05082566 can reduce 40% of follicular lymphomas (FL) with minimal side effects similar to PD-1 inhibitors; it has been used in combination with other drugs, including e.g., anti-PD-1 antibody, or anti-OX40 antibody. Preliminary experimental evidence also shows that cancer-targeting drugs can increase the expression of CD137 on the surface of NK cells. Thus, if anti-CD137 antibody is administered in combination with cancer-targeting drugs, it can enhance the killing effect of NK cells and improve the therapeutic effect.

A detailed description of CD137 and its function can be found, e.g., in Wen et al., “4-1BB ligand-mediated costimulation of human T cells induces CD4 and CD8 T cell expansion, cytokine production, and the development of cytolytic effector function,” The Journal of Immunology 168.10 (2002): 4897-4906; Broll et al., “CD137 expression in tumor vessel walls: high correlation with malignant tumors,” American journal of clinical pathology 115.4 (2001): 543-549; and Palazón et al., “Agonist anti-CD137 mAb act on tumor endothelial cells to enhance recruitment of activated T lymphocytes,” Cancer research 71.3 (2011): 801-811; Kang, et al., “Anti-CD137 suppresses tumor growth by blocking reverse signaling by CD137 ligand.” Cancer research (2017): canres-0610; each of which is incorporated by reference in its entirety.

The present disclosure provides anti-4-1BB antibodies, antigen-binding fragments thereof, and methods of using these anti-4-1BB antibodies and antigen-binding fragments to inhibit tumor growth and to treat cancers.

### Antibodies and Antigen Binding Fragments

The present disclosure provides anti-4-1BB antibodies and antigen-binding fragments thereof that comprise complementary determining regions (CDRs), heavy chain variable regions, light chain variable regions, heavy chains, or light chains described herein.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or subclasses including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. A more recent definition for CDRs is the IMGT definition. The IMGT definition is based on the IMGT database which curates nucleotide sequence information for immunoglobulins (IG), T-cell receptors (TcR) and Major Histocompatibility Complex (MHC) molecules. It proposes a uniform numbering system for IG and TcR sequences, based on aligning more than 5000 IG and TcR variable region sequences, taking into account and combining the Kabat definition of FRs and CDRs, structural data and Chothia's characterization of the hypervariable loops. The IMGT numbering scheme does not differentiate between various immunoglobulins (i.e., IG or TcR), the chain type (i.e., heavy or light) or the species. These methods for identifying the CDR regions and various definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); Lefranc, et al., “IMGT unique numbering for immunoglobulin and T cell receptor variable domains and Ig superfamily V-like domains.” Developmental & Comparative Immunology 27.1 (2003): 55-77; Kunik, et al., “Structural consensus among antibodies defines the antigen binding site.” PLoS computational biology 8.2 (2012): e1002388; each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgG4, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFV has one heavy chain variable domain, and one light chain variable domain. In some embodiments, the scFV has two heavy chain variable domains, and two light chain variable domains. In some embodiments, the scFV has two antigen binding regions, and the two antigen binding regions can bind to the respective target antigens.

### Anti-4-1BB Antibodies and Antigen-Binding Fragments

The disclosure provides antibodies and antigen-binding fragments thereof that specifically bind to 4-1BB. The antibodies and antigen-binding fragments described herein are capable of binding to 4-1BB and can promote 4-1BB signaling pathway thus increase immune response.

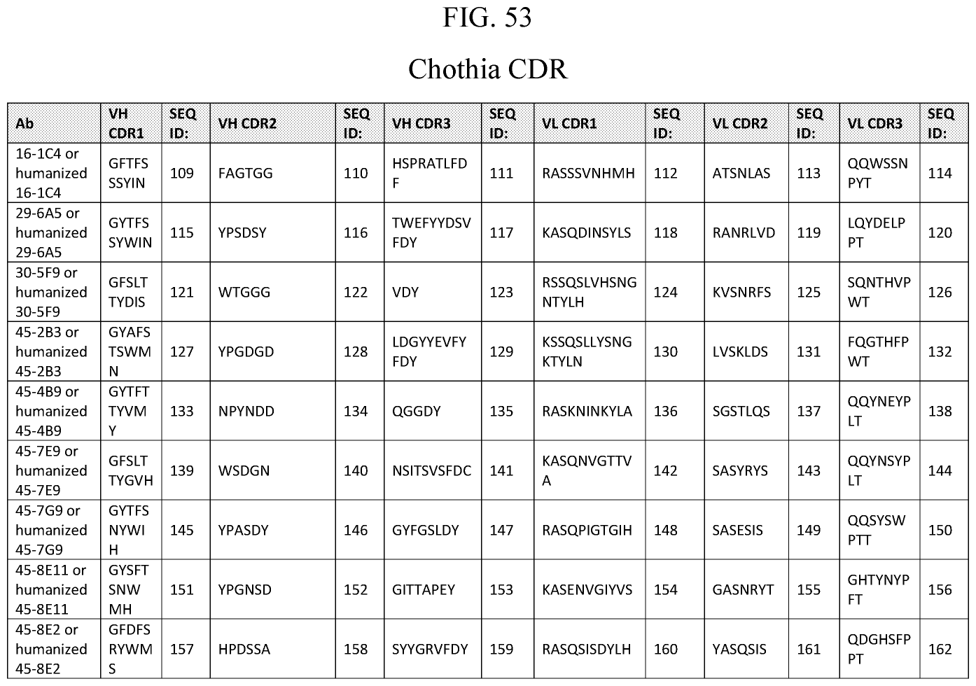

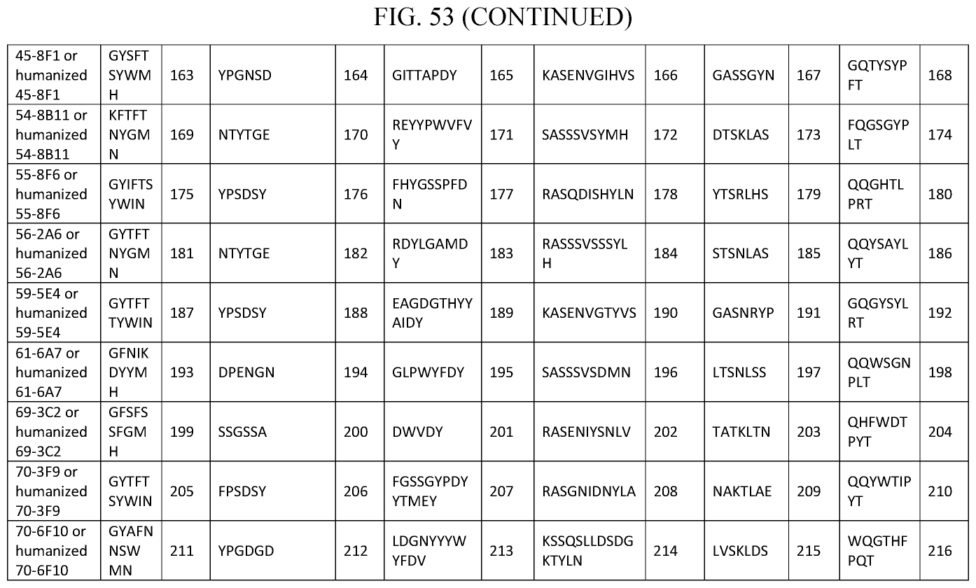

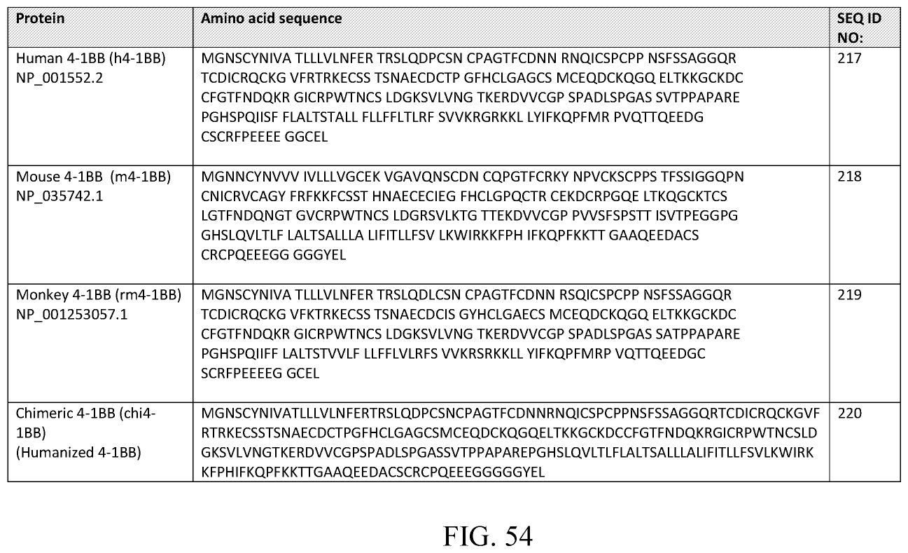

The disclosure provides several anti-4-1BB antibodies, e.g., 16-1C4 (“1C4”), 29-6A5 (“6A5”), 30-5F9 (“5F9”), 45-2B3 (“2B3”), 45-4B9 (“4B9”), 45-7E9 (“7E9”), 45-7G9 (“7G9”), 45-8E11 (“8E11”), 45-8E2 (“8E2”), 45-8F1 (“8F1”), 54-8B11 (“8B11”), 55-8F6 (“8F6”), 56-2A6 (“2A6”), 59-5E4 (“5E4”), 61-6A7 (“6A7”), 69-3C2 (“3C2”), 70-3F9 (“3F9”), and 70-6F10 (“6F10”), including e.g., mouse antibodies, chimeric antibodies thereof, and the humanized antibodies thereof. The CDR sequences (Kabat definition) and the sequences of the heavy chain variable region and light chain variable region of some of the disclosed antibodies are shown in the table below. The CDR sequences (Chothia definition) are provided in FIG. 53.

TABLE 1

| | VH | VH | VH | VL | VL | VL | | |
|---|---|---|---|---|---|---|---|---|
| Ab | CDR1 | CDR2 | CDR3 | CDR1 | CDR2 | CDR3 | VH | VL |
| 16-1C4 | 1 | 2 | 3 | 4 | 5 | 6 | 243 | 244 |
| 29-6A5 | 7 | 8 | 9 | 10 | 11 | 12 | 245 | 246 |
| 30-5F9 | 13 | 14 | 15 | 16 | 17 | 18 | 247 | 248 |
| 45-2B3 | 19 | 20 | 21 | 22 | 23 | 24 | 249 | 250 |
| 45-4B9 | 25 | 26 | 27 | 28 | 29 | 30 | 251 | 252 |
| 45-7E9 | 31 | 32 | 33 | 34 | 35 | 36 | 253 | 254 |
| 45-7G9 | 37 | 38 | 39 | 40 | 41 | 42 | 255 | 256 |
| 45-8E11 | 43 | 44 | 45 | 46 | 47 | 48 | 257 | 258 |
| 45-8E2 | 49 | 50 | 51 | 52 | 53 | 54 | 259 | 260 |
| 45-8F1 | 55 | 56 | 57 | 58 | 59 | 60 | 261 | 262 |
| 54-8B11 | 61 | 62 | 63 | 64 | 65 | 66 | 263 | 264 |
| 55-8F6 | 67 | 68 | 69 | 70 | 71 | 72 | 265 | 266 |
| 56-2A6 | 73 | 74 | 75 | 76 | 77 | 78 | 267 | 268 |
| 59-5E4 | 79 | 80 | 81 | 82 | 83 | 84 | 269 | 270 |
| 61-6A7 | 85 | 86 | 87 | 88 | 89 | 90 | 271 | 272 |
| 69-3C2 | 91 | 92 | 93 | 94 | 95 | 96 | 273 | 274 |
| 70-3F9 | 97 | 98 | 99 | 100 | 101 | 102 | 275 | 276 |
| 70-6F10 | 103 | 104 | 105 | 106 | 107 | 108 | 277 | 278 |

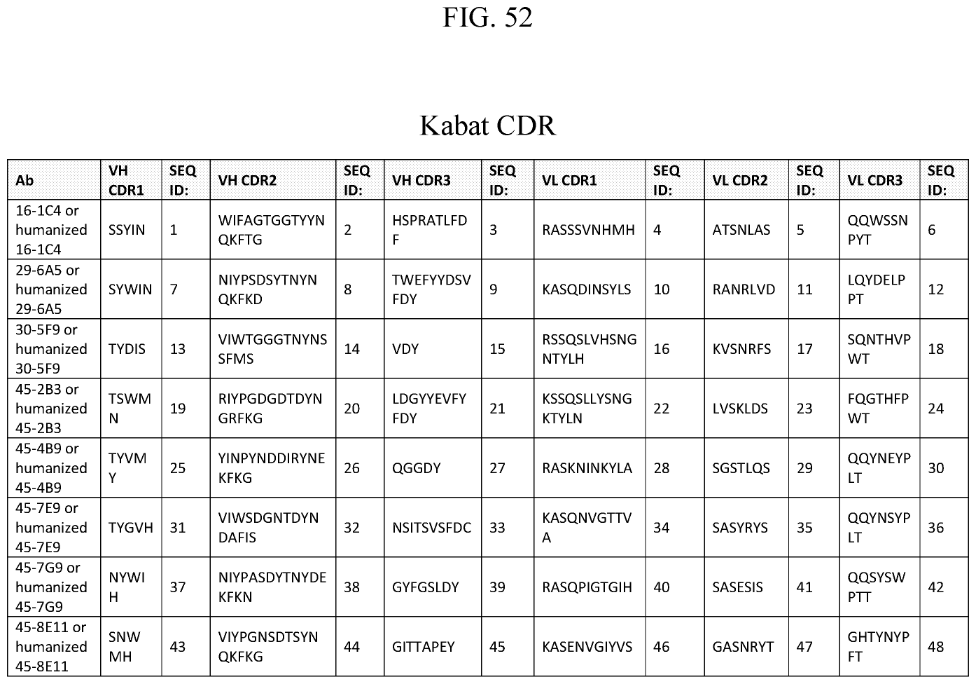

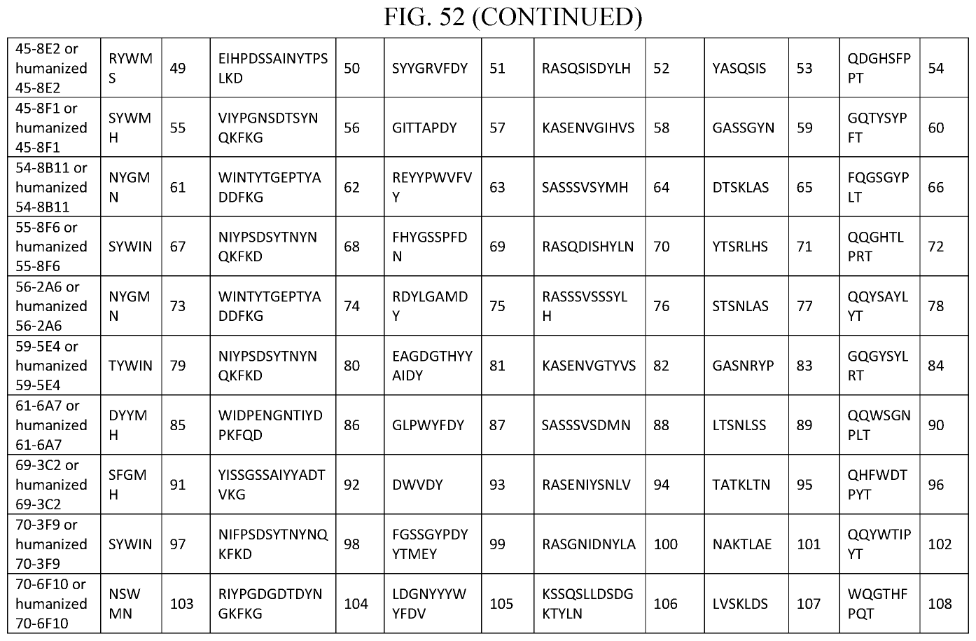

For example, as shown in the table above, FIG. 52, and FIG. 53, the CDR sequences for 1C4, and 1C4 derived antibodies (e.g., chimeric antibodies or humanized antibodies) include CDRs of the heavy chain variable domain, SEQ ID NOs: 1-3, and CDRs of the light chain variable domain, SEQ ID NOs: 4-6 as defined by Kabat numbering. The CDRs can also be defined by Chothia system. Under the Chothia numbering, the CDR sequences of the heavy chain variable domain are set forth in SEQ ID NOs: 109-111 and CDR sequences of the light chain variable domain are set forth in SEQ ID NOs: 112-114.

Similarly, the CDR sequences for 6A5, and 6A5 derived antibodies include CDRs of the heavy chain variable domain, SEQ ID NOs: 7-9, and CDRs of the light chain variable domain, SEQ ID NOs: 10-12, as defined by Kabat numbering. Under Chothia numbering, the CDR sequences of the heavy chain variable domain are set forth in SEQ ID NOs: 115-117, and CDRs of the light chain variable domain are set forth in SEQ ID NOs: 118-120.

The CDR sequences for 5F9, and 5F9 derived antibodies include CDRs of the heavy chain variable domain, SEQ ID NOs: 13-15, and CDRs of the light chain variable domain, SEQ ID NOs: 16-18, as defined by Kabat numbering. Under Chothia numbering, the CDR sequences of the heavy chain variable domain are set forth in SEQ ID NOs: 121-123, and CDRs of the light chain variable domain are set forth in SEQ ID NOs: 124-126.

Similarly, the CDR sequences (Kabat) and the VH and VL sequences for 2B3, 4B9, 7E9, 7G9, 8E11, 8E2, 8F1, 8B11, 8F6, 2A6, 5E4, 6A7, 3C2, 3F9, 6F10, and the antibodies derived from these antibodies are provided in Table 1. The CDR sequence (Chothia) for 2B3, 4B9, 7E9, 7G9, 8E11, 8E2, 8F1, 8B11, 8F6, 2A6, 5E4, 6A7, 3C2, 3F9, 6F10, and the antibodies derived from these antibodies can be found in FIG. 53.

The amino acid sequences for heavy chain variable regions and light variable regions of the humanized antibodies are also provided. As there are different ways to humanize a mouse antibody (e.g., a sequence can be modified with different amino acid substitutions), the heavy chain and the light chain of an antibody can have more than one version of humanized sequences. The amino acid sequences for the heavy chain variable regions of humanized 1C4 antibody are set forth in SEQ ID NOs: 221-224. The amino acid sequences for the light chain variable regions of humanized 1C4 antibody are set forth in SEQ ID NOs: 225-228. Any of these heavy chain variable region sequences (SEQ ID NO: 221-224) can be paired with any of these light chain variable region sequences (SEQ ID NO: 225-228).

Similarly, the amino acid sequences for the heavy chain variable region of humanized 6A5 antibody are set forth in SEQ ID NOs: 229-231. The amino acid sequences for the light chain variable region of humanized 6A5 antibody are set forth in SEQ ID NOs: 232-235. Any of these heavy chain variable region sequences (SEQ ID NO: 229-231) can be paired with any of these light chain variable region sequences (SEQ ID NO: 232-235).

The amino acid sequences for the heavy chain variable region of humanized 5F9 antibody are set forth in SEQ ID NOs: 236-238. The amino acid sequences for the light chain variable region of humanized 6A5 antibody are set forth in SEQ ID NOs: 239-242. Any of these heavy chain variable region sequences (SEQ ID NO: 236-238) can be paired with any of these light chain variable region sequences (SEQ ID NO: 239-242).

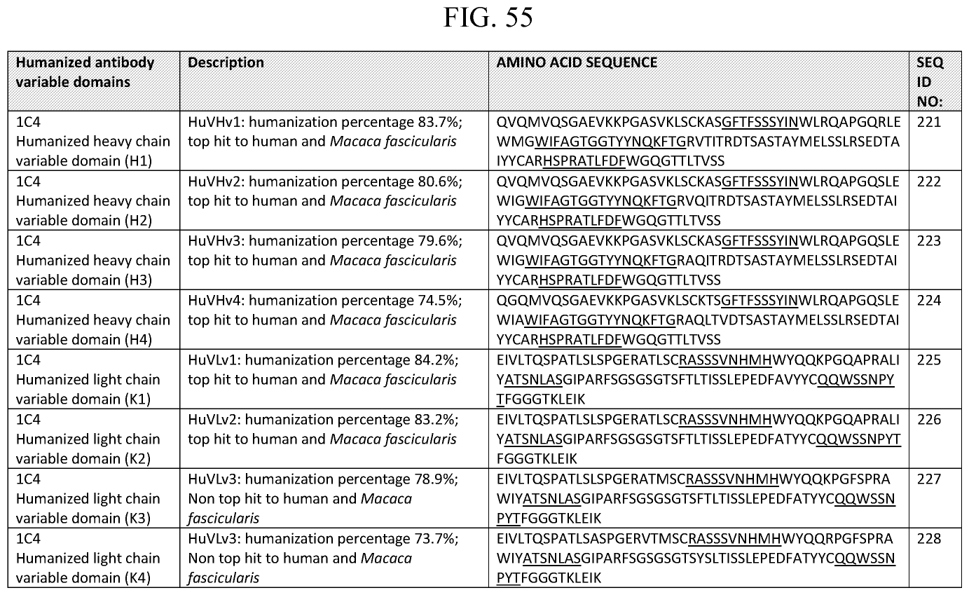

As shown in FIGS. 55-57, humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, Tim D., et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

The present disclosure also provides chimeric antibodies. These chimeric antibodies have VH and VL from mouse antibodies. However, the constant domains of these chimeric antibodies are from human antibodies (e.g., human IgG1, human IgG2, human IgG3, or human IgG4). These chimeric antibodies are labeled as mHvKv-IgG.

Some of the chimeric antibodies and humanized antibodies that are described in the present disclosure are shown in the table below.

TABLE 2

| | | VH | VL | |
|---|---|---|---|---|
| | Antibody | SEQ ID | SEQ ID | |
| Type | name | NO: | NO: | Constant regions |
| Chimeric | 1C4- | 243 | 244 | Human IgG (e.g., IgG1, |
| antibody | mHyKy- | | | IgG2, IgG3, or IgG4) |
| based on | IgG | | | |
| 1C4 | | | | |
| Humanized | 1C4-H1K1- | 221 | 225 | Human IgG (e.g., IgG1, |
| antibodies | IgG | | | IgG2, IgG3, or IgG4) |
| based on | 1C4-H1K2- | 221 | 226 | Human IgG (e.g., IgG1, |
| 1C4 | IgG | | | IgG2, IgG3, or IgG4) |
| | 1C4-H1K3- | 221 | 227 | Human IgG (e.g., IgG1, |
| | IgG | | | IgG2, IgG3, or IgG4) |
| | 1C4-H1K4- | 221 | 228 | Human IgG (e.g., IgG1, |
| | IgG | | | IgG2, IgG3, or IgG4) |
| | 1C4-H2K1- | 222 | 225 | Human IgG (e.g., IgG1, |
| | IgG | | | IgG2, IgG3, or IgG4) |
| | 1C4-H2K2- | 222 | 226 | Human IgG (e.g., IgG1, |
| | IgG | | | IgG2, IgG3, or IgG4) |
| | 1C4-H2K3- | 222 | 227 | Human IgG (e.g., IgG1, |
| | IgG | | | IgG2, IgG3, or IgG4) |
| | 1C4-H2K4- | 222 | 228 | Human IgG (e.g., IgG1, |
| | IgG | | | IgG2, IgG3, or IgG4) |
| | 1C4-H3K1- | 223 | 225 | Human IgG (e.g., IgG1, |
| | IgG | | | IgG2, IgG3, or IgG4) |
| | 1C4-H3K2- | 223 | 226 | Human IgG (e.g., IgG1, |
| | IgG | | | IgG2, IgG3, or IgG4) |
| | 1C4-H3K3- | 223 | 227 | Human IgG (e.g., IgG1, |
| | IgG | | | IgG2, IgG3, or IgG4) |
| | 1C4-H3K4- | 223 | 228 | Human IgG (e.g., IgG1, |
| | IgG | | | IgG2, IgG3, or IgG4) |
| | 1C4-H4K1- | 224 | 225 | Human IgG (e.g., IgG1, |
| | IgG | | | IgG2, IgG3, or IgG4) |
| | 1C4-H4K2- | 224 | 226 | Human IgG (e.g., IgG1, |
| | IgG | | | IgG2, IgG3, or IgG4) |
| | 1C4-H4K3- | 224 | 227 | Human IgG (e.g., IgG1, |
| | IgG | | | IgG2, IgG3, or IgG4) |
| | 1C4-H4K4- | 224 | 228 | Human IgG (e.g., IgG1, |
| | IgG | | | IgG2, IgG3, or IgG4) |
| Chimeric | 6A5- | 245 | 246 | Human IgG (e.g., IgG1, |
| antibody | mHvKv- | | | IgG2, IgG3, or IgG4) |
| based on | IgG | | | |
| 6A5 | | | | |
| Humanized | 6A5-H1K1- | 229 | 232 | Human IgG (e.g., IgG1, |
| antibodies | IgG | | | IgG2, IgG3, or IgG4) |
| based on | 6A5-H1K2- | 229 | 233 | Human IgG (e.g., IgG1, |
| 6A5 | IgG | | | IgG2, IgG3, or IgG4) |
| | 6A5-H1K3- | 229 | 234 | Human IgG (e.g., IgG1, |
| | IgG | | | IgG2, IgG3, or IgG4) |
| | 6A5-H1K4- | 229 | 235 | Human IgG (e.g., IgG1, |
| | IgG | | | IgG2, IgG3, or IgG4) |
| | 6A5-H2K1- | 230 | 232 | Human IgG (e.g., IgG1, |
| | IgG | | | IgG2, IgG3, or IgG4) |
| | 6A5-H2K2- | 230 | 233 | Human IgG (e.g., IgG1, |
| | IgG | | | IgG2, IgG3, or IgG4) |
| | 6A5-H2K3- | 230 | 234 | Human IgG (e.g., IgG1, |
| | IgG | | | IgG2, IgG3, or IgG4) |
| | 6A5-H2K4- | 230 | 235 | Human IgG (e.g., IgG1, |
| | IgG | | | IgG2, IgG3, or IgG4) |
| | 6A5-H3K1- | 231 | 232 | Human IgG (e.g., IgG1, |
| | IgG | | | IgG2, IgG3, or IgG4) |
| | 6A5-H3K2- | 231 | 233 | Human IgG (e.g., IgG1, |
| | IgG | | | IgG2, IgG3, or IgG4) |
| | 6A5-H3K3- | 231 | 234 | Human IgG (e.g., IgG1, |
| | IgG | | | IgG2, IgG3, or IgG4) |
| | 6A5-H3K4- | 231 | 235 | Human IgG (e.g., IgG1, |
| | IgG | | | IgG2, IgG3, or IgG4) |
| Chimeric | 5F9- | 247 | 248 | Human IgG (e.g., IgG1, |
| antibody | mHyKy- | | | IgG2, IgG3, or IgG4) |
| based on | IgG | | | |
| 5F9 | | | | |
| Humanized | 5F9-H1K1- | 236 | 239 | Human IgG (e.g., IgG1, |
| antibodies | IgG | | | IgG2, IgG3, or IgG4) |
| based on | 5F9-H1K2- | 236 | 240 | Human IgG (e.g., IgG1, |
| 5F9 | IgG | | | IgG2, IgG3, or IgG4) |
| | 5F9-H1K3- | 236 | 241 | Human IgG (e.g., IgG1, |
| | IgG | | | IgG2, IgG3, or IgG4) |
| | 5F9-H1K4- | 236 | 242 | Human IgG (e.g., IgG1, |
| | IgG | | | IgG2, IgG3, or IgG4) |
| | 5F9-H2K1- | 237 | 239 | Human IgG (e.g., IgG1, |
| | IgG | | | IgG2, IgG3, or IgG4) |
| | 5F9-H2K2- | 237 | 240 | Human IgG (e.g., IgG1, |
| | IgG | | | IgG2, IgG3, or IgG4) |
| | 5F9-H2K3- | 237 | 241 | Human IgG (e.g., IgG1, |
| | IgG | | | IgG2, IgG3, or IgG4) |
| | 5F9-H2K4- | 237 | 242 | Human IgG (e.g., IgG1, |
| | IgG | | | IgG2, IgG3, or IgG4) |
| | 5F9-H3K1- | 238 | 239 | Human IgG (e.g., IgG1, |
| | IgG | | | IgG2, IgG3, or IgG4) |
| | 5F9-H3K2- | 238 | 240 | Human IgG (e.g., IgG1, |
| | IgG | | | IgG2, IgG3, or IgG4) |
| | 5F9-H3K3- | 238 | 241 | Human IgG (e.g., IgG1, |
| | IgG | | | IgG2, IgG3, or IgG4) |
| | 5F9-H3K4- | 238 | 242 | Human IgG (e.g., IgG1, |
| | IgG | | | IgG2, IgG3, or IgG4) |

Furthermore, in some embodiments, the antibodies or antigen-binding fragments thereof described herein can also contain one, two, or three heavy chain variable region CDRs selected from the group of SEQ ID NOs: 1-3, SEQ ID NOs: 7-9, SEQ ID NOs: 13-15, SEQ ID NOs: 19-21, SEQ ID NOs: 25-27, SEQ ID NOs: 31-33, SEQ ID NOs: 37-39, SEQ ID NOs: 43-45, SEQ ID NOs: 49-51, SEQ ID NOs: 55-57, SEQ ID NOs: 61-63, SEQ ID NOs: 67-69, SEQ ID NOs: 73-75, SEQ ID NOs: 79-81, SEQ ID NOs: 85-87, SEQ ID NOs: 91-93, SEQ ID NOs: 97-99, or SEQ ID NOs: 103-105; and/or one, two, or three light chain variable region CDRs selected from the group of SEQ ID NOs: 4-6, SEQ ID NOs: 10-12, SEQ ID NOs: 16-18, SEQ ID NOs: 22-24, SEQ ID NOs: 28-30, SEQ ID NOs: 34-36, SEQ ID NOs: 40-42, SEQ ID NOs: 46-48, SEQ ID NOs: 52-54, SEQ ID NOs: 58-60, SEQ ID NOs: 64-66, SEQ ID NOs: 70-72, SEQ ID NOs: 76-78, SEQ ID NOs: 82-84, SEQ ID NOs: 88-90, SEQ ID NOs: 94-96, SEQ ID NOs: 100-102, or SEQ ID NOs: 106-108.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 52 (Kabat CDR) and FIG. 53 (Chothia CDR).

In some embodiments, the antibody or an antigen-binding fragment described herein can contain a heavy chain variable domain containing one, two, or three of the CDRs selected from SEQ ID NOs: 1-3, SEQ ID NOs: 7-9, SEQ ID NOs: 13-15, SEQ ID NOs: 19-21, SEQ ID NOs: 25-27, SEQ ID NOs: 31-33, SEQ ID NOs: 37-39, SEQ ID NOs: 43-45, SEQ ID NOs: 49-51, SEQ ID NOs: 55-57, SEQ ID NOs: 61-63, SEQ ID NOs: 67-69, SEQ ID NOs: 73-75, SEQ ID NOs: 79-81, SEQ ID NOs: 85-87, SEQ ID NOs: 91-93, SEQ ID NOs: 97-99, or SEQ ID NOs: 103-105 with zero, one or two amino acid insertions, deletions, or substitutions on one, two, or three of the selected CDRs. For example, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a light chain variable domain containing one, two, or three of the CDRs selected from SEQ ID NOs: 4-6, SEQ ID NOs: 10-12, SEQ ID NOs: 16-18, SEQ ID NOs: 22-24, SEQ ID NOs: 28-30, SEQ ID NOs: 34-36, SEQ ID NOs: 40-42, SEQ ID NOs: 46-48, SEQ ID NOs: 52-54, SEQ ID NOs: 58-60, SEQ ID NOs: 64-66, SEQ ID NOs: 70-72, SEQ ID NOs: 76-78, SEQ ID NOs: 82-84, SEQ ID NOs: 88-90, SEQ ID NOs: 94-96, SEQ ID NOs: 100-102, or SEQ ID NOs: 106-108 with zero, one or two amino acid insertions, deletions, or substitutions on one, two, or three of the selected CDRs. For example, the antibody or an antigen-binding fragment described herein can contain a light chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the CDRs with zero, one or two amino acid insertions, deletions, or substitutions are shown in Table 1, Table 3, FIG. 52, and FIG. 53.

The insertions, deletions, and substitutions can be within the CDR sequence, or at one or both terminal ends of the CDR sequence.

The disclosure also provides antibodies or antigen-binding fragments thereof that bind to 4-1BB, wherein the antibodies or antigen-binding fragments have VH CDR1, VH CDR2, VH CDR3, VL CDR1, VL CDR2, and VL CDR3. In some embodiments, the sequences for VH CDR1, VH CDR2, VH CDR3, VL CDR1, VL CDR2, and VL CDR3 are determined based on various CDR definitions known in the art, e.g., the Kabat definition, the Chothia definition, or the IMGT definition. The sequences for VH CDR1, VH CDR2, VH CDR3, VL CDR1, VL CDR2, and VL CDR3 are set forth in Table 1, Table 3, FIG. 52, and FIG. 53.

The disclosure also provides antibodies or antigen-binding fragments thereof that bind to 4-1BB. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence and the selected VL sequence is in Table 1 and Table 3. In some embodiments, the selected VH sequence is SEQ ID NO: 221, 222, 223, 224, or 243, and the selected VL sequence is SEQ ID NO: 225, 226, 227, 228, or 244. In some embodiments, the selected VH sequence is SEQ ID NO: 229, 230, 231, or 245 and the selected VL sequence is SEQ ID NO: 232, 233, 234, 235, or 246. In some embodiments, the selected VH sequence is SEQ ID NO: 236, 237, 238, or 247, and the selected VL sequence is SEQ ID NO: 239, 240, 241, 242, or 248.

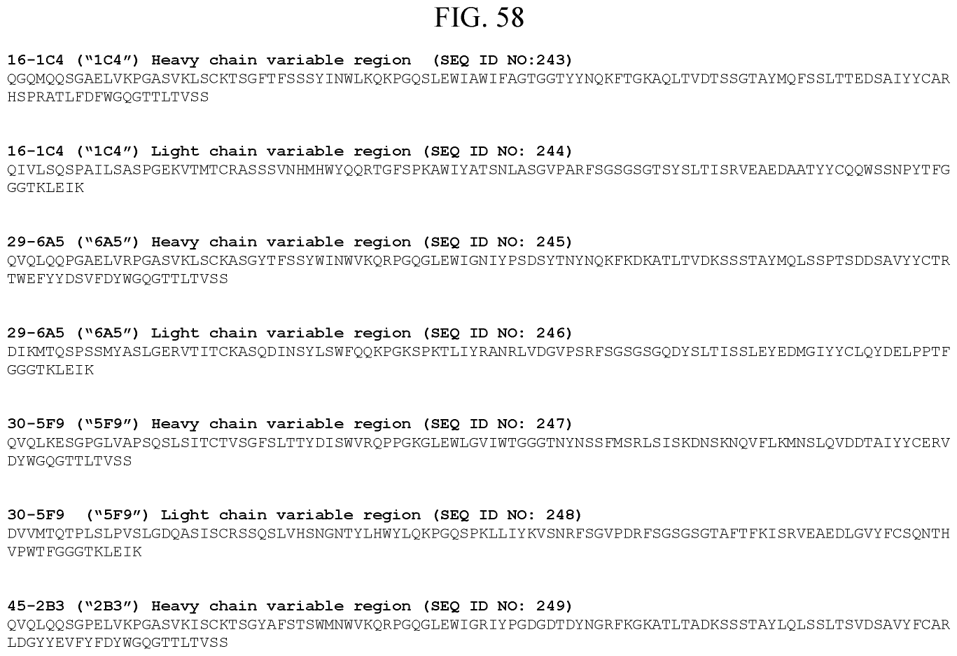

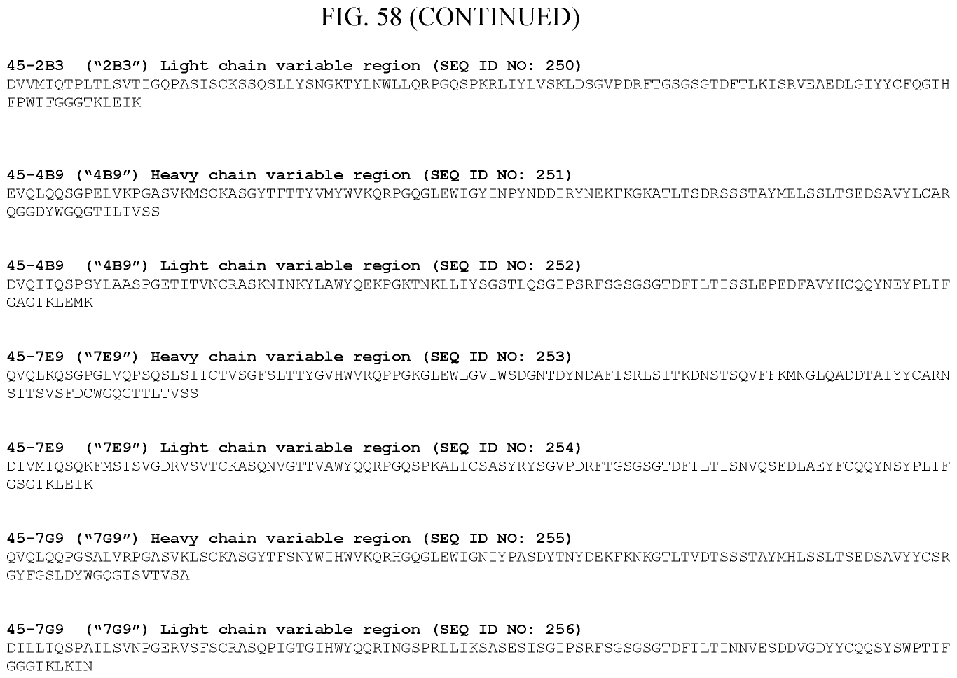

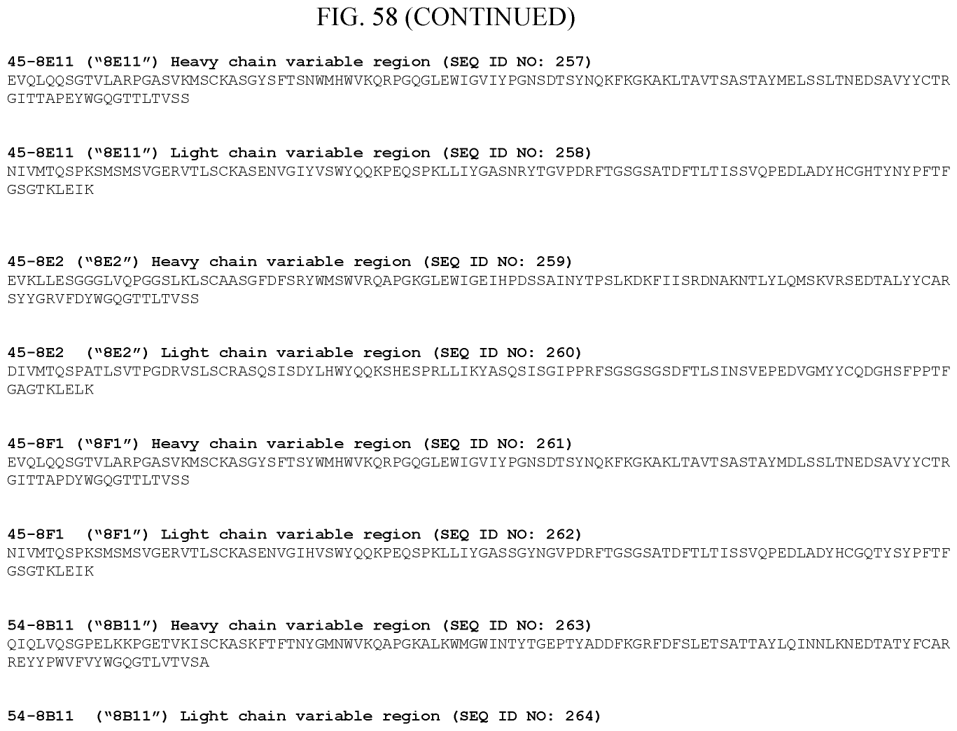

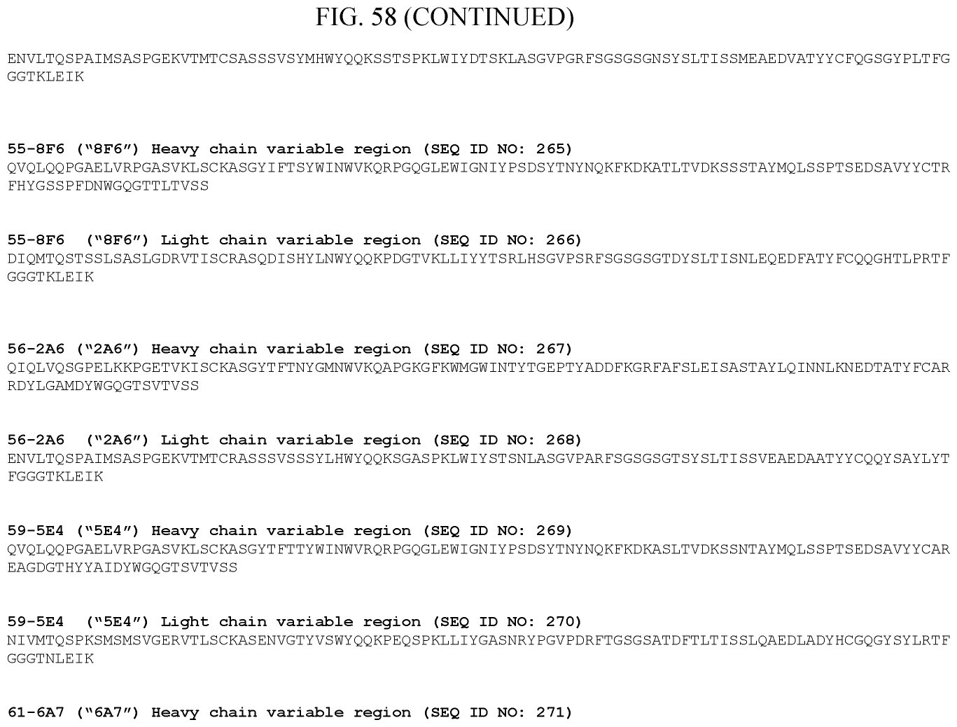

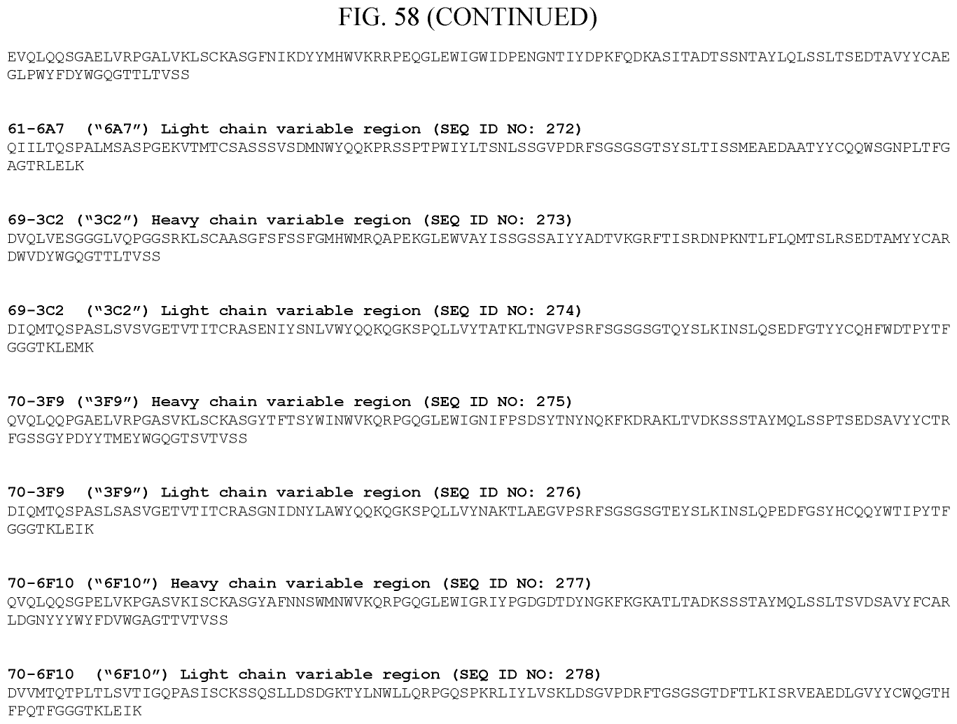

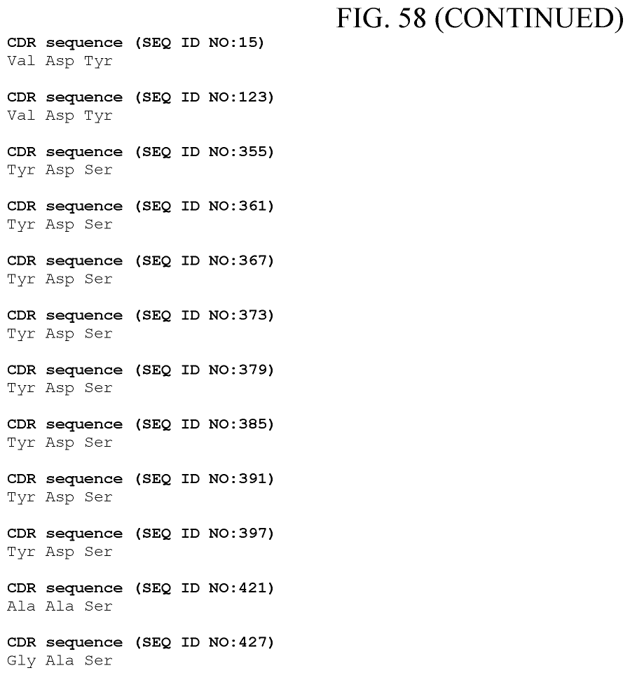

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in Table 1, Table 3, FIG. 52 or FIG. 53, or have sequences as shown in Table 1, Table 3, and FIGS. 55-58. When the polypeptides are paired with corresponding polypeptide (e.g., a corresponding heavy chain variable region or a corresponding light chain variable region), the paired polypeptides bind to 4-1BB (e.g., human 4-1BB).

In some aspects, the disclosure also provides an antibody or an antigen-binding fragment thereof, which cross-competes for binding to 4-1BB (e.g., human 4-1BB) with a reference antibody or antigen-binding fragment thereof (e.g., any anti-4-1BB antibody or antigen-binding fragment as described herein).

The anti-4-1BB antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), or of any subclass (e.g., IgG1, IgG2, IgG3, IgG4, IgA1, and IgA2). In some embodiments, the antibody or antigen-binding fragment thereof is an IgG antibody or antigen-binding fragment thereof. In some embodiments, the antibody or antigen-binding fragment thereof is an IgG1 antibody or antigen-binding fragment thereof.

Fragments of antibodies are suitable for use in the methods provided so long as they retain the desired affinity and specificity of the full-length antibody. Thus, a fragment of an antibody that binds to 4-1BB will retain an ability to bind to 4-1BB. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers can be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′ 2 homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Antibody Characteristics

In some embodiments, the antibodies or antigen-binding fragments thereof described herein can block the binding between 4-1BB and the 4-1BB ligand (4-1BBL).

In some embodiments, by binding to 4-1BB, the antibody can upregulates the immune response. Thus, in some embodiments, the antibodies or antigen-binding fragments thereof as described herein are 4-1BB agonist. In some embodiments, the antibodies or antigen-binding fragments thereof are 4-1BB antagonist.

In some embodiments, the antibodies or antigen-binding fragments thereof as described herein can increase immune response, activity or number of immune cells (e.g., T cells, natural killer (NK) cells, neutrophils, dendritic cells (DC) cells, macrophages, antigen-presenting cells, CD8+ and/or CD4+ T cells) by at least 10%, 20%, 30%, 40%, 50%, 60%, 70%, 80%, 90%, 100%, 2 folds, 3 folds, 5 folds, 10 folds, or 20 folds. In some embodiments, the antibodies or antigen-binding fragments thereof as described herein can decrease the activity or number of immune cells by at least 10%, 20%, 30%, 40%, 50%, 60%, 70%, 80%, 90%, 100%, 2 folds, 3 folds, 5 folds, 10 folds, or 20 folds.

In some implementations, the antibody (or antigen-binding fragments thereof) specifically binds to 4-1BB (e.g., human 4-1BB, monkey 4-1BB, mouse 4-1BB, and/or chimeric 4-1BB) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. In some embodiments, the antibody binds to human 4-1BB with KD less than or equal to about 0.9 nM, 0.8 nM, 0.7 nM, 0.6 nM, 0.5 nM, 0.4 nM, 0.3 nM, 0.2 nM, or 0.1 nM. In some embodiments, the antibody binds to human 4-1BB with KD less than or equal to about 0.5 nM or 0.4 nM.

General techniques for measuring the affinity of an antibody for an antigen include, e.g., ELISA, RIA, and surface plasmon resonance (SPR). In some embodiments, the antibody binds to human 4-1BB (SEQ ID NO: 217), monkey 4-1BB (e.g., Rhesus macaque 4-1BB, SEQ ID NO: 219), chimeric 4-1BB (SEQ ID NO: 220), and/or mouse 4-1BB (SEQ ID NO: 218). In some embodiments, the antibody does not bind to human 4-1BB (SEQ ID NO: 217), monkey 4-1BB (e.g., Rhesus macaque 4-1BB, SEQ ID NO: 219; or cynomolgus 4-1BB), chimeric 4-1BB (SEQ ID NO: 220), and/or mouse 4-1BB (SEQ ID NO: 218).

In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

As IgG can be described as a multi-domain protein, the melting curve sometimes shows two transitions, or three transitions, with a first denaturation temperature, Tm D1, and a second denaturation temperature Tm D2, and optionally a third denaturation temperature Tm D3. When there are two peaks, one peak indicates the denaturation of the Fc domains (Tm D1), and the other peak indicates the denaturation of the Fab domains (Tm D2).

In some embodiments, the antibodies or antigen binding fragments as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D3 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, Tm, Tm D1, Tm D2, Tm D3 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies or antigen binding fragments as described herein do not form aggregation when the temperate is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen-binding fragments thereof as described herein are 4-1BB agonist. In some embodiments, the antibodies or antigen-binding fragments thereof as described herein are 4-1BB antagonist. In some embodiments, the antibodies or antigen binding fragments can increase or decrease 4-1BB signal transduction in a target cell that expresses 4-1BB.

In some embodiments, the antibodies or antigen binding fragments can enhance immune cell function (e.g., CD8+ T cells, CD4+ T cells, B cells, natural killer cells, neutrophils, monocytes, macrophages, and/or dendritic cells), for example, by increasing immune cell proliferation and/or increasing cytokine production by the immune cell (e.g., as compared to proliferation and/or cytokine production prior to treatment with the antibodies or antigen binding fragments). In some embodiments, the cytokine is gamma interferon.

In some embodiments, the antibodies or antigen binding fragments increase number of intratumoral (infiltrating) CD8+ effector T cells (e.g., total number of CD8+ effector T cells, or e.g., percentage of CD8+ in CD45+ cells), e.g., as compared to number of intratumoral (infiltrating) CD8+T effector cells prior to treatment. In some embodiments, the antibodies or antigen binding fragments increase number of intratumoral (infiltrating) CD8+ effector T cells that express gamma interferon (e.g., percentage of CD8+ cells that express gamma interferon in total CD8+ cells), e.g., compared to number of intratumoral (infiltrating) CD8+ T cells that express gamma interferon prior to treatment with anti-human 4-1BB antibody.

In some embodiments, the antibodies or antigen binding fragments increase number of intratumoral (infiltrating) CD4+ effector T cells (e.g., total number of CD4+ effector T cells, or e.g., percentage of CD4+ cells in CD45+ cells), e.g., as compared to number of intratumoral (infiltrating) CD4+ T cells prior to treatment with antibodies or antigen binding fragments. In some embodiments, the antibodies or antigen binding fragments increase number of intratumoral (infiltrating) CD4+ effector T cells that express gamma interferon (e.g., total gamma interferon expressing CD4+ cells, or e.g., percentage of gamma interferon expressing CD4+ cells in total CD4+ cells), e.g., as compared to number of intratumoral (infiltrating) CD4+ T cells that express gamma interferon prior to treatment.

In some embodiments, the antibodies or antigen binding fragments enhance memory T cell function, for example by increasing memory T cell proliferation and/or increasing cytokine (e.g., gamma interferon) production by the memory T cell.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibodies or antigen binding fragments do not have a functional Fc region. For example, the antibodies or antigen binding fragments are Fab, Fab′, F(ab′)2, and Fv fragments.

### Anti-h4-1BB IgG1 Antibody

The present disclosure also shows that IgG1 subclass antibodies unexpectedly have much better tumor inhibitory effects than other subclasses. Based on the results and without wishing to be bound by theory, it has been hypothesized that the anti-h1BB antibody inhibits tumor growth primarily through antibody-dependent cell-mediated cytotoxicity (ADCC). FcγRIIIa is the major receptor involved in the activation of ADCC. IgG subclasses vary in their ability to bind to FcγRIIIa and this differential binding determines their ability to elicit a range of functional responses (e.g., ADCC). Thus, it may be due to the fact that IgG1 subclass may have a stronger binding affinity with FcγRIIIa, IgG1 antibodies have better tumor inhibitory effects than other subclasses. Therefore, in some embodiments, the present disclosure provides an IgG1 antibody (e.g., human IgG1 antibody) that has CDRs, VH, and VL as described herein.

In some embodiments, the IgG1 antibody or the antigen binding fragment thereof has CDRs (e.g., in Kabat definition, Chothia definition, or IMGT definition), VH and VL as shown in the table below. Thus, the present disclosure provides an IgG1 antibody (e.g., human IgG1 antibody or humanized IgG1 antibody) comprising a heavy chain variable region (VH) comprising complementarity determining regions (CDRs) 1, 2, and 3, wherein the VH CDR1 region comprises an amino acid sequence that is at least 80%, 85%, 90%, 95%, or 100% identical to a selected VH CDR1 amino acid sequence, the VH CDR2 region comprises an amino acid sequence that is at least 80%, 85%, 90%, 95%, or 100% identical to a selected VH CDR2 amino acid sequence, and the VH CDR3 region comprises an amino acid sequence that is at least 80%, 85%, 90%, 95%, or 100% identical to a selected VH CDR3 amino acid sequence; and a light chain variable region (VL) comprising CDRs 1, 2, and 3, wherein the VL CDR1 region comprises an amino acid sequence that is at least 80%, 85%, 90%, 95%, or 100% identical to a selected VL CDR1 amino acid sequence, the VL CDR2 region comprises an amino acid sequence that is at least 80%, 85%, 90%, 95%, or 100% identical to a selected VL CDR2 amino acid sequence, and the VL CDR3 region comprises an amino acid sequence that is at least 80%, 85%, 90%, 95%, or 100% identical to a selected VL CDR3 amino acid sequence, wherein the VH CDR1, VH CDR2, VH CDR3, VL CDR1, VL CDR2, VL CDR3 are set forth in Table 3. In some embodiments, the CDRs of the IgG1 antibody or the antigen binding fragment thereof are defined by Kabat definition, Chothia definition, or IMGT definition. In some embodiments, the VH CDR1, VH CDR2, VH CDR3, VL CDR1, VL CDR2, VL CDR3 (CDRs as defined by Kabat definition) of the IgG1 antibody or the antigen binding fragment thereof have sequences that are set forth in Table 3. In some embodiments, the VH CDR1, VH CDR2, VH CDR3, VL CDR1, VL CDR2, VL CDR3 (CDRs as defined by IMGT definition) of the IgG1 antibody or the antigen binding fragment thereof have sequences that are set forth in Table 3.

In some embodiments, the present disclosure provides an antibody or antigen-binding fragment thereof that binds to 4-1BB comprising a heavy chain variable region (VH) comprising an amino acid sequence that is at least 80%, 85%, 90%, 95%, or 100% identical to a selected VH sequence, and a light chain variable region (VL) comprising an amino acid sequence that is at least 80%, 85%, 90%, 95%, or 100% identical to a selected VL sequence, wherein the selected VH sequence and the selected VL sequence are set forth in the table below.

TABLE 3

| | VH CDR1 | VH CDR2 | VH CDR3 | VL CDR1 | VL CDR2 | VL CDR3 | VH | VL |
|---|---|---|---|---|---|---|---|---|
| Ab | SEQ ID: | SEQ ID: | SEQ ID: | SEQ ID: | SEQ ID: | SEQ ID: | SEQ ID: | SEQ ID: |
| AB1 | 279 | 280 | 281 | 282 | 283 | 284 | 459 | 460 |
| AB2 | 285 | 286 | 287 | 288 | 289 | 290 | 461 | 462 |
| AB3 | 291 | 292 | 293 | 294 | 295 | 296 | 463 | 464 |
| AB4 | 297 | 298 | 299 | 300 | 301 | 302 | 465 | 466 |
| AB5 | 303 | 304 | 305 | 306 | 307 | 308 | 467 | 468 |
| AB6 | 309 | 310 | 311 | 312 | 313 | 314 | 469 | 470 |
| AB7 | 315 | 316 | 317 | 318 | 319 | 320 | 471 | 472 |
| AB8 | 321 | 322 | 323 | 324 | 325 | 326 | 473 | 474 |
| AB9 | 327 | 328 | 329 | 330 | 331 | 332 | 475 | 476 |
| AB10 | 333 | 334 | 335 | 336 | 337 | 338 | 477 | 478 |
| AB11 | 339 | 340 | 341 | 342 | 343 | 344 | 479 | 480 |
| AB12 | 345 | 346 | 347 | 348 | 349 | 350 | 481 | 482 |
| AB13 | 351 | 352 | 353 | 354 | 355 | 356 | 483 | 484 |
| AB14 | 357 | 358 | 359 | 360 | 361 | 362 | 485 | 486 |
| AB15 | 363 | 364 | 365 | 366 | 367 | 368 | 487 | 488 |
| AB16 | 369 | 370 | 371 | 372 | 373 | 374 | 489 | 490 |
| AB17 | 375 | 376 | 377 | 378 | 379 | 380 | 491 | 492 |
| AB18 | 381 | 382 | 383 | 384 | 385 | 386 | 493 | 494 |
| AB19 | 387 | 388 | 389 | 390 | 391 | 392 | 495 | 496 |
| AB20 | 393 | 394 | 395 | 396 | 397 | 398 | 497 | 498 |
| AB21 | 399 | 400 | 401 | 402 | 403 | 404 | 499 | 500 |
| AB22 | 405 | 406 | 407 | 408 | 409 | 410 | 501 | 502 |
| AB23 | 411 | 412 | 413 | 414 | 415 | 416 | 503 | 504 |
| AB24 | 417 | 418 | 419 | 420 | 421 | 422 | 505 | 506 |
| AB25 | 423 | 424 | 425 | 426 | 427 | 428 | 507 | 508 |
| AB26 | 429 | 430 | 431 | 432 | 433 | 434 | 509 | 510 |
| AB27 | 435 | 436 | 437 | 438 | 439 | 440 | 511 | 512 |
| AB28 | 441 | 442 | 443 | 444 | 445 | 446 | 513 | 514 |
| AB29 | 447 | 448 | 449 | 450 | 451 | 452 | 515 | 516 |
| AB30 | 453 | 454 | 455 | 456 | 457 | 458 | 517 | 518 |

Furthermore, the antibodies as described herein can have various mutations. In some embodiments, the antibodies (e.g., IgG1 antibodies) can have N297A (EU numbering) mutation, FC-SI mutations (EU Numbering: F243L/R292P/Y300L/V305I/P396L), or the FC-V11 mutations (EU numbering: (G237D/P238D/H268D/P271G/A330R). In some embodiments, the Fc region of the antibodies as described herein can have one or more of the following mutations: N297A, F243L, R292P, Y300L, V305I, P396L, G237D, P238D, H268D, P271G, and A330R.

### Methods of Making Anti-4-1BB Antibodies

An isolated fragment of human 4-1BB can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of 4-1BB and encompasses an epitope of the protein such that an antibody raised against the peptide forms a specific immune complex with the protein. As described above, the full length sequence of human 4-1BB is known in the art (SEQ ID NO: 217).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human 4-1BB). The preparation can further include an adjuvant, such as Freund's complete or incomplete adjuvant, or a similar immunostimulatory agent.

Polyclonal antibodies can be prepared as described above by immunizing a suitable subject with a 4-1BB polypeptide, or an antigenic peptide thereof (e.g., part of 4-1BB) as an immunogen. The antibody titer in the immunized subject can be monitored over time by standard techniques, such as with an enzyme-linked immunosorbent assay (ELISA) using the immobilized 4-1BB polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., 4-1BB. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-4-1BB antibody will contain an amino acid sequence having at least 75%, 80%, 85%, 90%, 95%, 96%, 97%, 98%, or 99% percent identity with a sequence present in the light or heavy chain of the original antibody.

Identity with respect to an original sequence is usually the percentage of amino acid residues present within the candidate sequence that are identical with a sequence present within the human, humanized, or chimeric anti-4-1BB antibody or fragment, after aligning the sequences and introducing gaps, if necessary, to achieve the maximum percent sequence identity, and not considering any conservative substitutions as part of the sequence identity.

Additional modifications to the anti-4-1BB antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-4-1BB antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some aspects, the disclosure also provides the use of the antibodies or antigen fragments thereof described herein for manufacture of a medicament for cancer treatment.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lad and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y., and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Treatment

The antibodies or antibody or antigen-binding fragments thereof of the present disclosure can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the antibody or antigen-binding fragment thereof is an anti-4-1BB antibody or antigen-binding fragment thereof. In some embodiments, the antibody is an IgG1 anti-4-1BB antibody (e.g., human IgG1 anti-4-1BB antibody).

In some embodiments, the compositions and methods disclosed herein can be used for treatment of patients at risk for a cancer. Patients with cancer can be identified with various methods known in the art.

Furthermore, as the anti-4-1BB antibodies can promote immune response, the disclosure provides methods for treating infection in a subject. Types of infection include e.g., bacterial, fungal, viral, protozoan, and parasitic diseases. In some embodiments, the treatment can halt, slow, retard, or inhibit progression of the disease. In addition, 4-1BB antibody treatment (e.g., agonistic antibody or antagonistic antibody) can also be used to treat autoimmune disease, asthma, and additionally as a means to improve vaccination. These methods generally involve administering a therapeutically effective amount of an antibody or antigen-binding fragment thereof disclosed herein to a subject in need thereof.

As used herein, by an “effective amount” is meant an amount or dosage sufficient to effect beneficial or desired results including halting, slowing, retarding, or inhibiting progression of a disease, e.g., a cancer, or an autoimmune disease. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (D01) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

In some embodiments, the additional therapy is chemotherapy or chemoradiation. In some embodiments, the additional therapeutic agent is an anti-CTLA4 antibody (e.g., ipilimumab), an anti-CD20 antibody (e.g., rituximab), an anti-EGFR antibody (e.g., cetuximab), an anti-CD319 antibody (e.g., elotuzumab), or an anti-PD1 antibody (e.g., nivolumab).

In some embodiments, the additional therapeutic agent is an antibody that specifically binds to PD-1, CTLA-4, BTLA, PD-L1, CD27, CD28, CD40, CD47, CD137, CD154, TIGIT, TIM-3, GITR, or OX40.

The present disclosure also shows that the combination of anti-4-1BB IgG1 subclass antibodies with additional therapeutic agents have better tumor inhibitory effects than the combination of anti-4-1BB IgG4 subclass antibodies with some additional therapeutic agents. Thus, in one aspect, the present disclosure provides methods of treating a subject or killing tumors by administering to the subject a therapeutically effective amount of an anti-4-1BB IgG1 antibody or antigen-binding fragment thereof and an additional therapeutic agent. In some embodiments, the additional therapeutic agent is an anti-PD-1 antibody, an anti-CTLA4 antibody, or an-anti-OX40 antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 500 μg/kg to about 5 mg/kg; or about 500 μg/kg to about 2 mg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Mouse Anti-h4-1BB Antibodies

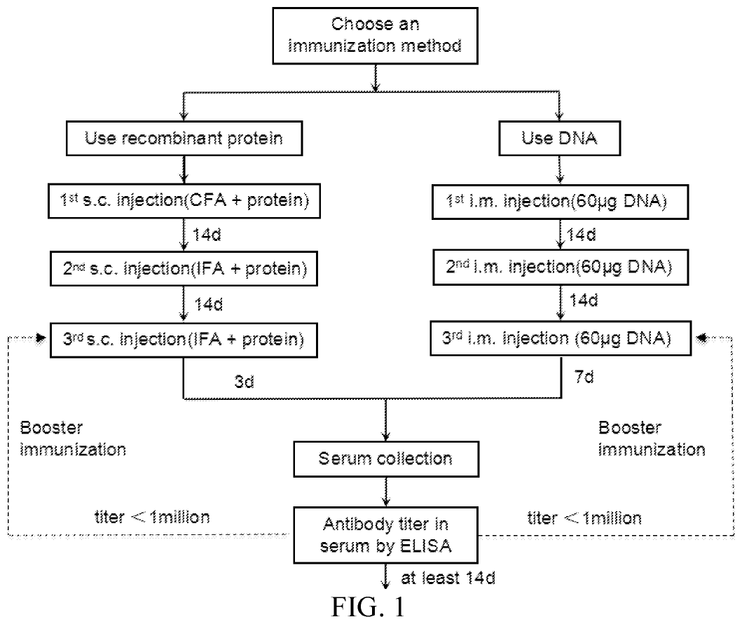

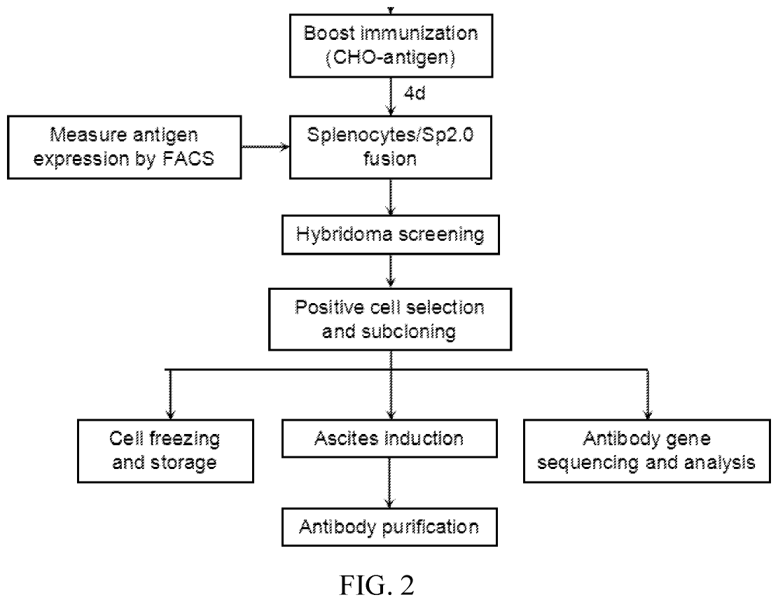

To generate mouse antibodies against human 4-1BB (h4-1BB; SEQ ID NO: 217), 6-8 weeks old female BALB/c mice were immunized with human 4-1BB. Anti-h4-1BB antibodies were collected by the methods as described below and shown in FIG. 1 and FIG. 2.

### Immunization of Mice

6-8 weeks old female BALB/c mice were immunized with His-tagged human 4-1BB proteins at 20 μg/mouse at a concentration of 100 μg/ml. The His-tagged human 4-1BB proteins were emulsified with adjuvant and injected at four positions on the back of the mice. For the first subcutaneous (s.c.) injection, the diluted antigen was emulsified with Complete Freund's Adjuvant (CFA) in equal volume. In the following subcutaneous injections, the protein was emulsified with Incomplete Freund's Adjuvant (IFA) in equal volume. Three days after the third injection or the booster immunization, blood (serum) was collected and analyzed for antibody titer using ELISA.

In another experiment, 6-8 weeks old female BALB/c mice were immunized by injecting the expression plasmid encoding human 4-1BB into the mice. The plasmids encoding the antigen were injected into the tibialis anterior muscle (intramuscular injection; i.m. injection) of the mice by using gene guns at the concentration of 1000 μg/ul at 60 μg per mouse. At least four injections were performed with at least 14 days between two injections. Blood (serum) was collected seven days after the last immunization and the serum was tested for antibody titer by ELISA.

Procedures to enhance immunization were also performed at least fourteen days after the previous immunization (either by injecting the plasmid or by injecting the proteins). CHO cells that express 4-1BB antigen on the surface were intravenously injected into the mice through tail veins. Spleen was then collected four days after the injection.

### Fusion of SP2/0 Cells and Spleen Cells

Spleen tissues were grinded. Spleen cells were first selected by CD3c Microbeads and anti-Mouse IgM Microbeads, and then fused with SP2/0 cells. The cells were then plated in 96-well plates with hypoxanthine-aminopterin-thymidine (HAT) medium.

### Primary Screening of Hybridoma

Primary screening of the hybridoma supernatant in the 96-well plates was performed using Fluorescence-Activated Cell Sorting (FACS) pursuant to standard procedures. Chinese hamster ovary (CHO) cells were added to 96-well plates (2×10⁴ cells per well) before the screening. 50 μl of supernatant was used. The antibodies that were used in experiments were
(1) Fluorescein (FITC)-conjugated AffiniPure F(ab)₂ Fragment Goat Anti-Mouse IgG, Fcγ Fragment Specific, and
(2) Alexa Fluor® 647-conjugated AffiniPure F(ab)2 Fragment Goat Anti-Human IgG, Fcγ Fragment Specific.

### Sub-Cloning

Sub-cloning was performed using ClonePix2. In short, the positive wells identified during the primary screening were transferred to semisolid medium, and IgG positive clones were identified and tested. FITC anti-mouse IgG Fc antibody was used.

### Ascites Fluid Antibodies

1×10⁶ positive hybridoma cells were injected intraperitoneally to B-NDG™ mice (Beijing Biocytogen, Beijing, China). Monoclonal antibodies were produced by growing hybridoma cells within the peritoneal cavity of the mouse. The hybridoma cells multiplied and produced ascites fluid in the abdomens of the mice. The fluid contained a high concentration of antibody which can be harvested for later use.

### Purification of Antibodies

Antibodies in ascites fluid were purified using GE AKTA protein chromatography (GE Healthcare, Chicago, Illinois, United States). At least 27 mouse antibodies were produced. A few antibodies were selected because of the desired properties. These selected mouse antibodies produced by the methods described above include e.g., 16-1C4 (“1C4”), 29-6A5 (“6A5”), 30-5F9 (“5F9”), 45-2B3 (“2B3”), 45-4B9 (“4B9”), 45-7E9 (“7E9”), 45-7G9 (“7G9”), 45-8E11 (“8E11”), 45-8E2 (“8E2”), 45-8F1 (“8F1”), 54-8B11 (“8B11”), 55-8F6 (“8F6”), 56-2A6 (“2A6”), 59-5E4 (“5E4”), 61-6A7 (“6A7”), 69-3C2 (“3C2”), 70-3F9 (“3F9”), and 70-6F10 (“6F10”), etc.

The VH, VL and CDR regions of the antibodies were determined. The heavy chain CDR1, CDR2, CDR3, and light chain CDR1, CDR2, and CDR3 amino acid sequences of these antibodies are shown in Table 1, FIG. 52 and FIG. 53.

### Example 2. Humanization of the Mouse Antibodies

The starting point for humanization was the mouse antibodies (e.g., 1C4, 6A5, and 5F9). The amino acid sequences for the heavy chain variable region and the light chain variable region of these mouse antibodies were determined.

Four humanized heavy chain variable region variants (SEQ ID NOs: 221-224) and four humanized light chain variable region variants (SEQ ID NOs: 225-228) for 1C4 were constructed, containing different modifications or substitutions.

Three humanized heavy chain variable region variants (SEQ ID NOs: 229-231) and four humanized light chain variable region variants (SEQ ID NOs: 232-235) for 6A5 were constructed, containing different modifications or substitutions.

Three humanized heavy chain variable region variants (SEQ ID NOs: 236-238) and four humanized light chain variable region variants (SEQ ID NOs: 239-242) for 5F9 were constructed, containing different modifications or substitutions.

These humanized heavy chain variable region variants can be combined with any of the light chain variable region variants derived from the same mouse antibody. For example, 1C4-H1 (SEQ ID NO: 221) can be combined with any humanized light chain variable region variant based on the same mouse antibody 1C4 (e.g., SEQ ID NO: 225-228), and the antibody will be labeled accordingly. For example, if 1C4-H1 is combined with 1C4-K3 (SEQ ID NO: 227), the antibody is labeled as 1C4-H1K3.

### Example 3. Binding Activity of Anti-h4-1BB Antibodies Against Human 4-1BB

The anti-h4-1BB antibodies were collected from mouse ascites fluid and purified by chromatography. 25 μl CHO cells transiently transfected with human 4-1BB were added to each well in a plate. The purified antibodies were titrated to final concentrations of 10, 1, 0.1, 0.01, 0.001 μg/ml. The titrated antibodies were added to each well at 4° C. and incubated for 30 minutes.

After being washed with phosphate-buffered saline (PBS) (1200 rpm, 5 minutes) twice, 50 μl of FITC labeled anti-mouse IgG Fc antibody (anti-mIgG Fc-FITC) at 1:100 dilution was added into each well, and incubated for 30 minutes at 4° C., followed by PBS wash (1200 rpm, 5 minutes). The signal for FITC was detected by flow cytometry.

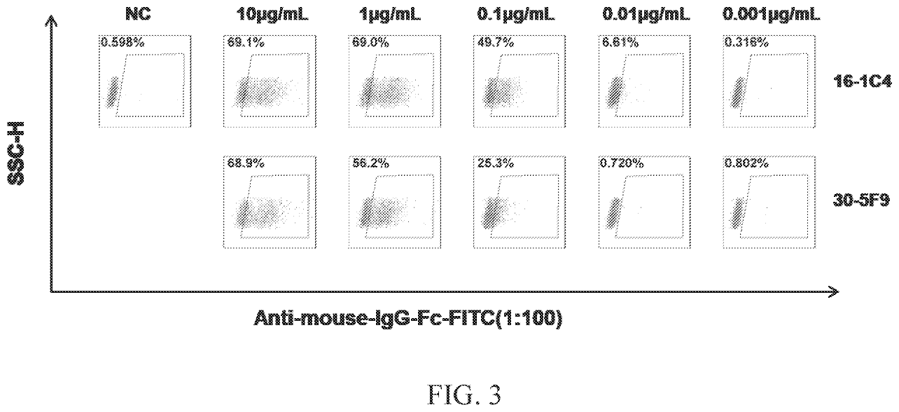

As shown in FIG. 3, 1C4 and 5F9 had strong binding activity with h4-1BB. In FIG. 3, NC stands for negative control.

The table below summarizes the percentage of tested cells that had FITC signals in the flow cytometry analysis. A higher percentage at a lower antibody concertation indicates a higher binding affinity.

TABLE 4

| Antibody | 10 μg/ml | 1 μg/ml | 0.1 μg/ml | 0.01 μg/ml | 0.001 μg/ml |
|---|---|---|---|---|---|
| 16-1C4 | 69.1% | 69.0% | 49.7% | 6.61% | 0.316% |
| 29-6A5 | 80.5% | 73.4% | 70.8% | 47.4% | NA |
| 30-5F9 | 68.9% | 56.2% | 25.3% | 0.720% | 0.802% |
| 45-2B3 | 62.2% | 51.8% | 12.0% | 0.622% | 0.504% |
| 45-4B9 | 56.2% | 49.3% | 10.4% | 1.20% | 1.86% |
| 45-7E9 | 57.6% | 58.4% | 50.0% | 12.7% | 0.703% |
| 45-7G9 | 57.9% | 55.1% | 25.9% | 0.960% | 1.00% |
| 45-8E2 | 58.9% | 60.1% | 49.8% | 9.06% | 1.52% |
| 45-8F1 | 58.6% | 56.1% | 43.9% | 6.21% | 1.38% |
| 54-8B11 | 49.8% | 45.0% | 16.5% | 0.445% | 0.111% |
| 55-8F6 | 53.6% | 52.3% | 36.1% | 0.571% | 0.375% |
| 56-2A6 | 46.6% | 40.9% | 17.1% | 0.890% | 0.250% |
| 61-6A7 | 63.0% | 60.1% | 51.1% | 17.0% | 3.43% |
| 69-3C2 | 58.5% | 62.9% | 52.3% | 26.2% | 0.183% |
| 70-3F9 | 56.7% | 56.4% | 9.06% | 3.37% | 1.25% |
| 70-6F10 | 52.9% | 23.7% | 1.14% | 0.913% | 0.962% |

### Example 4. Cross-Reactivity of Anti-h4-1BB Antibodies Against Monkey, Mouse, and Human-Mouse Chimeric 4-1BB

In each experiment, the CHO cells were transfected with mouse 4-1BB (m4-1BB, SEQ ID NO: 218), monkey (Rhesus macaque) 4-1BB (r4-1BB, SEQ ID NO: 219), or chimeric (mouse and human) 4-1BB (c4-1BB, SEQ ID NO: 220).

25 μl CHO cells were added to each well. 25 μl purified anti-h4-1BB antibodies (1 μg/ml) were added to each well and were incubated at 4° C. for 30 minutes.

After being washed with PBS (1200 rmp, 5 min) twice, 50 μl of FITC labeled anti-mouse IgG Fc antibody (anti-mIgG Fc-FITC) was added into each well at 1:100 dilution, followed by incubating at 4° C. for 30 minutes, and then PBS wash (1200 rmp, 5 min). In some cases, PE labeled anti-mouse IgG Fc antibody (anti-mIgG Fc-PE) was used to label the antibody instead, and was added into each tested well at 1:500 dilution. The signals for FITC and PE were determined by flow cytometry.

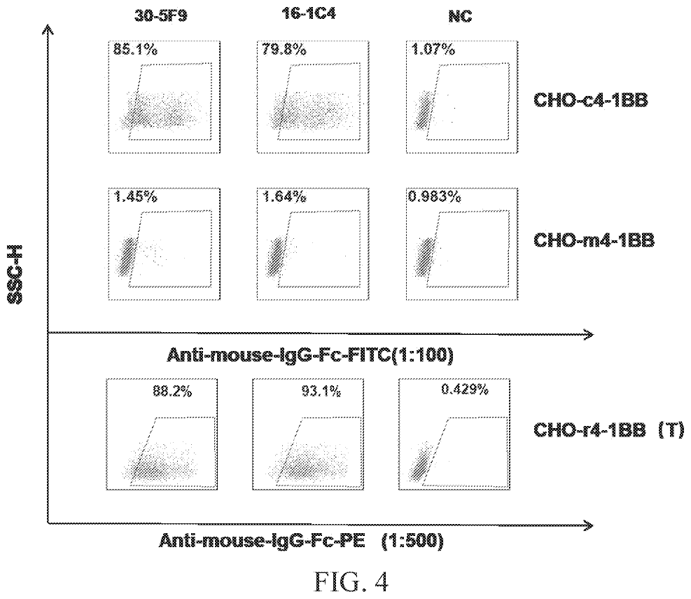

As shown in FIG. 4, 1C4 and 5F9 did not cross react with mouse 4-1BB, but had strong cross reactivity with monkey 4-1BB (r4-1BB) and strong cross reactivity with chimeric 4-1BB (c4-1BB). In FIG. 4, NC stands for negative control.

The cross reactivity for the tested antibodies with monkey (r4-1BB), mouse (m4-1BB), and human-mouse chimeric 4-1BB (c4-1BB) is summarized in the table below.

TABLE 5

| | h4-1BB | r4-1BB | m4-1BB | c4-1BB |
|---|---|---|---|---|
| | (human) | (Monkey) | (Mouse) | (Chimeric) |
| 16-1C4 | Yes | Yes | No | Yes |
| 30-5F9 | Yes | Yes | No | Yes |
| 29-4A10 | Yes | Yes | No | Yes |
| 29-5F10 | Yes | Yes | No | Yes |
| 29-6A5 | Yes | Yes | No | Yes |
| 45-4B9 | Yes | Yes | No | Yes |
| 45-8F1 | Yes | Yes | No | Yes |
| 45-2B3 | Yes | Yes | No | Yes |
| 45-2C11 | Yes | Yes | No | Yes |
| 45-7E9 | Yes | Yes | No | Yes |
| 45-7G9 | Yes | Yes | No | Yes |
| 45-8E2 | Yes | Yes | No | Yes |
| 54-8B11 | Yes | Yes | No | Yes |
| 54-1A11 | Yes | Yes | No | Yes |
| 55-1E3 | Yes | Yes | No | Yes |
| 55-8H5 | Yes | Yes | No | Yes |
| 55-8F6 | Yes | Yes | No | Yes |
| 56-1G6 | Yes | No | No | Yes |
| 61-6A7 | Yes | Yes | No | Yes |
| 58-4B8 | Yes | Yes | No | Yes |
| 56-2A6 | Yes | Very weak | No | Yes |
| 69-3C2 | Yes | Yes | No | Yes |
| 70-3F9 | Yes | Yes | No | Yes |
| 70-6F10 | Yes | Yes | No | Yes |
| 69-4B11 | Yes | Yes | No | Yes |

### Example 5. Binding Affinity of Anti-h4-1BB Antibodies

The binding affinity of the anti-h4-1BB antibodies were measured using surface plasmon resonance (SPR) using Biacore (Biacore, INC, Piscataway N.J.) T200 biosensor equipped with pre-immobilized Protein A sensor chips.

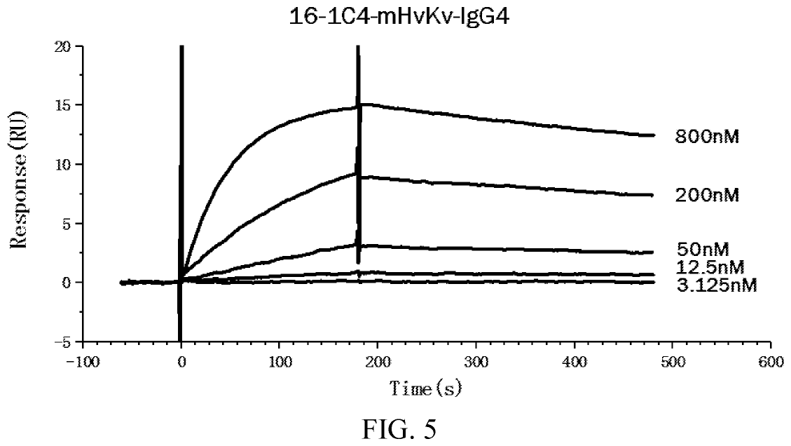

Chimeric anti-h4-1BB antibody 16-1C4-mHvKv-IgG4 (1 μg/mL) were injected into Biacore T200 biosensor at 10 μL/min for 20 seconds to achieve to a desired protein density (about 51.6 response units (RU)). Histidine-tagged human 4-1BB proteins (h4-1BB-His) at the concentration of 800, 200, 50, 12.5, 3.125 nM were then injected at 30 μL/min for 180 seconds. Dissociation was monitored for 300 seconds. The chip was regenerated after the last injection of each titration with Glycine (pH 2.0, 30 μL/min for 5 seconds). The result for 16-1C4-mHvKv-IgG4 is shown in FIG. 5.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore T200 Evaluation Software 3.0. Affinities were calculated from the quotient of the kinetic rate constants (KD=koff/kon).

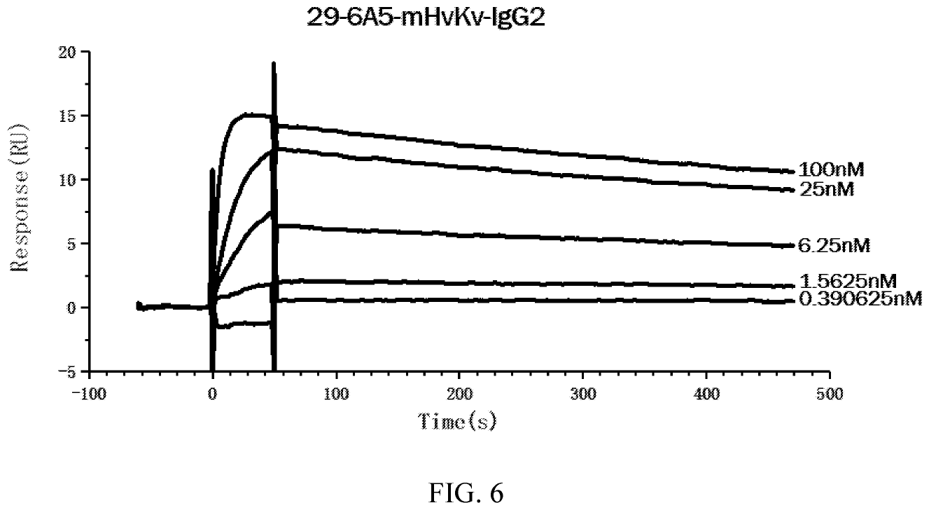

As a person of ordinary skill in the art would understand, the same method with appropriate adjustments for parameters (e.g., antibody concentration) was performed for each tested antibody. For example, the results of 29-6A5-mHvKv-IgG2 are shown in FIG. 6. The results for the tested antibodies are summarized in the table below.

TABLE 6

| | Association rate | Dissociation rate | Affinity |
|---|---|---|---|
| Anti-h4-1BB antibodies | kon (1/Ms) | koff (1/s) | KD (M) |
| 16-1C4-mHvKv-IgG4 | 2.44E+04 | 5.02E−04 | 2.05E−08 |
| 29-6A5-mHvKv-IgG2 | 1.93E+06 | 7.15E−04 | 3.71E−10 |
| 30-5F9-mHvKv-IgG4 | 2.49E+05 | 2.47E−03 | 9.91E−09 |
| 45-2B3-mHvKv-IgG2 | 2.77E+04 | 1.38E−04 | 4.96E−09 |
| 45-4B9-mHvKv-IgG2 | 2.72E+04 | 2.98E−03 | 1.10E−07 |
| 45-7E9-m HvKv-IgG2 | 6.00E+05 | 9.16E−03 | 1.53E−08 |
| 45-7G9-mHvKv-IgG2 | 1.77E+05 | 4.10E−03 | 2.31E−08 |
| 45-8E11-m HvKv-IgG2 | 2.14E+05 | 1.27E−04 | 5.97E−10 |
| 45-8E2-m HvKv-IgG2 | 3.23E+05 | 4.13E−04 | 1.28E−09 |
| 45-8F1-mHvKv-IgG2 | 4.70E+04 | 2.93E−03 | 6.23E−08 |
| 54-8B11-mHvKv-IgG2 | 4.17E+05 | 5.02E−03 | 1.20E−08 |
| 55-8F6-mHvKv-IgG2 | 1.57E+06 | 3.41E−03 | 2.18E−09 |
| 56-2A6-mHvKv-IgG2 | 1.01E+05 | 1.41E−03 | 1.40E−08 |
| 59-5E4-m HvKv -IgG2 | 1.06E+06 | 3.31E−04 | 3.13E−10 |
| 61-6A7-mHvKv-IgG2 | 1.43E+06 | 5.13E−04 | 3.59E−10 |
| 69-3C2-mHvKv-IgG2 | 1.16E+06 | 5.21E−04 | 4.50E−10 |
| 70-3F9-mHvKv-IgG2 | 9.99E+05 | 1.59E−04 | 1.60E−10 |
| 70-6F10-mHvKv-IgG2 | 2.41E+04 | 2.63E−04 | 1.09E−08 |
| urelumab | 6.81E+04 | 3.02E−03 | 4.43E−08 |
| utomllumab | 1.55E+06 | 1.07E−02 | 6.93E−09 |
| 1C4-H1K1-IgG4 | 2.67E+04 | 6.16E−04 | 2.31E−08 |
| 1C4-H1K2-IgG4 | 2.75E+04 | 6.05E−04 | 2.20E−08 |
| 1C4-H1K3-IgG4 | 2.85E+04 | 6.23E−04 | 2.18E−08 |
| 1C4-H1K4-IgG4 | 2.57E+04 | 6.14E−04 | 2.39E−08 |
| 1C4-H2K1-IgG4 | 2.59E+04 | 5.02E−04 | 1.94E−08 |
| 1C4-H2K2-IgG4 | 2.58E+04 | 6.46E−04 | 2.51E−08 |
| 1C4-H2K3-IgG4 | 2.85E+04 | 5.02E−04 | 1.76E−08 |
| 1C4-H2K4-IgG4 | 2.52E+04 | 5.21E−04 | 2.07E−08 |
| 1C4-H3K2-IgG4 | 2.77E+04 | 4.53E−04 | 1.63E−08 |
| 1C4-H3K3-IgG4 | 3.12E+04 | 5.33E−04 | 1.71E−08 |
| 1C4-H3K4-IgG4 | 3.00E+04 | 4.76E−04 | 1.59E−08 |
| 1C4-H4K1-IgG4 | 2.84E+04 | 5.16E−04 | 1.82E−08 |
| 1C4-H4K2-IgG4 | 2.96E+04 | 4.90E−04 | 1.66E−08 |
| 1C4-H4K3-IgG4 | 3.29E+04 | 5.56E−04 | 1.69E−08 |
| 1C4-H4 K4-IgG4 | 2.90E+04 | 5.60E−04 | 1.93E−08 |
| 5F9-H1K1-IgG4 | 2.22E+05 | 2.53E−03 | 1.14E−08 |
| 5F9-H1K2-IgG4 | 2.05E+05 | 2.47E−03 | 1.20E−08 |
| 5F9-H1K3-IgG4 | 2.06E+05 | 2.08E−03 | 1.01E−08 |
| 5F9-H1K4-IgG4 | 2.46E+05 | 2.44E−03 | 9.95E−09 |
| 5F9-H2K1-IgG4 | 2.02E+05 | 2.25E−03 | 1.12E−08 |
| 5F9-H2K2-IgG4 | 1.94E+05 | 2.61E−03 | 1.34E−08 |
| 5F9-H2K3-IgG4 | 2.12E+05 | 2.07E−03 | 9.81E−09 |
| 5F9-H2K4-IgG4 | 2.16E+05 | 2.69E−03 | 1.24E−08 |
| 5F9-H3K1-IgG4 | 1.98E+05 | 2.42E−03 | 1.22E−08 |
| 5F9-H3K2-IgG4 | 1.88E+05 | 2.58E−03 | 1.37E−08 |
| 5F9-H3K3-IgG4 | 2.10E+05 | 2.20E−03 | 1.05E−08 |
| 5F9-H3K4-IgG4 | 2.36E+05 | 2.70E−03 | 1.14E−08 |
| 6A5-H1K1-IgG2 | 2.85E+13 | 3.33E+04 | 1.17E−09 |
| 6A5-H1K2-IgG2 | 2.38E+06 | 6.62E−04 | 2.78E−10 |
| 6A5-H1K3-IgG2 | 2.22E+06 | 4.75E−04 | 2.14E−10 |
| 6A5-H1K4-IgG2 | 2.16E+06 | 6.93E−04 | 3.21E−10 |
| 6A5-H2K1-IgG2 | 6.68E+05 | 1.55E−03 | 2.32E−09 |
| 6A5-H2K2-IgG2 | 2.33E+06 | 6.52E−04 | 2.80E−10 |
| 6A5-H2K3-IgG2 | 2.28E+06 | 5.04E−04 | 2.21E−10 |
| 6A5-H2K4-IgG2 | 2.24E+06 | 5.81E−04 | 2.60E−10 |
| 6A5-H3K1-IgG2 | 6.50E+05 | 1.81E−03 | 2.78E−09 |
| 6A5-H3K2-IgG2 | 2.05E+06 | 8.25E−04 | 4.02E−10 |
| 6A5-H3K3-IgG2 | 2.14E+06 | 5.38E−04 | 2.52E−10 |
| 6A5-H3K4-IgG2 | 1.87E+06 | 7.47E−04 | 4.00E−10 |

As described in Example 1, 16-1C4, 29-6A5, 30-5F9, 45-2B3, 45-4B9, 45-7E9, 45-7G9, 45-8E11, 45-8E2, 45-8F1, 54-8B11, 55-8F6, 56-2A6, 59-5E4, 61-6A7, 69-3C2, 70-3F9, and 70-6F10 are mouse anti-h4-1BB antibodies. Based on these mouse anti-h4-1BB antibodies, the chimeric anti-h4-1BB antibodies including e.g., 16-1C4-mHvKv-IgG4, 29-6A5-mHvKv-IgG2, 30-5F9-mHvKv-IgG4, 45-2B3-mHvKv-IgG2, 45-4B9-mHvKv-IgG2, 45-7E9-mHvKv-IgG2, 45-7G9-mHvKv-IgG2, 45-8E11-mHvKv-IgG2, 45-8E2-mHvKv-IgG2, 45-8F1-mHvKv-IgG2, 54-8B11-mHvKv-IgG2, 55-8F6-mHvKv-IgG2, 56-2A6-mHvKv-IgG2, 59-5E4-mHvKv-IgG2, 61-6A7-mHvKv-IgG2, 69-3C2-mHvKv-IgG2, 70-3F9-mHvKv-IgG2, and 70-6F10-mHvKv-IgG2 were generated. The chimeric antibodies have the heavy chain variable domain and the light chain variable domain from the corresponding mouse anti-h4-1BB antibodies, and the constant domains from human IgG antibodies (including, e.g., the CL, CH1, CH2, and CH3 domains).

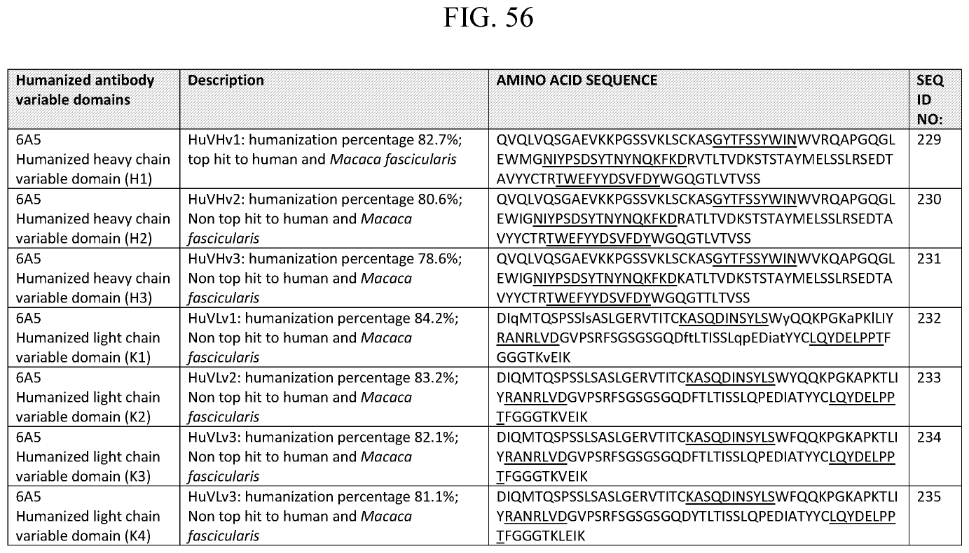

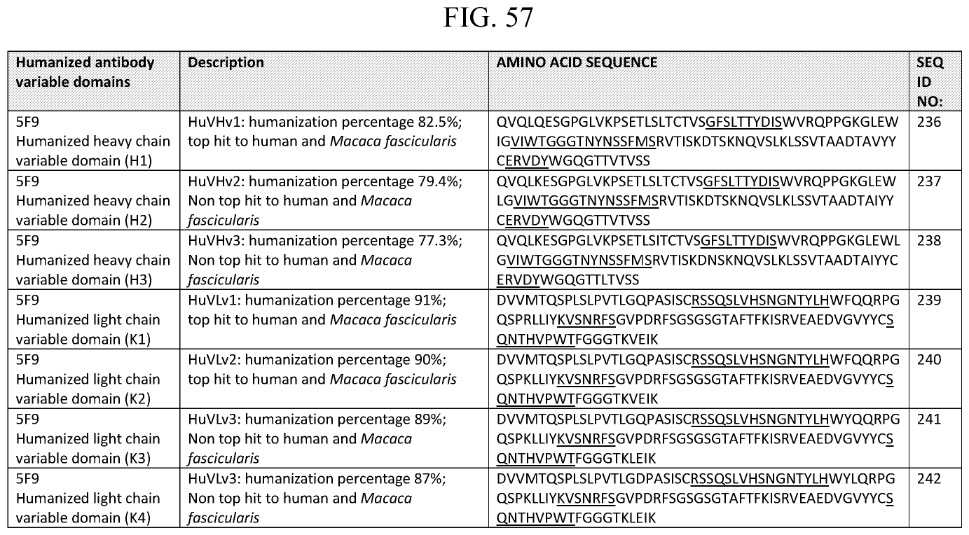

The tested antibodies also include humanized antibodies, e.g., 1C4-H1K1-IgG4, 1C4-H1K2-IgG4, 1C4-H1K3-IgG4, 1C4-H1K4-IgG4, 1C4-H2K1-IgG4, 1C4-H2K2-IgG4, 1C4-H2K3-IgG4, 1C4-H2K4-IgG4, 1C4-H3K1-IgG4, 1C4-H3K2-IgG4, 1C4-H3K3-IgG4, 1C4-H3K4-IgG4, 1C4-H4K1-IgG4, 1C4-H4K2-IgG4, 1C4-H4K3-IgG4, 1C4-H4K4-IgG4, 5F9-H1K1-IgG4, 5F9-H1K2-IgG4, 5F9-H1K3-IgG4, 5F9-H1K4-IgG4, 5F9-H2K1-IgG4, 5F9-H2K2-IgG4, 5F9-H2K3-IgG4, 5F9-H2K4-IgG4, 5F9-H3K1-IgG4, 5F9-H3K2-IgG4, 5F9-H3K3-IgG4, 5F9-H3K4-IgG4, 6A5-H1K1-IgG2, 6A5-H1K2-IgG2, 6A5-H1K3-IgG2, 6A5-H1K4-IgG2, 6A5-H2K1-IgG2, 6A5-H2K2-IgG2, 6A5-H2K3-IgG2, 6A5-H2K4-IgG2, 6A5-H3K1-IgG2, 6A5-H3K2-IgG2, 6A5-H3K3-IgG2, and 6A5-H3K4-IgG2, etc. The humanized antibodies have human antibody constant domains (including, e.g., the CL, CH1, CH2, and CH3 domains). The humanized variable domains of the heavy chain are numbered H1, H2, H3 etc.; and the humanized variable domains of the light chain are numbered K1, K2, K3 etc. The sequences of the humanized variable domains are summarized in FIG. 55, FIG. 56, and FIG. 57. For example, 1C4-H1K1-IgG4 is based on the mouse antibody 1C4 and has the humanized heavy chain variable domain H1 (SEQ ID NO: 221) and humanized light chain variable domain K1 (SEQ ID NO: 225). Similarly, 5F9-H1K2-IgG4 is based on mouse antibody 5F9 and has humanized heavy chain variable domain H1 (SEQ ID NO: 236) and humanized light chain variable domain K2 (SEQ ID NO: 240).

### Example 6. Thermal Stability of Anti-h4-1BB Antibodies

Thermofluor assay was performed using the Protein Thermal Shift™ Dye Kit (Thermo Fisher Scientific) and QuantStudio™ 5 Real Time PCR Systems (Thermo Fisher Scientific). This assay measured thermostability using a fluorescent dye that binds to hydrophobic patches exposed as the protein unfolds.

The experiments were performed according to the manufacturer's protocol. 2 μL of antibody, 10.5 μL of water, 5 μL of Protein Thermal Shift buffer, and 2.5 μL of diluted Protein Thermal Shift Dye were mixed. Samples were heated to 25° C. at 1.6° C./second, and then heated to 99° C. at 0.05° C./second.

The table below summarizes the Tm for several humanized or chimeric anti-h4-1BB antibodies.

TABLE 7

| | | Type | |
|---|---|---|---|
| | Variable | (constant | Thermal stability |
| Antibody | Domains | domains) | (Tm D2 (Fab)° C.) |
| 1C4-H1K1-IgG4 | 1C4 H1K1 | Human IgG4 | 80.60 |
| 1C4-H1K2-IgG4 | 1C4 H1K2 | Human IgG4 | 80.38 |
| 16-1C4-mHvKv-IgG1 | 1C4 mHvKv | Human IgG1 | 78.09 |
| 16-1C4-mHvKv-IgG2 | 1C4 mHvKv | Human IgG2 | 78.16 |
| 16-1C4-mHvKv-IgG4 | 1C4 mHvKv | Human IgG4 | 77.79 |
| 5F9-H1K1-IgG4 | 5F9 H1K1 | Human IgG4 | 80.23 |
| 5F9-H1K2-IgG4 | 5F9 H1K2 | Human IgG4 | 80.08 |
| 30-5F9-mHvKv-IgG2 | 5F9 mHvKv | Human IgG2 | 80.60 |
| 30-5F9-mHvKv-IgG4 | 5F9 mHvKv | Human IgG4 | 80.01 |
| 6A5-H2K2-IgG2 | 6A5 H2K2 | Human IgG2 | 88.66 |
| 6A5-H2K3-IgG2 | 6A5 H2K3 | Human IgG2 | 88.29 |
| 6A5-H1K3-IgG2 | 6A5 H1K3 | Human IgG2 | 88.43 |
| 6A5-H1K2-IgG2 | 6A5 H1K2 | Human IgG2 | 88.36 |
| Utomilumab | Human | Human IgG2 | 72.61 |
| Urelumab | Human | Human IgG4 | 59.90 |

The results show that the anti-h4-1BB antibodies described herein have a higher Tm than utomilumab and urelumab.

### Example 7. In Vivo Testing of Mouse Anti-h4-1BB Antibodies

In order to test the anti-h4-1BB antibodies in vivo and to predict the effects of these antibodies in human, a humanized 4-1BB mouse model was generated. The humanized 4-1BB mouse model was engineered to express a chimeric 4-1BB protein (SEQ ID NO: 220) wherein a part of the extracellular region of the mouse 4-1BB protein was replaced with the corresponding human 4-1BB extracellular region. The amino acid residues 1-183 of mouse 4-1BB (SEQ ID NO: 218) were replaced by amino acid residues 1-184 of human 4-1BB (SEQ ID NO: 217). The humanized mouse model (B-h4-1BB) provides a new tool for testing new therapeutic treatments in a clinical setting by significantly decreasing the difference between clinical outcome in human and in ordinary mice expressing mouse 4-1BB. A detailed description regarding humanized 4-1BB mouse model can be found in PCT/CN2017/120388, which is incorporated herein by reference in its entirety.

The anti-h4-1BB antibodies were tested for their effect on tumor growth in vivo in a model of colon carcinoma. About 5×10⁵ MC-38 cancer tumor cells (colon adenocarcinoma cell) were injected subcutaneously in B-h4-1BB mice. When the tumors in the mice reached a volume of 150±50 mm³, the mice were randomly placed into different groups based on the volume of the tumor.

The mice were then injected with physiological saline (PS) and anti-h4-1BB antibodies by intraperitoneal (i.p.) administration.

The injected volume was calculated based on the weight of the mouse at 1 mg/kg or 3 mg/kg. The length of the long axis and the short axis of the tumor were measured and the volume of the tumor was calculated as 0.5×(long axis)×(short axis)². The weight of the mice was also measured before the injection, when the mice were placed into different groups (before the first antibody injection), twice a week during the antibody injection period, and before euthanization.

The tumor growth inhibition percentage (TGI %) was calculated using the following formula: TGI (%)=[1−(Ti−T0)/(Vi−V0)]×100. Ti is the average tumor volume in the treatment group on day i. T0 is the average tumor volume in the treatment group on day zero. Vi is the average tumor volume in the control group on day i. V0 is the average tumor volume in the control group on day zero.

T-test was performed for statistical analysis. A TGI % higher than 60% indicates clear suppression of tumor growth. P<0.05 is a threshold to indicate significant difference.

### In Vivo Results for Mouse Anti-h4-1BB Antibodies 16-1C4 (“1C4”) and 30-5F9 (“5F9”)

In each group, B-h4-1BB mice were injected with physiological saline (PS) (G1), 1 mg/kg Urelumab (G2), 3 mg/kg Urelumab (G3), 1 mg/kg of the mouse anti-h4-1BB antibody 16-1C4 (G4), 3 mg/kg of 16-1C4 (G5), 1 mg/kg of the mouse anti-h4-1BB antibody 30-5F9 (G6) or 3 mg/kg of 30-5F9 (G7).

TABLE 8

| | | | | | | Total |
|---|---|---|---|---|---|---|
| | | | | | | No. of |
| | No. | | Dosage | | | admini- |
| Group | of mice | Antibodies | (mg/kg) | Route | Frequency | stration |
| G1 | 5 | PS (control) | — | i.p. | Day 1, 4/wk | 6 |
| G2 | 5 | Urelumab | 1 mg/kg | i.p. | Day 1, 4/wk | 6 |
| G3 | 5 | Urelumab | 3 mg/kg | i.p. | Day 1, 4/wk | 6 |
| G4 | 5 | 16-1C4 | 1 mg/kg | i.p. | Day 1, 4/wk | 6 |
| G5 | 5 | 16-1C4 | 3 mg/kg | i.p. | Day 1, 4/wk | 6 |
| G6 | 5 | 30-5F9 | 1 mg/kg | i.p. | Day 1, 4/wk | 6 |
| G7 | 5 | 30-5F9 | 3 mg/kg | i.p. | Day 1, 4/wk | 6 |

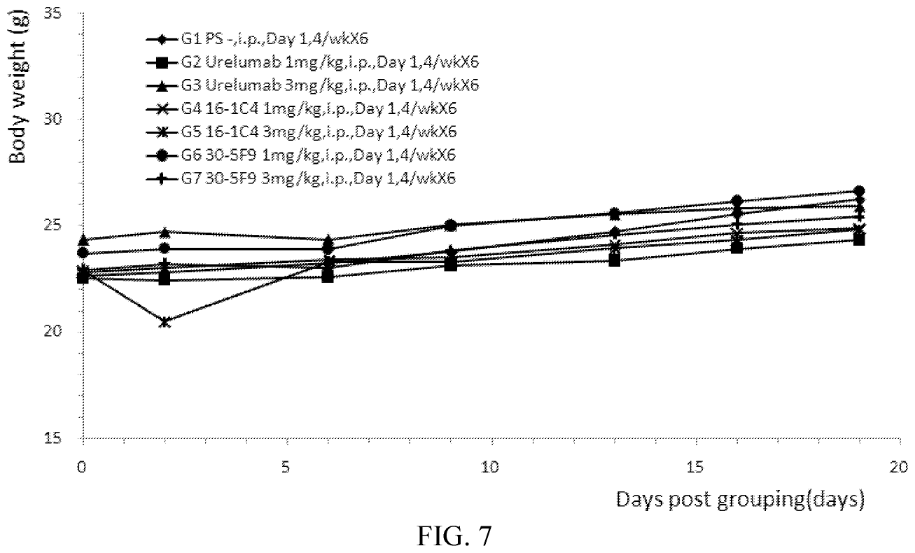

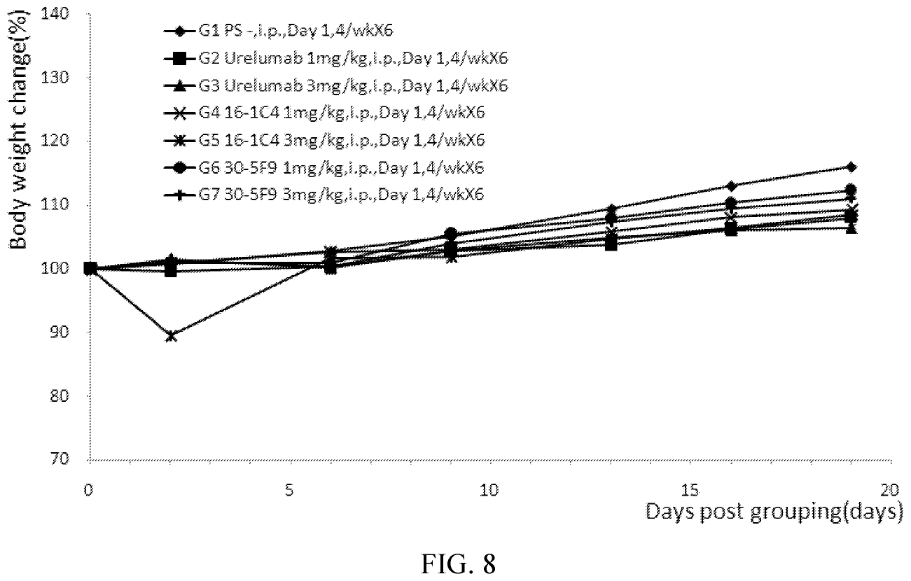

The weight of the mice was monitored during the entire treatment period. The average weight of mice in different groups all increased to different extents (FIG. 7, and FIG. 8). No obvious difference in weight was observed among different groups at the end of the treatment periods. The results showed that 1C4 and 6A5 were well tolerated and were not obviously toxic to the mice.

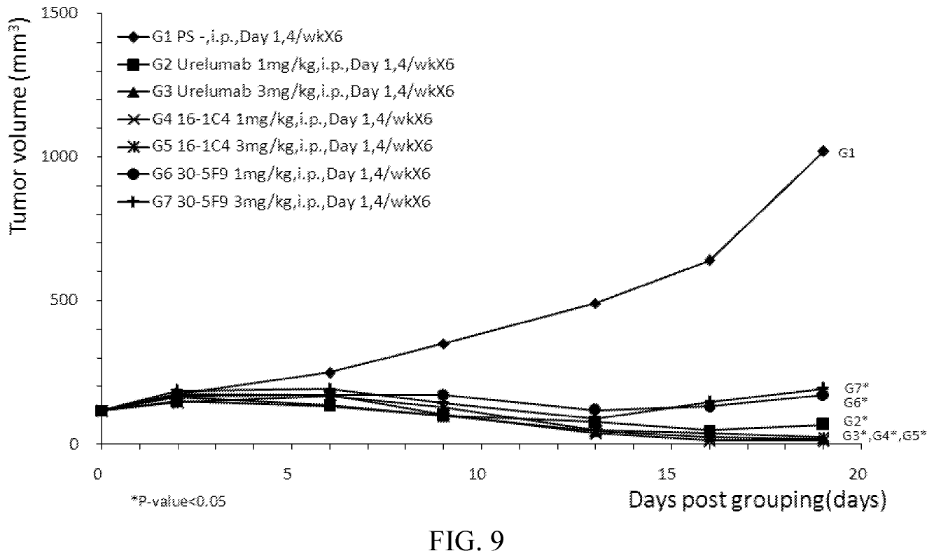

The tumor size in groups treated with urelumab, 1C4, or 6A5 all decreased (FIG. 9).

The TGI % at day 19 (19 days after grouping) was also calculated as shown in the table below.

TABLE 9

| | | Tumor volume(mm³) | | | | | | P value | |
|---|---|---|---|---|---|---|---|---|---|
| | | Day | Day | Day | Day | | | Body | Tumor |
| | | 0 | 6 | 13 | 19 | Survival | TGI % | weight | Volume |
| Control | G1 | 117 ± 17 | 250 ± 40 | 491 ± 75 | 1020 ± 244 | 5/5 | n.a. | n.a. | n.a. |
| Treat | G2 | 117 ± 17 | 133 ± 51 | 77 ± 42 | 69 ± 47 | 5/5 | 105.4% | 0.101 | 0.005 |
| | G3 | 117 ± 19 | 137 ± 58 | 46 ± 30 | 19 ± 15 | 5/5 | 110.9% | 0.793 | 0.003 |
| | G4 | 118 ± 18 | 170 ± 44 | 39 ± 27 | 12 ± 9 | 5/5 | 111.7% | 0.241 | 0.003 |
| | G5 | 117 ± 22 | 169 ± 34 | 49 ± 20 | 26 ± 13 | 5/5 | 110.1% | 0.145 | 0.004 |
| | G6 | 117 ± 19 | 174 ± 61 | 119 ± 42 | 170 ± 77 | 5/5 | 94.1% | 0.759 | 0.010 |
| | G7 | 118 ± 18 | 192 ± 71 | 90 ± 55 | 194 ± 137 | 5/5 | 91.5% | 0.479 | 0.018 |

The results showed that anti-h4-1BB antibodies 1C4 and 5F9 significantly inhibited tumor growth. Furthermore, 1C4 and 5F9 had similar TGI % at 1 mg/kg and 3 mg/kg. This result suggests that a higher dosage of 1C4 and 5F9 does not clearly improve tumor inhibitory effects.

### In Vivo Results for Mouse Anti-h4-1BB Antibodies 29-6A5 (“6A5”), 29-4A10 (“4A10”), 29-5F10 (“5F10”), 45-8F1 (“8F1”), and 45-4B9 (“4B9”)

B-h4-1BB mice were injected with physiological saline (PS) as a control (G1), 3 mg/kg 29-6A5 (G2), 3 mg/kg 29-4A10 (G3), 3 mg/kg 29-5F10 (G4), 3 mg/kg 45-8F1 (G5), and 3 mg/kg 45-4B9 (G6). 3 mg/kg urelumab was also administered to the mice for comparison purpose (G7).

TABLE 10

| | | | | | | Total |
|---|---|---|---|---|---|---|
| | | | | | | No. of |
| | No. | | Dosage | | | admini- |
| Group | of mice | Antibodies | (mg/kg) | Route | Frequency | stration |
| G1 | 5 | PS (control) | — | i.p. | Day 1, 4/wk | 6 |
| G2 | 5 | 29-6A5 | 3 mg/kg | i.p. | Day 1, 4/wk | 6 |
| G3 | 5 | 29-4A10 | 3 mg/kg | i.p. | Day 1, 4/wk | 6 |
| G4 | 5 | 29-5F10 | 3 mg/kg | i.p. | Day 1, 4/wk | 6 |
| G5 | 5 | 45-8F1 | 3 mg/kg | i.p. | Day 1, 4/wk | 6 |
| G6 | 5 | 45-4B9 | 3 mg/kg | i.p. | Day 1, 4/wk | 6 |
| G7 | 5 | Urelumab | 3 mg/kg | i.p. | Day 1, 4/wk | 6 |

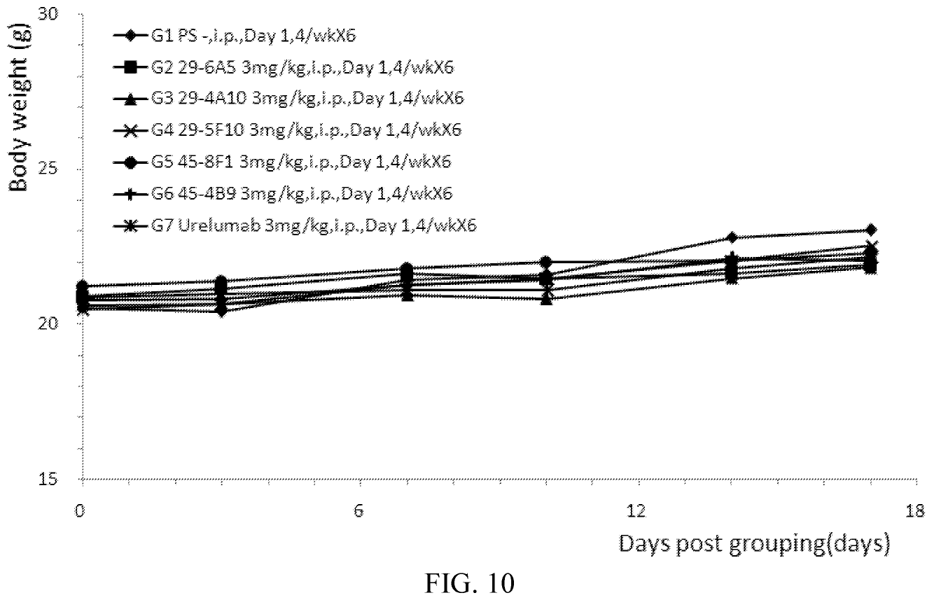

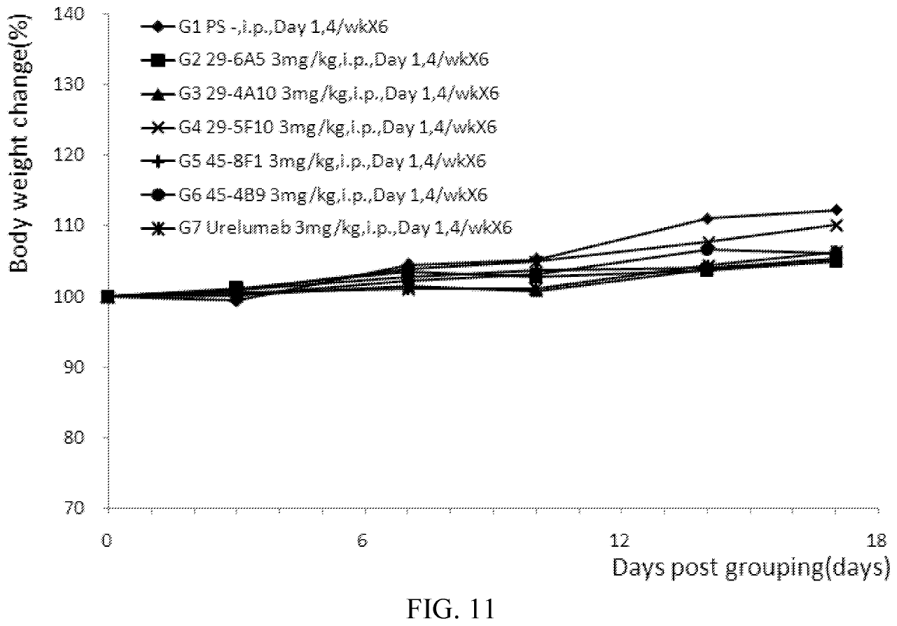

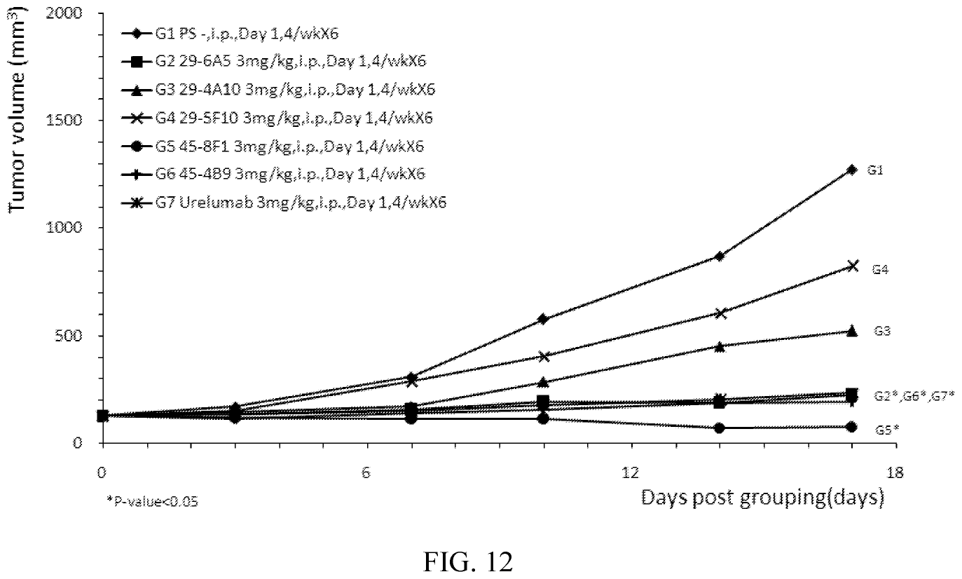

The weight of the mice was monitored during the entire treatment period. The weight of mice in different groups all increased at the end of the treatment period (FIG. 10, and FIG. 11). No obvious difference in weight was observed among different groups. The results showed that these anti-h4-1BB antibodies were well tolerated and were not obviously toxic to the mice. The tumor size in groups treated with 29-6A5, 29-4A10, 29-5F10, 45-8F1, and 45-4B9 is shown in FIG. 12.

The TGI % at day 17 (17 days after grouping) was also calculated as shown in the table below.

TABLE 11

| | | Tumor volume(mm³) | | | | | | P value | |
|---|---|---|---|---|---|---|---|---|---|
| | | Day | Day | Day | Day | | | Body | Tumor |
| | | 0 | 7 | 14 | 17 | Survival | TGI % | weight | Volume |
| Control | G1 | 131 ± 7 | 310 ± 56 | 873 ± 190 | 1274 ± 282 | 5/5 | n.a. | n.a. | n.a. |
| Treat | G2 | 131 ± 7 | 161 ± 34 | 191 ± 67 | 228 ± 87 | 5/5 | 91.5% | 0.267 | 0.008 |
| | G3 | 131 ± 6 | 173 ± 40 | 452 ± 222 | 524 ± 242 | 5/5 | 65.6% | 0.455 | 0.078 |
| | G4 | 132 ± 10 | 293 ± 64 | 608 ± 115 | 824 ± 132 | 5/5 | 39.4% | 0.597 | 0.186 |
| | G5 | 131 ± 7 | 116 ± 24 | 72 ± 25 | 78 ± 35 | 5/5 | 104.7% | 0.481 | 0.003 |
| | G6 | 131 ± 8 | 140 ± 29 | 190 ± 63 | 197 ± 67 | 5/5 | 94.3% | 0.226 | 0.006 |
| | G7 | 131 ± 10 | 151 ± 40 | 204 ± 99 | 239 ± 131 | 5/5 | 90.6% | 0.287 | 0.010 |

The results showed that anti-h4-1BB antibodies 6A5, 4A10, 8F1, 4B9 all inhibited tumor growth. Furthermore, 6A5 and 8F1 had higher TGI % than the other tested antibodies (e.g., Urelumab).

### In Vivo Results for Mouse Anti-h4-1BB Antibodies 45-8E2 (“8E2”), 45-7E9 (“7E9”), 45-7G9 (“7G9”), 45-2B3 (“2B3”), and 45-2C11 (“2C11”)

B-h4-1BB mice were injected with physiological saline (PS) as a control (G1), 1 mg/kg 45-8E2 (G2), 1 mg/kg 45-7E9 (G3), 1 mg/kg 45-7G9 (G4), 1 mg/kg 45-2B3 (G5), and 1 mg/kg 45-2C11 (G6). 1 mg/kg Urelumab was also administered to the mice for comparison purpose (G7).

TABLE 12

| | | | | | | Total |
|---|---|---|---|---|---|---|
| | | | | | | No. of |
| | No. | | Dosage | | | admini- |
| Group | of mice | Antibodies | (mg/kg) | Route | Frequency | stration |
| G1 | 5 | PS (control) | — | i.p. | Day 2, 5/wk | 6 |
| G2 | 5 | 45-8E2 | 1 mg/kg | i.p. | Day 2, 5/wk | 6 |
| G3 | 5 | 45-7E9 | 1 mg/kg | i.p. | Day 2, 5/wk | 6 |
| G4 | 5 | 45-7G9 | 1 mg/kg | i.p. | Day 2, 5/wk | 6 |
| G5 | 5 | 45-2B3 | 1 mg/kg | i.p. | Day 2, 5/wk | 6 |
| G6 | 5 | 45-2C11 | 1 mg/kg | i.p. | Day 2, 5/wk | 6 |
| G7 | 5 | Urelumab | 1 mg/kg | i.p. | Day 2, 5/wk | 6 |

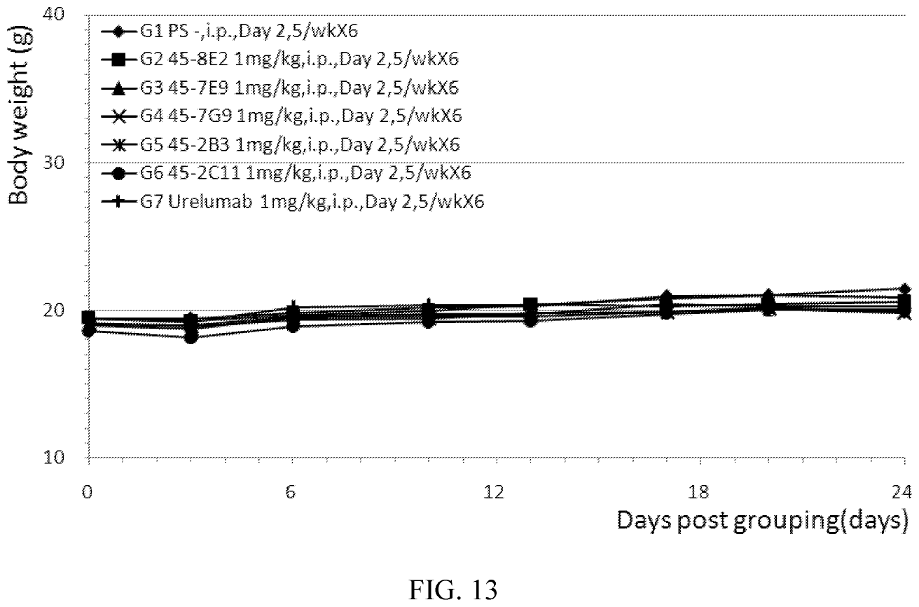

The weight of the mice was monitored during the entire treatment period. The average weight of mice in different groups all increased at the end of the treatment period (FIG. 13, and FIG. 14). No significant difference in weight was observed among different groups. The results showed that these anti-h4-1BB antibodies were well tolerated and were not obviously toxic to the mice.

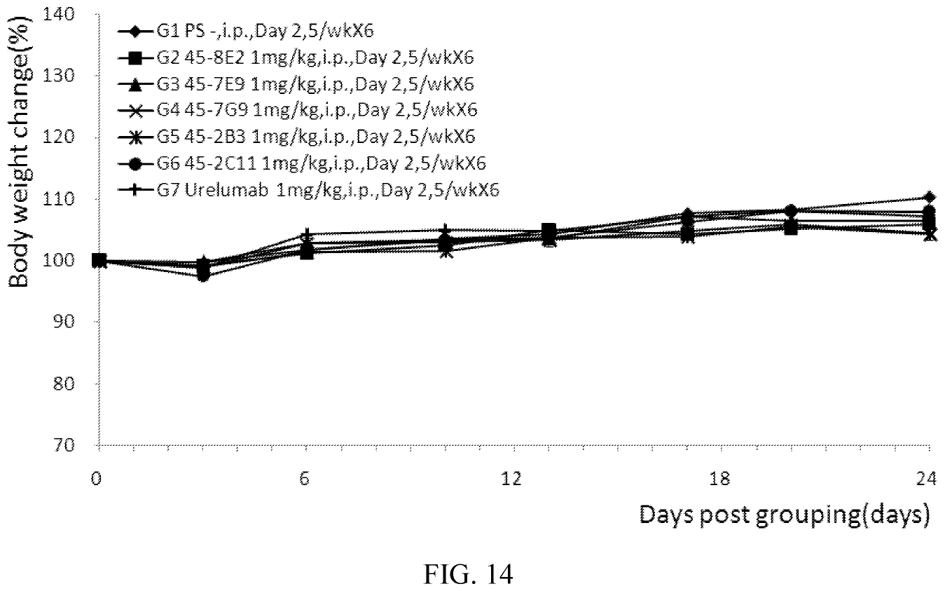

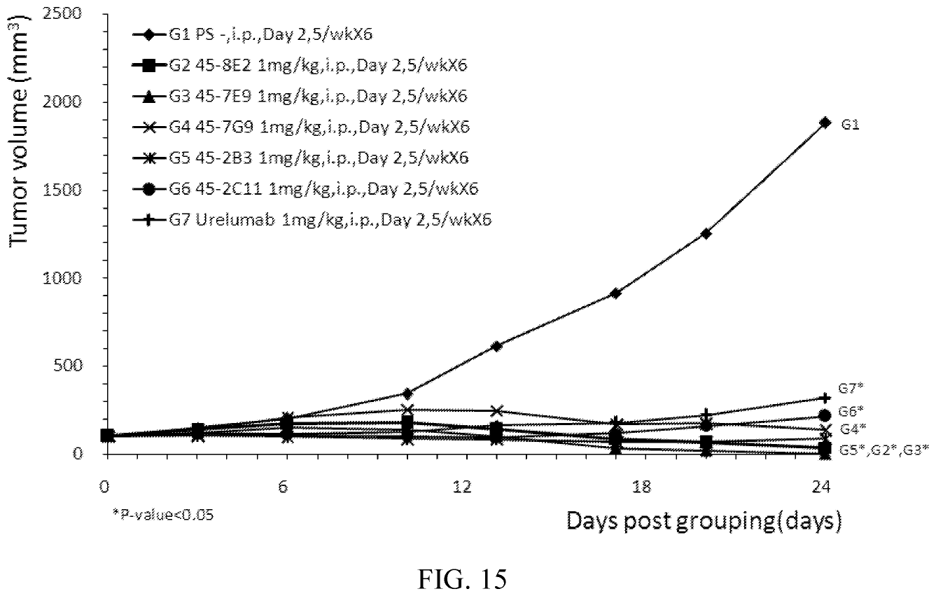

As compared to the control group, 45-8E2, 45-7E9, 45-7G9, 45-2B3, and 45-2C11 all inhibited tumor growth (FIG. 15). The TGI % at day 24 (24 days after grouping) was also calculated as shown in the table below.

TABLE 13

| | | Tumor volume(mm³) | | | | | | P value | |
|---|---|---|---|---|---|---|---|---|---|
| | | Day | Day | Day | Day | | | Body | Tumor |
| | | 0 | 10 | 17 | 24 | Survival | TGI % | weight | Volume |
| Control | G1 | 106 ± 16 | 346 ± 20 | 916 ± 102 | 1882 ± 180 | 5/5 | n.a. | n.a. | n.a. |
| Treat | G2 | 106 ± 11 | 182 ± 67 | 91 ± 53 | 41 ± 37 | 5/5 | 103.7% | 0.573 | 8.5E−06 |
| | G3 | 106 ± 10 | 142 ± 13 | 36 ± 12 | 5 ± 3 | 5/5 | 105.7% | 0.405 | 6.3E−06 |
| | G4 | 106 ± 9 | 256 ± 42 | 170 ± 34 | 141 ± 39 | 5/5 | 98.0% | 0.304 | 1.3E−05 |
| | G5 | 106 ± 11 | 90 ± 21 | 73 ± 38 | 90 ± 54 | 5/5 | 100.9% | 0.251 | 1.2E−05 |
| | G6 | 106 ± 12 | 101 ± 28 | 122 ± 53 | 218 ± 100 | 5/5 | 93.7% | 0.384 | 4.1E−05 |
| | G7 | 106 ± 15 | 129 ± 60 | 181 ± 144 | 323 ± 265 | 5/5 | 87.8% | 0.735 | 0.001 |

The results showed that anti-h4-1BB antibodies 45-8E2, 45-7E9, 45-7G9, 45-2B3, and 45-2C11 all significantly inhibited tumor growth. Furthermore, 45-8E2, 45-7E9, 45-7G9, 45-2B3, and 45-2C11 all had higher TGI % than Urelumab.

### In Vivo Results for Mouse Anti-h4-1BB Antibodies 16-1C4 (“1C4”), 55-8F6 (“8F6”), and 54-8B11 (“8B11”)

B-h4-1BB mice were injected with physiological saline (PS) as a control (G1), 1 mg/kg 16-1C4 (G2), 1 mg/kg 55-8F6 (G3), and 1 mg/kg 54-8B11 (G4). 1 mg/kg Urelumab was also administered to the mice for comparison purpose (G5).

TABLE 14

| | | | | | | Total |
|---|---|---|---|---|---|---|
| | | | | | | No. of |
| | No. | | Dosage | | | admini- |
| Group | of mice | Antibodies | (mg/kg) | Route | Frequency | stration |
| G1 | 5 | PS (control) | — | i.p. | Day 2, 5/wk | 6 |
| G2 | 5 | 16-1C4 | 1 mg/kg | i.p. | Day 2, 5/wk | 6 |
| G3 | 5 | 55-8F6 | 1 mg/kg | i.p. | Day 2, 5/wk | 6 |
| G4 | 5 | 54-8B11 | 1 mg/kg | i.p. | Day 2, 5/wk | 6 |
| G5 | 5 | Urelumab | 1 mg/kg | i.p. | Day 2, 5/wk | 6 |

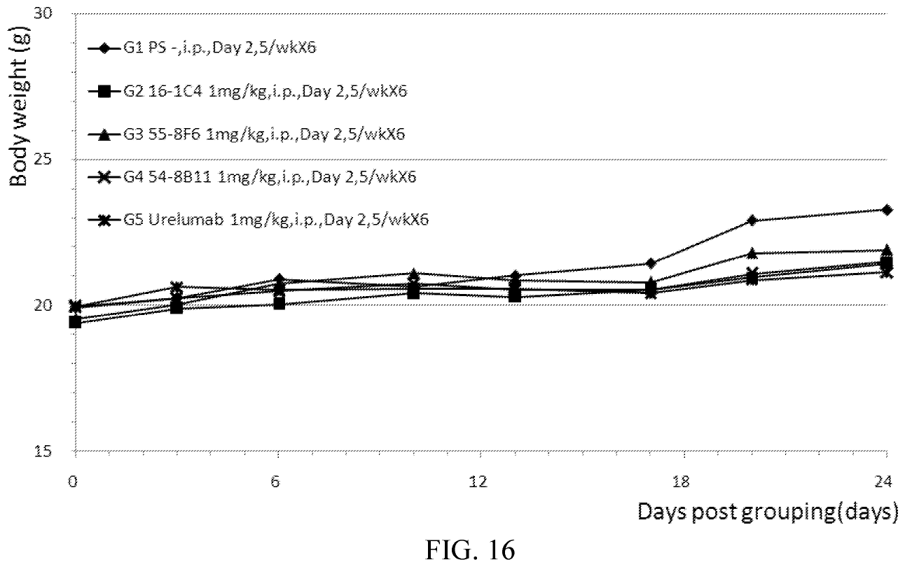

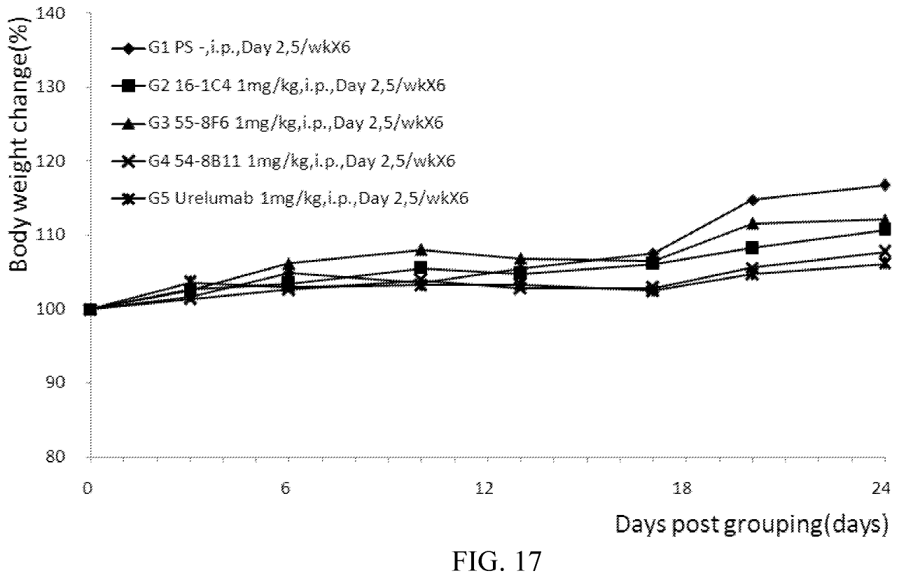

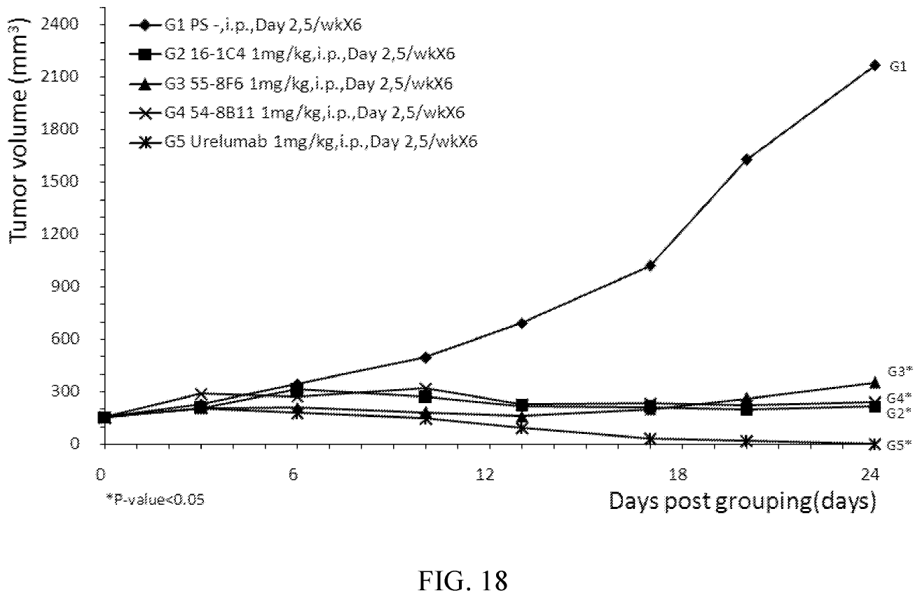

The weight of the mice was monitored during the entire treatment period. The average weight of mice in different groups all increased at the end of the treatment period (FIG. 16, and FIG. 17). No obvious difference in weight was observed among different groups. The results showed that these anti-h4-1BB antibodies were well tolerated and were not obviously toxic to the mice. As compared to the control group, 16-1C4, 55-8F6, and 54-8B11 all inhibited tumor growth (FIG. 18).

The TGI % at day 24 (24 days after grouping) was also calculated as shown in the table below.

TABLE 15

| | | Tumor volume(mm³) | | | | | | P value | |
|---|---|---|---|---|---|---|---|---|---|
| | | Day | Day | Day | Day | | | Body | Tumor |
| | | 0 | 10 | 17 | 24 | Survival | TGI % | weight | Volume |
| Control | G1 | 153 ± 22 | 495 ± 72 | 1019 ± 127 | 2166 ± 268 | 5/5 | n.a. | n.a. | n.a. |
| Treat | G2 | 153 ± 20 | 268 ± 55 | 210 ± 130 | 214 ± 181 | 5/5 | 96.9% | 0.104 | 3.1E−04 |
| | G3 | 153 ± 21 | 180 ± 59 | 200 ± 111 | 352 ± 220 | 5/5 | 90.1% | 0.149 | 7.9E−04 |
| | G4 | 153 ± 24 | 317 ± 82 | 237 ± 111 | 240 ± 164 | 5/5 | 95.7% | 0.051 | 2.8E−04 |
| | G5 | 152 ± 21 | 147 ± 19 | 30 ± 10 | 0 ± 0 | 5/5 | 107.6% | 0.024 | 4.0E−05 |

The results showed that anti-h4-1BB antibodies 16-1C4, 55-8F6, and 54-8B11 all significantly inhibited tumor growth.

### In Vivo Results for Mouse Anti-h4-1BB Antibodies 54-1A11 (“1A11”), 55-1E3 (“1E3”), 55-8H5 (“8H5”), and 56-2A6 (“2A6”)

B-h4-1BB mice were injected with physiological saline (PS) (G1), 1 mg/kg 54-1A11 (G2), 1 mg/kg 55-1E3 (G3), 1 mg/kg 55-8H5 (G4), and 1 mg/kg 56-2A6 (G5). 1 mg/kg Urelumab was also administered to the mice for comparison purpose (G6).

TABLE 16

| | | | | | | Total |
|---|---|---|---|---|---|---|
| | | | | | | No. of |
| | No. | | Dosage | | | admini- |
| Group | of mice | Antibodies | (mg/kg) | Route | Frequency | stration |
| G1 | 5 | PS (control) | — | i.p. | Day 1, 4/wk | 6 |
| G2 | 5 | 54-1A11 | 1 mg/kg | i.p. | Day 1, 4/wk | 6 |
| G3 | 5 | 55-1E3 | 1 mg/kg | i.p. | Day 1, 4/wk | 6 |
| G4 | 5 | 55-8H5 | 1 mg/kg | i.p. | Day 1, 4/wk | 6 |
| G5 | 5 | 56-2A6 | 1 mg/kg | i.p. | Day 1, 4/wk | 6 |
| G6 | 5 | Urelumab | 1 mg/kg | i.p. | Day 1, 4/wk | 6 |

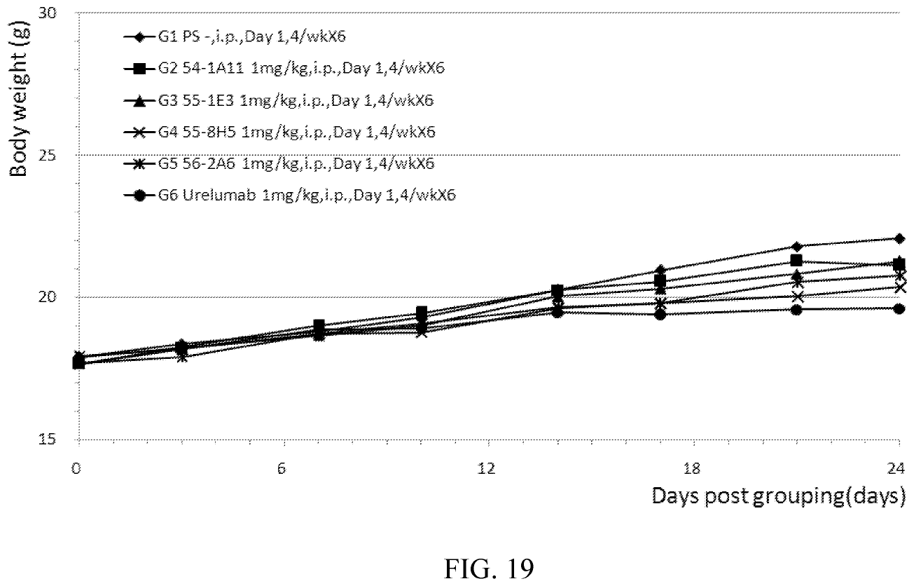

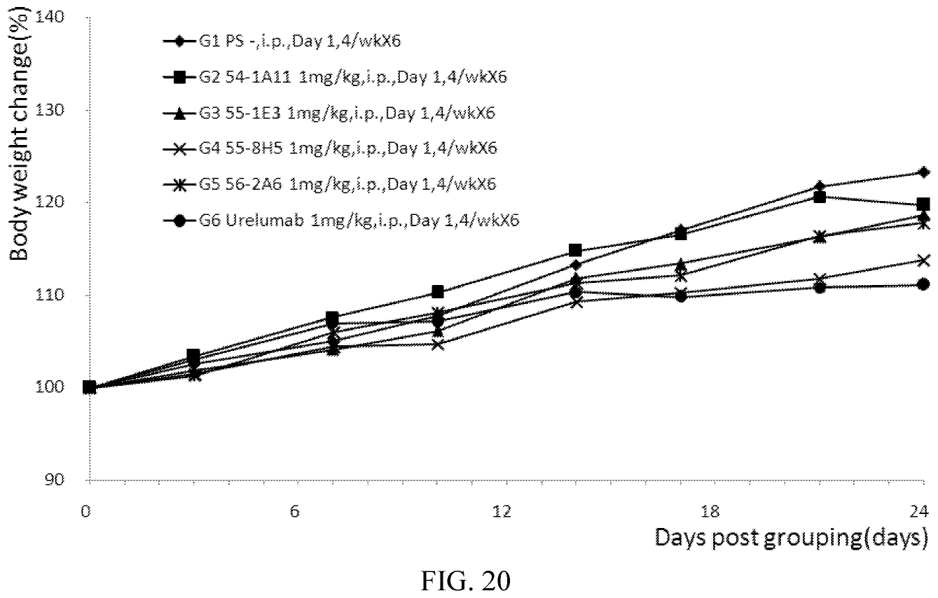

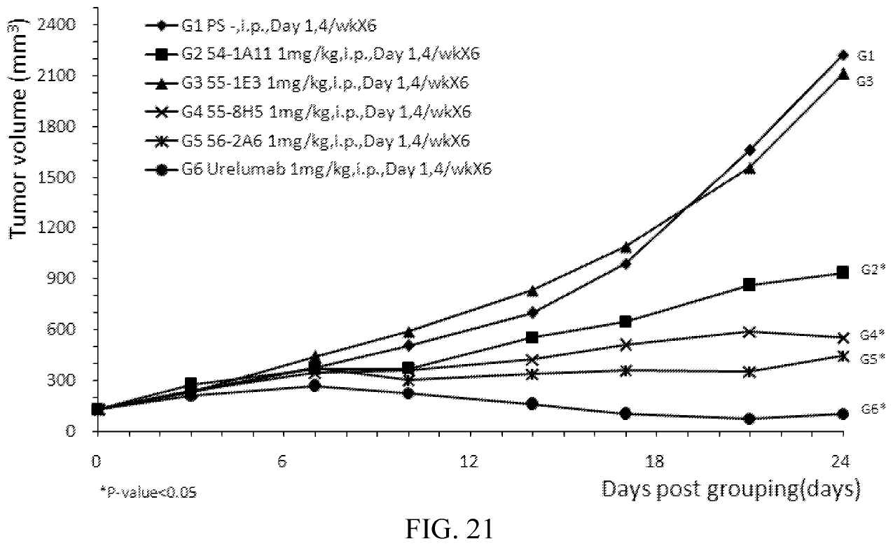

The weight of the mice was monitored during the entire treatment period. The average weight of mice in different groups all increased at the end of the treatment period (FIG. 19, and FIG. 20). No obvious difference in weight was observed among different groups. The results showed that these anti-h4-1BB antibodies were well tolerated and were not obviously toxic to the mice. The tumor size for each group is shown in FIG. 21.

The TGI % at day 24 (24 days after grouping) was also calculated as shown in the table below.

TABLE 17

| | | Tumor volume(mm³) | | | | | | P value | |
|---|---|---|---|---|---|---|---|---|---|
| | | Day | Day | Day | Day | | | Body | Tumor |
| | | 0 | 10 | 17 | 24 | Survival | TGI % | weight | Volume |
| Control | G1 | 131 ± 5 | 505 ± 92 | 991 ± 162 | 2226 ± 264 | 5/5 | n.a. | n.a. | n.a. |
| Treat | G2 | 132 ± 6 | 371 ± 42 | 646 ± 85 | 936 ± 190 | 5/5 | 61.6% | 0.419 | 0.004 |
| | G3 | 131 ± 10 | 590 ± 121 | 1091 ± 195 | 2113 ± 331 | 5/5 | 5.4% | 0.542 | 0.796 |
| | G4 | 132 ± 9 | 359 ± 120 | 510 ± 149 | 556 ± 159 | 5/5 | 79.7% | 0.155 | 6.3E−04 |
| | G5 | 131 ± 5 | 307 ± 28 | 364 ± 41 | 447 ± 132 | 5/5 | 84.9% | 0.247 | 0.008 |
| | G6 | 131 ± 8 | 222 ± 21 | 105 ± 18 | 102 ± 44 | 5/5 | 101.4% | 0.065 | 0.002 |

The results showed that anti-h4-1BB antibodies 54-1A11, 55-8H5, and 56-2A6 all significantly inhibited tumor growth. 55-1E3 was not effective in inhibiting tumor growth.

### In Vivo Results for Mouse Anti-h4-1BB Antibodies 58-4B8 (“4B8”), 69-3C2 (“3C2”), and 69-4B11 (“4B11”)

B-h4-1BB mice were injected with physiological saline (PS) (G1), 1 mg/kg 58-4B8 (G2), 1 mg/kg 69-3C2 (G3), and 1 mg/kg 69-4B11 (G4). 1 mg/kg Urelumab was also administered to the mice for comparison purpose (G5).

TABLE 18

| | | | | | | Total |
|---|---|---|---|---|---|---|
| | | | | | | No. of |
| | No. | | Dosage | | | admini- |
| Group | of mice | Antibodies | (mg/kg) | Route | Frequency | stration |
| G1 | 5 | PS (control) | — | i.p. | Day 1, 4/wk | 6 |
| G2 | 5 | 58-4B8 | 1 mg/kg | i.p. | Day 1, 4/wk | 6 |
| G3 | 5 | 69-3C2 | 1 mg/kg | i.p. | Day 1, 4/wk | 6 |
| G4 | 5 | 69-4B11 | 1 mg/kg | i.p. | Day 1, 4/wk | 6 |
| G5 | 5 | Urelumab | 1 mg/kg | i.p. | Day 1, 4/wk | 6 |

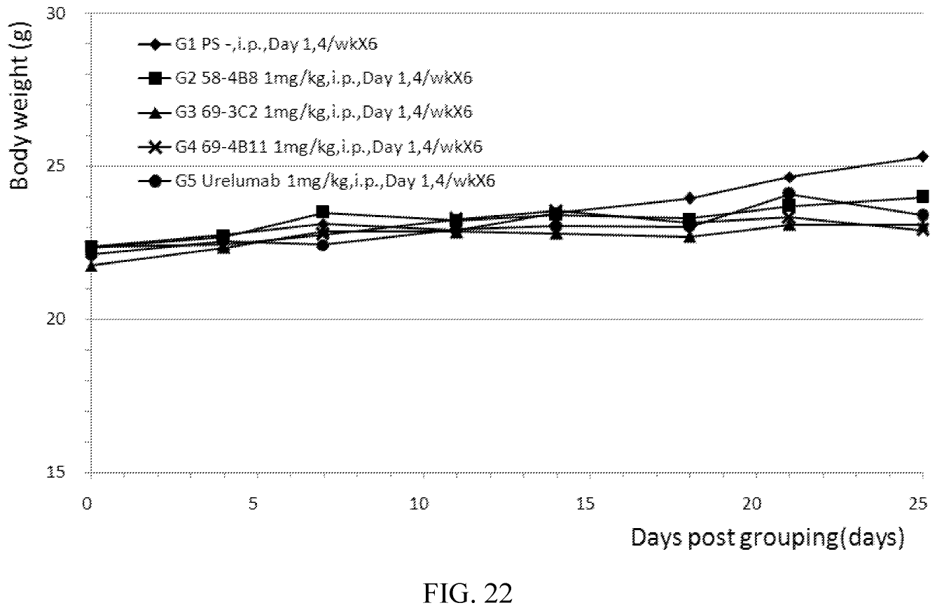

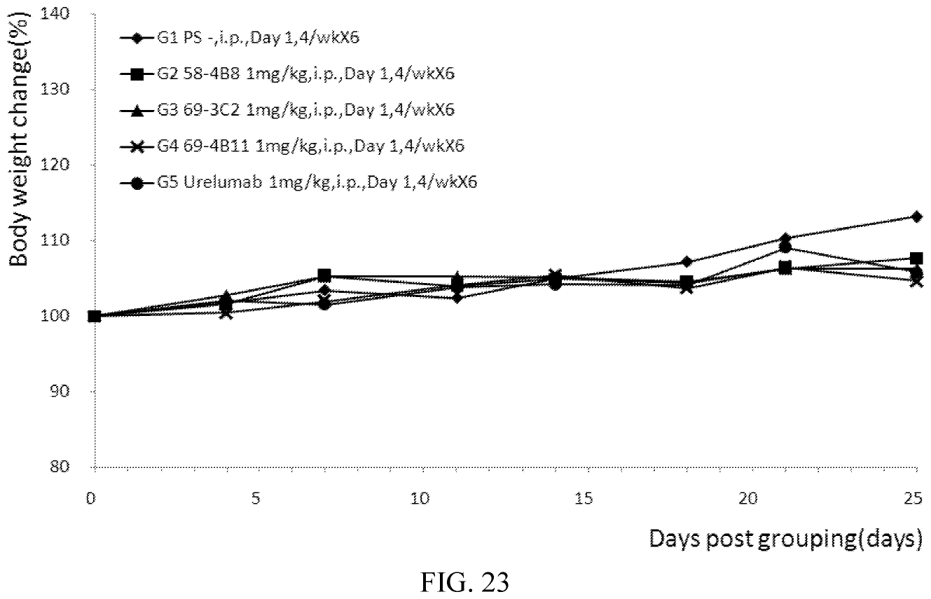

The weight of the mice was monitored during the entire treatment period. The weight of mice in different groups all increased (FIG. 22, and FIG. 23). No obvious difference in weight was observed among different groups. The results showed that these anti-h4-1BB antibodies were well tolerated and were not obviously toxic to the mice. The tumor size for each group is shown in FIG. 24.

The TGI % at day 25 (25 days after grouping) was also calculated as shown in the table below.

TABLE 19

| | | Tumor volume(mm³) | | | | | | P value | |
|---|---|---|---|---|---|---|---|---|---|
| | | Day | Day | Day | Day | | | Body | Tumor |
| | | 0 | 11 | 18 | 25 | Survival | TGI % | weight | Volume |
| Control | G1 | 111 ± 7 | 505 ± 65 | 1212 ± 119 | 2535 ± 230 | 5/5 | n.a. | n.a. | n.a. |
| Treat | G2 | 111 ± 8 | 448 ± 100 | 562 ± 158 | 728 ± 228 | 5/5 | 74.6% | 0.244 | 5.2E−04 |
| | G3 | 112 ± 9 | 286 ± 104 | 209 ± 112 | 118 ± 76 | 5/5 | 99.8% | 0.052 | 8.7E−06 |
| | G4 | 111 ± 6 | 311 ± 102 | 286 ± 169 | 369 ± 295 | 5/5 | 89.4% | 0.120 | 6.1E−04 |
| | G5 | 112 ± 7 | 289 ± 84 | 257 ± 109 | 371 ± 221 | 5/5 | 89.3% | 0.111 | 1.4E−04 |

The results showed that anti-h4-1BB antibodies 58-4B8, 69-3C2, and 69-4B11 all significantly inhibited tumor growth.

### Example 8. In Vivo Testing of Chimeric Anti-h4-1BB Antibodies

The chimeric anti-h4-1BB antibodies were tested in 4-1BB humanized mice (B-h4-1BB) to determine their effect on tumor growth in vivo. MC-38 cancer tumor cells (colon adenocarcinoma cell) were injected subcutaneously in B-h4-1BB mice. When the tumors in the mice reached a volume of 150±50 mm³, the mice were randomly placed into different groups based on the volume of the tumor.

### In Vivo Results for 30-5F9-mHvKv-IgG2, 30-5F9-mHvKv-IgG4, 16-1C4-mHvKv-IgG2, 16-1C4-mHvKv-IgG4, 16-1C4-mHvKv-IgG1

B-h4-1BB mice were injected with physiological saline (PS) (G1), 30-5F9-mHvKv-IgG2 (G2), 30-5F9-mHvKv-IgG4 (G3), 16-1C4-mHvKv-IgG2 (G4), 16-1C4-mHvKv-IgG4 (G5), 16-1C4-mHvKv-IgG1 (G6), 30-5F9 (G7), 16-1C4 (G8), utomilumab (G9), and urelumab (G10).

TABLE 20

| | No. of | | Dosage | | | Total No. of |
|---|---|---|---|---|---|---|
| Group | mice | Antibodies | (mg/kg) | Route | Frequency | administration |
| G1 | 5 | PS (control) | — | i.p. | Day 1, 4/wk | 6 |
| G2 | 5 | 30-5F9-mHvKv-IgG2 | 1 mg/kg | i.p. | Day 1, 4/wk | 6 |
| G3 | 5 | 30-5F9-mHvKv-IgG4 | 1 mg/kg | i.p. | Day 1, 4/wk | 6 |
| G4 | 5 | 16-1C4-mHvKv-IgG2 | 1 mg/kg | i.p. | Day 1, 4/wk | 6 |
| G5 | 5 | 16-1C4-mHvKv-IgG4 | 1 mg/kg | i.p. | Day 1, 4/wk | 6 |
| G6 | 5 | 16-1C4-mHvKv-IgG1 | 1 mg/kg | i.p. | Day 1, 5/wk | 6 |
| G7 | 5 | 30-5F9 | 1 mg/kg | i.p. | Day 1, 4/wk | 6 |
| G8 | 5 | 16-1C4 | 1 mg/kg | i.p. | Day 1, 4/wk | 6 |
| G9 | 5 | Utomilumab | 1 mg/kg | i.p. | Day 1, 4/wk | 6 |
| G10 | 5 | Urelumab | 1 mg/kg | i.p. | Day 1, 4/wk | 6 |

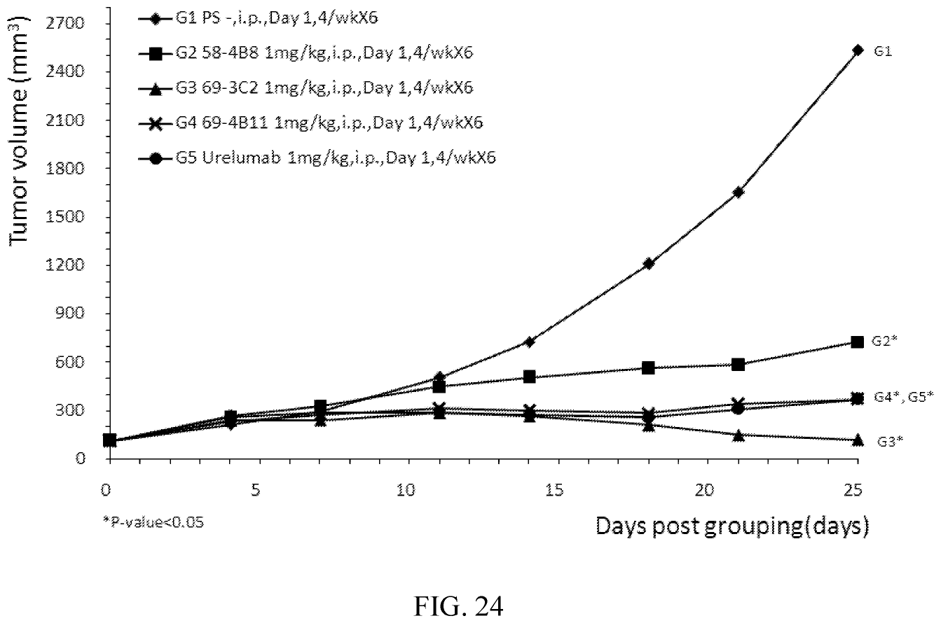

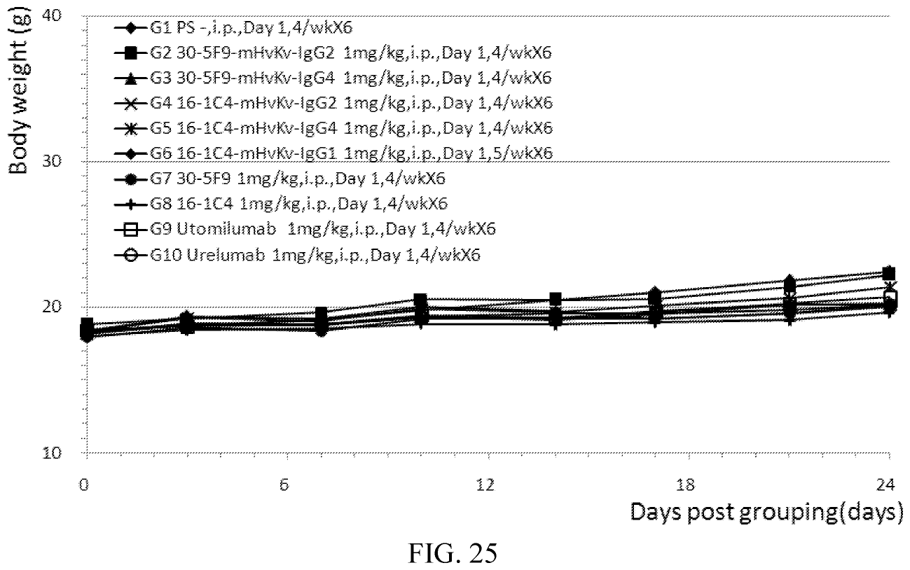

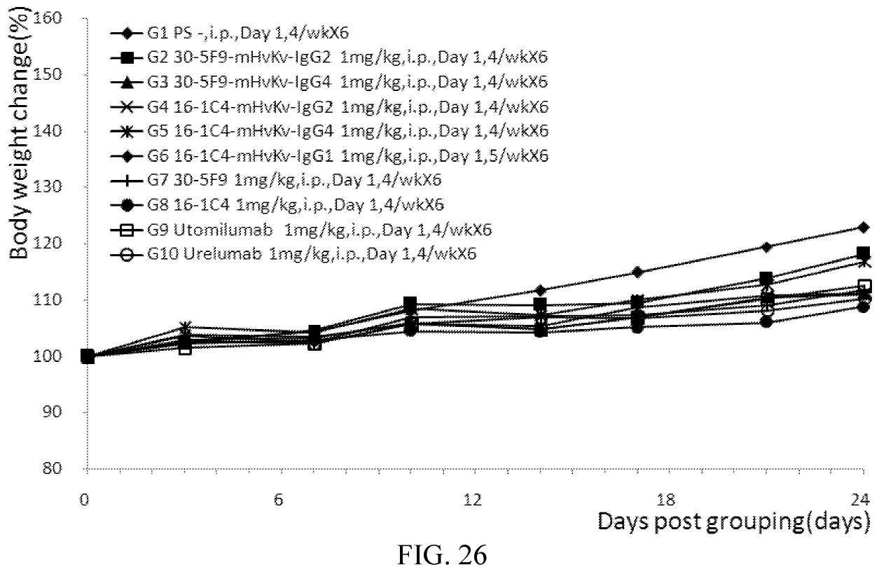

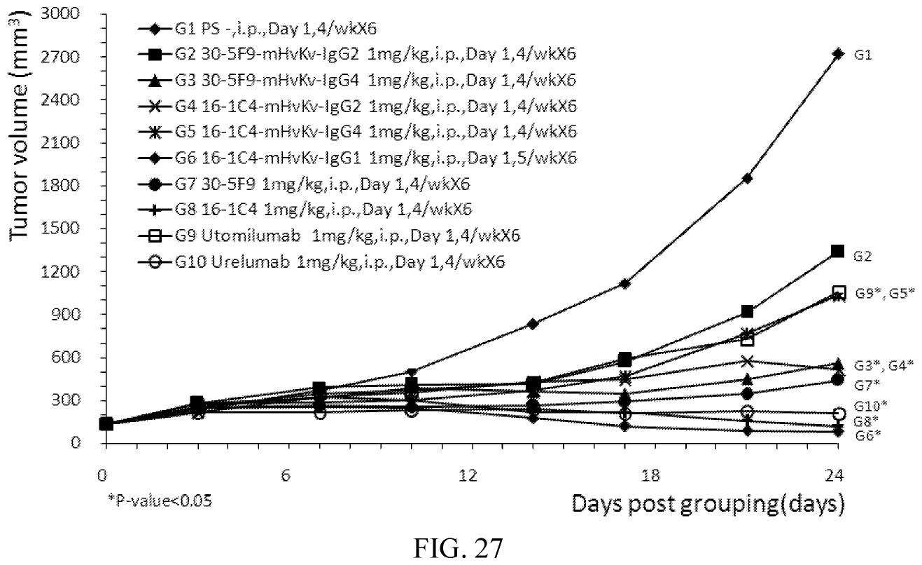

The weight of the mice was monitored during the entire treatment period. The average weight of mice in different groups all increased at the end of the treatment period (FIG. 25, and FIG. 26). No obvious difference in weight was observed among different groups. The results showed that these anti-h4-1BB antibodies were well tolerated and were not obviously toxic to the mice. The tumor size for each group is shown in FIG. 27.

The TGI % at day 24 (24 days after grouping) was also calculated as shown in the table below.

TABLE 21

| | | Tumor volume(mm³) | | | | | | P value | |
|---|---|---|---|---|---|---|---|---|---|
| | | Day | Day | Day | Day | | | Body | Tumor |
| | | 0 | 10 | 17 | 24 | Survival | TGI % | weight | Volume |
| Control | G1 | 138 ± 6 | 503 ± 87 | 1122 ± 225 | 2723 ± 546 | 5/5 | n.a. | n.a. | n.a. |
| Treat | G2 | 138 ± 14 | 411 ± 187 | 571 ± 368 | 1342 ± 1063 | 5/5 | 53.4% | 0.756 | 0.281 |
| | G3 | 138 ± 12 | 391 ± 31 | 347 ± 73 | 560 ± 257 | 5/5 | 83.7% | 0.075 | 0.007 |
| | G4 | 138 ± 9 | 354 ± 167 | 445 ± 259 | 517 ± 267 | 5/5 | 85.3% | 0.024 | 0.007 |
| | G5 | 138 ± 6 | 302 ± 60 | 472 ± 173 | 1036 ± 458 | 5/5 | 65.3% | 0.398 | 0.045 |
| | G6 | 138 ± 8 | 253 ± 40 | 122 ± 42 | 85 ± 30 | 5/5 | 102.0% | 0.036 | 0.001 |
| | G7 | 138 ± 12 | 255 ± 30 | 294 ± 87 | 442 ± 182 | 5/5 | 88.2% | 0.004 | 0.004 |
| | G8 | 138 ± 9 | 300 ± 117 | 217 ± 120 | 123 ± 70 | 5/5 | 100.6% | 0.020 | 0.001 |
| | G9 | 138 ± 7 | 372 ± 33 | 595 ± 18 | 1062 ± 98 | 5/5 | 64.3% | 0.019 | 0.017 |
| | G10 | 138 ± 8 | 235 ± 53 | 211 ± 125 | 210 ± 169 | 5/5 | 97.2% | 0.044 | 0.002 |

The results showed that all of these anti-h4-1BB antibodies can inhibit tumor growth. Particularly, The IgG1 subclass of 16-1C4-mHvKv (G6) had a higher TGI % than the IgG2 subclass (G4) and the IgG4 subclass (G5).

### In Vivo Results for 16-1C4-mHvKv-IgG2, 29-6A5-mHvKv-IgG2, 30-5F9-mHvKv-IgG2, 45-2B3-mHvKv-IgG2, 45-7E9-mHvKv-IgG2, 45-7G9-mHvKv-IgG2, 45-8E2-mHvKv-IgG2, and 45-8F1-mHvKv-IgG2

B-h4-1BB mice were injected with physiological saline (PS) (G1), 16-1C4-mHvKv-IgG2 (G2), 29-6A5-mHvKv-IgG2 (G3), 30-5F9-mHvKv-IgG2 (G4), 45-2B3-mHvKv-IgG2 (G5), 45-7E9-mHvKv-IgG2 (G6), 45-7G9-mHvKv-IgG2 (G7), 45-8E2-mHvKv-IgG2 (G8), 45-8F1-mHvKv-IgG2 (G9), and Urelumab (G10).

TABLE 22

| | No. of | | Dosage | | | Total No. of |
|---|---|---|---|---|---|---|
| Group | mice | Antibodies | (mg/kg) | Route | Frequency | administration |
| G1 | 5 | PS (control) | — | i.p. | Day 2,5/wk | 6 |
| G2 | 5 | 16-1C4-mHvKv-IgG2 | 1 mg/kg | i.p. | Day 2, 5/wk | 6 |
| G3 | 5 | 29-6A5-mHvKv-IgG2 | 1 mg/kg | i.p. | Day 2, 5/wk | 6 |
| G4 | 5 | 30-5F9-mHvKv-IgG2 | 1 mg/kg | i.p. | Day 2, 5/wk | 6 |
| G5 | 5 | 45-2B3-mHvKv-IgG2 | 1 mg/kg | i.p. | Day 2, 5/wk | 6 |
| G6 | 5 | 45-7E9-mHvKv-IgG2 | 1 mg/kg | i.p. | Day 2, 5/wk | 6 |
| G7 | 5 | 45-7G9-mHvKv-IgG2 | 1 mg/kg | i.p. | Day 2, 5/wk | 6 |
| G8 | 5 | 45-8E2-mHvKv-IgG2 | 1 mg/kg | i.p. | Day 2, 5/wk | 6 |
| G9 | 5 | 45-8F1-mHvKv-IgG2 | 1 mg/kg | i.p. | Day 2, 5/wk | 6 |
| G10 | 5 | Urelumab | 1 mg/kg | i.p. | Day 2, 5/wk | 6 |

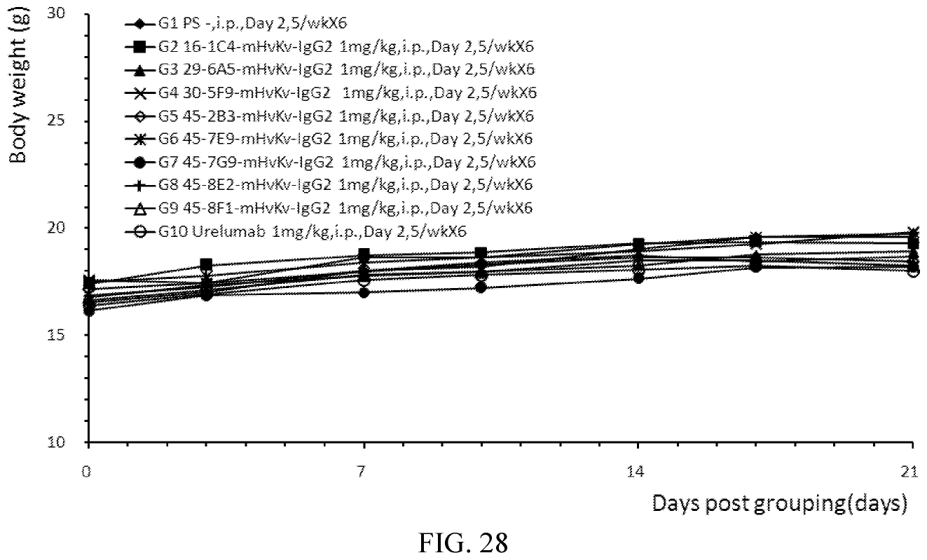

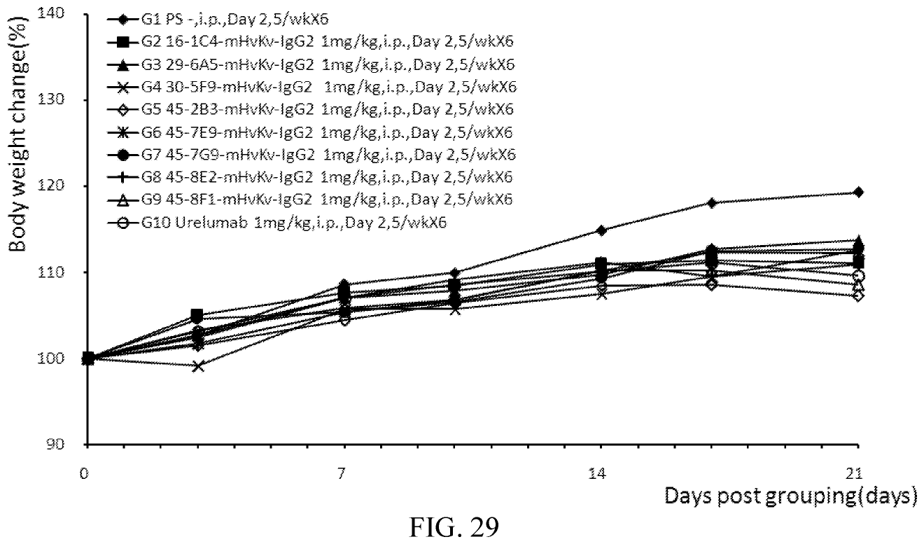

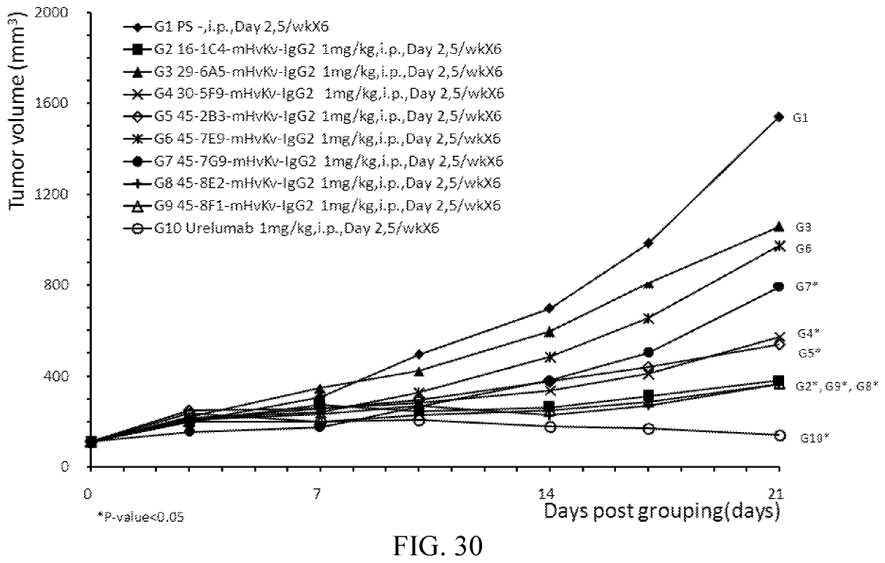

The weight of the mice was monitored during the entire treatment period. The average weight of mice in different groups all increased at the end of the treatment period (FIG. 28, and FIG. 29). No obvious difference in weight was observed among different groups. The results showed that these anti-h4-1BB antibodies were well tolerated and were not obviously toxic to the mice. The tumor size for each group is shown in FIG. 30.

The TGI % at day 21 (21 days after grouping) was also calculated as shown in the table below.

TABLE 23

| | | Tumor volume(mm³) | | | | | | P value | |
|---|---|---|---|---|---|---|---|---|---|
| | | Day | Day | Day | Day | | | Body | Tumor |
| | | 0 | 7 | 14 | 21 | Survival | TGI % | weight | Volume |
| Control | G1 | 111 ± 4 | 308 ± 41 | 698 ± 95 | 1543 ± 239 | 5/5 | n.a. | n.a. | n.a. |
| Treat | G2 | 111 ± 8 | 275 ± 43 | 262 ± 91 | 381 ± 195 | 5/5 | 75.3% | 0.586 | 0.006 |
| | G3 | 111 ± 6 | 348 ± 59 | 596 ± 122 | 1060 ± 194 | 5/5 | 31.3% | 0.423 | 0.156 |
| | G4 | 111 ± 8 | 258 ± 37 | 338 ± 39 | 574 ± 94 | 5/5 | 62.8% | 0.960 | 0.005 |
| | G5 | 111 ± 6 | 258 ± 27 | 378 ± 59 | 539 ± 107 | 5/5 | 65.1% | 0.332 | 0.005 |
| | G6 | 111 ± 7 | 241 ± 51 | 487 ± 245 | 974 ± 598 | 5/5 | 36.9% | 0.869 | 0.403 |
| | G7 | 111 ± 5 | 175 ± 24 | 382 ± 82 | 794 ± 194 | 5/5 | 48.6% | 0.233 | 0.041 |
| | G8 | 111 ± 8 | 233 ± 33 | 229 ± 63 | 366 ± 181 | 5/5 | 76.3% | 0.345 | 0.004 |
| | G9 | 111 ± 7 | 201 ± 33 | 252 ± 106 | 367 ± 227 | 5/5 | 76.2% | 0.173 | 0.007 |
| | G10 | 111 ± 7 | 198 ± 49 | 180 ± 72 | 140 ± 76 | 5/5 | 90.9% | 0.104 | 0.001 |

The results showed that all of these chimeric antibodies can inhibit tumor growth to different extents.

### Example 9. In Vivo Testing of Humanized Anti-h4-1BB Antibodies

The humanized anti-h4-1BB antibodies were tested in 4-1BB humanized mice (B-h4-1BB) to demonstrate their effect on tumor growth in vivo. MC-38 cancer tumor cells (colon adenocarcinoma cell) were injected subcutaneously in B-h4-1BB mice. When the tumors in the mice reached a volume of 150±50 mm³, the mice were randomly placed into different groups based on the volume of the tumor.

### In Vivo Results for Humanized Anti-4-1BB Antibodies 6A5-H1K2-IgG2, 6A5-H1K3-IgG2, and 6A5-H2K2-IgG2

In G1 group, B-h4-1BB mice were injected with physiological saline (PS) as a control. In the treatment groups, 6A5-H1K2-IgG2 (G2), 6A5-H1K3-IgG2 (G3), 6A5-H2K2-IgG2 (G4), 6A5 (G5), and urelumab (G6) were administered to the mice.

TABLE 24

| | No. of | | | | | Total No. of |
|---|---|---|---|---|---|---|
| Group | mice | Antibodies | Dosage (mg/kg) | Route | Frequency | administration |
| G1 | 9 | PS (control) | — | i.p. | Day 1, 4/wk | 6 |
| G2 | 9 | 6A5-H1K2-IgG2 | 1 mg/kg | i.p. | Day 1, 4/wk | 6 |
| G3 | 9 | 6A5-H1K3-IgG2 | 1 mg/kg | i.p. | Day 1, 4/wk | 6 |
| G4 | 9 | 6A5-H2K2-IgG2 | 1 mg/kg | i.p. | Day 1, 4/wk | 6 |
| G5 | 9 | 29-6A5 | 1 mg/kg | i.p. | Day 1, 4/wk | 6 |
| G6 | 9 | Urelumab | l mg/kg | i.p. | Day 1, 4/wk | 6 |

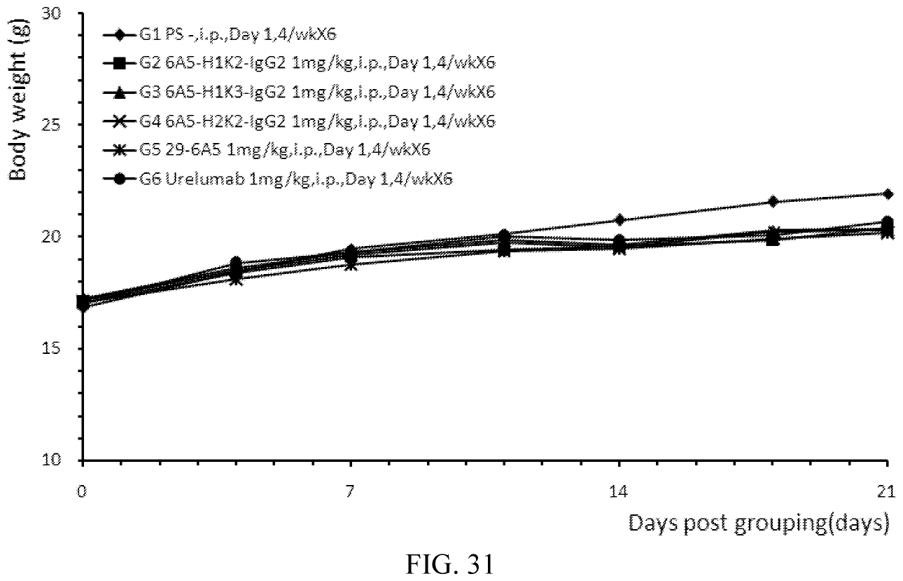

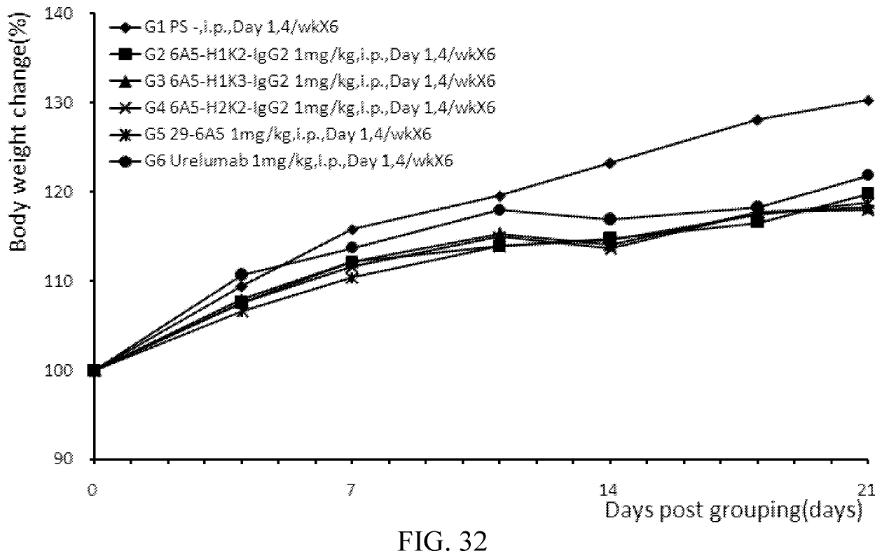

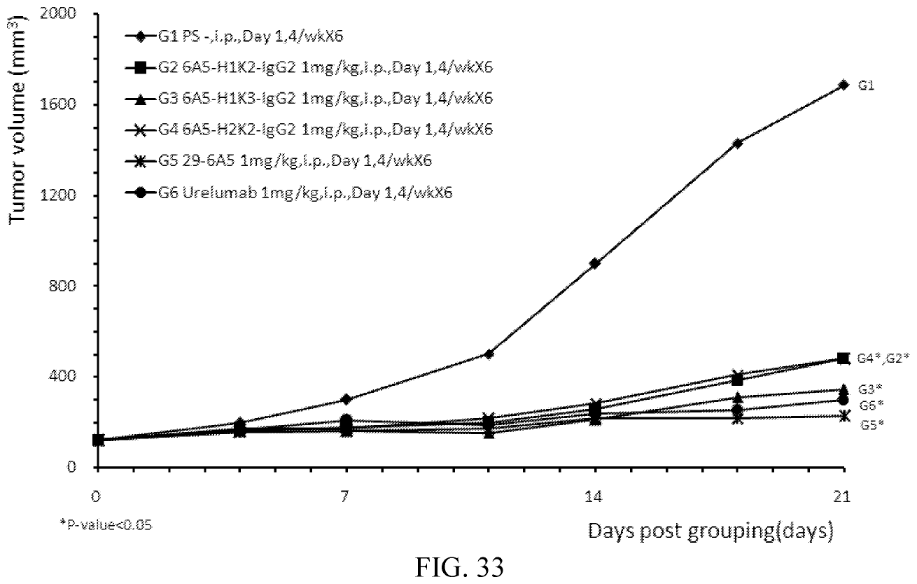

The weight of the mice was monitored during the entire treatment period. The average weight of mice in different groups all increased at the end of the treatment period (FIG. 31, and FIG. 32). No obvious difference in weight was observed among different groups. The results showed that these anti-h4-1BB antibodies were well tolerated and were not obviously toxic to the mice. The tumor size for each group is shown in FIG. 33.

The TGI % at day 21 (21 days after grouping) was also calculated as shown in the table below.

TABLE 25

| | | Tumor volume(mm³) | | | | | | P value | |
|---|---|---|---|---|---|---|---|---|---|
| | | Day | Day | Day | Day | | | Body | Tumor |
| | | 0 | 7 | 14 | 21 | Survival | TGI % | weight | Volume |
| Control | G1 | 119 ± 6 | 300 ± 52 | 900 ± 202 | 1688 ± 341 | 9/9 | n.a. | n.a. | n.a. |
| Treat | G2 | 119 ± 7 | 179 ± 18 | 257 ± 49 | 480 ± 132 | 9/9 | 71.6% | 0.086 | 0.005 |
| | G3 | 119 ± 7 | 160 ± 20 | 211 ± 29 | 346 ± 61 | 9/9 | 79.5% | 0.084 | 1.4E−03 |
| | G4 | 119 ± 7 | 172 ± 26 | 284 ± 56 | 480 ± 118 | 9/9 | 71.6% | 0.111 | 0.004 |
| | G5 | 119 ± 6 | 160 ± 29 | 219 ± 66 | 227 ± 97 | 9/9 | 86.6% | 0.065 | 8.1E−04 |
| | G6 | 119 ± 7 | 210 ± 29 | 239 ± 65 | 298 ± 130 | 9/9 | 82.3% | 0.131 | 0.002 |

The results showed that all of these humanized anti-h4-1BB antibodies (G2, G3, and G4) can inhibit tumor growth.

### In Vivo Results for Humanized Anti-4-1BB Antibodies 1C4-H1K1-IgG4, 1C4-H1K2-IgG4, 5F9-H1K1-IgG4, and 30-5F9-H1K2-IgG4

In G1 group, B-h4-1BB mice were injected with physiological saline (PS) as a control. In treatment groups, 1C4-H1K1-IgG4 (G2), 1C4-H1K2-IgG4 (G3), 5F9-H1K1-IgG4 (G4), 5F9-H1K2-IgG4 (G5), 16-1C4 (G6), 30-5F9 (G7) and urelumab (G8) were administered to the mice.

TABLE 26

| | No. of | | | | | Total No. of |
|---|---|---|---|---|---|---|
| Group | mice | Antibodies | Dosage (mg/kg) | Route | Frequency | administration |
| G1 | 5 | PS (control) | — | i.p. | Day 1, 4/wk | 6 |
| G2 | 5 | 1C4-H1K1-IgG4 | 1 mg/kg | i.p. | Day 2, 5/wk | 6 |
| G3 | 5 | 1C4-H1K2-IgG4 | 1 mg/kg | i.p. | Day 2, 5/wk | 6 |
| G4 | 5 | 5F9-H1K1-IgG4 | 1 mg/kg | i.p. | Day 2, 5/wk | 6 |
| G5 | 5 | 5F9-H1K2-IgG4 | 1 mg/kg | i.p. | Day 2, 5/wk | 6 |
| G6 | 5 | 16-1C4 | 1 mg/kg | i.p. | Day 2, 5/wk | 6 |
| G7 | 5 | 30-5F9 | 1 mg/kg | i.p. | Day 2, 5/wk | 6 |
| G8 | 5 | Urelumab | 1 mg/kg | i.p. | Day 2, 5/wk | 6 |

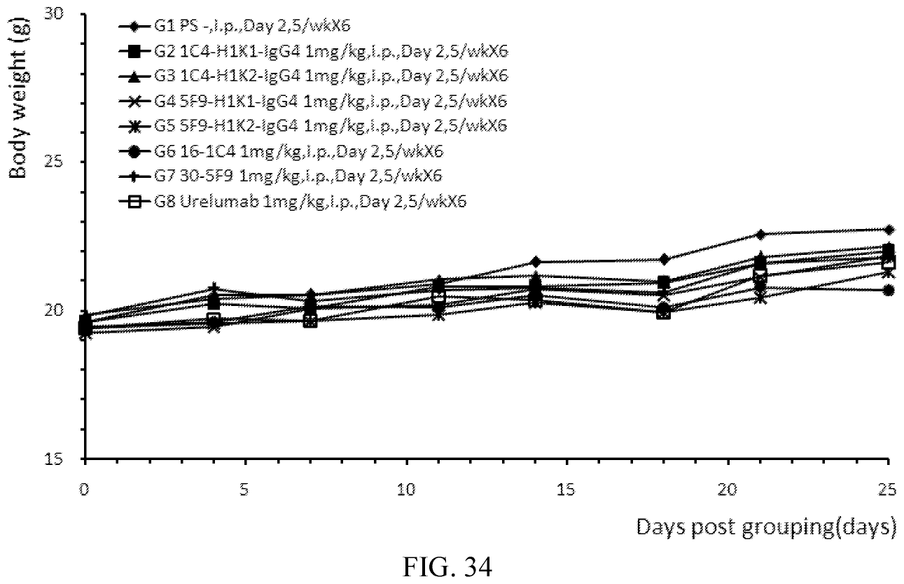

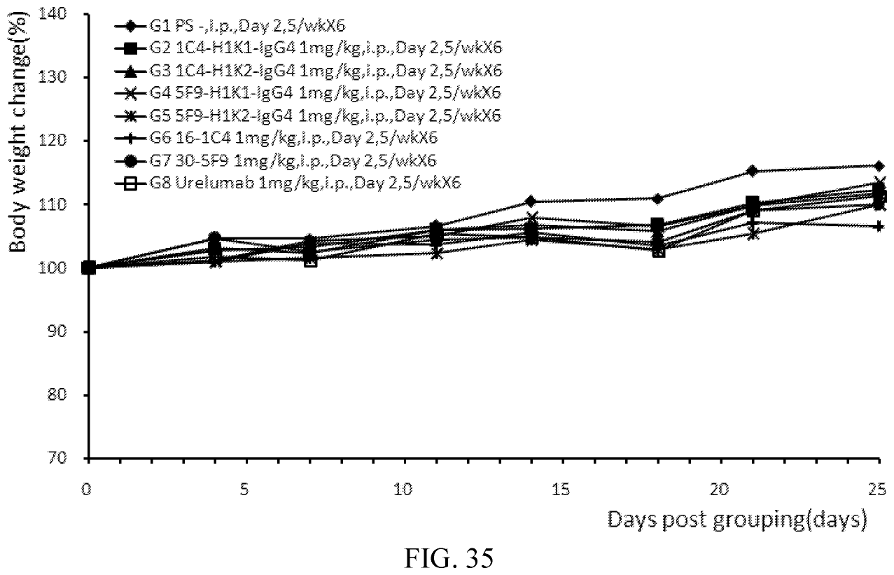

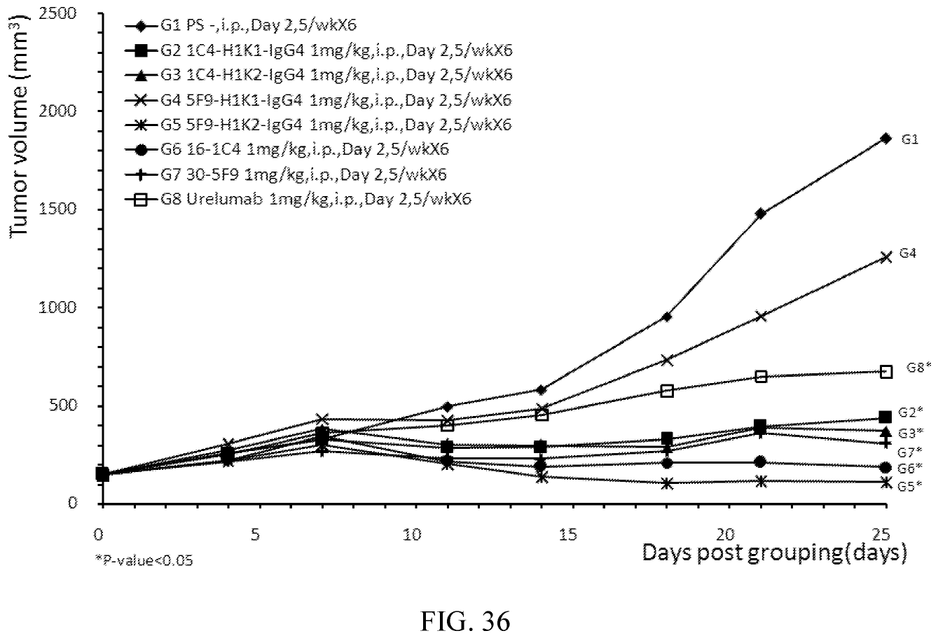

The weight of the mice was monitored during the entire treatment period. The average weight of mice in different groups all increased at the end of the treatment period (FIG. 34, and FIG. 35). No obvious difference in weight was observed among different groups. The results showed that these anti-h4-1BB antibodies were well tolerated and were not obviously toxic to the mice. The tumor size for each group is shown in FIG. 36.

The TGI % at day 25 (25 days after grouping) was also calculated as shown in the table below:

TABLE 27

| | | Tumor volume(mm³) | | | | | | P value | |
|---|---|---|---|---|---|---|---|---|---|
| | | Day | Day | Day | Day | | | Body | Tumor |
| | | 0 | 11 | 18 | 25 | Survival | TGI % | weight | Volume |
| Control | G1 | 150 ± 5 | 495 ± 88 | 955 ± 236 | 1863 ± 340 | 5/5 | n.a. | n.a. | n.a. |
| Treat | G2 | 150 ± 6 | 287 ± 20 | 330 ± 86 | 436 ± 166 | 5/5 | 76.6% | 0.579 | 0.005 |
| | G3 | 150 ± 6 | 302 ± 22 | 289 ± 57 | 371 ± 115 | 5/5 | 80.1% | 0.640 | 0.003 |
| | G4 | 150 ± 6 | 426 ± 91 | 733 ± 268 | 1259 ± 609 | 5/5 | 32.4% | 0.538 | 0.412 |
| | G5 | 150 ± 6 | 201 ± 46 | 107 ± 65 | 109 ± 83 | 5/5 | 94.2% | 0.270 | 1.0E−03 |
| | G6 | 150 ± 6 | 214 ± 28 | 208 ± 38 | 186 ± 70 | 5/5 | 90.0% | 0.117 | 1.3E−03 |
| | G7 | 150 ± 5 | 232 ± 28 | 266 ± 79 | 307 ± 140 | 5/5 | 83.5% | 0.456 | 0.003 |
| | G8 | 150 ± 7 | 400 ± 89 | 576 ± 171 | 672 ± 294 | 5/5 | 63.9% | 0.381 | 0.029 |

The results showed that these humanized anti-h4-1BB antibodies have different tumor inhibitory effects.

### Example 10. In Vivo Testing of IgG1, IgG2, and IgG4 Antibodies

MC-38 cancer tumor cells (colon adenocarcinoma cell) were injected subcutaneously in B-h4-1BB mice. When the tumors in the mice reached a volume of 150±50 mm³, the mice were randomly placed into different groups based on the volume of the tumor.

In G1 group, B-h4-1BB mice were injected with physiological saline (PS) as a control. Chimeric anti-h4-1BB antibodies 16-1C4-mHvKv-IgG1 (G2), 16-1C4-mHvKv-IgG2 (G3), 16-1C4-mHvKv-IgG4 (G4), mouse anti-h4-1BB antibodies 16-1C4 (G5), and urelumab (G6) were administered to the mice.

TABLE 28

| | No. of | | Dosage | | | Total No. of |
|---|---|---|---|---|---|---|
| Group | mice | Antibodies | (mg/kg) | Route | Frequency | administration |
| G1 | 7 | PS (control) | — | i.p. | Day 1, 4/wk | 6 |
| G2 | 7 | 16-1C4-mHvKv-IgG1 | 1 mg/kg | i.p. | Day 1, 4/wk | 6 |
| G3 | 7 | 16-1C4-mHvKv-IgG2 | 1 mg/kg | i.p. | Day 1, 4/wk | 6 |
| G4 | 7 | 16-1C4-mHvKv-IgG4 | 1 mg/kg | i.p. | Day 1, 4/wk | 6 |
| G5 | 7 | 16-1C4 | 1 mg/kg | i.p. | Day 1, 4/wk | 6 |
| G6 | 7 | Urelumab | 1 mg/kg | i.p. | Day 1, 4/wk | 6 |

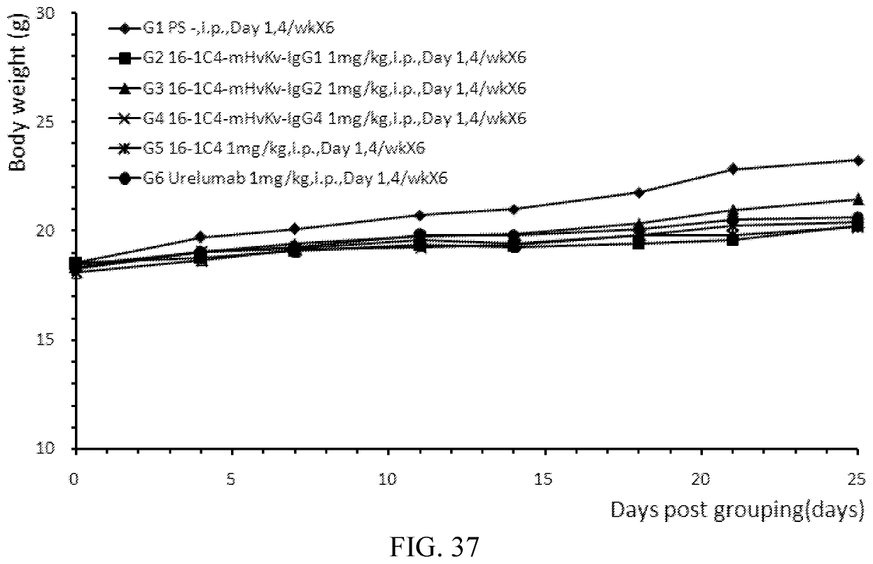

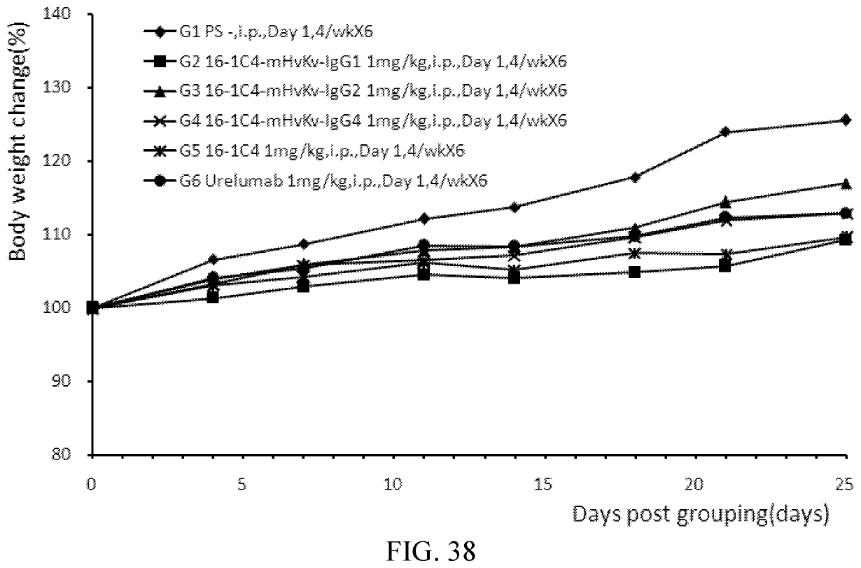

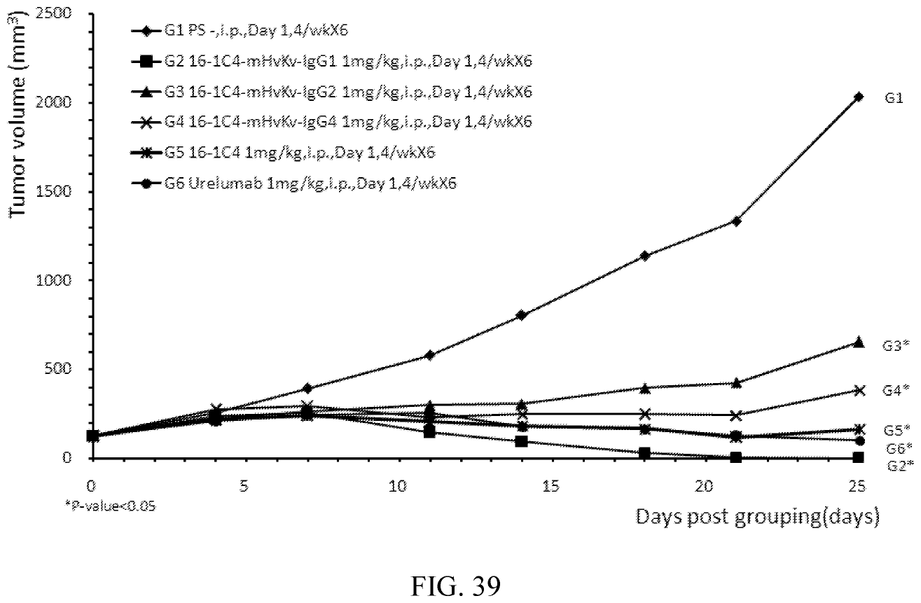

The weight of the mice was monitored during the entire treatment period. The average weight of mice in different groups all increased at the end of the treatment period (FIG. 37, and FIG. 38). No obvious difference in weight was observed among different groups. The results showed that these anti-h4-1BB antibodies were well tolerated and were not obviously toxic to the mice. The tumor size for each group is shown in FIG. 39.

The TGI % at day 25 (25 days after grouping) was also calculated as shown in the table below.

TABLE 29

| | | Tumor volume(mm³) | | | | | | P value | |
|---|---|---|---|---|---|---|---|---|---|
| | | Day | Day | Day | Day | | | Body | Tumor |
| | | 0 | 11 | 18 | 25 | Survival | TGI % | weight | Volume |
| Control | G1 | 124 ± 7 | 580 ± 74 | 1139 ± 121 | 2035 ± 176 | 7/7 | n.a. | n.a. | n.a. |
| Treat | G2 | 125 ± 7 | 147 ± 34 | 28 ± 14 | 0 ± 0 | 7/7 | 100.0% | 0.001 | 7.4E−08 |
| | G3 | 125 ± 6 | 297 ± 75 | 396 ± 148 | 655 ± 250 | 7/7 | 67.8% | 0.052 | 7.1E−04 |
| | G4 | 125 ± 6 | 230 ± 42 | 247 ± 73 | 383 ± 150 | 7/7 | 81.2% | 0.001 | 1.2E−05 |
| | G5 | 124 ± 7 | 205 ± 52 | 166 ± 92 | 164 ± 109 | 7/7 | 91.9% | 0.001 | 1.1E−06 |
| | G6 | 124 ± 7 | 254 ± 89 | 167 ± 102 | 102 ± 63 | 7/7 | 95.0% | 0.001 | 2.5E−07 |

The results showed that all of these anti-h4-1BB antibodies inhibited tumor growth. Particularly, IgG1 subclass had higher TGI % than the IgG2 and IgG4 subclasses.

### Example 11. In Vivo Testing of Chimeric Anti-4-1BB IgG2 Antibodies

MC-38 cancer tumor cells (colon adenocarcinoma cell) were injected subcutaneously in B-h4-1BB mice. When the tumors in the mice reached a volume of 150±50 mm³, the mice were randomly placed into different groups based on the volume of the tumor.

In G1 group, B-h4-1BB mice were injected with physiological saline (PS) as a control. In treatment groups, chimeric anti-h4-1BB antibodies 54-8B11-mHvKv-IgG2 (G2), 55-8F6-mHvKv-IgG2 (G3), 56-2A6-mHvKv-IgG2 (G4), 69-3C2-mHvKv-IgG2 (G5), 61-6A7-mHvKv-IgG2 (G6), 70-6F10-mHvKv-IgG2 (G7), 70-3F9-mHvKv-IgG2 (G8), 45-4B9-mHvkv-IgG2 (G9), and also urelumab (G10) were administered to the mice through intraperitoneal (i.p.) injection.

TABLE 30

| | No. of | | Dosage | | | Total No. of |
|---|---|---|---|---|---|---|
| Group | mice | Antibodies | (mg/kg) | Route | Frequency | administration |
| G1 | 5 | PS (control) | — | i.p. | Day 2, 5/wk | 6 |
| G2 | 5 | 54-8B11-mHvKv-IgG2 | 1 mg/kg | i.p. | Day 2, 5/wk | 6 |
| G3 | 5 | 55-8F6-mHvKv-IgG2 | 1 mg/kg | i.p. | Day 2, 5/wk | 6 |
| G4 | 5 | 56-2A6-mHvKv-IgG2 | 1 mg/kg | i.p. | Day 2, 5/wk | 6 |
| G5 | 5 | 69-3C2-mHvKv-IgG2 | 1 mg/kg | i.p. | Day 2, 5/wk | 6 |
| G6 | 5 | 61-6A7-mHvKv-IgG2 | 1 mg/kg | i.p. | Day 2, 5/wk | 6 |
| G7 | 5 | 70-6F10-mHvKv-IgG2 | 1 mg/kg | i.p. | Day 2, 5/wk | 6 |
| G8 | 5 | 70-3F9-mHvKv-IgG2 | 1 mg/kg | i.p. | Day 2, 5/wk | 6 |
| G9 | 5 | 45-4B9-mHvkv-IgG2 | 1 mg/kg | i.p. | Day 2, 5/wk | 6 |
| G10 | 5 | Urelumab | 1 mg/kg | i.p. | Day 2, 5/wk | 6 |

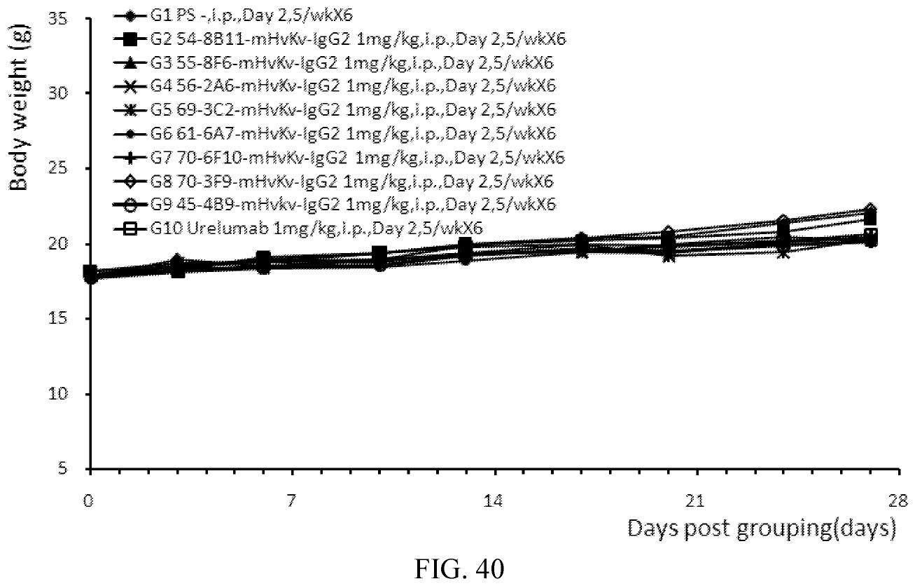

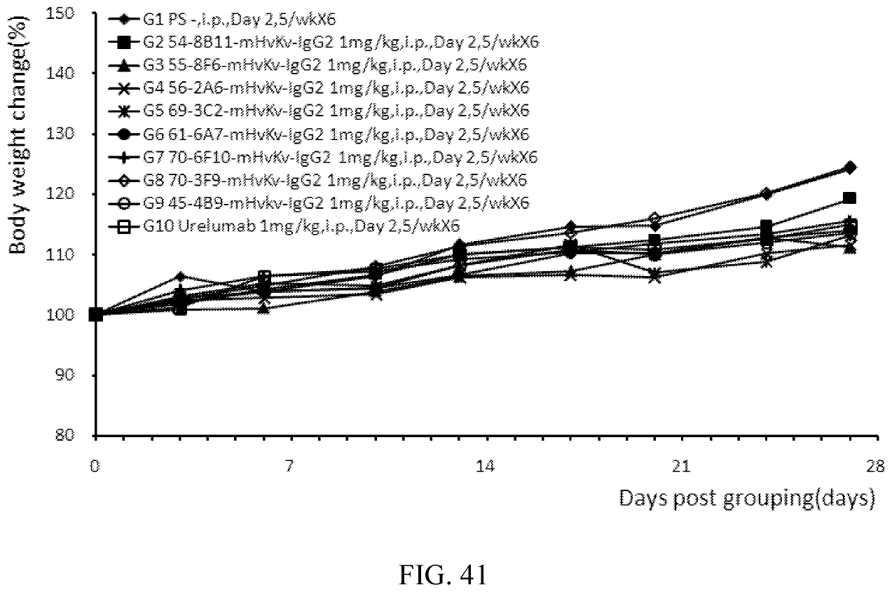

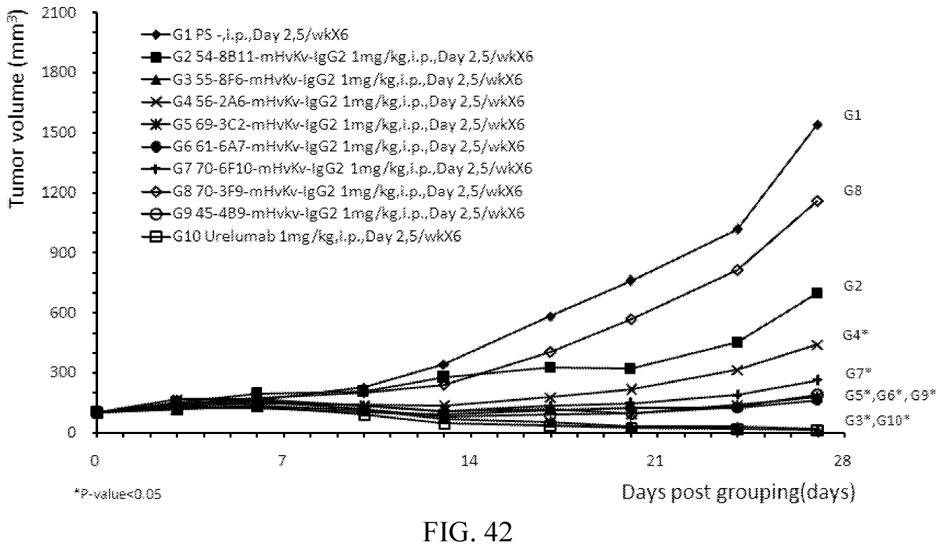

The weight of the mice was monitored during the entire treatment period. The average weight of mice in different groups all increased at the end of the treatment period (FIG. 40, and FIG. 41). No obvious difference in weight was observed among the different groups. The results showed that these anti-h4-1BB antibodies were well tolerated and were not obviously toxic to the mice. The tumor size for each group is shown in FIG. 42.

The TGI % at day 27 (27 days after grouping) was also calculated as shown in the table below.

TABLE 31

| | | Tumor volume(mm³) | | | | | | P value | |
|---|---|---|---|---|---|---|---|---|---|
| | | Day | Day | Day | Day | | | Body | Tumor |
| | | 0 | 10 | 20 | 27 | Survival | TGI % | weight | Volume |
| Control | G1 | 105 ± 4 | 229 ± 46 | 760 ± 219 | 1542 ± 477 | 5/5 | n.a. | n.a. | n.a. |
| Treat | G2 | 105 ± 9 | 207 ± 40 | 320 ± 111 | 699 ± 221 | 5/5 | 58.7% | 0.610 | 0.147 |
| | G3 | 105 ± 5 | 114 ± 8 | 32 ± 15 | 18 ± 18 | 5/5 | 106.0% | 0.084 | 0.013 |
| | G4 | 105 ± 9 | 141 ± 36 | 217 ± 123 | 444 ± 305 | 5/5 | 76.4% | 0.021 | 0.088 |
| | G5 | 105 ± 6 | 138 ± 49 | 100 ± 64 | 178 ± 128 | 5/5 | 94.9% | 0.072 | 0.025 |
| | G6 | 105 ± 9 | 105 ± 10 | 123 ± 38 | 164 ± 63 | 5/5 | 95.9% | 0.016 | 0.021 |
| | G7 | 105 ± 11 | 135 ± 39 | 148 ± 65 | 262 ± 117 | 5/5 | 89.1% | 0.217 | 0.031 |
| | G8 | 105 ± 3 | 203 ± 45 | 569 ± 265 | 1160 ± 440 | 5/5 | 26.6% | 0.765 | 0.572 |
| | G9 | 105 ± 7 | 120 ± 48 | 96 ± 78 | 189 ± 180 | 5/5 | 94.2% | 0.070 | 0.029 |
| | G10 | 105 ± 6 | 90 ± 19 | 29 ± 12 | 17 ± 11 | 5/5 | 106.1% | 0.056 | 0.007 |

The results showed that these chimeric anti-h4-1BB antibodies had different tumor inhibitory effects.

### Example 12. In Vivo Testing of Humanized and Chimeric Anti-4-1BB IgG2 Antibodies

MC-38 cancer tumor cells were injected subcutaneously in B-h4-1BB mice. When the tumors in the mice reached a volume of 150±50 mm³, the mice were randomly placed into different groups based on the volume of the tumor, and were administered with different antibodies as shown in the table below.

TABLE 32

| | No. of | | Dosage | | | Total No. of |
|---|---|---|---|---|---|---|
| Group | mice | Antibodies | (mg/kg) | Route | Frequency | administration |
| G1 | 8 | PS (control) | — | i.p. | Day 1, 4/wk | 6 |
| G2 | 8 | 6A5-H1K2-IgG2 | 1 mg/kg | i.p. | Day 1, 4/wk | 6 |
| G3 | 8 | 6A5-H1K3-IgG2 | 1 mg/kg | i.p. | Day 1, 4/wk | 6 |
| G4 | 8 | 1C4-H1K1-IgG2 | 1 mg/kg | i.p. | Day 1, 4/wk | 6 |
| G5 | 8 | 1C4-H1K2-IgG2 | 1 mg/kg | i.p. | Day 1, 4/wk | 6 |
| G6 | 8 | 5F9-H1K1-IgG2 | 1 mg/kg | i.p. | Day 1, 4/wk | 6 |
| G7 | 8 | 5F9-H1K2-IgG2 | 1 mg/kg | i.p. | Day 1, 4/wk | 6 |
| G8 | 8 | 16-1C4-mHvKv-IgG2 | 1 mg/kg | i.p. | Day 1, 4/wk | 6 |
| G9 | 8 | 29-6A5-mHvKv-IgG2 | 1 mg/kg | i.p. | Day 1, 4/wk | 6 |
| G10 | 8 | 30-5F9-mHvKv-IgG2 | 1 mg/kg | i.p. | Day 1, 4/wk | 6 |
| G11 | 8 | Urelumab | 1 mg/kg | i.p. | Day 1, 4/wk | 6 |

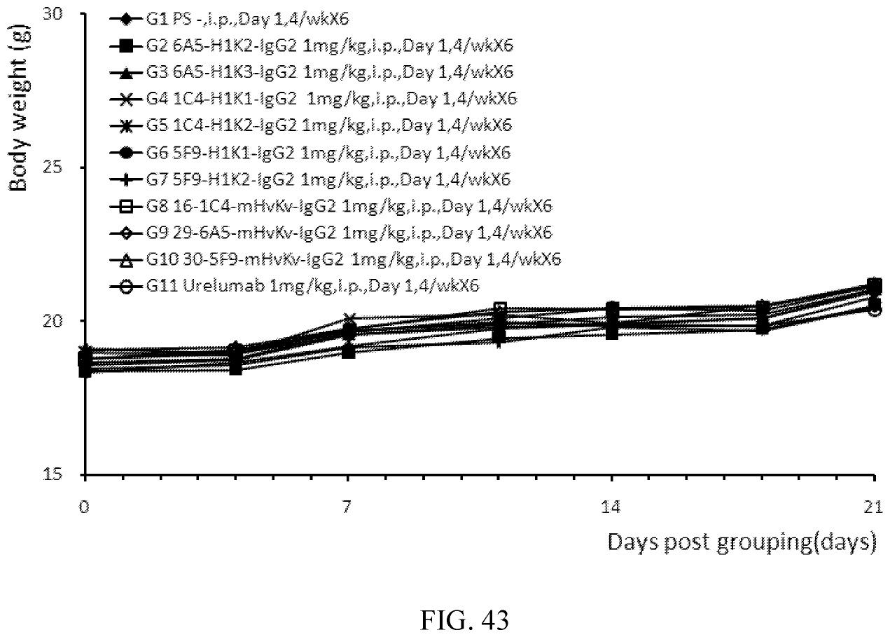

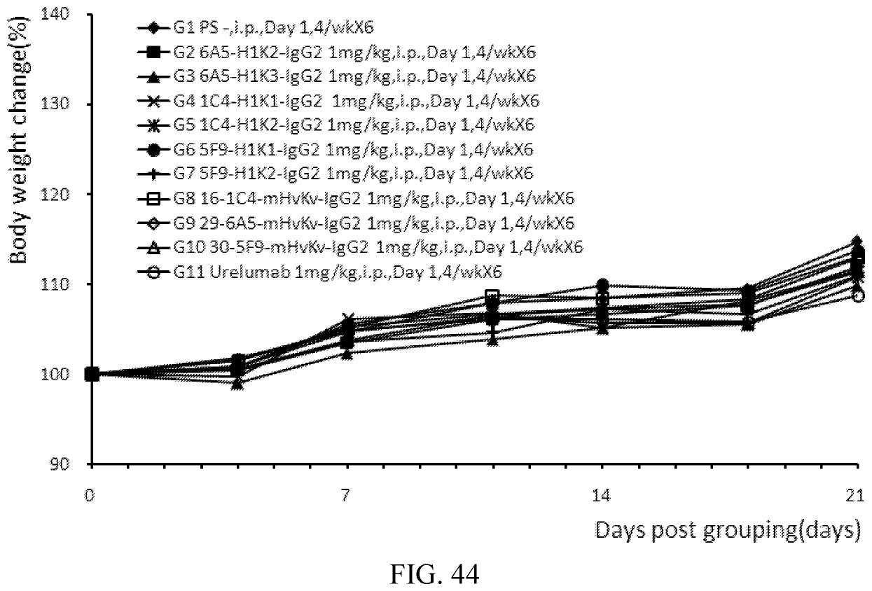

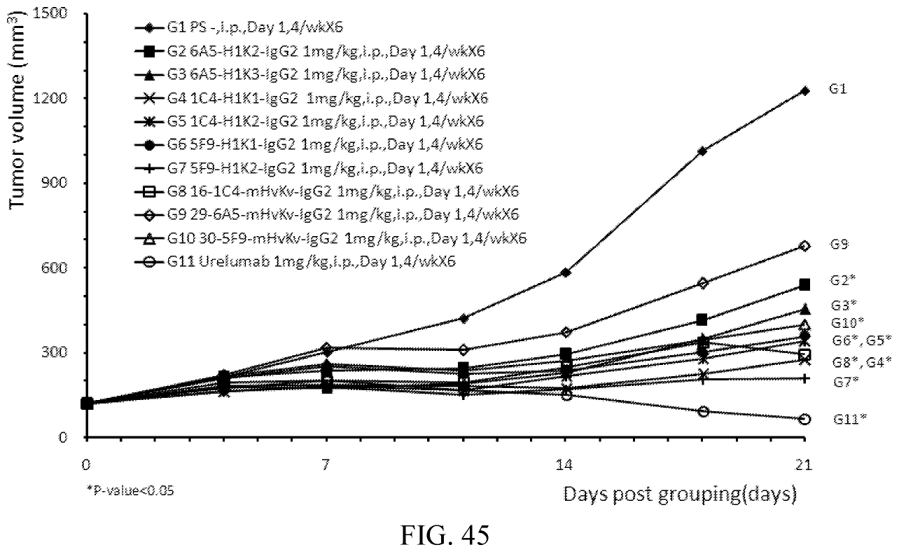

The weight of the mice was monitored during the entire treatment period. The average weight of mice in different groups all increased at the end of the treatment period (FIG. 43, and FIG. 44). No obvious difference in weight was observed among different groups. The results showed that these anti-h4-1BB antibodies were well tolerated and were not obviously toxic to the mice. The tumor size for each group is shown in FIG. 45.

The TGI % at day 21 (21 days after grouping) was also calculated as shown in the table below.

TABLE 33

| | | Tumor volume(mm³) | | | | | | P value | |
|---|---|---|---|---|---|---|---|---|---|
| | | Day | Day | Day | Day | | | Body | Tumor |
| | | 0 | 6 | 13 | 17 | Survival | TGI % | weight | Volume |
| Control | G1 | 121 ± 6 | 301 ± 27 | 585 ± 111 | 1227 ± 198 | 8/8 | n.a. | n.a. | n.a. |
| Treat | G2 | 121 ± 8 | 236 ± 27 | 296 ± 31 | 540 ± 89 | 8/8 | 62.1% | 0.458 | 0.007 |
| | G3 | 121 ± 7 | 176 ± 28 | 230 ± 81 | 455 ± 222 | 8/8 | 69.8% | 0.981 | 0.021 |
| | G4 | 121 ± 7 | 188 ± 28 | 177 ± 31 | 275 ± 75 | 8/8 | 86.1% | 0.897 | 5.1E−04 |
| | G5 | 121 ± 8 | 184 ± 27 | 216 ± 50 | 340 ± 92 | 8/8 | 80.2% | 0.772 | 1.2E−03 |
| | G6 | 121 ± 7 | 251 ± 26 | 237 ± 43 | 359 ± 89 | 8/8 | 78.5% | 0.868 | 1.3E−03 |
| | G7 | 121 ± 6 | 180 ± 16 | 171 ± 37 | 210 ± 49 | 8/8 | 92.0% | 0.461 | 2.0E−04 |
| | G8 | 121 ± 7 | 203 ± 25 | 248 ± 72 | 296 ± 116 | 8/8 | 84.2% | 0.806 | 1.2E−03 |
| | G9 | 121 ± 7 | 319 ± 55 | 374 ± 100 | 680 ± 232 | 8/8 | 49.5% | 0.910 | 0.094 |
| | G10 | 121 ± 7 | 262 ± 26 | 273 ± 43 | 399 ± 101 | 8/8 | 74.8% | 0.803 | 0.002 |
| | G11 | 121 ± 8 | 202 ± 29 | 152 ± 41 | 67 ± 26 | 8/8 | 104.9% | 0.353 | 4.6E−05 |

The results showed that these humanized and chimeric antibodies had different tumor inhibitory effects.

### Example 13. Human Anti-4-1BB IgG1 Antibodies have Better Efficacy than Other IgG Subclasses

Based on results from 16-1C4-mHvKv-IgG1 as described above, it was hypothesized that for the same VH and VL, the IgG1 subclass have better tumor inhibitory effects than the other IgG subclasses. Different subclasses of anti-h4-1BB IgG antibodies were tested in 4-1BB humanized mice (B-h4-1BB) to determine their effect on tumor growth in vivo. Furthermore, IgG1 antibodies with N297A (EU numbering) mutation, IgG1 antibodies with FC-SI mutations (EU Numbering: F243L/R292P/Y300L/V305I/P396L), and IgG1 antibodies with FC-V11 mutations (G237D/P238D/H268D/P271G/A330R) were also tested. The N297A mutation can reduce ADCC effects. The FC-SI mutations can increase the binding affinities with almost all Fc receptors, particularly FcγRIIIA/IIA, and may increase ADCC effects. And the FC-V11 mutation can increase the binding affinity with FcγRIIB, but will not increase the binding affinity with FcγRIIA, and thus will reduce the binding affinity with FcγRIIIA.

MC-38 cancer tumor cells (colon adenocarcinoma cell) were injected subcutaneously in B-h4-1BB mice. When the tumors in the mice reached a volume of 150±50 mm³, the mice were randomly placed into different groups based on the volume of the tumor, and were administered with different antibodies as shown in the table below. Urelumab is a fully human IgG4 monoclonal antibody. The antibody urelumab-IgG1 has VH and VL from urelumab, and the constant domains are from IgG1 subclass. Similarly, the antibody urelumab-IgG2 has VH and VL form urelumab, and the constant domains are from IgG2 subclass. The antibody urelumab-IgG4 has constant domains from human IgG4.

TABLE 34

| | No. of | | Dosage | | | Total No. of |
|---|---|---|---|---|---|---|
| Group | mice | Antibodies | (mg/kg) | Route | Frequency | administration |
| G1 | 7 | PS (control) | — | i.p. | Day 2,5/wk | 4 |
| G2 | 7 | Urelumab-IgG1 | 1 mg/kg | i.p. | Day 2,5/wk | 4 |
| G3 | 7 | Urelumab-IgG2 | 1 mg/kg | i.p. | Day 2,5/wk | 4 |
| G4 | 7 | Urelumab-IgG4 | 1 mg/kg | i.p. | Day 2,5/wk | 4 |
| G5 | 7 | Urelumab-IgG1-N297A | 1 mg/kg | i.p. | Day 2,5/wk | 4 |
| G6 | 7 | Urelumab-IgG1-FC-SI | 1 mg/kg | i.p. | Day 2,5/wk | 4 |
| G7 | 7 | Urelumab-IgG1-FC-V11 | 1 mg/kg | i.p. | Day 2,5/wk | 4 |

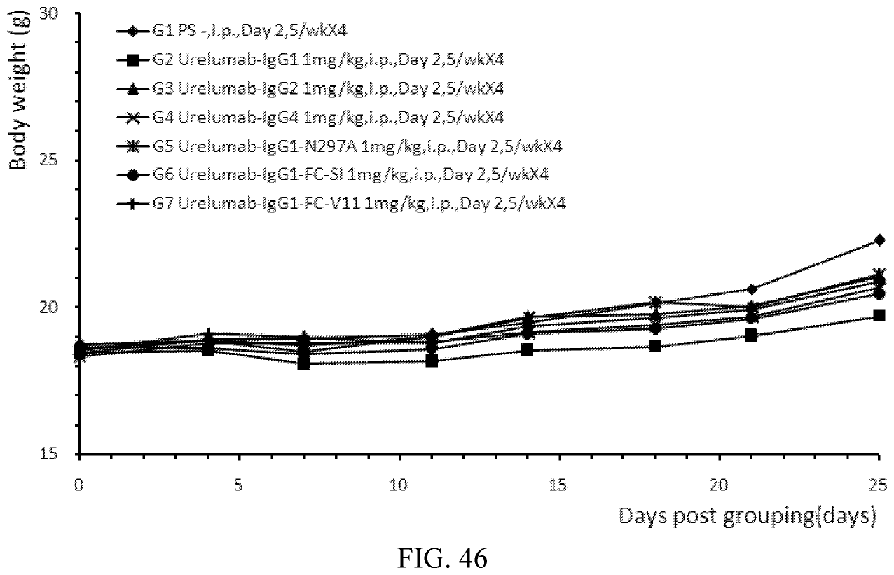

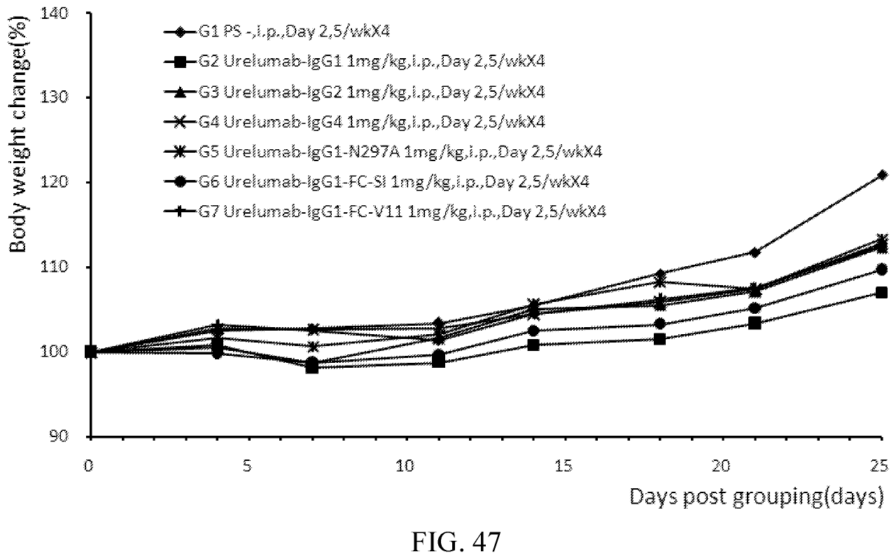

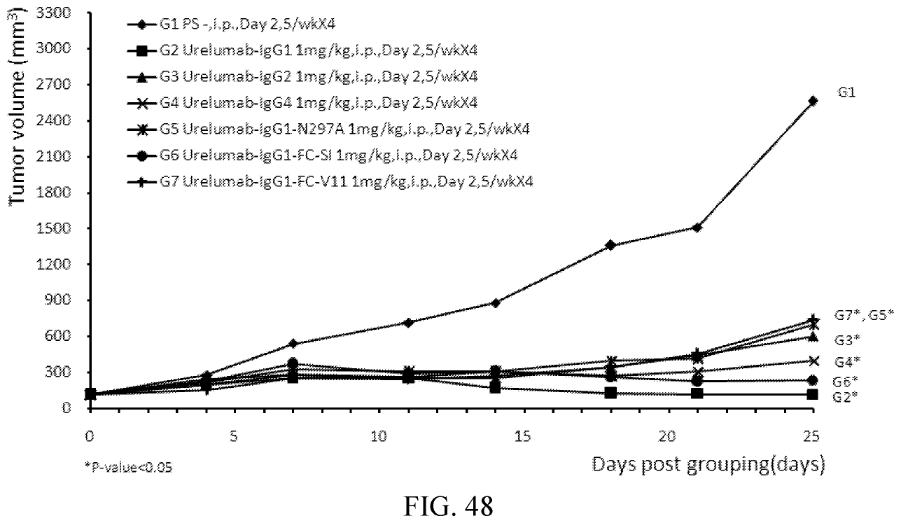

The weight of the mice was monitored during the entire treatment period. The average weight of mice in different groups all increased at the end of the treatment period (FIG. 46, and FIG. 47). No obvious difference in weight was observed among the different groups. The results showed that these anti-h4-1BB antibodies were well tolerated and were not obviously toxic to the mice. The tumor size for each group is shown in FIG. 48.

The TGI % at day 25 (25 days after grouping) was also calculated as shown in the table below.

TABLE 35

| | | Tumor volume (mm³) | | | | | | P value | |
|---|---|---|---|---|---|---|---|---|---|
| | | Day | Day | Day | Day | | | Body | Tumor |
| | | 0 | 11 | 18 | 25 | Survival | TGI % | weight | Volume |
| Control | G1 | 119 ± 5 | 718 ± 97 | 1358 ± 338 | 2562 ± 477 | 7/7 | n.a. | n.a. | n.a. |
| Treat | G2 | 119 ± 6 | 249 ± 40 | 128 ± 38 | 116 ± 50 | 7/7 | 100.1% | 0.007 | 2.6E−04 |
| | G3 | 119 ± 6 | 256 ± 48 | 340 ± 110 | 604 ± 195 | 7/7 | 80.1% | 0.071 | 0.003 |
| | G4 | 119 ± 7 | 262 ± 58 | 276 ± 64 | 398 ± 111 | 7/7 | 88.6% | 0.074 | 8.3E−04 |
| | G5 | 119 ± 5 | 305 ± 44 | 399 ± 91 | 697 ± 181 | 7/7 | 76.3% | 0.294 | 0.003 |
| | G6 | 119 ± 6 | 293 ± 65 | 258 ± 111 | 232 ± 138 | 7/7 | 95.4% | 0.004 | 5.2E−04 |
| | G7 | 119 ± 5 | 240 ± 31 | 347 ± 67 | 740 ± 188 | 7/7 | 74.6% | 0.078 | 0.004 |

The results confirmed that anti-h4-1BB IgG1 subclasses had higher tumor inhibitory effects as compared to IgG2 and IgG4 subclasses.

### Example 14. Chimeric Anti-4-1BB IgG1 Antibodies have Better Efficacy than Other IgG Subclasses

Experiments were repeated to test different subclasses of anti-h4-1BB IgG antibodies. MC-38 cancer tumor cells (colon adenocarcinoma cell) were injected subcutaneously in B-h4-1BB mice. When the tumors in the mice reached a volume of 150±50 mm³, the mice were randomly placed into different groups based on the volume of the tumor, and were administered with different antibodies once a week (QW) as shown in the table below.

TABLE 36

| | No. of | | Dosage | | | Total No. of |
|---|---|---|---|---|---|---|
| Group | mice | Antibodies | (mg/kg) | Route | Frequency | administration |
| G1 | 8 | PS (control) | — | i.p. | QW | 4 |
| G2 | 8 | 16-1C4-mHvKv-IgG1 | 1 mg/kg | i.p. | QW | 4 |
| G3 | 8 | 16-1C4-mHvKv-IgG2 | 1 mg/kg | i.p. | QW | 4 |
| G4 | 8 | 16-1C4-mHvKv-IgG4 | 1 mg/kg | i.p. | QW | 4 |
| G5 | 8 | 16-1C4-mHvKv-IgG1-FC-V11 | 1 mg/kg | i.p. | QW | 4 |
| G6 | 8 | 16-1C4-mHvKv-IgG1-FC-SI | 1 mg/kg | i.p. | QW | 4 |
| G7 | 8 | 16-1C4 | 1 mg/kg | i.p. | QW | 4 |
| G8 | 8 | Urelumab | 1 mg/kg | i.p. | QW | 4 |

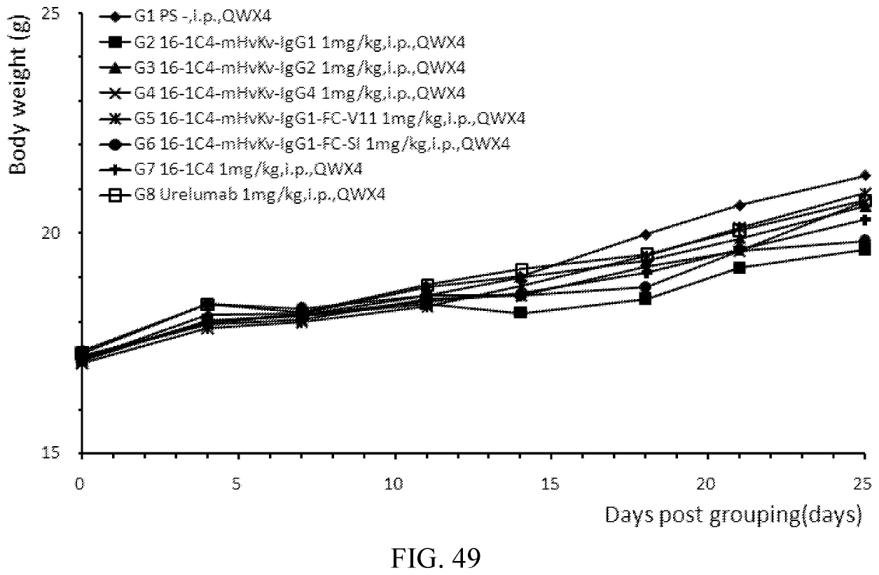

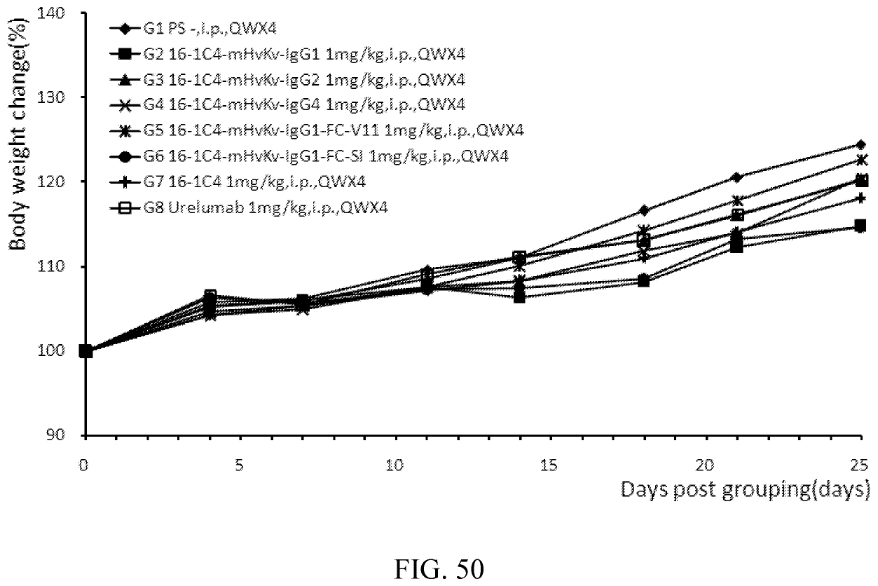

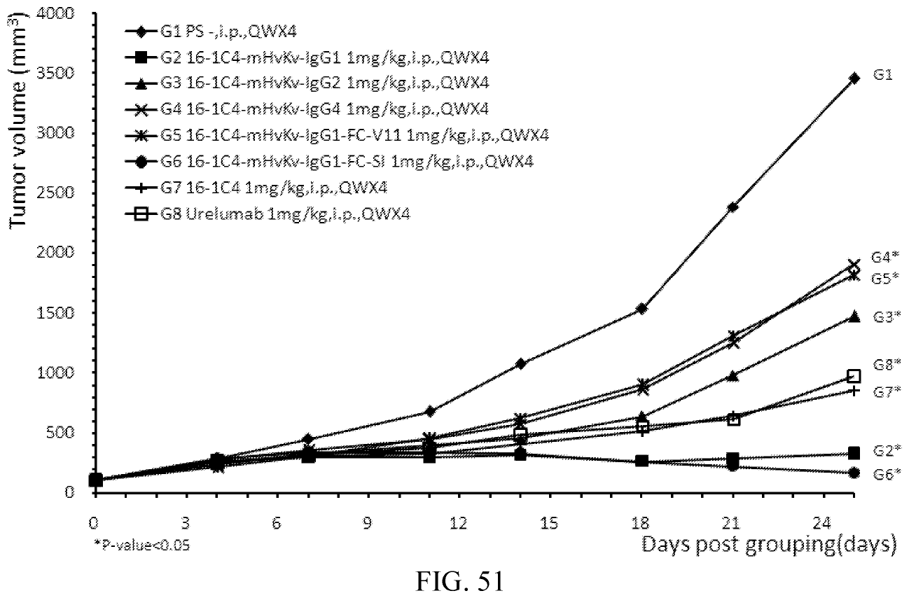

The weight of the mice was monitored during the entire treatment period. The average weight of mice in different groups all increased at the end of treatment period (FIG. 49, and FIG. 50). No obvious difference in weight was observed among the different groups. The results showed that these anti-h4-1BB antibodies were well tolerated and were not obviously toxic to the mice. The tumor size for each group is shown in FIG. 51.

The TGI % at day 25 (25 days after grouping) was also calculated as shown in the table below.

TABLE 37

| | | Tumor volume (mm³) | | | | | | P value | |
|---|---|---|---|---|---|---|---|---|---|
| | | Day | Day | Day | Day | | | Body | Tumor |
| | | 0 | 11 | 18 | 25 | Survival | TGI % | weight | Volume |
| Control | G1 | 111 ± 7 | 678 ± 151 | 1533 ± 293 | 3455 ± 435 | 8/8 | n.a. | n.a. | n.a. |
| Treat | G2 | 111 ± 6 | 299 ± 21 | 262 ± 62 | 328 ± 204 | 8/8 | 93.5% | 0.030 | 1.4E−05 |
| | G3 | 111 ± 7 | 396 ± 77 | 640 ± 170 | 1474 ± 414 | 8/8 | 59.3% | 0.378 | 0.005 |
| | G4 | 112 ± 5 | 445 ± 61 | 867 ± 165 | 1912 ± 452 | 8/8 | 46.2% | 0.454 | 0.027 |
| | G5 | 111 ± 6 | 454 ± 74 | 903 ± 176 | 1822 ± 352 | 8/8 | 48.8% | 0.570 | 0.011 |
| | G6 | 111 ± 6 | 337 ± 44 | 263 ± 52 | 164 ± 41 | 8/8 | 98.4% | 0.032 | 2.7E−06 |
| | G7 | 111 ± 7 | 330 ± 36 | 519 ± 133 | 860 ± 247 | 8/8 | 77.6% | 0.126 | 1.4E−04 |
| | G8 | 111 ± 5 | 380 ± 59 | 560 ± 132 | 970 ± 271 | 8/8 | 74.3% | 0.383 | 2.6E−04 |

The results again confirmed that IgG1 subclasses had higher tumor inhibitory effects as compared to IgG2 and IgG4 subclasses.

### Example 15. Chimeric and Human Anti-4-1BB IgG1 Antibodies have Better Efficacy than Other IgG Subclasses

Experiments were repeated to test different subclasses of chimeric and human anti-h4-1BB IgG antibodies. mIgG2A and human IgG1 have better ADCC effects, and mIgG1 and human IgG4 have weaker or no ADCC effects,

MC-38 cancer tumor cells (colon adenocarcinoma cell) were injected subcutaneously in B-h4-1BB mice. When the tumors in the mice reached a volume of 150±50 mm³, the mice were randomly placed into different groups based on the volume of the tumor, and were administered with different antibodies once a week (QW) as shown in the table below.

TABLE 38

| | No. of | | Dosage | | | Total No. of |
|---|---|---|---|---|---|---|
| Group | mice | Antibodies | (mg/kg) | Route | Frequency | administration |
| G1 | 8 | PS (control) | — | i.p. | QW | 3 |
| G2 | 8 | Urelumab-mIgG1 | 1 mg/kg | i.p. | QW | 3 |
| G3 | 8 | Urelumab-mIgG2A | 1 mg/kg | i.p. | QW | 3 |
| G4 | 8 | 16-1C4-mHvKv-mIgG1 | 1 mg/kg | i.p. | QW | 3 |
| G5 | 8 | 16-1C4-mHvKv-mIgG2A | 1 mg/kg | i.p. | QW | 3 |
| G6 | 8 | Urelumab | 1 mg/kg | i.p. | QW | 3 |
| G7 | 8 | Urelumab-IgG1 | 1 mg/kg | i.p. | QW | 3 |
| G8 | 8 | 16-1C4-mHvKv-IgG1 | 1 mg/kg | i.p. | QW | 3 |
| G9 | 8 | 16-1C4-mHvKv-IgG4 | 1 mg/kg | i.p. | QW | 3 |

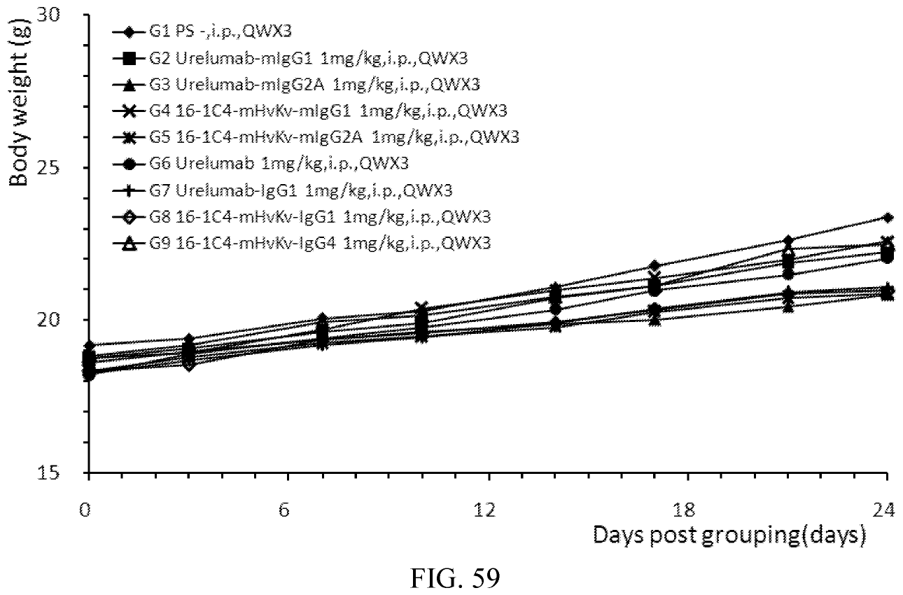

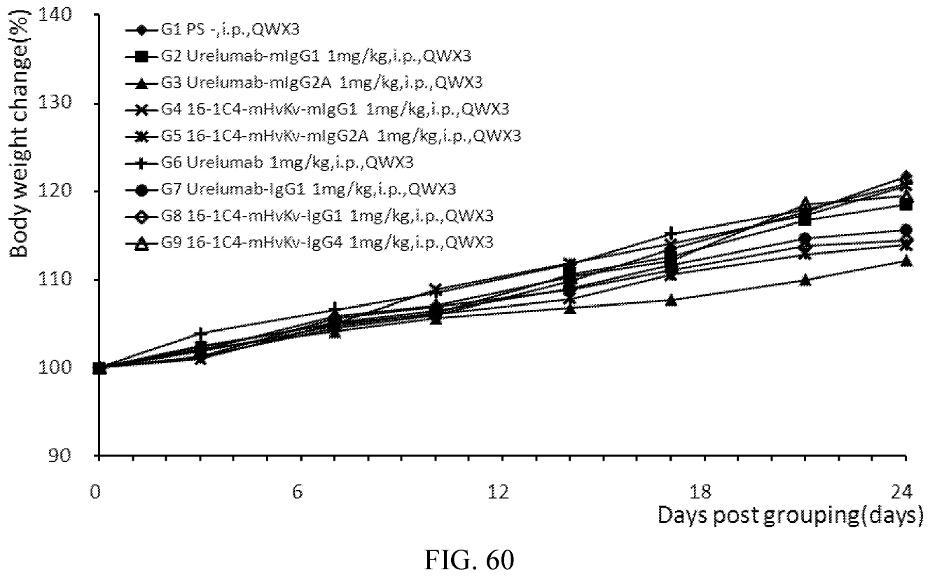

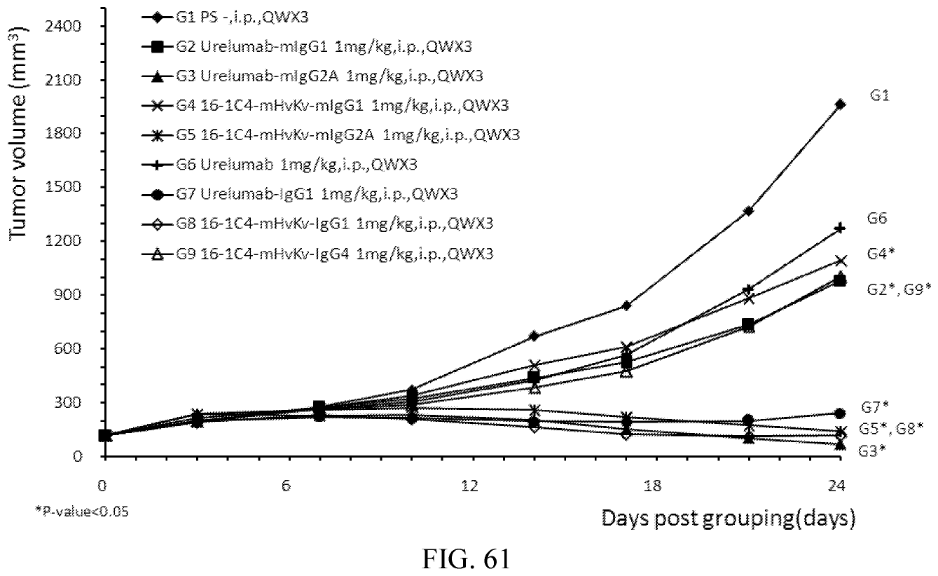

The weight of the mice was monitored during the entire treatment period. The average weight of mice in different groups all increased at the end of treatment period (FIG. 59, and FIG. 60). No obvious difference in weight was observed among the different groups. The results showed that these anti-h4-1BB antibodies were well tolerated and were not obviously toxic to the mice. The tumor size for each group is shown in FIG. 61

The TGI % at day 24 (24 days after grouping) was also calculated as shown in the table below.

TABLE 39

| | | Tumor volume (mm³) | | | | | | P value | |
|---|---|---|---|---|---|---|---|---|---|
| | | Day | Day | Day | Day | | | Body | Tumor |
| | | 0 | 10 | 17 | 24 | Survival | TGI % | weight | Volume |
| Control | G1 | 117 ± 4 | 374 ± 30 | 841 ± 104 | 1964 ± 149 | 8/8 | n.a. | n.a. | n.a. |
| Treat | G2 | 117 ± 4 | 321 ± 34 | 525 ± 87 | 979 ± 171 | 8/8 | 53.3% | 0.353 | 6.7E−04 |
| | G3 | 117 ± 4 | 234 ± 21 | 153 ± 29 | 70 ± 21 | 8/8 | 102.5% | 0.018 | 5.3E−09 |
| | G4 | 117 ± 4 | 341 ± 41 | 611 ± 91 | 1094 ± 183 | 8/8 | 47.1% | 0.429 | 0.002 |
| | G5 | 117 ± 4 | 274 ± 70 | 219 ± 53 | 139 ± 171 | 8/8 | 98.8% | 0.036 | 2.0E−08 |
| | G6 | 117 ± 5 | 306 ± 40 | 570 ± 122 | 1273 ± 312 | 8/8 | 37.4% | 0.249 | 0.065 |
| | G7 | 117 ± 6 | 213 ± 34 | 190 ± 51 | 243 ± 88 | 8/8 | 93.1% | 0.037 | 1.0E−07 |
| | G8 | 117 ± 11 | 208 ± 28 | 126 ± 38 | 116 ± 87 | 8/8 | 100.0% | 0.041 | 4.1E−08 |
| | G9 | 117 ± 15 | 287 ± 28 | 475 ± 76 | 1003 ± 215 | 8/8 | 52.0% | 0.414 | 0.003 |

The results again confirmed that mIgG2A or human IgG1 subclasses had higher tumor inhibitory effects as compared to mIgG1 or human IgG4 subclasses. Thus, the results show that anti-4-1BB antibodies inhibit tumor growth primarily through ADCC.

### Example 16. Anti-4-1BB IgG1 Antibodies had a Higher Tumor Inhibitory Effect as Compared to Anti-4-1BB IgG4 Antibodies

MC-38 cancer tumor cells (colon adenocarcinoma cell) were injected subcutaneously in B-h4-1BB mice. When the tumors in the mice reached a volume of 100˜150 mm³, the mice were randomly placed into different groups based on the volume of the tumor, and were administered with different antibodies as shown in the table below. As discussed above, Urelumab is a fully human IgG4 monoclonal antibody. The antibody urelumab-IgG1 has VH and VL from urelumab, and the constant domains are from IgG1 subclass.

TABLE 40

| | No. of | | Dosage | | | Total No. of |
|---|---|---|---|---|---|---|
| Group | mice | Antibodies | (mg/kg) | Route | Frequency | administration |
| G1 | 8 | PS (control) | — | i.p. | QW | 3 |
| G2 | 8 | Urelumab | 1 mg/kg | i.p. | QW | 3 |
| G3 | 8 | Urelumab-IgG1 | 1 mg/kg | i.p. | QW | 3 |
| G4 | 8 | 16-1C4-mHvKv-IgG1 | 1 mg/kg | i.p. | QW | 3 |
| G5 | 8 | 16-1C4-mHvKv-IgG4 | 1 mg/kg | i.p. | QW | 3 |
| G6 | 8 | 1C4-H1K1-IgG1 | 1 mg/kg | i.p. | QW | 3 |
| G7 | 8 | 1C4-H1K1-IgG4 | 1 mg/kg | i.p. | QW | 3 |
| G8 | 8 | 1C4-H1K2-IgG1 | 1 mg/kg | i.p. | QW | 3 |
| G9 | 8 | 1C4-H1K2-IgG4 | 1 mg/kg | i.p. | QW | 3 |
| G10 | 8 | 6A5-H1K2-IgG1 | 1 mg/kg | i.p. | QW | 3 |
| G11 | 8 | 5F9-H1K1-IgG1 | 1 mg/kg | i.p. | QW | 3 |
| G12 | 8 | 16-1C4-mHvKv- | 1 mg/kg | i.p. | QW | 3 |
| | | IgG1-FC-V11 | | | | |

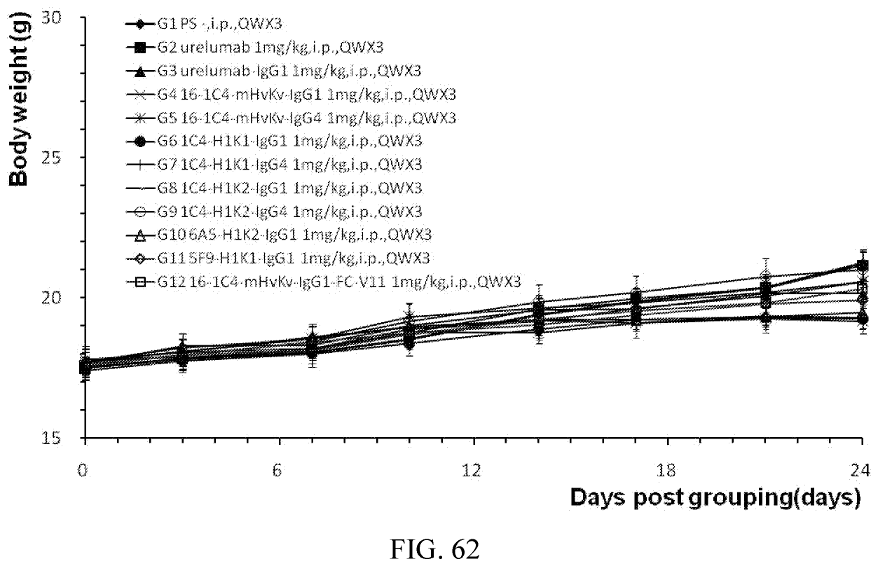

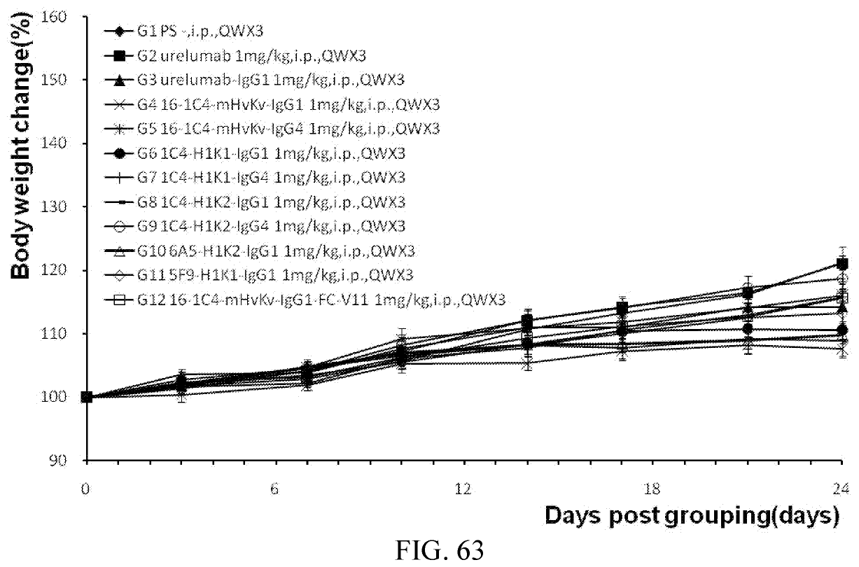

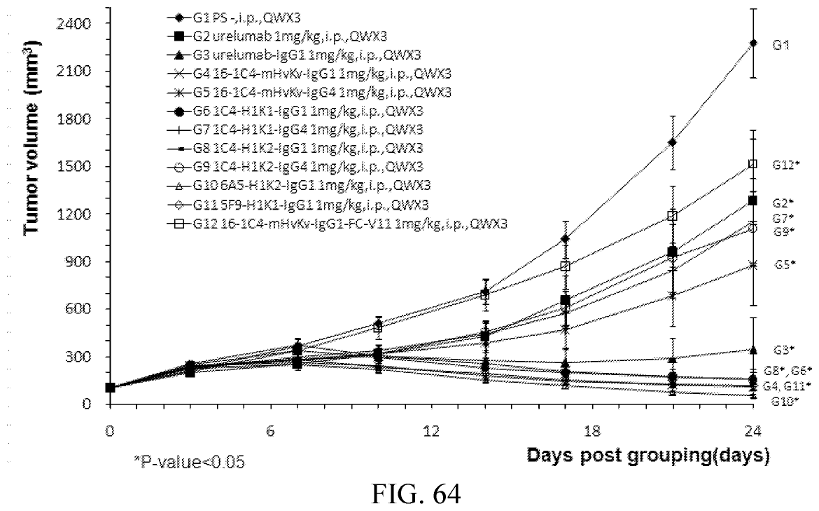

The weight of the mice was monitored during the entire treatment period. The average weight of mice in different groups all increased at the end of the treatment period (FIG. 62 and FIG. 63). No obvious differences in weight were observed among the different groups. The results showed that these anti-h4-1BB antibodies were well tolerated and were not obviously toxic to the mice. The tumor size for each group is shown in FIG. 64.

The TGI % at day 24 (24 days after grouping) was also calculated as shown in the table below.

TABLE 41

| | | Tumor volume (mm³) | | | | | | P value | |
|---|---|---|---|---|---|---|---|---|---|
| | | Day | Day | Day | Day | | | Body | Tumor |
| | | 0 | 10 | 17 | 24 | Survival | TGI % | weight | Volume |
| Control | G1 | 107 ± 3 | 512 ± 36 | 1042 ± 115 | 2283 ± 217 | 8/8 | n.a. | n.a. | n.a. |
| Treat | G2 | 107 ± 4 | 318 ± 55 | 655 ± 160 | 1289 ± 390 | 8/8 | 45.7% | 0.902 | 0.043 |
| | G3 | 107 ± 4 | 306 ± 47 | 267 ± 88 | 344 ± 205 | 8/8 | 89.1% | 0.133 | <0.001 |
| | G4 | 107 ± 4 | 240 ± 21 | 150 ± 21 | 109 ± 23 | 8/8 | 99.9% | 0.002 | <0.001 |
| | G5 | 107 ± 4 | 321 ± 59 | 471 ± 120 | 882 ± 254 | 8/8 | 64.4% | 0.439 | 0.001 |
| | G6 | 107 ± 3 | 296 ± 62 | 202 ± 54 | 161 ± 64 | 8/8 | 97.5% | 0.006 | <0.001 |
| | G7 | 107 ± 4 | 337 ± 42 | 577 ± 97 | 1157 ± 273 | 8/8 | 51.8% | 0.301 | 0.006 |
| | G8 | 107 ± 4 | 305 ± 30 | 209 ± 29 | 160 ± 48 | 8/8 | 97.6% | 0.020 | <0.001 |
| | G9 | 107 ± 3 | 322 ± 30 | 607 ± 107 | 1110 ± 235 | 8/8 | 53.9% | 0.770 | 0.003 |
| | G10 | 107 ± 3 | 221 ± 21 | 116 ± 12 | 58 ± 9 | 8/8 | 102.3% | 0.007 | <0.001 |
| | G11 | 107 ± 3 | 239 ± 22 | 157 ± 27 | 117 ± 33 | 8/8 | 99.6% | 0.109 | <0.001 |
| | G12 | 107 ± 3 | 484 ± 71 | 869 ± 137 | 1518 ± 217 | 8/8 | 35.2% | 0.151 | 0.026 |

The results confirmed that urelumab-IgG1 antibodies had higher tumor inhibitory effects as compared to urelumab (IgG4); 16-1C4-mHvKv-IgG1 antibodies had higher tumor inhibitory effects as compared to 16-1C4-mHvKv-IgG4 antibodies; 1C4-H1K1-IgG1 antibodies had higher tumor inhibitory effects as compared to 1C4-H1K1-IgG4 antibodies; 1C4-H1K2-IgG1 antibodies had higher tumor inhibitory effects as compared to 1C4-H1K2-IgG4 antibodies. In summary, the result showed that for the same antigen binding domains, IgG1 antibodies had a higher tumor inhibitory effect as compared to IgG4 antibodies.

### Example 17. Anti-h4-1BB Antibody Combination Therapies

MC-38 cancer tumor cells (colon adenocarcinoma cell) expressing human PD-L1 were injected subcutaneously in B-h4-1BB mice. When the tumors in the mice reached a volume of about 400 mm³, the mice were randomly placed into different groups based on the volume of the tumor, and were administered with different antibodies as shown in the table below. Keytruda® (Pembrolizumab) is a humanized anti-PD-1 IgG4 antibody.

TABLE 42

| | No. of | | Dosage | | | Total No. of |
|---|---|---|---|---|---|---|
| Group | mice | Antibodies | (mg/kg) | Route | Frequency | administration |
| G1 | 8 | PS (control) | — | i.p. | BIW | 6 |
| G2 | 8 | 1C4-H1K1-IgG1 | 0.3 mg/kg | i.p. | BIW | 6 |
| G3 | 8 | 1C4-H1K1-IgG4 | 0.3 mg/kg | i.p. | BIW | 6 |
| G4 | 8 | urelumab-IgG1 | 0.3 mg/kg | i.p. | BIW | 6 |
| G5 | 8 | urelumab-IgG4 | 0.3 mg/kg | i.p. | BIW | 6 |
| G6 | 8 | Keytruda ®-IgG4 | 0.3 mg/kg | i.p. | BIW | 6 |
| G7 | 8 | 1C4-H1K1-IgG1 + | 0.3 mg/kg + | i.p. | BIW | 6 |
| | | Keytruda ®-IgG4 | 0.3 mg/kg | | | |
| G8 | 8 | 1C4-H1K1-IgG4 + | 0.3 mg/kg + | i.p. | BIW | 6 |
| | | Keytruda ®-IgG4 | 0.3 mg/kg | | | |
| G9 | 8 | urelumab-IgG1 + | 0.3 mg/kg + | i.p. | BIW | 6 |
| | | Keytruda ®-IgG4 | 0.3 mg/kg | | | |
| G10 | 8 | urelumab-IgG4 + | 0.3 mg/kg + | i.p. | BIW | 6 |
| | | Keytruda ®-IgG4 | 0.3 mg/kg | | | |

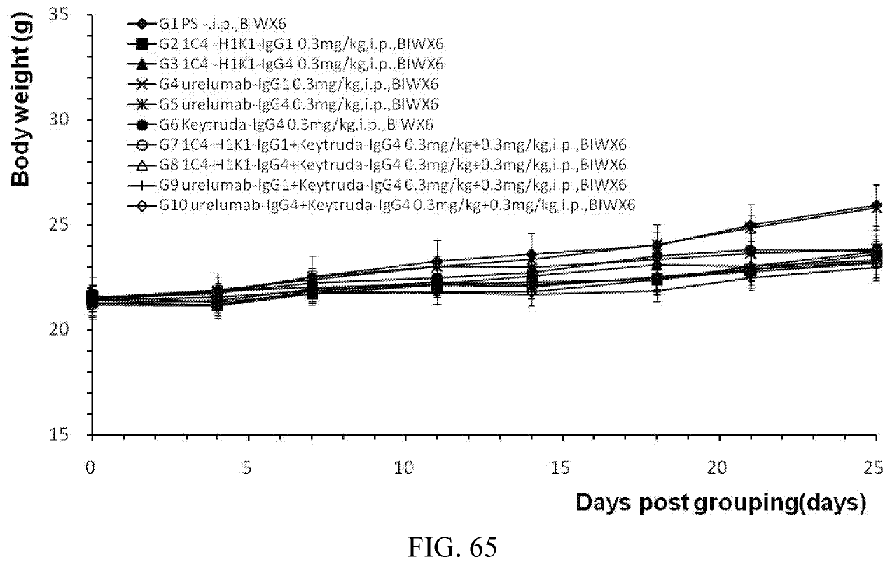

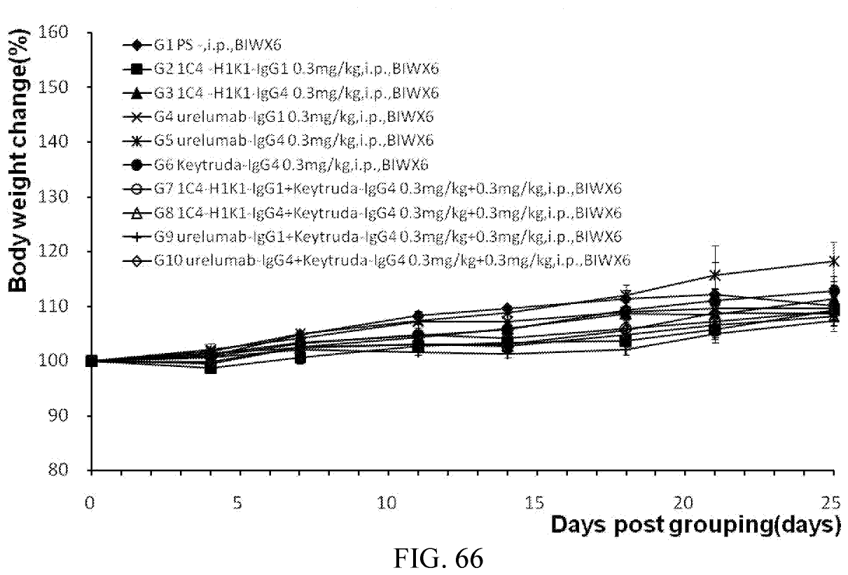

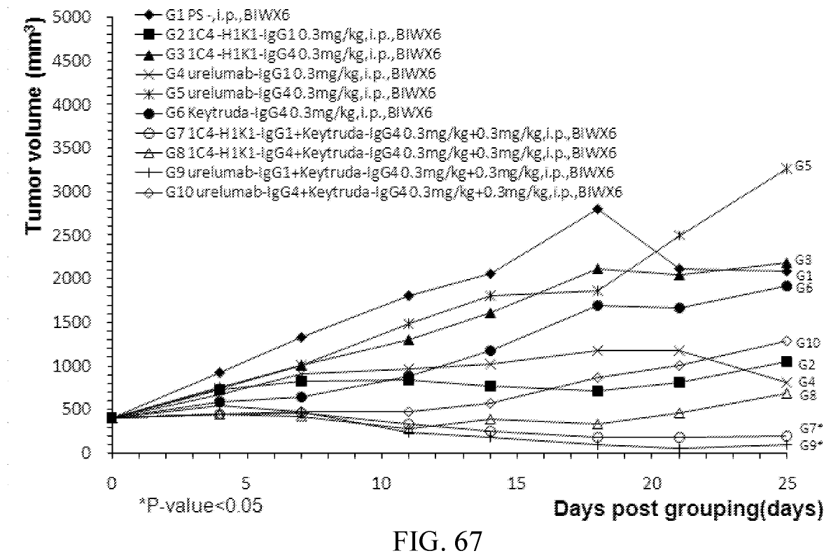

The weight of the mice was monitored during the entire treatment period. The average weight of mice in different groups all increased at the end of the treatment period (FIG. 65 and FIG. 66). No obvious differences in weight were observed among the different groups. The results showed that these anti-h4-1BB antibodies were well tolerated and were not obviously toxic to the mice. The tumor size for each group is shown in FIG. 67.

The TGI % at day 25 (25 days after grouping) was also calculated as shown in the table below.

TABLE 43

| | | Tumor volume (mm³) | | | | | | P value | |
|---|---|---|---|---|---|---|---|---|---|
| | | Day | Day | Day | Day | | | Body | Tumor |
| | | 0 | 11 | 18 | 25 | Survival | TGI % | weight | Volume |
| Control | G1 | 399 ± 20 | 1811 ± 275 | 2803 ± 508 | 2092 ± 769 | 2/8 | n.a. | n.a. | n.a. |
| Treat | G2 | 400 ± 23 | 840 ± 104 | 710 ± 187 | 1051 ± 471 | 8/8 | 61.5% | 0.124 | 0.342 |
| | G3 | 400 ± 22 | 1301 ± 202 | 2117 ± 452 | 2182 ± 578 | 4/8 | −5.3% | 0.317 | 0.932 |
| | G4 | 400 ± 24 | 972 ± 239 | 1175 ± 441 | 810 ± 366 | 6/8 | 75.8% | 0.147 | 0.142 |
| | G5 | 399 ± 29 | 1482 ± 228 | 1858 ± 257 | 3266 ± 554 | 5/8 | −69.3% | 0.953 | 0.299 |
| | G6 | 400 ± 29 | 882 ± 188 | 1692 ± 515 | 1916 ± 538 | 6/8 | 10.4% | 0.084 | 0.872 |
| | G7 | 399 ± 24 | 337 ± 51 | 175 ± 65 | 190 ± 125 | 8/8 | 112.4% | 0.166 | 0.001 |
| | G8 | 400 ± 22 | 284 ± 76 | 330 ± 155 | 691 ± 341 | 8/8 | 82.8% | 0.060 | 0.108 |
| | G9 | 400 ± 27 | 243 ± 65 | 93 ± 53 | 95 ± 95 | 8/8 | 118.0% | 0.043 | 0.001 |
| | G10 | 400 ± 24 | 475 ± 151 | 868 ± 379 | 1284 ± 705 | 7/8 | 47.8% | 0.139 | 0.588 |

The results confirmed that 1C4-H1K1-IgG1 antibodies had higher tumor inhibitory effects as compared to 1C4-H1K1-IgG4; and urelumab-IgG1 antibodies had higher tumor inhibitory effects as compared to urelumab-IgG4 antibodies. Furthermore, the results showed that the combination of anti-4-1BB antibodies with anti-PD-1 antibodies had higher tumor inhibitory effects as compared to the treatment using an anti-4-1 antibody alone or using an anti-PD-1 antibody alone. In addition, the combination of an anti-4-1BB IgG1 antibody with an anti-PD-1 antibody had higher tumor inhibitory effects as compared to the combination of an anti-4-1BB IgG4 antibody with an anti-PD-1 antibody.

### Example 18. In Vivo Testing with Melanoma Cancer Cells

B16-F10 cells (melanoma cell line) expressing human PD-L1 were injected subcutaneously in B-h4-1BB mice. When the tumors in the mice reached a volume of 100˜150 mm³, the mice were randomly placed into different groups based on the volume of the tumor, and were administered with different antibodies as shown in the table below.

TABLE 44

| | No. of | | Dosage | | | Total No. of |
|---|---|---|---|---|---|---|
| Group | mice | Antibodies | (mg/kg) | Route | Frequency | administration |
| G1 | 8 | PS (control) | — | i.p. | BIW | 4 |
| G2 | 8 | 1C4-H1K1IgG1 | 3 mg/kg | i.p. | BIW | 4 |
| G3 | 8 | Keytruda ®-IgG4 | 3 mg/kg | i.p. | BIW | 4 |
| G4 | 8 | 1C4-H1K1-IgG1 + | 3 mg/kg + | i.p. | BIW | 4 |
| | | Keytruda ®-IgG4 | 3 mg/kg | | | |

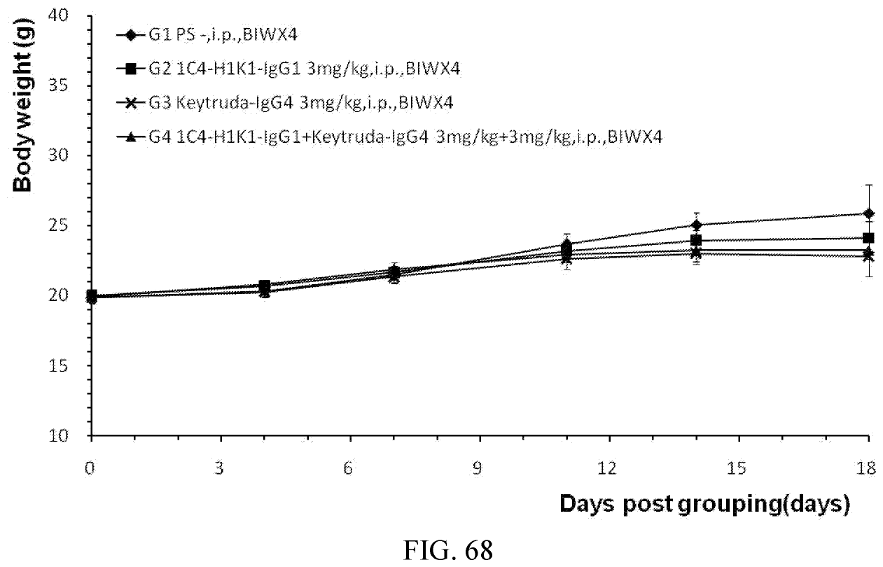

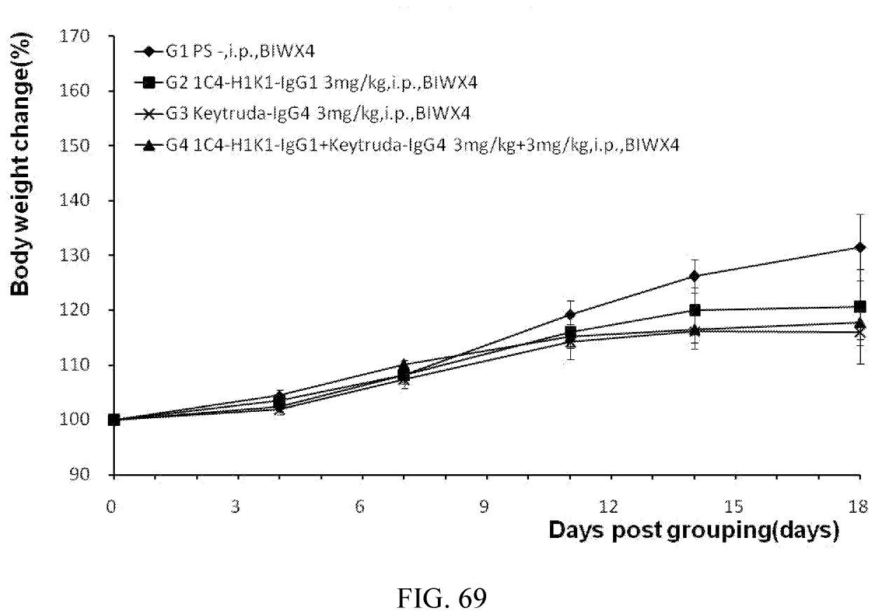

The weight of the mice was monitored during the entire treatment period. The average weight of mice in different groups all increased at the end of the treatment period (FIG. 68, and FIG. 69). No obvious differences in weight were observed among the different groups. The results showed that these anti-h4-1BB antibodies were well tolerated and were not obviously toxic to the mice. The tumor size for each group is shown in FIG. 70.

The TGI % at day 18 (18 days after grouping) was also calculated as shown in the table below.

TABLE 45

| | | Tumor volume (mm³) | | | | | | P value | |
|---|---|---|---|---|---|---|---|---|---|
| | | Day | Day | Day | Day | | | Body | Tumor |
| | | 0 | 4 | 11 | 18 | Survival | TGI % | weight | Volume |
| Control | G1 | 112 ± 5 | 503 ± 85 | 2252 ± 280 | 4801 ± 643 | 4/8 | n.a. | n.a. | n.a. |
| Treat | G2 | 112 ± 6 | 522 ± 60 | 1779 ± 323 | 2543 ± 578 | 5/8 | 48.1% | 0.450 | 0.035 |
| | G3 | 112 ± 5 | 445 ± 73 | 1497 ± 545 | 1003 ± 622 | 4/8 | 81.0% | 0.257 | 0.005 |
| | G4 | 112 ± 5 | 521 ± 55 | 1166 ± 243 | 1216 ± 401 | 7/8 | 76.5% | 0.144 | 0.001 |

The results showed that anti-h4-1BB IgG1 antibodies had excellent tumor inhibitory effects against melanoma cancer cells, and the combination with anti-h4-1BB IgG1 antibodies with anti-PD-1 antibody can further improve tumor inhibitory effects. While TGI % in G3 was 81.0%, the TGI % was calculated based on the only 4 surviving mice and the survival rate in G3 was only 4/8. In contrast, the survival rate in G4 was 7/8. The results showed that the combination treatment significantly improved the survival rate.

### Example 19. In Vivo Testing with Lymphoma Cancer Cells

EL4 cells (lymphoma cancer cells) were injected subcutaneously in B-h4-1BB mice. When the tumors in the mice reached a volume of 100˜150 mm³, the mice were randomly placed into different groups based on the volume of the tumor, and were administered with different antibodies as shown in the table below.

TABLE 46

| | No. of | | Dosage | | | Total No. of |
|---|---|---|---|---|---|---|
| Group | mice | Antibodies | (mg/kg) | Route | Frequency | administration |
| G1 | 8 | PS (control) | — | i.p. | BIW | 6 |
| G2 | 8 | 1C4-H1K1-IgG1 | 3 mg/kg | i.p. | BIW | 6 |
| G3 | 8 | Keytruda ®-IgG4 | 3 mg/kg | i.p. | BIW | 6 |
| G4 | 8 | 1C4-H1K1-IgG1 + | 3 mg/kg + | i.p. | BIW | 6 |
| | | Keytruda ®-IgG4 | 3 mg/kg | | | |

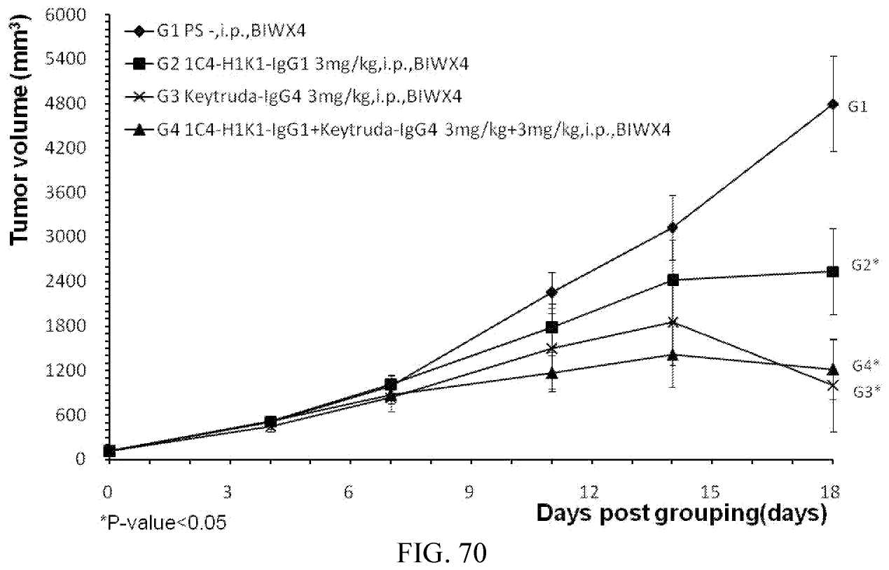

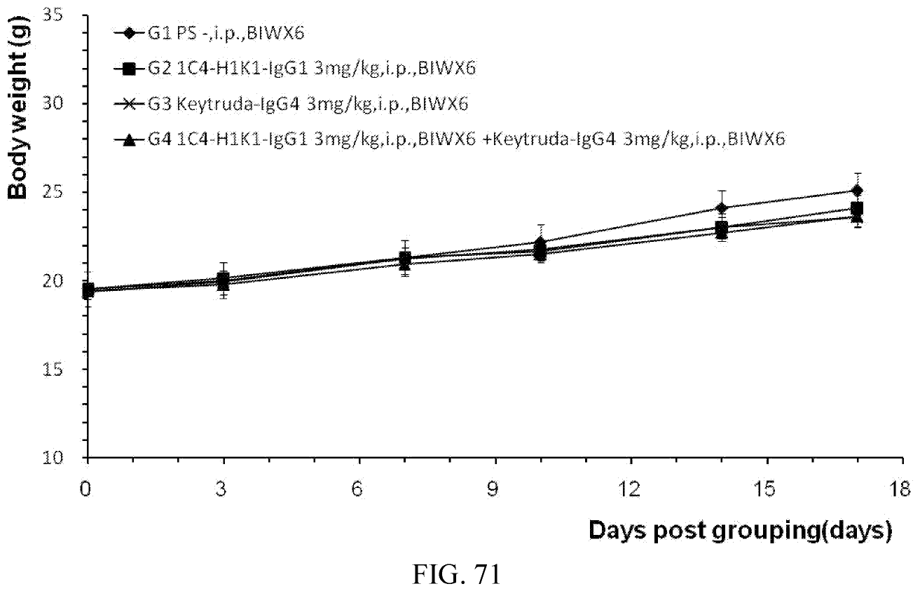

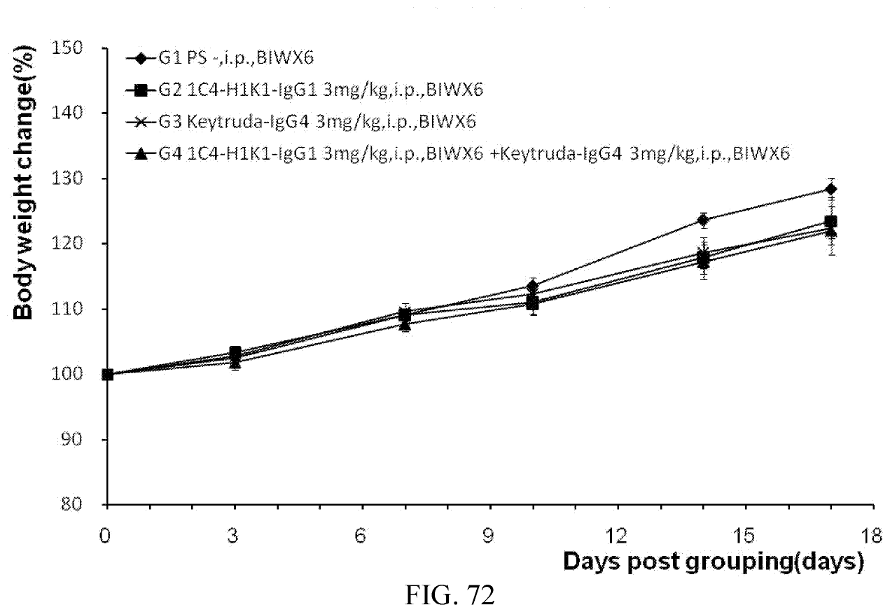

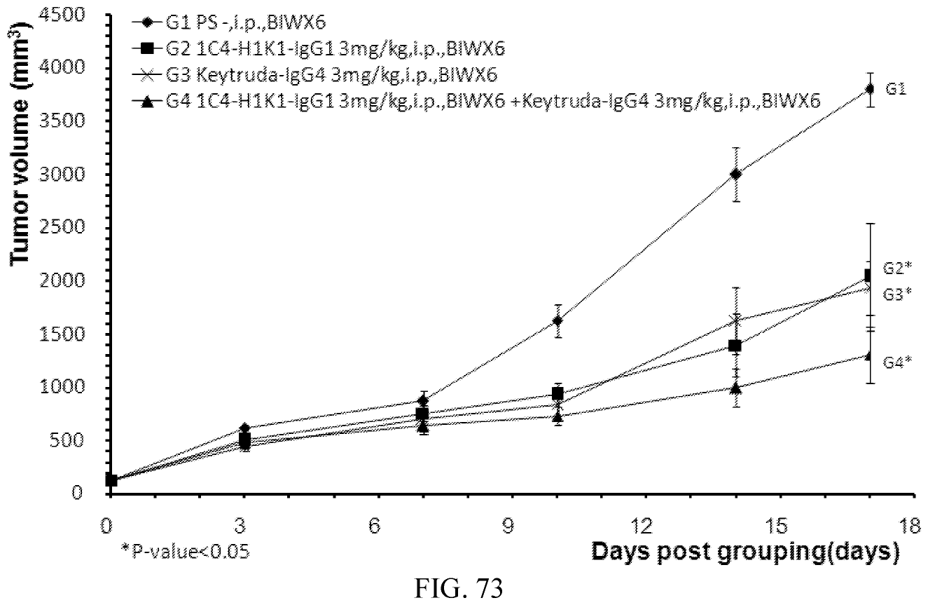

The weight of the mice was monitored during the entire treatment period. The average weight of mice in different groups all increased at the end of the treatment period (FIG. 71, and FIG. 72). No obvious difference in weight was observed among the different groups. The results showed that these anti-h4-1BB antibodies were well tolerated and were not obviously toxic to the mice. The tumor size for each group is shown in FIG. 73.

The TGI % at day 17 (17 days after grouping) was also calculated as shown in the table below.

TABLE 47

| | | Tumor volume (mm³) | | | | | | P value | |
|---|---|---|---|---|---|---|---|---|---|
| | | Day | Day | Day | Day | | | Body | Tumor |
| | | 0 | 3 | 10 | 17 | Survival | TGI % | weight | Volume |
| Control | G1 | 129 ± 1 | 622 ± 23 | 1626 ± 158 | 3799 ± 157 | 5/8 | n.a. | n.a. | n.a. |
| Treat | G2 | 129 ± 2 | 502 ± 54 | 931 ± 115 | 2038 ± 507 | 8/8 | 48.0% | 0.379 | 0.022 |
| | G3 | 129 ± 2 | 445 ± 42 | 842 ± 127 | 1933 ± 256 | 7/8 | 50.8% | 0.123 | <0.001 |
| | G4 | 129 ± 1 | 477 ± 52 | 733 ± 85 | 1306 ± 264 | 8/8 | 67.9% | 0.134 | <0.001 |

The results showed that anti-h4-1BB IgG1 antibodies had excellent tumor inhibitory effects against lymphoma cancer cells, and the combination with anti-h4-1BB IgG1 antibodies with anti-PD-1 antibody can further improve tumor inhibitor effects.

### Example 20. Further Analysis of Effects of Humanized Anti-4-1BB Antibodies

MC-38 cancer tumor cells (colon adenocarcinoma cell) were injected subcutaneously in B-h4-1BB mice. When the tumors in the mice reached a volume of about 200±50 mm³, the mice were randomly placed into different groups based on the volume of the tumor, and were administered with different antibodies as shown in the table below.

TABLE 48

| | No. of | | Dosage | | | Total No. of |
|---|---|---|---|---|---|---|
| Group | mice | Antibodies | (mg/kg) | Route | Frequency | administration |
| G1 | 5 | PS (control) | — | i.p. | QD | 1 |
| G2 | 5 | 1C4-H1K1-IgG1 | 3 mg/kg | i.p. | QD | 1 |
| G3 | 5 | 1C4-H1K1-IgG4 | 3 mg/kg | i.p. | QD | 1 |
| G4 | 5 | Urelumab-IgG1 | 3 mg/kg | i.p. | QD | 1 |
| G5 | 5 | Urelumab-IgG4 | 3 mg/kg | i.p. | QD | 1 |

Two days after the administration, the mice were sacrificed. The tumor samples and the spleen samples were collected for further analysis. The weight of the mice was also recorded as shown in the table below. No obvious differences in weight were observed among the different groups.

TABLE 49

| | | Body weight (g) | | | P value |
|---|---|---|---|---|---|
| | | Day 0 | Day 2 | Survival | Body weight |
| Control | G1 | 19.4 ± 0.5 | 19.7 ± 0.5 | 5/5 | n.a. |
| Treat | G2 | 19.6 ± 0.5 | 19.4 ± 0.5 | 5/5 | 0.699 |
| | G3 | 19.1 ± 0.3 | 19.7 ± 0.3 | 5/5 | 1.000 |
| | G4 | 18.8 ± 0.5 | 19.0 ± 0.6 | 5/5 | 0.408 |
| | G5 | 20.0 ± 0.5 | 20.0 ± 0.5 | 5/5 | 0.684 |

The collected tumor and spleen samples were grinded and treated with digestive enzymes. The mixture was incubated at 37° C. for 60 minutes, filtered, washed, and re-suspended as a single-cell suspension.

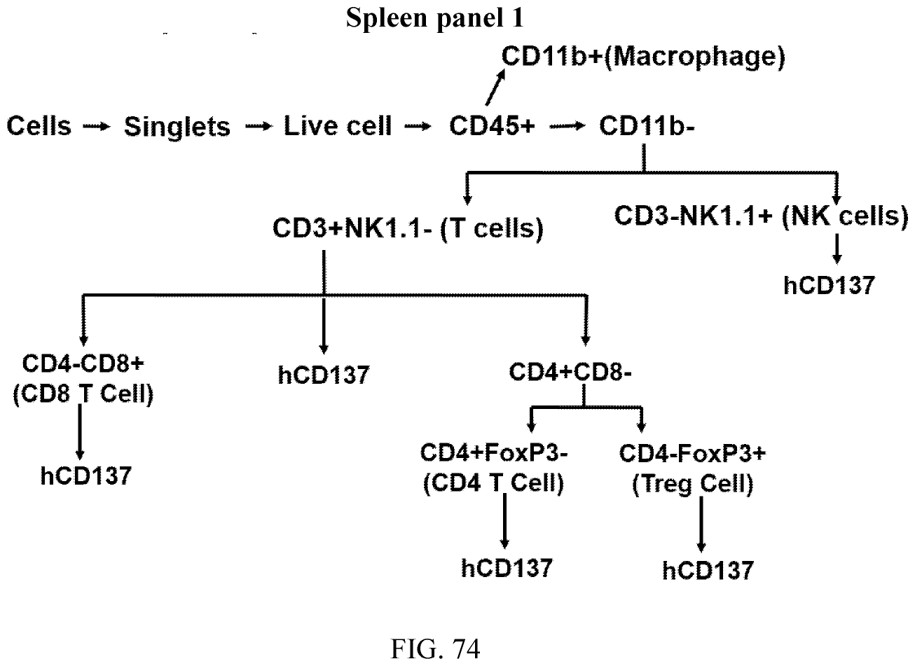

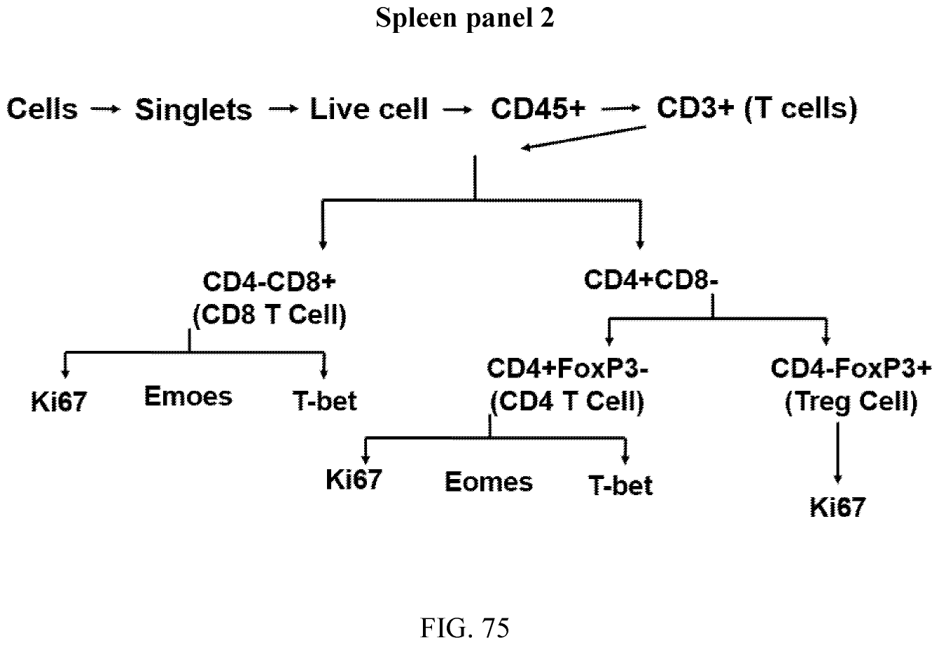

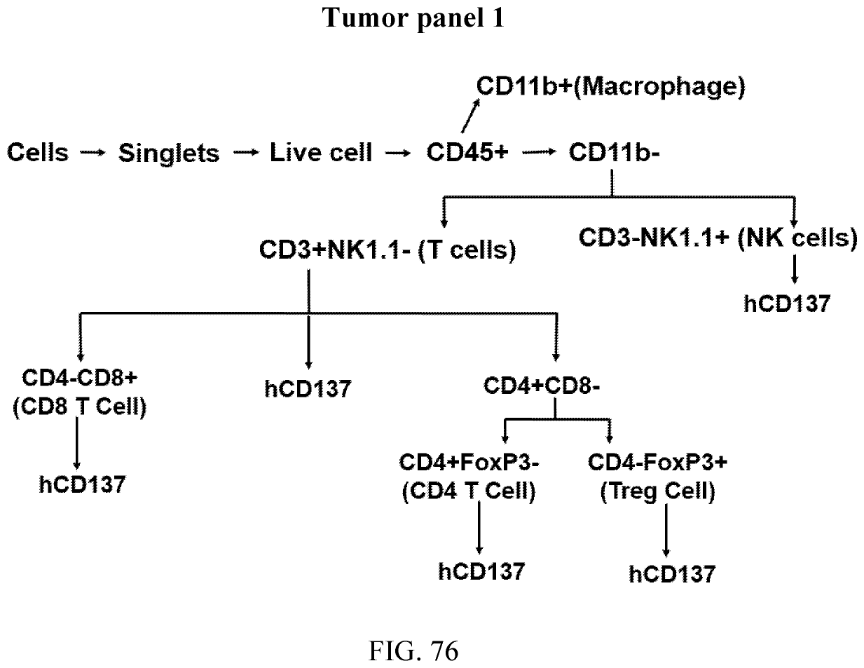

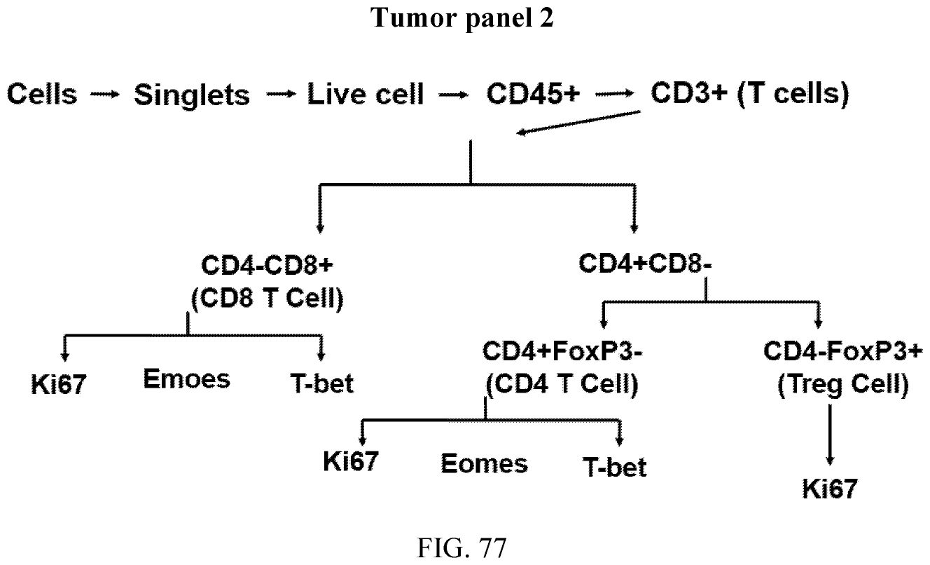

The cells were incubated with fluorescent-labeled antibodies and were analyzed by flow cytometry. The spleen samples were processed by the procedures as shown in FIG. 74 and FIG. 75. The tumor samples were processed by the procedures as shown in FIG. 76 and FIG. 77.

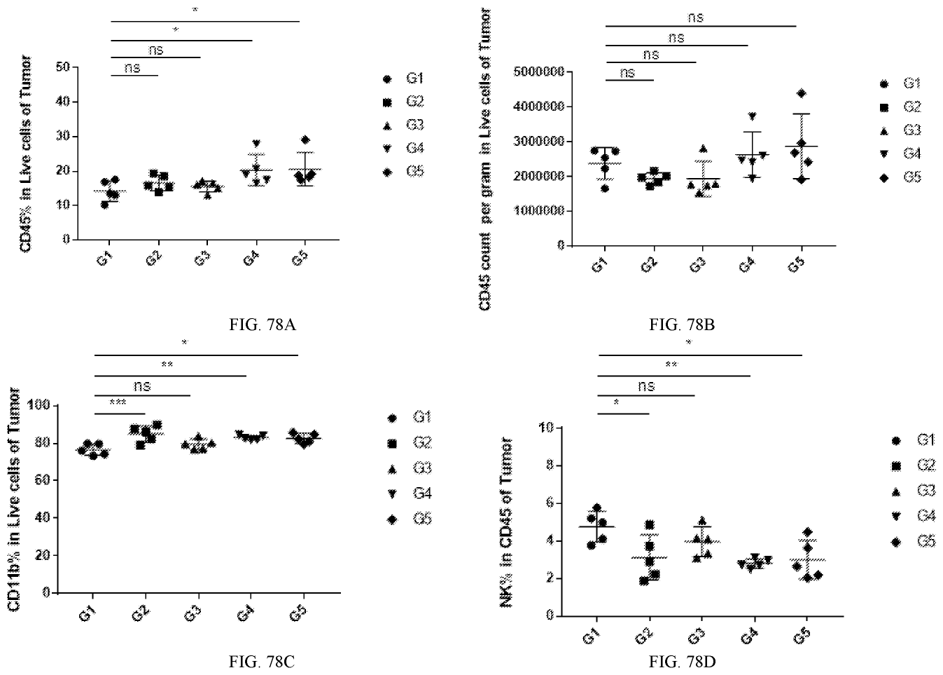

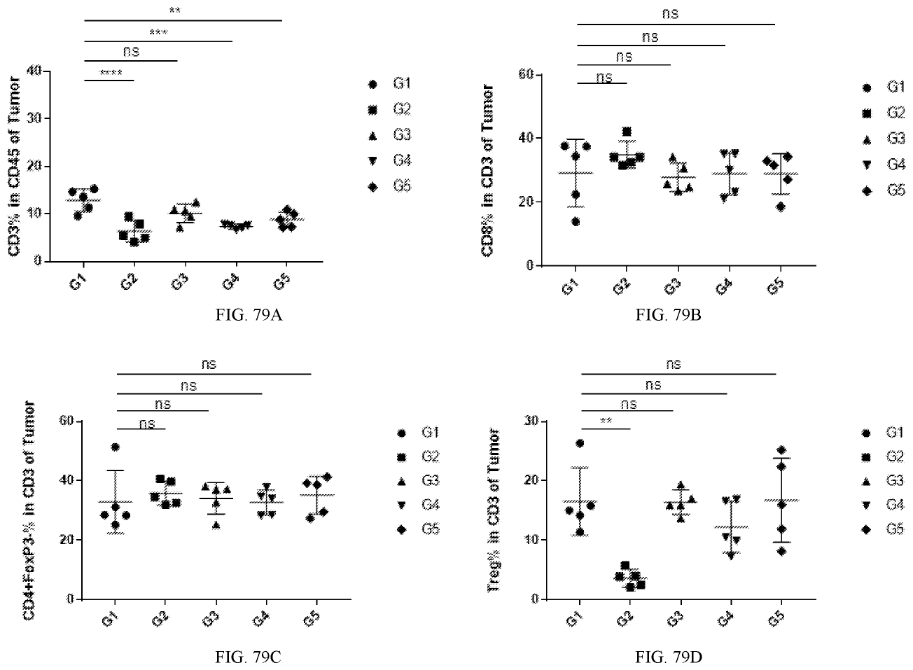

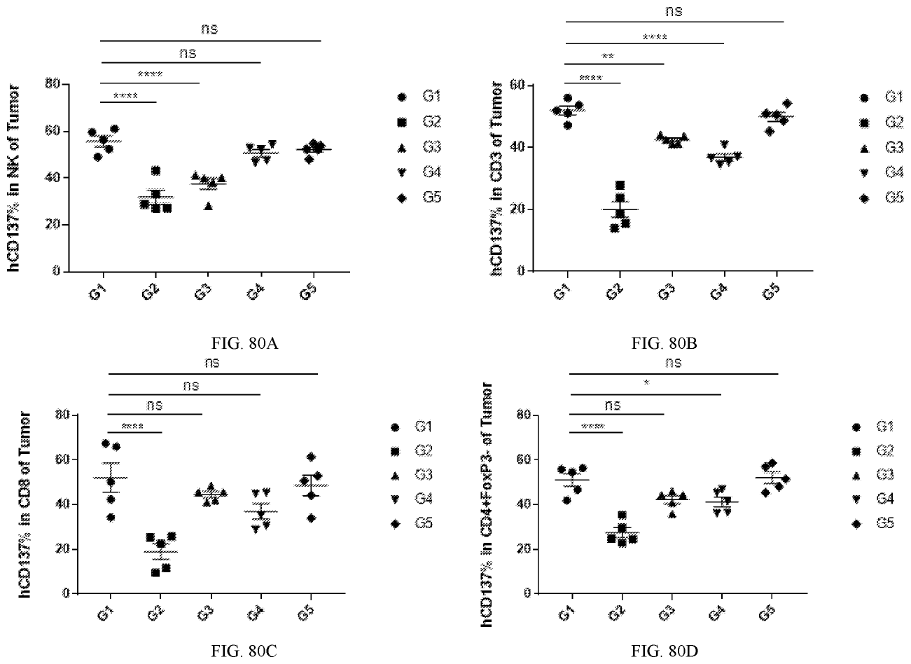

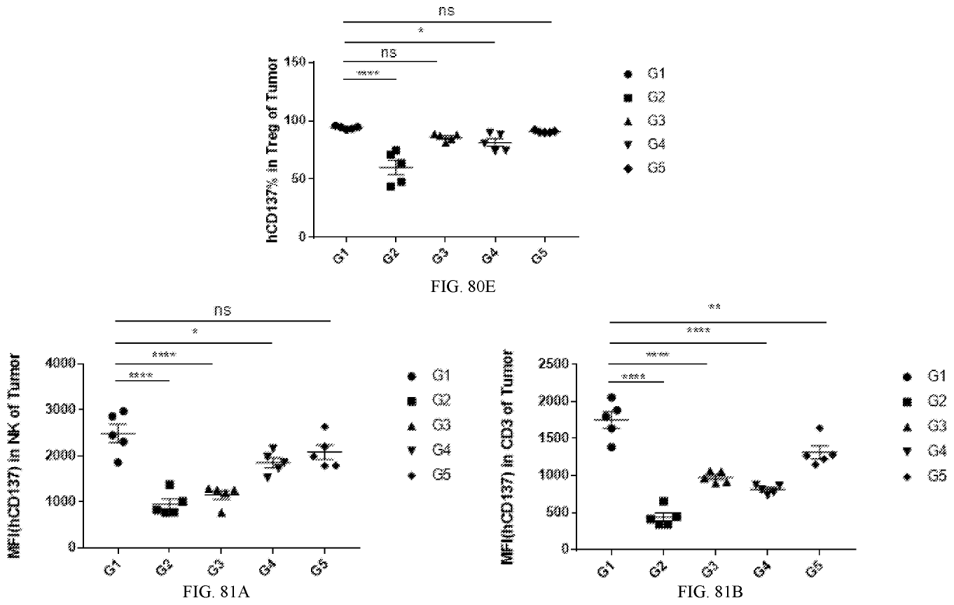

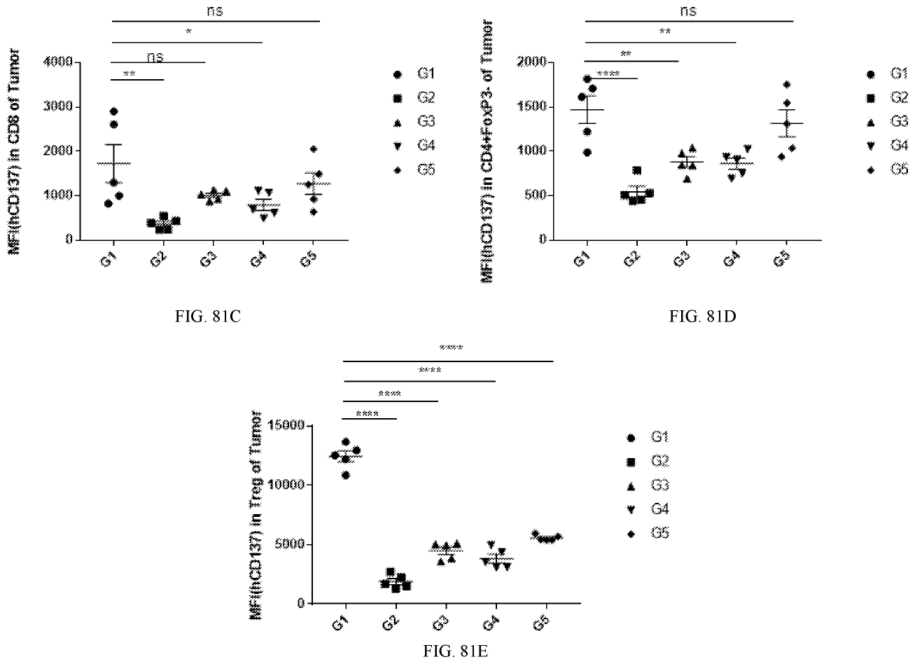

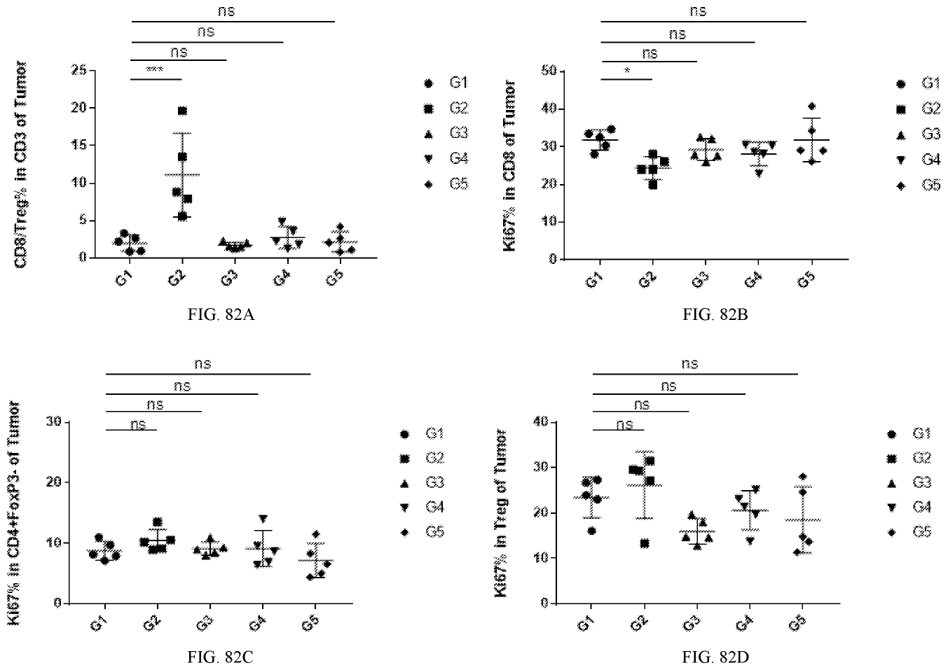

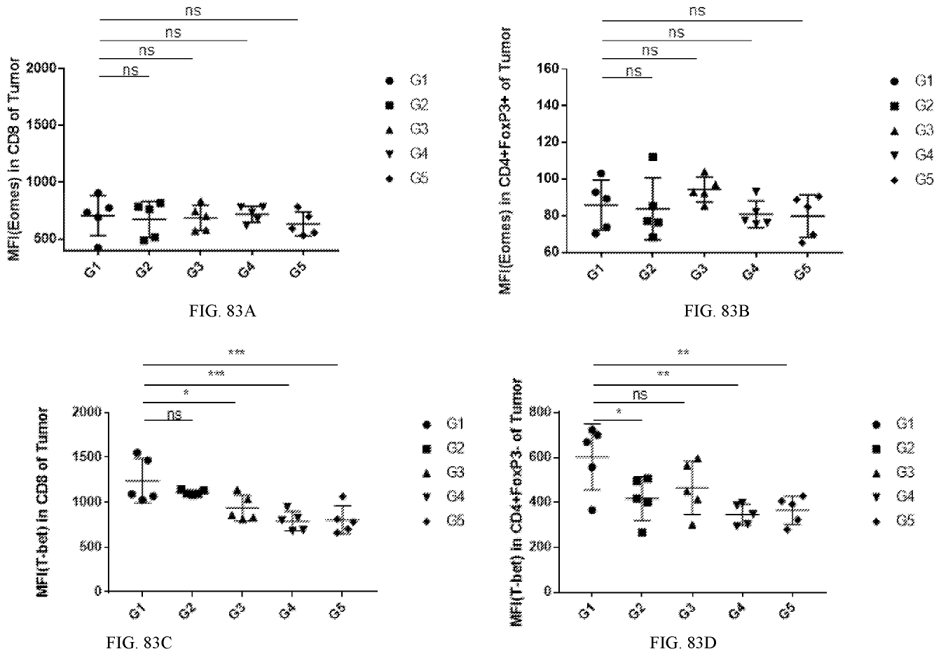

The results are shown in FIGS. 78-83. As shown in FIG. 79D, anti-4-1BB IgG1 antibodies can effectively decrease the percentage of Treg cells in tumors, thereby increasing immune response. The result is also consistent with the result in FIG. 82A, wherein a lower ratio of CD8 to Treg is associated with an unfavorable outcome. In addition, the results in FIG. 82A also explain that the 1C4 anti-4-1BB antibody has a higher tumor inhibitory effects as compared to Urelumab.

### Example 21. More Testing on Anti-h4-1BB IgG1 Antibodies

Experiments are repeated to test different subclasses of anti-h4-1BB IgG antibodies.

MC-38 cancer tumor cells (colon adenocarcinoma cell) are injected subcutaneously in B-h4-1BB mice. When the tumors in the mice reach a volume of 150±50 mm³, the mice are randomly placed into different groups based on the volume of the tumor (e.g., n=5), and are administered with IgG1, IgG2, and IgG4 versions of antibodies selected from Table 1, Table 2, and/or Table 4 through i.p. administration twice a week with 1 mg/kg or 3 mg/kg dosage. A total number of 6 administrations is performed. Physiological saline (PS) is administered to one group of mice as a control.

The weight of the mice and the tumor size are monitored during the entire treatment period. The TGI % at the end of the treatment period is calculated.

It is expected that anti-h4-1BB IgG1 subclasses have higher TGI %, thus better tumor inhibitory effects as compared to IgG2 and IgG4 subclasses.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody or antigen-binding fragment thereof that binds to 4-1BB (TNF Receptor Superfamily Member 9) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively;
(2) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(3) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 16, 17, 18, respectively;
(4) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(5) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 28, 29, 30, respectively;
(6) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 34, 35, 36, respectively;
(7) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 40, 41, 42, respectively;
(8) the selected VH CDRs 1, 2, 3 amino acid sequences are set forth in SEQ ID NOs: 43, 44, 45, respectively, and the selected VL CDRs 1, 2, 3 amino acid sequences are set forth in SEQ ID NOs: 46, 47, 48, respectively;
(9) the selected VH CDRs 1, 2, 3 amino acid sequences are set forth in SEQ ID NOs: 49, 50, 51, respectively, and the selected VL CDRs 1, 2, 3 amino acid sequences are set forth in SEQ ID NOs: 52, 53, 54, respectively;
(10) the selected VH CDRs 1, 2, 3 amino acid sequences are set forth in SEQ ID NOs: 55, 56, 57, respectively, and the selected VL CDRs 1, 2, 3 amino acid sequences are set forth in SEQ ID NOs: 58, 59, 60, respectively;
(11) the selected VH CDRs 1, 2, 3 amino acid sequences are set forth in SEQ ID NOs: 61, 62, 63, respectively, and the selected VL CDRs 1, 2, 3 amino acid sequences are set forth in SEQ ID NOs: 64, 65, 66, respectively;
(12) the selected VH CDRs 1, 2, 3 amino acid sequences are set forth in SEQ ID NOs: 67, 68, 69, respectively, and the selected VL CDRs 1, 2, 3 amino acid sequences are set forth in SEQ ID NOs: 70, 71, 72, respectively;
(13) the selected VH CDRs 1, 2, 3 amino acid sequences are set forth in SEQ ID NOs: 73, 74, 75, respectively, and the selected VL CDRs 1, 2, 3 amino acid sequences are set forth in SEQ ID NOs: 76, 77, 78, respectively;
(14) the selected VH CDRs 1, 2, 3 amino acid sequences are set forth in SEQ ID NOs: 79, 80, 81, respectively, and the selected VL CDRs 1, 2, 3 amino acid sequences are set forth in SEQ ID NOs: 82, 83, 84, respectively;
(15) the selected VH CDRs 1, 2, 3 amino acid sequences are set forth in SEQ ID NOs: 85, 86, 87, respectively, and the selected VL CDRs 1, 2, 3 amino acid sequences are set forth in SEQ ID NOs: 88, 89, 90, respectively;
(16) the selected VH CDRs 1, 2, 3 amino acid sequences are set forth in SEQ ID NOs: 91, 92, 93, respectively, and the selected VL CDRs 1, 2, 3 amino acid sequences are set forth in SEQ ID NOs: 94, 95, 96, respectively;
(17) the selected VH CDRs 1, 2, 3 amino acid sequences are set forth in SEQ ID NOs: 97, 98, 99, respectively, and the selected VL CDRs 1, 2, 3 amino acid sequences are set forth in SEQ ID NOs: 100, 101, 102, respectively;
(18) the selected VH CDRs 1, 2, 3 amino acid sequences are set forth in SEQ ID NOs: 103, 104, 105, respectively, and the selected VL CDRs 1, 2, 3 amino acid sequences are set forth in SEQ ID NOs: 106, 107, 108, respectively;
(19) the selected VH CDRs 1, 2, 3 amino acid sequences are set forth in SEQ ID NOs: 109, 110, 111, respectively, and the selected VL CDRs 1, 2, 3 amino acid sequences are set forth in SEQ ID NOs: 112, 113, 114, respectively;
(20) the selected VH CDRs 1, 2, 3 amino acid sequences are set forth in SEQ ID NOs: 115, 116, 117, respectively, and the selected VL CDRs 1, 2, 3 amino acid sequences are set forth in SEQ ID NOs: 118, 119, 120, respectively;
(21) the selected VH CDRs 1, 2, 3 amino acid sequences are set forth in SEQ ID NOs: 121, 122, 123, respectively, and the selected VL CDRs 1, 2, 3 amino acid sequences are set forth in SEQ ID NOs: 124, 125, 126, respectively;
(22) the selected VH CDRs 1, 2, 3 amino acid sequences are set forth in SEQ ID NOs: 127, 128, 129, respectively, and the selected VL CDRs 1, 2, 3 amino acid sequences are set forth in SEQ ID NOs: 130, 131, 132, respectively;
(23) the selected VH CDRs 1, 2, 3 amino acid sequences are set forth in SEQ ID NOs: 133, 134, 135, respectively, and the selected VL CDRs 1, 2, 3 amino acid sequences are set forth in SEQ ID NOs: 136, 137, 138, respectively;
(24) the selected VH CDRs 1, 2, 3 amino acid sequences are set forth in SEQ ID NOs: 139, 140, 141, respectively, and the selected VL CDRs 1, 2, 3 amino acid sequences are set forth in SEQ ID NOs: 142, 143, 144, respectively;
(25) the selected VH CDRs 1, 2, 3 amino acid sequences are set forth in SEQ ID NOs: 145, 146, 147, respectively, and the selected VL CDRs 1, 2, 3 amino acid sequences are set forth in SEQ ID NOs: 148, 149, 150, respectively;
(26) the selected VH CDRs 1, 2, 3 amino acid sequences are set forth in SEQ ID NOs: 151, 152, 153, respectively, and the selected VL CDRs 1, 2, 3 amino acid sequences are set forth in SEQ ID NOs: 154, 155, 156, respectively;
(27) the selected VH CDRs 1, 2, 3 amino acid sequences are set forth in SEQ ID NOs: 157, 158, 159, respectively, and the selected VL CDRs 1, 2, 3 amino acid sequences are set forth in SEQ ID NOs: 160, 161, 162, respectively;
(28) the selected VH CDRs 1, 2, 3 amino acid sequences are set forth in SEQ ID NOs: 163, 164, 165, respectively, and the selected VL CDRs 1, 2, 3 amino acid sequences are set forth in SEQ ID NOs: 166, 167, 168, respectively;
(29) the selected VH CDRs 1, 2, 3 amino acid sequences are set forth in SEQ ID NOs: 169, 170, 171, respectively, and the selected VL CDRs 1, 2, 3 amino acid sequences are set forth in SEQ ID NOs: 172, 173, 174, respectively;
(30) the selected VH CDRs 1, 2, 3 amino acid sequences are set forth in SEQ ID NOs: 175, 176, 177, respectively, and the selected VL CDRs 1, 2, 3 amino acid sequences are set forth in SEQ ID NOs: 178, 179, 180, respectively;
(31) the selected VH CDRs 1, 2, 3 amino acid sequences are set forth in SEQ ID NOs: 181, 182, 183, respectively, and the selected VL CDRs 1, 2, 3 amino acid sequences are set forth in SEQ ID NOs: 184, 185, 186, respectively;
(32) the selected VH CDRs 1, 2, 3 amino acid sequences are set forth in SEQ ID NOs: 187, 188, 189, respectively, and the selected VL CDRs 1, 2, 3 amino acid sequences are set forth in SEQ ID NOs: 190, 191, 192, respectively;
(33) the selected VH CDRs 1, 2, 3 amino acid sequences are set forth in SEQ ID NOs: 193, 194, 195, respectively, and the selected VL CDRs 1, 2, 3 amino acid sequences are set forth in SEQ ID NOs: 196, 197, 198, respectively;
(34) the selected VH CDRs 1, 2, 3 amino acid sequences are set forth in SEQ ID NOs: 199, 200, 201, respectively, and the selected VL CDRs 1, 2, 3 amino acid sequences are set forth in SEQ ID NOs: 202, 203, 204, respectively;
(35) the selected VH CDRs 1, 2, 3 amino acid sequences are set forth in SEQ ID NOs: 205, 206, 207, respectively, and the selected VL CDRs 1, 2, 3 amino acid sequences are set forth in SEQ ID NOs: 208, 209, 210, respectively; and
(36) the selected VH CDRs 1, 2, 3 amino acid sequences are set forth in SEQ ID NOs: 211, 212, 213, respectively, and the selected VL CDRs 1, 2, 3 amino acid sequences are set forth in SEQ ID NOs: 214, 215, 216, respectively.

2. The antibody or antigen-binding fragment thereof of claim 1, wherein the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 4, 5, and 6, respectively.

3. The antibody or antigen-binding fragment thereof of claim 1, wherein the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 10, 11, and 12, respectively.

4. The antibody or antigen-binding fragment thereof of claim 1, wherein the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively.

5. The antibody or antigen-binding fragment thereof of claim 1, wherein the antibody or antigen-binding fragment specifically binds to human 4-1BB.

6. The antibody or antigen-binding fragment thereof of claim 1, wherein the antibody or antigen-binding fragment is a humanized antibody or antigen-binding fragment thereof.

7. The antibody or antigen-binding fragment thereof claim 1, wherein the antibody or antigen-binding fragment is a single-chain variable fragment (scFv) or a multi-specific antibody.

8. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 225, 226, 227, 228, or 244 binds to 4-1BB;
(2) an immunoglobulin light chain or a fragment thereof comprising a VL comprising CDRs 1, 2, and 3 comprising the amino acid sequences set forth in SEQ ID NOs: 4, 5, and 6, respectively, and wherein the VL, when paired with a VH comprising the amino acid sequence set forth in SEQ ID NO: 221, 222, 223, 224, or 243 binds to 4-1BB;
(3) an immunoglobulin heavy chain or a fragment thereof comprising a heavy chain variable region (VH) comprising CDRs 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 232, 233, 234, 235, or 246 binds to 4-1BB;
(4) an immunoglobulin light chain or a fragment thereof comprising a VL comprising CDRs 1, 2, and 3 comprising the amino acid sequences set forth in SEQ ID NOs: 10, 11, and 12, respectively, and wherein the VL, when paired with a VH comprising the amino acid sequence set forth in SEQ ID NO: 229, 230, 231, or 245 binds to 4-1BB;
(5) an immunoglobulin heavy chain or a fragment thereof comprising a heavy chain variable region (VH) comprising CDRs 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 239, 240, 241, 242, or 248 binds to 4-1BB;
(6) an immunoglobulin light chain or a fragment thereof comprising a VL comprising CDRs 1, 2, and 3 comprising the amino acid sequences set forth in SEQ ID NOs: 16, 17, and 18, respectively, and wherein the VL, when paired with a VH comprising the amino acid sequence set forth in SEQ ID NO: 236, 237, 238, or 247 binds to 4-1BB;
(7) an immunoglobulin heavy chain or a fragment thereof comprising a VH comprising CDRs 1, 2, and 3 comprising the amino acid sequences set forth in SEQ ID NOs: 19, 20, 21, respectively, and wherein the VH, when paired with a VL comprising the amino acid sequence set forth in SEQ ID NO: 250 binds to 4-1BB;
(8) an immunoglobulin light chain or a fragment thereof comprising a VL comprising CDRs 1, 2, and 3 comprising the amino acid sequences set forth in SEQ ID NOs: 22, 23, 24, respectively, and wherein the VL, when paired with a VH comprising the amino acid sequence set forth in SEQ ID NO: 249 binds to 4-1BB;
(9) an immunoglobulin heavy chain or a fragment thereof comprising a VH comprising CDRs 1, 2, and 3 comprising the amino acid sequences set forth in SEQ ID NOs: 25, 26, 27, respectively, and wherein the VH, when paired with a VL comprising the amino acid sequence set forth in SEQ ID NO: 252 binds to 4-1BB;
(10) an immunoglobulin light chain or a fragment thereof comprising a VL comprising CDRs 1, 2, and 3 comprising the amino acid sequences set forth in SEQ ID NOs: 28, 29, 30, respectively, and wherein the VL, when paired with a VH comprising the amino acid sequence set forth in SEQ ID NO: 251 binds to 4-1BB;
(11) an immunoglobulin heavy chain or a fragment thereof comprising a VH comprising CDRs 1, 2, and 3 comprising the amino acid sequences set forth in SEQ ID NOs: 31, 32, 33, respectively, and wherein the VH, when paired with a VL comprising the amino acid sequence set forth in SEQ ID NO: 254 binds to 4-1BB;
(12) an immunoglobulin light chain or a fragment thereof comprising a VL comprising CDRs 1, 2, and 3 comprising the amino acid sequences set forth in SEQ ID NOs: 34, 35, 36, respectively, and wherein the VL, when paired with a VH comprising the amino acid sequence set forth in SEQ ID NO: 253 binds to 4-1BB;
(13) an immunoglobulin heavy chain or a fragment thereof comprising a VH comprising CDRs 1, 2, and 3 comprising the amino acid sequences set forth in SEQ ID NOs: 37, 38, 39, respectively, and wherein the VH, when paired with a VL comprising the amino acid sequence set forth in SEQ ID NO: 256 binds to 4-1BB;
(14) an immunoglobulin light chain or a fragment thereof comprising a VL comprising CDRs 1, 2, and 3 comprising the amino acid sequences set forth in SEQ ID NOs: 40, 41, 42, respectively, and wherein the VL, when paired with a VH comprising the amino acid sequence set forth in SEQ ID NO: 255 binds to 4-1BB;
(15) an immunoglobulin heavy chain or a fragment thereof comprising a VH comprising CDRs 1, 2, and 3 comprising the amino acid sequences set forth in SEQ ID NOs: 43, 44, 45, respectively, and wherein the VH, when paired with a VL comprising the amino acid sequence set forth in SEQ ID NO: 258 binds to 4-1BB;
(16) an immunoglobulin light chain or a fragment thereof comprising a VL comprising CDRs 1, 2, and 3 comprising the amino acid sequences set forth in SEQ ID NOs: 46, 47, 48, respectively, and wherein the VL, when paired with a VH comprising the amino acid sequence set forth in SEQ ID NO: 257 binds to 4-1BB;
(17) an immunoglobulin heavy chain or a fragment thereof comprising a VH comprising CDRs 1, 2, and 3 comprising the amino acid sequences set forth in SEQ ID NOs: 49, 50, 51, respectively, and wherein the VH, when paired with a VL comprising the amino acid sequence set forth in SEQ ID NO: 260 binds to 4-1BB;
(18) an immunoglobulin light chain or a fragment thereof comprising a VL comprising CDRs 1, 2, and 3 comprising the amino acid sequences set forth in SEQ ID NOs: 52, 53, 54, respectively, and wherein the VL, when paired with a VH comprising the amino acid sequence set forth in SEQ ID NO: 259 binds to 4-1BB;
(19) an immunoglobulin heavy chain or a fragment thereof comprising a VH comprising CDRs 1, 2, and 3 comprising the amino acid sequences set forth in SEQ ID NOs: 55, 56, 57, respectively, and wherein the VH, when paired with a VL comprising the amino acid sequence set forth in SEQ ID NO: 262 binds to 4-1BB;
(20) an immunoglobulin light chain or a fragment thereof comprising a VL comprising CDRs 1, 2, and 3 comprising the amino acid sequences set forth in SEQ ID NOs: 58, 59, 60, respectively, and wherein the VL, when paired with a VH comprising the amino acid sequence set forth in SEQ ID NO: 261 binds to 4-1BB;
(21) an immunoglobulin heavy chain or a fragment thereof comprising a VH comprising CDRs 1, 2, and 3 comprising the amino acid sequences set forth in SEQ ID NOs: 61, 62, 63, respectively, and wherein the VH, when paired with a VL comprising the amino acid sequence set forth in SEQ ID NO: 264 binds to 4-1BB;
(22) an immunoglobulin light chain or a fragment thereof comprising a VL comprising CDRs 1, 2, and 3 comprising the amino acid sequences set forth in SEQ ID NOs: 64, 65, 66, respectively, and wherein the VL, when paired with a VH comprising the amino acid sequence set forth in SEQ ID NO: 263 binds to 4-1BB;
(23) an immunoglobulin heavy chain or a fragment thereof comprising a VH comprising CDRs 1, 2, and 3 comprising the amino acid sequences set forth in SEQ ID NOs: 67, 68, 69, respectively, and wherein the VH, when paired with a VL comprising the amino acid sequence set forth in SEQ ID NO: 266 binds to 4-1BB;
(24) an immunoglobulin light chain or a fragment thereof comprising a VL comprising CDRs 1, 2, and 3 comprising the amino acid sequences set forth in SEQ ID NOs: 70, 71, 72, respectively, and wherein the VL, when paired with a VH comprising the amino acid sequence set forth in SEQ ID NO: 265 binds to 4-1BB;
(25) an immunoglobulin heavy chain or a fragment thereof comprising a VH comprising CDRs 1, 2, and 3 comprising the amino acid sequences set forth in SEQ ID NOs: 73, 74, 75, respectively, and wherein the VH, when paired with a VL comprising the amino acid sequence set forth in SEQ ID NO: 268 binds to 4-1BB;
(26) an immunoglobulin light chain or a fragment thereof comprising a VL comprising CDRs 1, 2, and 3 comprising the amino acid sequences set forth in SEQ ID NOs: 76, 77, 78, respectively, and wherein the VL, when paired with a VH comprising the amino acid sequence set forth in SEQ ID NO: 267 binds to 4-1BB;
(27) an immunoglobulin heavy chain or a fragment thereof comprising a VH comprising CDRs 1, 2, and 3 comprising the amino acid sequences set forth in SEQ ID NOs: 79, 80, 81, respectively, and wherein the VH, when paired with a VL comprising the amino acid sequence set forth in SEQ ID NO: 270 binds to 4-1BB;
(28) an immunoglobulin light chain or a fragment thereof comprising a VL comprising CDRs 1, 2, and 3 comprising the amino acid sequences set forth in SEQ ID NOs: 82, 83, 84, respectively, and wherein the VL, when paired with a VH comprising the amino acid sequence set forth in SEQ ID NO: 269 binds to 4-1BB;
(29) an immunoglobulin heavy chain or a fragment thereof comprising a VH comprising CDRs 1, 2, and 3 comprising the amino acid sequences set forth in SEQ ID NOs: 85, 86, 87, respectively, and wherein the VH, when paired with a VL comprising the amino acid sequence set forth in SEQ ID NO: 272 binds to 4-1BB;
(30) an immunoglobulin light chain or a fragment thereof comprising a VL comprising CDRs 1, 2, and 3 comprising the amino acid sequences set forth in SEQ ID NOs: 88, 89, 90, respectively, and wherein the VL, when paired with a VH comprising the amino acid sequence set forth in SEQ ID NO: 271 binds to 4-1BB;
(31) an immunoglobulin heavy chain or a fragment thereof comprising a VH comprising CDRs 1, 2, and 3 comprising the amino acid sequences set forth in SEQ ID NOs: 91, 92, 93, respectively, and wherein the VH, when paired with a VL comprising the amino acid sequence set forth in SEQ ID NO: 274 binds to 4-1BB;
(32) an immunoglobulin light chain or a fragment thereof comprising a VL comprising CDRs 1, 2, and 3 comprising the amino acid sequences set forth in SEQ ID NOs: 94, 95, 96, respectively, and wherein the VL, when paired with a VH comprising the amino acid sequence set forth in SEQ ID NO: 273 binds to 4-1BB;
(33) an immunoglobulin heavy chain or a fragment thereof comprising a VH comprising CDRs 1, 2, and 3 comprising the amino acid sequences set forth in SEQ ID NOs: 97, 98, 99, respectively, and wherein the VH, when paired with a VL comprising the amino acid sequence set forth in SEQ ID NO: 276 binds to 4-1BB;
(34) an immunoglobulin light chain or a fragment thereof comprising a VL comprising CDRs 1, 2, and 3 comprising the amino acid sequences set forth in SEQ ID NOs: 100, 101, 102, respectively, and wherein the VL, when paired with a VH comprising the amino acid sequence set forth in SEQ ID NO: 275 binds to 4-1BB;
(35) an immunoglobulin heavy chain or a fragment thereof comprising a VH comprising CDRs 1, 2, and 3 comprising the amino acid sequences set forth in SEQ ID NOs: 103, 104, 105, respectively, and wherein the VH, when paired with a VL comprising the amino acid sequence set forth in SEQ ID NO: 278 binds to 4-1BB; or
(36) an immunoglobulin light chain or a fragment thereof comprising a VL comprising CDRs 1, 2, and 3 comprising the amino acid sequences set forth in SEQ ID NOs: 106, 107, 108, respectively, and wherein the VL, when paired with a VH comprising the amino acid sequence set forth in SEQ ID NO: 277 binds to 4-1BB.

9.-18. (canceled)

19. A vector comprising one or more of the nucleic acids of claim 8.

20.-21. (canceled)

22. A cell comprising one or more of the nucleic acids of claim 8.

23.-26. (canceled)

27. A method of producing an antibody or an antigen-binding fragment thereof, the method comprising
culturing the cell of claim 22 under conditions sufficient for the cell to produce the antibody or the antigen-binding fragment.

28. The antibody or antigen-binding fragment thereof of claim 1, wherein the
VH comprises an amino acid sequence that is at least 90% identical to a selected VH sequence, and the VL comprises an amino acid sequence that is at least 90% identical to a selected VL sequence, wherein the selected VH sequence and the selected VL sequence are one of the following:
(1) the selected VH sequence is SEQ ID NOs: 221, 222, 223, 224, or 243, and the selected VL sequence is SEQ ID NOs: 225, 226, 227, 228, or 244;
(2) the selected VH sequence is SEQ ID NOs: 229, 230, 231, or 245, and the selected VL sequence is SEQ ID NOs: 232, 233, 234, 235, or 246;
(3) the selected VH sequence is SEQ ID NO: 236, 237, 238, or 247, and the selected VL sequence is SEQ ID NO: 239, 240, 241, 242, or 248;
(4) the selected VH sequence is SEQ ID NO: 249, and the selected VL sequence is SEQ ID NO: 250;
(5) the selected VH sequence is SEQ ID NO: 251, and the selected VL sequence is SEQ ID NO: 252;
(6) the selected VH sequence is SEQ ID NO: 253, and the selected VL sequence is SEQ ID NO: 254;
(7) the selected VH sequence is SEQ ID NO: 255, and the selected VL sequence is SEQ ID NO: 256;
(8) the selected VH sequence is SEQ ID NO: 257, and the selected VL sequence is SEQ ID NO: 258;
(9) the selected VH sequence is SEQ ID NO: 259, and the selected VL sequence is SEQ ID NO: 260;
(10) the selected VH sequence is SEQ ID NO: 261, and the selected VL sequence is SEQ ID NO: 262;
(11) the selected VH sequence is SEQ ID NO: 263, and the selected VL sequence is SEQ ID NO: 264;
(12) the selected VH sequence is SEQ ID NO: 265, and the selected VL sequence is SEQ ID NO: 266;
(13) the selected VH sequence is SEQ ID NO: 267, and the selected VL sequence is SEQ ID NO: 268;
(14) the selected VH sequence is SEQ ID NO: 269, and the selected VL sequence is SEQ ID NO: 270;
(15) the selected VH sequence is SEQ ID NO: 271, and the selected VL sequence is SEQ ID NO: 272;
(16) the selected VH sequence is SEQ ID NO: 273, and the selected VL sequence is SEQ ID NO: 274;
(17) the selected VH sequence is SEQ ID NO: 275, and the selected VL sequence is SEQ ID NO: 276; and
(18) the selected VH sequence is SEQ ID NO: 277, and the selected VL sequence is SEQ ID NO: 278.

29.-33. (canceled)

34. An antibody-drug conjugate comprising the antibody or antigen-binding fragment thereof of claim 1 covalently bound to a therapeutic agent.

35. (canceled)

36. A method of treating a subject having cancer, the method comprising administering a therapeutically effective amount of a composition comprising the antibody or antigen-binding fragment thereof of claim 1 to the subject.

37. The method of claim 36, wherein the subject has a solid tumor, breast cancer, oropharyngeal cancer, ovarian cancer, B cell lymphoma, Non-Hodgkin's lymphoma, non-small cell lung cancer (NSCLC), melanoma, B-cell non-Hodgkin lymphoma, colorectal cancer, hematologic malignancy, head and neck cancer, bladder cancer, or multiple myeloma.

38.-39. (canceled)

40. A method of decreasing the rate of tumor growth, the method comprising
administering to a subject in need thereof an effective amount of a composition comprising an antibody or antigen-binding fragment thereof of claim 1.

41. A method of killing a tumor cell, the method comprising administering to a subject in need thereof an effective amount of a composition comprising the antibody or antigen-binding fragment thereof of claim 1.

42. A pharmaceutical composition comprising the antibody or antigen-binding fragment thereof of claim 1, and a pharmaceutically acceptable carrier.

43.-45. (canceled)

46. The method of claim 52, wherein the anti-4-1BB IgG1 antibody or antigen-binding fragment thereof comprises:
(1) a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, wherein the selected VH CDRs 1, 2, and 3 amino acid sequences and the selected VL CDRs, 1, 2, and 3 amino acid sequences are selected from one of the antibodies as set forth in Table 3; or
(2) a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are selected from one of the antibodies as set forth in Table 3.

47.-51. (canceled)

52. A method of treating a subject having cancer, the method comprising administering to the subject a therapeutically effective amount of an anti-4-1BB IgG1 antibody or antigen-binding fragment thereof and
(1) a therapeutically effective amount of an anti-PD-1 antibody or antigen-binding fragment thereof; or
(2) a therapeutically effective amount of an anti-CTLA4 antibody or antigen-binding fragment thereof.

53.-57. (canceled)


SEQUENCE LISTING (518 sequences)

SEQ ID NO: 1 - protein, 5 aa, synthetic construct
SSYIN

SEQ ID NO: 2 - protein, 17 aa, synthetic construct
WIFAGTGGTYYNQKFTG

SEQ ID NO: 3 - protein, 10 aa, synthetic construct
HSPRATLFDF

SEQ ID NO: 4 - protein, 10 aa, synthetic construct
RASSSVNHMH

SEQ ID NO: 5 - protein, 7 aa, synthetic construct
ATSNLAS

SEQ ID NO: 6 - protein, 9 aa, synthetic construct
QQWSSNPYT

SEQ ID NO: 7 - protein, 5 aa, synthetic construct
SYWIN

SEQ ID NO: 8 - protein, 17 aa, synthetic construct
NIYPSDSYTNYNQKFKD

SEQ ID NO: 9 - protein, 12 aa, synthetic construct
TWEFYYDSVFDY

SEQ ID NO: 10 - protein, 11 aa, synthetic construct
KASQDINSYLS

SEQ ID NO: 11 - protein, 7 aa, synthetic construct
RANRLVD

SEQ ID NO: 12 - protein, 9 aa, synthetic construct
LQYDELPPT

SEQ ID NO: 13 - protein, 5 aa, synthetic construct
TYDIS

SEQ ID NO: 14 - protein, 16 aa, synthetic construct
VIWTGGGTNYNSSFMS

SEQ ID NO: 15 - skipped

SEQ ID NO: 16 - protein, 16 aa, synthetic construct
RSSQSLVHSNGNTYLH

SEQ ID NO: 17 - protein, 7 aa, synthetic construct
KVSNRFS

SEQ ID NO: 18 - protein, 9 aa, synthetic construct
SQNTHVPWT

SEQ ID NO: 19 - protein, 5 aa, synthetic construct
TSWMN

SEQ ID NO: 20 - protein, 17 aa, synthetic construct
RIYPGDGDTDYNGRFKG

SEQ ID NO: 21 - protein, 12 aa, synthetic construct
LDGYYEVFYFDY

SEQ ID NO: 22 - protein, 16 aa, synthetic construct
KSSQSLLYSNGKTYLN

SEQ ID NO: 23 - protein, 7 aa, synthetic construct
LVSKLDS

SEQ ID NO: 24 - protein, 9 aa, synthetic construct
FQGTHFPWT

SEQ ID NO: 25 - protein, 5 aa, synthetic construct
TYVMY

SEQ ID NO: 26 - protein, 17 aa, synthetic construct
YINPYNDDIRYNEKFKG

SEQ ID NO: 27 - protein, 5 aa, synthetic construct
QGGDY

SEQ ID NO: 28 - protein, 11 aa, synthetic construct
RASKNINKYLA

SEQ ID NO: 29 - protein, 7 aa, synthetic construct
SGSTLQS

SEQ ID NO: 30 - protein, 9 aa, synthetic construct
QQYNEYPLT

SEQ ID NO: 31 - protein, 5 aa, synthetic construct
TYGVH

SEQ ID NO: 32 - protein, 16 aa, synthetic construct
VIWSDGNTDYNDAFIS

SEQ ID NO: 33 - protein, 10 aa, synthetic construct
NSITSVSFDC

SEQ ID NO: 34 - protein, 11 aa, synthetic construct
KASQNVGTTVA

SEQ ID NO: 35 - protein, 7 aa, synthetic construct
SASYRYS

SEQ ID NO: 36 - protein, 9 aa, synthetic construct
QQYNSYPLT

SEQ ID NO: 37 - protein, 5 aa, synthetic construct
NYWIH

SEQ ID NO: 38 - protein, 17 aa, synthetic construct
NIYPASDYTNYDEKFKN

SEQ ID NO: 39 - protein, 8 aa, synthetic construct
GYFGSLDY

SEQ ID NO: 40 - protein, 11 aa, synthetic construct
RASQPIGTGIH

SEQ ID NO: 41 - protein, 7 aa, synthetic construct
SASESIS

SEQ ID NO: 42 - protein, 9 aa, synthetic construct
QQSYSWPTT

SEQ ID NO: 43 - protein, 5 aa, synthetic construct
SNWMH

SEQ ID NO: 44 - protein, 17 aa, synthetic construct
VIYPGNSDTSYNQKFKG

SEQ ID NO: 45 - protein, 8 aa, synthetic construct
GITTAPEY

SEQ ID NO: 46 - protein, 11 aa, synthetic construct
KASENVGIYVS

SEQ ID NO: 47 - protein, 7 aa, synthetic construct
GASNRYT

SEQ ID NO: 48 - protein, 9 aa, synthetic construct
GHTYNYPFT

SEQ ID NO: 49 - protein, 5 aa, synthetic construct
RYWMS

SEQ ID NO: 50 - protein, 17 aa, synthetic construct
EIHPDSSAINYTPSLKD

SEQ ID NO: 51 - protein, 9 aa, synthetic construct
SYYGRVFDY

SEQ ID NO: 52 - protein, 11 aa, synthetic construct
RASQSISDYLH

SEQ ID NO: 53 - protein, 7 aa, synthetic construct
YASQSIS

SEQ ID NO: 54 - protein, 9 aa, synthetic construct
QDGHSFPPT

SEQ ID NO: 55 - protein, 5 aa, synthetic construct
SYWMH

SEQ ID NO: 56 - protein, 17 aa, synthetic construct
VIYPGNSDTSYNQKFKG

SEQ ID NO: 57 - protein, 8 aa, synthetic construct
GITTAPDY

SEQ ID NO: 58 - protein, 11 aa, synthetic construct
KASENVGIHVS

SEQ ID NO: 59 - protein, 7 aa, synthetic construct
GASSGYN

SEQ ID NO: 60 - protein, 9 aa, synthetic construct
GQTYSYPFT

SEQ ID NO: 61 - protein, 5 aa, synthetic construct
NYGMN

SEQ ID NO: 62 - protein, 17 aa, synthetic construct
WINTYTGEPTYADDFKG

SEQ ID NO: 63 - protein, 10 aa, synthetic construct
REYYPWVFVY

SEQ ID NO: 64 - protein, 10 aa, synthetic construct
SASSSVSYMH

SEQ ID NO: 65 - protein, 7 aa, synthetic construct
DTSKLAS

SEQ ID NO: 66 - protein, 9 aa, synthetic construct
FQGSGYPLT

SEQ ID NO: 67 - protein, 5 aa, synthetic construct
SYWIN

SEQ ID NO: 68 - protein, 17 aa, synthetic construct
NIYPSDSYTNYNQKFKD

SEQ ID NO: 69 - protein, 10 aa, synthetic construct
FHYGSSPFDN

SEQ ID NO: 70 - protein, 11 aa, synthetic construct
RASQDISHYLN

SEQ ID NO: 71 - protein, 7 aa, synthetic construct
YTSRLHS

SEQ ID NO: 72 - protein, 9 aa, synthetic construct
QQGHTLPRT

SEQ ID NO: 73 - protein, 5 aa, synthetic construct
NYGMN

SEQ ID NO: 74 - protein, 17 aa, synthetic construct
WINTYTGEPTYADDFKG

SEQ ID NO: 75 - protein, 9 aa, synthetic construct
RDYLGAMDY

SEQ ID NO: 76 - protein, 12 aa, synthetic construct
RASSSVSSSYLH

SEQ ID NO: 77 - protein, 7 aa, synthetic construct
STSNLAS

SEQ ID NO: 78 - protein, 9 aa, synthetic construct
QQYSAYLYT

SEQ ID NO: 79 - protein, 5 aa, synthetic construct
TYWIN

SEQ ID NO: 80 - protein, 17 aa, synthetic construct
NIYPSDSYTNYNQKFKD

SEQ ID NO: 81 - protein, 13 aa, synthetic construct
EAGDGTHYYAIDY

SEQ ID NO: 82 - protein, 11 aa, synthetic construct
KASENVGTYVS

SEQ ID NO: 83 - protein, 7 aa, synthetic construct
GASNRYP

SEQ ID NO: 84 - protein, 9 aa, synthetic construct
GQGYSYLRT

SEQ ID NO: 85 - protein, 5 aa, synthetic construct
DYYMH

SEQ ID NO: 86 - protein, 17 aa, synthetic construct
WIDPENGNTIYDPKFQD

SEQ ID NO: 87 - protein, 8 aa, synthetic construct
GLPWYFDY

SEQ ID NO: 88 - protein, 10 aa, synthetic construct
SASSSVSDMN

SEQ ID NO: 89 - protein, 7 aa, synthetic construct
LTSNLSS

SEQ ID NO: 90 - protein, 9 aa, synthetic construct
QQWSGNPLT

SEQ ID NO: 91 - protein, 5 aa, synthetic construct
SFGMH

SEQ ID NO: 92 - protein, 17 aa, synthetic construct
YISSGSSAIYYADTVKG

SEQ ID NO: 93 - protein, 5 aa, synthetic construct
DWVDY

SEQ ID NO: 94 - protein, 11 aa, synthetic construct
RASENIYSNLV

SEQ ID NO: 95 - protein, 7 aa, synthetic construct
TATKLTN

SEQ ID NO: 96 - protein, 9 aa, synthetic construct
QHFWDTPYT

SEQ ID NO: 97 - protein, 5 aa, synthetic construct
SYWIN

SEQ ID NO: 98 - protein, 17 aa, synthetic construct
NIFPSDSYTNYNQKFKD

SEQ ID NO: 99 - protein, 14 aa, synthetic construct
FGSSGYPDYYTMEY

SEQ ID NO: 100 - protein, 11 aa, synthetic construct
RASGNIDNYLA

SEQ ID NO: 101 - protein, 7 aa, synthetic construct
NAKTLAE

SEQ ID NO: 102 - protein, 9 aa, synthetic construct
QQYWTIPYT

SEQ ID NO: 103 - protein, 5 aa, synthetic construct
NSWMN

SEQ ID NO: 104 - protein, 17 aa, synthetic construct
RIYPGDGDTDYNGKFKG

SEQ ID NO: 105 - protein, 12 aa, synthetic construct
LDGNYYYWYFDV

SEQ ID NO: 106 - protein, 16 aa, synthetic construct
KSSQSLLDSDGKTYLN

SEQ ID NO: 107 - protein, 7 aa, synthetic construct
LVSKLDS

SEQ ID NO: 108 - protein, 9 aa, synthetic construct
WQGTHFPQT

SEQ ID NO: 109 - protein, 10 aa, synthetic construct
GFTFSSSYIN

SEQ ID NO: 110 - protein, 6 aa, synthetic construct
FAGTGG

SEQ ID NO: 111 - protein, 10 aa, synthetic construct
HSPRATLFDF

SEQ ID NO: 112 - protein, 10 aa, synthetic construct
RASSSVNHMH

SEQ ID NO: 113 - protein, 7 aa, synthetic construct
ATSNLAS

SEQ ID NO: 114 - protein, 9 aa, synthetic construct
QQWSSNPYT

SEQ ID NO: 115 - protein, 10 aa, synthetic construct
GYTFSSYWIN

SEQ ID NO: 116 - protein, 6 aa, synthetic construct
YPSDSY

SEQ ID NO: 117 - protein, 12 aa, synthetic construct
TWEFYYDSVFDY

SEQ ID NO: 118 - protein, 11 aa, synthetic construct
KASQDINSYLS

SEQ ID NO: 119 - protein, 7 aa, synthetic construct
RANRLVD

SEQ ID NO: 120 - protein, 9 aa, synthetic construct
LQYDELPPT

SEQ ID NO: 121 - protein, 10 aa, synthetic construct
GFSLTTYDIS

SEQ ID NO: 122 - protein, 5 aa, synthetic construct
WTGGG

SEQ ID NO: 123 - skipped

SEQ ID NO: 124 - protein, 16 aa, synthetic construct
RSSQSLVHSNGNTYLH

SEQ ID NO: 125 - protein, 7 aa, synthetic construct
KVSNRFS

SEQ ID NO: 126 - protein, 9 aa, synthetic construct
SQNTHVPWT

SEQ ID NO: 127 - protein, 10 aa, synthetic construct
GYAFSTSWMN

SEQ ID NO: 128 - protein, 6 aa, synthetic construct
YPGDGD

SEQ ID NO: 129 - protein, 12 aa, synthetic construct
LDGYYEVFYFDY

SEQ ID NO: 130 - protein, 16 aa, synthetic construct
KSSQSLLYSNGKTYLN

SEQ ID NO: 131 - protein, 7 aa, synthetic construct
LVSKLDS

SEQ ID NO: 132 - protein, 9 aa, synthetic construct
FQGTHFPWT

SEQ ID NO: 133 - protein, 10 aa, synthetic construct
GYTFTTYVMY

SEQ ID NO: 134 - protein, 6 aa, synthetic construct
NPYNDD

SEQ ID NO: 135 - protein, 5 aa, synthetic construct
QGGDY

SEQ ID NO: 136 - protein, 11 aa, synthetic construct
RASKNINKYLA

SEQ ID NO: 137 - protein, 7 aa, synthetic construct
SGSTLQS

SEQ ID NO: 138 - protein, 9 aa, synthetic construct
QQYNEYPLT

SEQ ID NO: 139 - protein, 10 aa, synthetic construct
GFSLTTYGVH

SEQ ID NO: 140 - protein, 5 aa, synthetic construct
WSDGN

SEQ ID NO: 141 - protein, 10 aa, synthetic construct
NSITSVSFDC

SEQ ID NO: 142 - protein, 11 aa, synthetic construct
KASQNVGTTVA

SEQ ID NO: 143 - protein, 7 aa, synthetic construct
SASYRYS

SEQ ID NO: 144 - protein, 9 aa, synthetic construct
QQYNSYPLT

SEQ ID NO: 145 - protein, 10 aa, synthetic construct
GYTFSNYWIH

SEQ ID NO: 146 - protein, 6 aa, synthetic construct
YPASDY

SEQ ID NO: 147 - protein, 8 aa, synthetic construct
GYFGSLDY

SEQ ID NO: 148 - protein, 11 aa, synthetic construct
RASQPIGTGIH

SEQ ID NO: 149 - protein, 7 aa, synthetic construct
SASESIS

SEQ ID NO: 150 - protein, 9 aa, synthetic construct
QQSYSWPTT

SEQ ID NO: 151 - protein, 10 aa, synthetic construct
GYSFTSNWMH

SEQ ID NO: 152 - protein, 6 aa, synthetic construct
YPGNSD

SEQ ID NO: 153 - protein, 8 aa, synthetic construct
GITTAPEY

SEQ ID NO: 154 - protein, 11 aa, synthetic construct
KASENVGIYVS

SEQ ID NO: 155 - protein, 7 aa, synthetic construct
GASNRYT

SEQ ID NO: 156 - protein, 9 aa, synthetic construct
GHTYNYPFT

SEQ ID NO: 157 - protein, 10 aa, synthetic construct
GFDFSRYWMS

SEQ ID NO: 158 - protein, 6 aa, synthetic construct
HPDSSA

SEQ ID NO: 159 - protein, 9 aa, synthetic construct
SYYGRVFDY

SEQ ID NO: 160 - protein, 11 aa, synthetic construct
RASQSISDYLH

SEQ ID NO: 161 - protein, 7 aa, synthetic construct
YASQSIS

SEQ ID NO: 162 - protein, 9 aa, synthetic construct
QDGHSFPPT

SEQ ID NO: 163 - protein, 10 aa, synthetic construct
GYSFTSYWMH

SEQ ID NO: 164 - protein, 6 aa, synthetic construct
YPGNSD

SEQ ID NO: 165 - protein, 8 aa, synthetic construct
GITTAPDY

SEQ ID NO: 166 - protein, 11 aa, synthetic construct
KASENVGIHVS

SEQ ID NO: 167 - protein, 7 aa, synthetic construct
GASSGYN

SEQ ID NO: 168 - protein, 9 aa, synthetic construct
GQTYSYPFT

SEQ ID NO: 169 - protein, 10 aa, synthetic construct
KFTFTNYGMN

SEQ ID NO: 170 - protein, 6 aa, synthetic construct
NTYTGE

SEQ ID NO: 171 - protein, 10 aa, synthetic construct
REYYPWVFVY

SEQ ID NO: 172 - protein, 10 aa, synthetic construct
SASSSVSYMH

SEQ ID NO: 173 - protein, 7 aa, synthetic construct
DTSKLAS

SEQ ID NO: 174 - protein, 9 aa, synthetic construct
FQGSGYPLT

SEQ ID NO: 175 - protein, 10 aa, synthetic construct
GYIFTSYWIN

SEQ ID NO: 176 - protein, 6 aa, synthetic construct
YPSDSY

SEQ ID NO: 177 - protein, 10 aa, synthetic construct
FHYGSSPFDN

SEQ ID NO: 178 - protein, 11 aa, synthetic construct
RASQDISHYLN

SEQ ID NO: 179 - protein, 7 aa, synthetic construct
YTSRLHS

SEQ ID NO: 180 - protein, 9 aa, synthetic construct
QQGHTLPRT

SEQ ID NO: 181 - protein, 10 aa, synthetic construct
GYTFTNYGMN

SEQ ID NO: 182 - protein, 6 aa, synthetic construct
NTYTGE

SEQ ID NO: 183 - protein, 9 aa, synthetic construct
RDYLGAMDY

SEQ ID NO: 184 - protein, 12 aa, synthetic construct
RASSSVSSSYLH

SEQ ID NO: 185 - protein, 7 aa, synthetic construct
STSNLAS

SEQ ID NO: 186 - protein, 9 aa, synthetic construct
QQYSAYLYT

SEQ ID NO: 187 - protein, 10 aa, synthetic construct
GYTFTTYWIN

SEQ ID NO: 188 - protein, 6 aa, synthetic construct
YPSDSY

SEQ ID NO: 189 - protein, 13 aa, synthetic construct
EAGDGTHYYAIDY

SEQ ID NO: 190 - protein, 11 aa, synthetic construct
KASENVGTYVS

SEQ ID NO: 191 - protein, 7 aa, synthetic construct
GASNRYP

SEQ ID NO: 192 - protein, 9 aa, synthetic construct
GQGYSYLRT

SEQ ID NO: 193 - protein, 10 aa, synthetic construct
GFNIKDYYMH

SEQ ID NO: 194 - protein, 6 aa, synthetic construct
DPENGN

SEQ ID NO: 195 - protein, 8 aa, synthetic construct
GLPWYFDY

SEQ ID NO: 196 - protein, 10 aa, synthetic construct
SASSSVSDMN

SEQ ID NO: 197 - protein, 7 aa, synthetic construct
LTSNLSS

SEQ ID NO: 198 - protein, 9 aa, synthetic construct
QQWSGNPLT

SEQ ID NO: 199 - protein, 10 aa, synthetic construct
GFSFSSFGMH

SEQ ID NO: 200 - protein, 6 aa, synthetic construct
SSGSSA

SEQ ID NO: 201 - protein, 5 aa, synthetic construct
DWVDY

SEQ ID NO: 202 - protein, 11 aa, synthetic construct
RASENIYSNLV

SEQ ID NO: 203 - protein, 7 aa, synthetic construct
TATKLTN

SEQ ID NO: 204 - protein, 9 aa, synthetic construct
QHFWDTPYT

SEQ ID NO: 205 - protein, 10 aa, synthetic construct
GYTFTSYWIN

SEQ ID NO: 206 - protein, 6 aa, synthetic construct
FPSDSY

SEQ ID NO: 207 - protein, 14 aa, synthetic construct
FGSSGYPDYYTMEY

SEQ ID NO: 208 - protein, 11 aa, synthetic construct
RASGNIDNYLA

SEQ ID NO: 209 - protein, 7 aa, synthetic construct
NAKTLAE

SEQ ID NO: 210 - protein, 9 aa, synthetic construct
QQYWTIPYT

SEQ ID NO: 211 - protein, 10 aa, synthetic construct
GYAFNNSWMN

SEQ ID NO: 212 - protein, 6 aa, synthetic construct
YPGDGD

SEQ ID NO: 213 - protein, 12 aa, synthetic construct
LDGNYYYWYFDV

SEQ ID NO: 214 - protein, 16 aa, synthetic construct
KSSQSLLDSDGKTYLN

SEQ ID NO: 215 - protein, 7 aa, synthetic construct
LVSKLDS

SEQ ID NO: 216 - protein, 9 aa, synthetic construct
WQGTHFPQT

SEQ ID NO: 217 - protein, 255 aa, Homo sapiens
  REGION 1..255: note = misc_feature - Human 41-BB
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 218 - protein, 256 aa, Mus musculus
  REGION 1..256: note = misc_feature - Mouse 41-BB
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 219 - protein, 254 aa, Macaca mulatta
  REGION 1..254: note = Monkey
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 220 - protein, 257 aa, synthetic construct
  REGION 1..257: note = Chimeric 4-1BB
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSLQVLTLFLALTSALLLALIFITLLFSVLKWIRKKFPHIFKQPFKKTTGAAQEEDACSCRCPQEEEGGGGGYEL

SEQ ID NO: 221 - protein, 119 aa, synthetic construct
  REGION 1..119: note = heavy chain variable domain (VH)
QVQMVQSGAEVKKPGASVKLSCKASGFTFSSSYINWLRQAPGQRLEWMGWIFAGTGGTYYNQKFTGRVTITRDTSASTAYMELSSLRSEDTAIYYCARHSPRATLFDFWGQGTTLTVSS

SEQ ID NO: 222 - protein, 119 aa, synthetic construct
  REGION 1..119: note = heavy chain variable domain (VH)
QVQMVQSGAEVKKPGASVKLSCKASGFTFSSSYINWLRQAPGQSLEWIGWIFAGTGGTYYNQKFTGRVQITRDTSASTAYMELSSLRSEDTAIYYCARHSPRATLFDFWGQGTTLTVSS

SEQ ID NO: 223 - protein, 119 aa, synthetic construct
  REGION 1..119: note = heavy chain variable domain (VH)
QVQMVQSGAEVKKPGASVKLSCKASGFTFSSSYINWLRQAPGQSLEWIGWIFAGTGGTYYNQKFTGRAQITRDTSASTAYMELSSLRSEDTAIYYCARHSPRATLFDFWGQGTTLTVSS

SEQ ID NO: 224 - protein, 119 aa, synthetic construct
  REGION 1..119: note = heavy chain variable domain (VH)
QGQMVQSGAEVKKPGASVKLSCKTSGFTFSSSYINWLRQAPGQSLEWIAWIFAGTGGTYYNQKFTGRAQLTVDTSASTAYMELSSLRSEDTAIYYCARHSPRATLFDFWGQGTTLTVSS

SEQ ID NO: 225 - protein, 106 aa, synthetic construct
  REGION 1..106: note = light chain variable domain (VL)
EIVLTQSPATLSLSPGERATLSCRASSSVNHMHWYQQKPGQAPRALIYATSNLASGIPARFSGSGSGTSFTLTISSLEPEDFAVYYCQQWSSNPYTFGGGTKLEIK

SEQ ID NO: 226 - protein, 106 aa, synthetic construct
  REGION 1..106: note = light chain variable domain (VL)
EIVLTQSPATLSLSPGERATLSCRASSSVNHMHWYQQKPGQAPRALIYATSNLASGIPARFSGSGSGTSFTLTISSLEPEDFATYYCQQWSSNPYTFGGGTKLEIK

SEQ ID NO: 227 - protein, 106 aa, synthetic construct
  REGION 1..106: note = light chain variable domain (VL)
EIVLTQSPATLSLSPGERATMSCRASSSVNHMHWYQQKPGFSPRAWIYATSNLASGIPARFSGSGSGTSFTLTISSLEPEDFATYYCQQWSSNPYTFGGGTKLEIK

SEQ ID NO: 228 - protein, 106 aa, synthetic construct
  REGION 1..106: note = light chain variable domain (VL)
EIVLTQSPATLSASPGERVTMSCRASSSVNHMHWYQQRPGFSPRAWIYATSNLASGIPARFSGSGSGTSYSLTISSLEPEDFATYYCQQWSSNPYTFGGGTKLEIK

SEQ ID NO: 229 - protein, 121 aa, synthetic construct
  REGION 1..121: note = VH
QVQLVQSGAEVKKPGSSVKLSCKASGYTFSSYWINWVRQAPGQGLEWMGNIYPSDSYTNYNQKFKDRVTLTVDKSTSTAYMELSSLRSEDTAVYYCTRTWEFYYDSVFDYWGQGTLVTVSS

SEQ ID NO: 230 - protein, 121 aa, synthetic construct
  REGION 1..121: note = VH
QVQLVQSGAEVKKPGSSVKLSCKASGYTFSSYWINWVRQAPGQGLEWIGNIYPSDSYTNYNQKFKDRATLTVDKSTSTAYMELSSLRSEDTAVYYCTRTWEFYYDSVFDYWGQGTLVTVSS

SEQ ID NO: 231 - protein, 121 aa, synthetic construct
  REGION 1..121: note = VH
QVQLVQSGAEVKKPGSSVKLSCKASGYTFSSYWINWVKQAPGQGLEWIGNIYPSDSYTNYNQKFKDKATLTVDKSTSTAYMELSSLRSEDTAVYYCTRTWEFYYDSVFDYWGQGTTLTVSS

SEQ ID NO: 232 - protein, 107 aa, synthetic construct
  REGION 1..107: note = VL
DIQMTQSPSSLSASLGERVTITCKASQDINSYLSWYQQKPGKAPKLLIYRANRLVDGVPSRFSGSGSGQDFTLTISSLQPEDIATYYCLQYDELPPTFGGGTKVEIK

SEQ ID NO: 233 - protein, 107 aa, synthetic construct
  REGION 1..107: note = VL
DIQMTQSPSSLSASLGERVTITCKASQDINSYLSWYQQKPGKAPKTLIYRANRLVDGVPSRFSGSGSGQDFTLTISSLQPEDIATYYCLQYDELPPTFGGGTKVEIK

SEQ ID NO: 234 - protein, 107 aa, synthetic construct
  REGION 1..107: note = VL
DIQMTQSPSSLSASLGERVTITCKASQDINSYLSWFQQKPGKAPKTLIYRANRLVDGVPSRFSGSGSGQDFTLTISSLQPEDIATYYCLQYDELPPTFGGGTKVEIK

SEQ ID NO: 235 - protein, 107 aa, synthetic construct
  REGION 1..107: note = VL
DIQMTQSPSSLSASLGERVTITCKASQDINSYLSWFQQKPGKAPKTLIYRANRLVDGVPSRFSGSGSGQDYTLTISSLQPEDIATYYCLQYDELPPTFGGGTKLEIK

SEQ ID NO: 236 - protein, 111 aa, synthetic construct
  REGION 1..111: note = VH
QVQLQESGPGLVKPSETLSLTCTVSGFSLTTYDISWVRQPPGKGLEWIGVIWTGGGTNYNSSFMSRVTISKDTSKNQVSLKLSSVTAADTAVYYCERVDYWGQGTTVTVSS

SEQ ID NO: 237 - protein, 111 aa, synthetic construct
  REGION 1..111: note = VH
QVQLKESGPGLVKPSETLSLTCTVSGFSLTTYDISWVRQPPGKGLEWLGVIWTGGGTNYNSSFMSRVTISKDTSKNQVSLKLSSVTAADTAIYYCERVDYWGQGTTVTVSS

SEQ ID NO: 238 - protein, 111 aa, synthetic construct
  REGION 1..111: note = VH
QVQLKESGPGLVKPSETLSITCTVSGFSLTTYDISWVRQPPGKGLEWLGVIWTGGGTNYNSSFMSRVTISKDNSKNQVSLKLSSVTAADTAIYYCERVDYWGQGTTLTVSS

SEQ ID NO: 239 - protein, 112 aa, synthetic construct
  REGION 1..112: note = VL
DVVMTQSPLSLPVTLGQPASISCRSSQSLVHSNGNTYLHWFQQRPGQSPRLLIYKVSNRFSGVPDRFSGSGSGTAFTFKISRVEAEDVGVYYCSQNTHVPWTFGGGTKVEIK

SEQ ID NO: 240 - protein, 112 aa, synthetic construct
  REGION 1..112: note = VL
DVVMTQSPLSLPVTLGQPASISCRSSQSLVHSNGNTYLHWFQQRPGQSPKLLIYKVSNRFSGVPDRFSGSGSGTAFTFKISRVEAEDVGVYYCSQNTHVPWTFGGGTKVEIK

SEQ ID NO: 241 - protein, 112 aa, synthetic construct
  REGION 1..112: note = VL
DVVMTQSPLSLPVTLGQPASISCRSSQSLVHSNGNTYLHWYQQRPGQSPKLLIYKVSNRFSGVPDRFSGSGSGTAFTFKISRVEAEDVGVYYCSQNTHVPWTFGGGTKLEIK

SEQ ID NO: 242 - protein, 112 aa, synthetic construct
  REGION 1..112: note = VL
DVVMTQSPLSLPVTLGDPASISCRSSQSLVHSNGNTYLHWYLQRPGQSPKLLIYKVSNRFSGVPDRFSGSGSGTAFTFKISRVEAEDVGVYYCSQNTHVPWTFGGGTKLEIK

SEQ ID NO: 243 - protein, 119 aa, synthetic construct
  REGION 1..119: note = VH
QGQMQQSGAELVKPGASVKLSCKTSGFTFSSSYINWLKQKPGQSLEWIAWIFAGTGGTYYNQKFTGKAQLTVDTSSGTAYMQFSSLTTEDSAIYYCARHSPRATLFDFWGQGTTLTVSS

SEQ ID NO: 244 - protein, 106 aa, synthetic construct
  REGION 1..106: note = VL
QIVLSQSPAILSASPGEKVTMTCRASSSVNHMHWYQQRTGFSPKAWIYATSNLASGVPARFSGSGSGTSYSLTISRVEAEDAATYYCQQWSSNPYTFGGGTKLEIK

SEQ ID NO: 245 - protein, 121 aa, synthetic construct
  REGION 1..121: note = VH
QVQLQQPGAELVRPGASVKLSCKASGYTFSSYWINWVKQRPGQGLEWIGNIYPSDSYTNYNQKFKDKATLTVDKSSSTAYMQLSSPTSDDSAVYYCTRTWEFYYDSVFDYWGQGTTLTVSS

SEQ ID NO: 246 - protein, 107 aa, synthetic construct
  REGION 1..107: note = VL
DIKMTQSPSSMYASLGERVTITCKASQDINSYLSWFQQKPGKSPKTLIYRANRLVDGVPSRFSGSGSGQDYSLTISSLEYEDMGIYYCLQYDELPPTFGGGTKLEIK

SEQ ID NO: 247 - protein, 111 aa, synthetic construct
  REGION 1..111: note = VH
QVQLKESGPGLVAPSQSLSITCTVSGFSLTTYDISWVRQPPGKGLEWLGVIWTGGGTNYNSSFMSRLSISKDNSKNQVFLKMNSLQVDDTAIYYCERVDYWGQGTTLTVSS

SEQ ID NO: 248 - protein, 112 aa, synthetic construct
  REGION 1..112: note = VL
DVVMTQTPLSLPVSLGDQASISCRSSQSLVHSNGNTYLHWYLQKPGQSPKLLIYKVSNRFSGVPDRFSGSGSGTAFTFKISRVEAEDLGVYFCSQNTHVPWTFGGGTKLEIK

SEQ ID NO: 249 - protein, 121 aa, synthetic construct
  REGION 1..121: note = VH
QVQLQQSGPELVKPGASVKISCKTSGYAFSTSWMNWVKQRPGQGLEWIGRIYPGDGDTDYNGRFKGKATLTADKSSSTAYLQLSSLTSVDSAVYFCARLDGYYEVFYFDYWGQGTTLTVSS

SEQ ID NO: 250 - protein, 112 aa, synthetic construct
  REGION 1..112: note = VL
DVVMTQTPLTLSVTIGQPASISCKSSQSLLYSNGKTYLNWLLQRPGQSPKRLIYLVSKLDSGVPDRFTGSGSGTDFTLKISRVEAEDLGIYYCFQGTHFPWTFGGGTKLEIK

SEQ ID NO: 251 - protein, 114 aa, synthetic construct
  REGION 1..114: note = VH
EVQLQQSGPELVKPGASVKMSCKASGYTFTTYVMYWVKQRPGQGLEWIGYINPYNDDIRYNEKFKGKATLTSDRSSSTAYMELSSLTSEDSAVYLCARQGGDYWGQGTILTVSS

SEQ ID NO: 252 - protein, 107 aa, synthetic construct
  REGION 1..107: note = VL
DVQITQSPSYLAASPGETITVNCRASKNINKYLAWYQEKPGKTNKLLIYSGSTLQSGIPSRFSGSGSGTDFTLTISSLEPEDFAVYHCQQYNEYPLTFGAGTKLEMK

SEQ ID NO: 253 - protein, 118 aa, synthetic construct
  REGION 1..118: note = VH
QVQLKQSGPGLVQPSQSLSITCTVSGFSLTTYGVHWVRQPPGKGLEWLGVIWSDGNTDYNDAFISRLSITKDNSTSQVFFKMNGLQADDTAIYYCARNSITSVSFDCWGQGTTLTVSS

SEQ ID NO: 254 - protein, 107 aa, synthetic construct
  REGION 1..107: note = VL
DIVMTQSQKFMSTSVGDRVSVTCKASQNVGTTVAWYQQRPGQSPKALICSASYRYSGVPDRFTGSGSGTDFTLTISNVQSEDLAEYFCQQYNSYPLTFGSGTKLEIK

SEQ ID NO: 255 - protein, 117 aa, synthetic construct
  REGION 1..117: note = VH
QVQLQQPGSALVRPGASVKLSCKASGYTFSNYWIHWVKQRHGQGLEWIGNIYPASDYTNYDEKFKNKGTLTVDTSSSTAYMHLSSLTSEDSAVYYCSRGYFGSLDYWGQGTSVTVSA

SEQ ID NO: 256 - protein, 107 aa, synthetic construct
  REGION 1..107: note = VL
DILLTQSPAILSVNPGERVSFSCRASQPIGTGIHWYQQRTNGSPRLLIKSASESISGIPSRFSGSGSGTDFTLTINNVESDDVGDYYCQQSYSWPTTFGGGTKLKIN

SEQ ID NO: 257 - protein, 117 aa, synthetic construct
  REGION 1..117: note = VH
EVQLQQSGTVLARPGASVKMSCKASGYSFTSNWMHWVKQRPGQGLEWIGVIYPGNSDTSYNQKFKGKAKLTAVTSASTAYMELSSLTNEDSAVYYCTRGITTAPEYWGQGTTLTVSS

SEQ ID NO: 258 - protein, 107 aa, synthetic construct
  REGION 1..107: note = VL
NIVMTQSPKSMSMSVGERVTLSCKASENVGIYVSWYQQKPEQSPKLLIYGASNRYTGVPDRFTGSGSATDFTLTISSVQPEDLADYHCGHTYNYPFTFGSGTKLEIK

SEQ ID NO: 259 - protein, 118 aa, synthetic construct
  REGION 1..118: note = VH
EVKLLESGGGLVQPGGSLKLSCAASGFDFSRYWMSWVRQAPGKGLEWIGEIHPDSSAINYTPSLKDKFIISRDNAKNTLYLQMSKVRSEDTALYYCARSYYGRVFDYWGQGTTLTVSS

SEQ ID NO: 260 - protein, 107 aa, synthetic construct
  REGION 1..107: note = VL
DIVMTQSPATLSVTPGDRVSLSCRASQSISDYLHWYQQKSHESPRLLIKYASQSISGIPPRFSGSGSGSDFTLSINSVEPEDVGMYYCQDGHSFPPTFGAGTKLELK

SEQ ID NO: 261 - protein, 117 aa, synthetic construct
  REGION 1..117: note = VH
EVQLQQSGTVLARPGASVKMSCKASGYSFTSYWMHWVKQRPGQGLEWIGVIYPGNSDTSYNQKFKGKAKLTAVTSASTAYMDLSSLTNEDSAVYYCTRGITTAPDYWGQGTTLTVSS

SEQ ID NO: 262 - protein, 107 aa, synthetic construct
  REGION 1..107: note = VL
NIVMTQSPKSMSMSVGERVTLSCKASENVGIHVSWYQQKPEQSPKLLIYGASSGYNGVPDRFTGSGSATDFTLTISSVQPEDLADYHCGQTYSYPFTFGSGTKLEIK

SEQ ID NO: 263 - protein, 119 aa, synthetic construct
  REGION 1..119: note = VH
QIQLVQSGPELKKPGETVKISCKASKFTFTNYGMNWVKQAPGKALKWMGWINTYTGEPTYADDFKGRFDFSLETSATTAYLQINNLKNEDTATYFCARREYYPWVFVYWGQGTLVTVSA

SEQ ID NO: 264 - protein, 106 aa, synthetic construct
  REGION 1..106: note = VL
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGGGTKLEIK

SEQ ID NO: 265 - protein, 119 aa, synthetic construct
  REGION 1..119: note = VH
QVQLQQPGAELVRPGASVKLSCKASGYIFTSYWINWVKQRPGQGLEWIGNIYPSDSYTNYNQKFKDKATLTVDKSSSTAYMQLSSPTSEDSAVYYCTRFHYGSSPFDNWGQGTTLTVSS

SEQ ID NO: 266 - protein, 107 aa, synthetic construct
  REGION 1..107: note = VL
DIQMTQSTSSLSASLGDRVTISCRASQDISHYLNWYQQKPDGTVKLLIYYTSRLHSGVPSRFSGSGSGTDYSLTISNLEQEDFATYFCQQGHTLPRTFGGGTKLEIK

SEQ ID NO: 267 - protein, 118 aa, synthetic construct
  REGION 1..118: note = VH
QIQLVQSGPELKKPGETVKISCKASGYTFTNYGMNWVKQAPGKGFKWMGWINTYTGEPTYADDFKGRFAFSLEISASTAYLQINNLKNEDTATYFCARRDYLGAMDYWGQGTSVTVSS

SEQ ID NO: 268 - protein, 108 aa, synthetic construct
  REGION 1..108: note = VL
ENVLTQSPAIMSASPGEKVTMTCRASSSVSSSYLHWYQQKSGASPKLWIYSTSNLASGVPARFSGSGSGTSYSLTISSVEAEDAATYYCQQYSAYLYTFGGGTKLEIK

SEQ ID NO: 269 - protein, 122 aa, synthetic construct
  REGION 1..122: note = VH
QVQLQQPGAELVRPGASVKLSCKASGYTFTTYWINWVRQRPGQGLEWIGNIYPSDSYTNYNQKFKDKASLTVDKSSNTAYMQLSSPTSEDSAVYYCAREAGDGTHYYAIDYWGQGTSVTVSS

SEQ ID NO: 270 - protein, 107 aa, synthetic construct
  REGION 1..107: note = VL
NIVMTQSPKSMSMSVGERVTLSCKASENVGTYVSWYQQKPEQSPKLLIYGASNRYPGVPDRFTGSGSATDFTLTISSLQAEDLADYHCGQGYSYLRTFGGGTNLEIK

SEQ ID NO: 271 - protein, 117 aa, synthetic construct
  REGION 1..117: note = VH
EVQLQQSGAELVRPGALVKLSCKASGFNIKDYYMHWVKRRPEQGLEWIGWIDPENGNTIYDPKFQDKASITADTSSNTAYLQLSSLTSEDTAVYYCAEGLPWYFDYWGQGTTLTVSS

SEQ ID NO: 272 - protein, 106 aa, synthetic construct
  REGION 1..106: note = VL
QIILTQSPALMSASPGEKVTMTCSASSSVSDMNWYQQKPRSSPTPWIYLTSNLSSGVPDRFSGSGSGTSYSLTISSMEAEDAATYYCQQWSGNPLTFGAGTRLELK

SEQ ID NO: 273 - protein, 114 aa, synthetic construct
  REGION 1..114: note = VH
DVQLVESGGGLVQPGGSRKLSCAASGFSFSSFGMHWMRQAPEKGLEWVAYISSGSSAIYYADTVKGRFTISRDNPKNTLFLQMTSLRSEDTAMYYCARDWVDYWGQGTTLTVSS

SEQ ID NO: 274 - protein, 107 aa, synthetic construct
  REGION 1..107: note = VL
DIQMTQSPASLSVSVGETVTITCRASENIYSNLVWYQQKQGKSPQLLVYTATKLTNGVPSRFSGSGSGTQYSLKINSLQSEDFGTYYCQHFWDTPYTFGGGTKLEMK

SEQ ID NO: 275 - protein, 123 aa, synthetic construct
  REGION 1..123: note = VH
QVQLQQPGAELVRPGASVKLSCKASGYTFTSYWINWVKQRPGQGLEWIGNIFPSDSYTNYNQKFKDRAKLTVDKSSSTAYMQLSSPTSEDSAVYYCTRFGSSGYPDYYTMEYWGQGTSVTVSS

SEQ ID NO: 276 - protein, 107 aa, synthetic construct
  REGION 1..107: note = VL
DIQMTQSPASLSASVGETVTITCRASGNIDNYLAWYQQKQGKSPQLLVYNAKTLAEGVPSRFSGSGSGTEYSLKINSLQPEDFGSYHCQQYWTIPYTFGGGTKLEIK

SEQ ID NO: 277 - protein, 121 aa, synthetic construct
  REGION 1..121: note = VH
QVQLQQSGPELVKPGASVKISCKASGYAFNNSWMNWVKQRPGQGLEWIGRIYPGDGDTDYNGKFKGKATLTADKSSSTAYMQLSSLTSVDSAVYFCARLDGNYYYWYFDVWGAGTTVTVSS

SEQ ID NO: 278 - protein, 112 aa, synthetic construct
  REGION 1..112: note = VL
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 279 - protein, 5 aa, synthetic construct
GYYWS

SEQ ID NO: 280 - protein, 16 aa, synthetic construct
EINHGGYVTYNPSLES

SEQ ID NO: 281 - protein, 13 aa, synthetic construct
DYGPGNYDWYFDL

SEQ ID NO: 282 - protein, 11 aa, synthetic construct
RASQSVSSYLA

SEQ ID NO: 283 - protein, 7 aa, synthetic construct
DASNRAT

SEQ ID NO: 284 - protein, 11 aa, synthetic construct
QQRSNWPPALT

SEQ ID NO: 285 - protein, 5 aa, synthetic construct
DYWMS

SEQ ID NO: 286 - protein, 17 aa, synthetic construct
DIKNDGSYTNYAPSLTN

SEQ ID NO: 287 - protein, 5 aa, synthetic construct
ELTGT

SEQ ID NO: 288 - protein, 17 aa, synthetic construct
KSSQSLLSSGNQKNYLA

SEQ ID NO: 289 - protein, 7 aa, synthetic construct
YASTRQS

SEQ ID NO: 290 - protein, 9 aa, synthetic construct
LQYDRYPFT

SEQ ID NO: 291 - protein, 6 aa, synthetic construct
STYWIS

SEQ ID NO: 292 - protein, 17 aa, synthetic construct
KIYPGDSYTNYSPSFQG

SEQ ID NO: 293 - protein, 8 aa, synthetic construct
RGYGIFDY

SEQ ID NO: 294 - protein, 11 aa, synthetic construct
SGDNIGDQYAH

SEQ ID NO: 295 - protein, 7 aa, synthetic construct
QDKNRPS

SEQ ID NO: 296 - protein, 11 aa, synthetic construct
ATYTGFGSLAV

SEQ ID NO: 297 - protein, 5 aa, synthetic construct
DYYMN

SEQ ID NO: 298 - protein, 19 aa, synthetic construct
YISSSASGSTIYYADSVKG

SEQ ID NO: 299 - protein, 13 aa, synthetic construct
VVPAGSGWRWFDP

SEQ ID NO: 300 - protein, 14 aa, synthetic construct
TGISSDVGAYDYVS

SEQ ID NO: 301 - protein, 7 aa, synthetic construct
EVSKRPS

SEQ ID NO: 302 - protein, 11 aa, synthetic construct
SSHAGSNNFYV

SEQ ID NO: 303 - protein, 7 aa, synthetic construct
SNSAAWN

SEQ ID NO: 304 - protein, 18 aa, synthetic construct
RTYYRSKWYNDYAPSVES

SEQ ID NO: 305 - protein, 11 aa, synthetic construct
DPPYVLSTFDI

SEQ ID NO: 306 - protein, 13 aa, synthetic construct
TRSSGNIASFYVQ

SEQ ID NO: 307 - protein, 7 aa, synthetic construct
EDDQRPS

SEQ ID NO: 308 - protein, 9 aa, synthetic construct
QSYDTNNVI

SEQ ID NO: 309 - protein, 7 aa, synthetic construct
SNSAAWN

SEQ ID NO: 310 - protein, 18 aa, synthetic construct
RTYYRSRWYSDYASSVES

SEQ ID NO: 311 - protein, 11 aa, synthetic construct
DPPYVLSTFDI

SEQ ID NO: 312 - protein, 13 aa, synthetic construct
TRSSGNIASFYVQ

SEQ ID NO: 313 - protein, 7 aa, synthetic construct
EDDQRPS

SEQ ID NO: 314 - protein, 9 aa, synthetic construct
QSYDTNNVI

SEQ ID NO: 315 - protein, 7 aa, synthetic construct
SDSAAWN

SEQ ID NO: 316 - protein, 18 aa, synthetic construct
RTYYRSKWYNDYAPSVES

SEQ ID NO: 317 - protein, 11 aa, synthetic construct
DPPYVLSTFDI

SEQ ID NO: 318 - protein, 13 aa, synthetic construct
TRSSGNIASFYVQ

SEQ ID NO: 319 - protein, 7 aa, synthetic construct
EDDQRPS

SEQ ID NO: 320 - protein, 9 aa, synthetic construct
QSYDTNNVI

SEQ ID NO: 321 - protein, 7 aa, synthetic construct
SNSAAWN

SEQ ID NO: 322 - protein, 18 aa, synthetic construct
RTYYRSKWYNDYAPSVES

SEQ ID NO: 323 - protein, 11 aa, synthetic construct
DPPYVLSTFDI

SEQ ID NO: 324 - protein, 13 aa, synthetic construct
TRSSGNIASFYVQ

SEQ ID NO: 325 - protein, 7 aa, synthetic construct
DDDQRPS

SEQ ID NO: 326 - protein, 9 aa, synthetic construct
QSYDTNNVI

SEQ ID NO: 327 - protein, 7 aa, synthetic construct
SNSAAWN

SEQ ID NO: 328 - protein, 18 aa, synthetic construct
RTYYRSKWYNDYAPSVES

SEQ ID NO: 329 - protein, 11 aa, synthetic construct
DPPYVLSTFDI

SEQ ID NO: 330 - protein, 13 aa, synthetic construct
TRSSGNIASFYVQ

SEQ ID NO: 331 - protein, 7 aa, synthetic construct
EDDQRPS

SEQ ID NO: 332 - protein, 9 aa, synthetic construct
QSYDTNKVI

SEQ ID NO: 333 - protein, 7 aa, synthetic construct
SNSAAWN

SEQ ID NO: 334 - protein, 18 aa, synthetic construct
RTYYRSKWYNDYAPSVES

SEQ ID NO: 335 - protein, 11 aa, synthetic construct
DPPYVLSTFDI

SEQ ID NO: 336 - protein, 13 aa, synthetic construct
TRSSGNIASFYVQ

SEQ ID NO: 337 - protein, 7 aa, synthetic construct
EDDQRPS

SEQ ID NO: 338 - protein, 9 aa, synthetic construct
QSYDINKVI

SEQ ID NO: 339 - protein, 7 aa, synthetic construct
SNSAAWN

SEQ ID NO: 340 - protein, 18 aa, synthetic construct
RTYYRSKWYNDYAPSVES

SEQ ID NO: 341 - protein, 11 aa, synthetic construct
DPPYVLSTFDI

SEQ ID NO: 342 - protein, 13 aa, synthetic construct
TRSSGNIASFYVQ

SEQ ID NO: 343 - protein, 7 aa, synthetic construct
DDDQRPS

SEQ ID NO: 344 - protein, 9 aa, synthetic construct
QSYDINKVI

SEQ ID NO: 345 - protein, 8 aa, synthetic construct
GYMPDGYY

SEQ ID NO: 346 - protein, 8 aa, synthetic construct
INPRTGGT

SEQ ID NO: 347 - protein, 13 aa, synthetic construct
AREGAAFRLELDA

SEQ ID NO: 348 - protein, 6 aa, synthetic construct
NIGSKS

SEQ ID NO: 349 - skipped

SEQ ID NO: 350 - protein, 9 aa, synthetic construct
QVWDSSSVV

SEQ ID NO: 351 - protein, 8 aa, synthetic construct
GYSFTGYY

SEQ ID NO: 352 - protein, 8 aa, synthetic construct
VNPKSGGT

SEQ ID NO: 353 - protein, 13 aa, synthetic construct
AREGWARRIDLDE

SEQ ID NO: 354 - protein, 6 aa, synthetic construct
NIGSKS

SEQ ID NO: 355 - skipped

SEQ ID NO: 356 - protein, 9 aa, synthetic construct
QVWDSSSVV

SEQ ID NO: 357 - protein, 8 aa, synthetic construct
GYSFSGYY

SEQ ID NO: 358 - protein, 8 aa, synthetic construct
VNPMSGGT

SEQ ID NO: 359 - protein, 13 aa, synthetic construct
AREGMAMRLELDK

SEQ ID NO: 360 - protein, 6 aa, synthetic construct
NIGSKS

SEQ ID NO: 361 - skipped

SEQ ID NO: 362 - protein, 9 aa, synthetic construct
QVWDSSSVV

SEQ ID NO: 363 - protein, 8 aa, synthetic construct
GYNFGGYY

SEQ ID NO: 364 - protein, 8 aa, synthetic construct
VNPHSGGT

SEQ ID NO: 365 - protein, 13 aa, synthetic construct
AREGEAWGLDLDL

SEQ ID NO: 366 - protein, 6 aa, synthetic construct
NIGSKS

SEQ ID NO: 367 - skipped

SEQ ID NO: 368 - protein, 9 aa, synthetic construct
QVWDSSSVV

SEQ ID NO: 369 - protein, 8 aa, synthetic construct
GYNWTGYY

SEQ ID NO: 370 - protein, 8 aa, synthetic construct
INPMAGGT

SEQ ID NO: 371 - protein, 13 aa, synthetic construct
AREGWARGVELDM

SEQ ID NO: 372 - protein, 6 aa, synthetic construct
NIGSKS

SEQ ID NO: 373 - skipped

SEQ ID NO: 374 - protein, 9 aa, synthetic construct
QVWDSSSVV

SEQ ID NO: 375 - protein, 8 aa, synthetic construct
GYQMRGYY

SEQ ID NO: 376 - protein, 8 aa, synthetic construct
INPNSGGT

SEQ ID NO: 377 - protein, 13 aa, synthetic construct
AREGEAVGLDLDY

SEQ ID NO: 378 - protein, 6 aa, synthetic construct
NIGSKS

SEQ ID NO: 379 - skipped

SEQ ID NO: 380 - protein, 9 aa, synthetic construct
QVWDSSSVV

SEQ ID NO: 381 - protein, 8 aa, synthetic construct
GYNFGGYY

SEQ ID NO: 382 - protein, 8 aa, synthetic construct
VNPKSGGT

SEQ ID NO: 383 - protein, 13 aa, synthetic construct
AREGEAVGLDLDY

SEQ ID NO: 384 - protein, 6 aa, synthetic construct
NIGSKS

SEQ ID NO: 385 - skipped

SEQ ID NO: 386 - protein, 9 aa, synthetic construct
QVWDSSSVV

SEQ ID NO: 387 - protein, 8 aa, synthetic construct
GYTFTGYY

SEQ ID NO: 388 - protein, 8 aa, synthetic construct
INPNSGGT

SEQ ID NO: 389 - protein, 13 aa, synthetic construct
AREGEAVGLDLDY

SEQ ID NO: 390 - protein, 6 aa, synthetic construct
NIGSKS

SEQ ID NO: 391 - skipped

SEQ ID NO: 392 - protein, 9 aa, synthetic construct
QVWDSSSVV

SEQ ID NO: 393 - protein, 8 aa, synthetic construct
GYTFTGYY

SEQ ID NO: 394 - protein, 8 aa, synthetic construct
INPNSGGT

SEQ ID NO: 395 - protein, 13 aa, synthetic construct
AREGEAVGLDLDY

SEQ ID NO: 396 - protein, 6 aa, synthetic construct
NIGSKS

SEQ ID NO: 397 - skipped

SEQ ID NO: 398 - protein, 9 aa, synthetic construct
QVWDSSSVV

SEQ ID NO: 399 - protein, 10 aa, synthetic construct
GYTFTSYYIY

SEQ ID NO: 400 - protein, 17 aa, synthetic construct
NIWPGNGGTFYGEKFMG

SEQ ID NO: 401 - protein, 12 aa, synthetic construct
RPDYSGDDYFDY

SEQ ID NO: 402 - protein, 13 aa, synthetic construct
KLNSGNIGSYYVH

SEQ ID NO: 403 - protein, 7 aa, synthetic construct
RDDKRPD

SEQ ID NO: 404 - protein, 10 aa, synthetic construct
HSYDSTITPV

SEQ ID NO: 405 - protein, 10 aa, synthetic construct
DYTFNDYWVS

SEQ ID NO: 406 - protein, 17 aa, synthetic construct
EIYPNSGATNFNGKFRG

SEQ ID NO: 407 - protein, 9 aa, synthetic construct
EYTRDWFAY

SEQ ID NO: 408 - protein, 16 aa, synthetic construct
RSSQSLLDSDGNTYLY

SEQ ID NO: 409 - protein, 7 aa, synthetic construct
LVSNLGS

SEQ ID NO: 410 - protein, 9 aa, synthetic construct
MQPTHAPYT

SEQ ID NO: 411 - protein, 10 aa, synthetic construct
DYTFNDYWVS

SEQ ID NO: 412 - protein, 17 aa, synthetic construct
EIYPNSGATNFNGKFRG

SEQ ID NO: 413 - protein, 9 aa, synthetic construct
EYTRDWFAY

SEQ ID NO: 414 - protein, 16 aa, synthetic construct
RSSQSLLDSDGNTYLY

SEQ ID NO: 415 - protein, 7 aa, synthetic construct
LVSNLGS

SEQ ID NO: 416 - protein, 8 aa, synthetic construct
QPTHAPYT

SEQ ID NO: 417 - protein, 8 aa, synthetic construct
GFTFGYSY

SEQ ID NO: 418 - protein, 8 aa, synthetic construct
IGSGSSYT

SEQ ID NO: 419 - protein, 11 aa, synthetic construct
ARVYSSPGIDY

SEQ ID NO: 420 - protein, 6 aa, synthetic construct
QSISSY

SEQ ID NO: 421 - skipped

SEQ ID NO: 422 - protein, 10 aa, synthetic construct
QQYYTWVPFT

SEQ ID NO: 423 - protein, 8 aa, synthetic construct
GFNFGYSY

SEQ ID NO: 424 - protein, 8 aa, synthetic construct
IGSTSSHT

SEQ ID NO: 425 - protein, 11 aa, synthetic construct
ARVYSSPGIDY

SEQ ID NO: 426 - protein, 6 aa, synthetic construct
QSIGST

SEQ ID NO: 427 - skipped

SEQ ID NO: 428 - protein, 10 aa, synthetic construct
QQYYTWVPFT

SEQ ID NO: 429 - protein, 5 aa, synthetic construct
DYAMH

SEQ ID NO: 430 - protein, 17 aa, synthetic construct
GISTYNGDASYNQKFKG

SEQ ID NO: 431 - protein, 9 aa, synthetic construct
SNYGYAMDY

SEQ ID NO: 432 - protein, 17 aa, synthetic construct
KSSQSLLYSSNQKNYLA

SEQ ID NO: 433 - protein, 7 aa, synthetic construct
WASTRES

SEQ ID NO: 434 - protein, 9 aa, synthetic construct
QQYLSYPWT

SEQ ID NO: 435 - protein, 5 aa, synthetic construct
TYAMH

SEQ ID NO: 436 - protein, 17 aa, synthetic construct
YINPTTVYTIYNQKFKD

SEQ ID NO: 437 - protein, 9 aa, synthetic construct
LGGHWYFDV

SEQ ID NO: 438 - protein, 15 aa, synthetic construct
RASESVDNYGISFMN

SEQ ID NO: 439 - protein, 7 aa, synthetic construct
SASNQGS

SEQ ID NO: 440 - protein, 9 aa, synthetic construct
QQSKEVPWT

SEQ ID NO: 441 - protein, 5 aa, synthetic construct
GYWIE

SEQ ID NO: 442 - protein, 17 aa, synthetic construct
EILPGGGTVYYNEKFKG

SEQ ID NO: 443 - protein, 8 aa, synthetic construct
WGRSWFTH

SEQ ID NO: 444 - protein, 16 aa, synthetic construct
RSSQSIVHSNGNTYLE

SEQ ID NO: 445 - protein, 7 aa, synthetic construct
EVSNRFS

SEQ ID NO: 446 - protein, 9 aa, synthetic construct
FQGSHVPRT

SEQ ID NO: 447 - protein, 5 aa, synthetic construct
SYWMD

SEQ ID NO: 448 - protein, 17 aa, synthetic construct
NIYPDSGGTNYAEKFKS

SEQ ID NO: 449 - protein, 12 aa, synthetic construct
EEALGGYYELTY

SEQ ID NO: 450 - protein, 15 aa, synthetic construct
RASQSVSTSSYSYMH

SEQ ID NO: 451 - protein, 7 aa, synthetic construct
YASNLES

SEQ ID NO: 452 - protein, 9 aa, synthetic construct
QHSWEIPYT

SEQ ID NO: 453 - protein, 5 aa, synthetic construct
NYWLG

SEQ ID NO: 454 - protein, 17 aa, synthetic construct
DIYPGNGNNYYSEKFKD

SEQ ID NO: 455 - protein, 10 aa, synthetic construct
HGSFRSAMDY

SEQ ID NO: 456 - protein, 11 aa, synthetic construct
RASENIYSYLA

SEQ ID NO: 457 - protein, 7 aa, synthetic construct
NAKTLAD

SEQ ID NO: 458 - protein, 9 aa, synthetic construct
QHHYGTPPT

SEQ ID NO: 459 - protein, 121 aa, synthetic construct [sequence omitted]

SEQ ID NO: 460 - protein, 109 aa, synthetic construct [sequence omitted]

SEQ ID NO: 461 - protein, 114 aa, synthetic construct
  REGION 1..114: note = VH
EVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELTGTWGQGTMVTVSS

SEQ ID NO: 462 - protein, 113 aa, synthetic construct
  REGION 1..113: note = VL
DIVMTQSPDSLAVSLGERATINCKSSQSLLSSGNQKNYLAWYQQKPGQPPKLLIYYASTRQSGVPDRFSGSGSGTDFTLTISSLQAEDVAVYYCLQYDRYPFTFGQGTKLEIK

SEQ ID NO: 463 - protein, 116 aa, synthetic construct
  REGION 1..116: note = VH
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 464 - protein, 108 aa, synthetic construct
  REGION 1..108: note = VL
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 465 - protein, 124 aa, synthetic construct
  REGION 1..124: note = VH
EVQLVESGGGLVQPGGSLRLSCAASGFTVSDYYMNWIRQAPGKGLEWVSYISSSASGSTIYYADSVKGRFTISRDNANNSLYLHMDSLRAEDTAIYFCARVVPAGSGWRWFDPWGQGTLVTVSS

SEQ ID NO: 466 - protein, 111 aa, synthetic construct
  REGION 1..111: note = VL
QSVLIQPPSASGSPGQSVTISCTGISSDVGAYDYVSWYQQHPGKVPKLMIYEVSKRPSGVPDRFSGSKSGDTASLTVSGLQAEDEADYYCSSHAGSNNFYVFGTGTKLTVL

SEQ ID NO: 467 - protein, 123 aa, synthetic construct
  REGION 1..123: note = VH
QVQLQQSGPGLVKPSQTLSLTCAISGDSVSSNSAAWNWIRQSPSRGLEWLGRTYYRSKWYNDYAPSVESRITINPDTSKNQFSLQLSSVTPEDTAVYYCARDPPYVLSTFDIWGQGTMVTVSS

SEQ ID NO: 468 - protein, 110 aa, synthetic construct
  REGION 1..110: note = VL
NFMLTQPPSVSESPGKTVTISCTRSSGNIASFYVQWFQQRPGSSPTTVIYEDDQRPSGVPDRFSGSIDRSSNSASLTISGLTTDDEADYYCQSYDTNNVIFGGGTKLTVL

SEQ ID NO: 469 - protein, 123 aa, synthetic construct
  REGION 1..123: note = VH
QVQLQQSGPGLVKPSQTLSLTCAISGDSVSSNSAAWNWIRQSPSRGLEWLGRTYYRSRWYSDYASSVESRITINPDTSKNQFSLQLSSVTPEDTAVYYCARDPPYVLSTFDIWGQGTMVTVSS

SEQ ID NO: 470 - protein, 110 aa, synthetic construct; identical to SEQ ID NO: 468
  REGION 1..110: note = VL

SEQ ID NO: 471 - protein, 123 aa, synthetic construct
  REGION 1..123: note = VH
QVQLQQSGPGLVKPSQTLSLTCAISGDSVSSDSAAWNWIRQSPSRGLEWLGRTYYRSKWYNDYAPSVESRITINPDTSKNQFSLQLSSVTPEDTAVYYCARDPPYVLSTFDIWGQGTMVTVSS

SEQ ID NO: 472 - protein, 110 aa, synthetic construct; identical to SEQ ID NO: 468
  REGION 1..110: note = VL

SEQ ID NO: 473 - protein, 123 aa, synthetic construct; identical to SEQ ID NO: 467
  REGION 1..123: note = VH

SEQ ID NO: 474 - protein, 110 aa, synthetic construct
  REGION 1..110: note = VL
NFMLTQPPSVSESPGKTVTISCTRSSGNIASFYVQWFQQRPGSSPTTVIYDDDQRPSGVPDRFSGSIDRSSNSASLTISGLTTDDEADYYCQSYDTNNVIFGGGTKLTVL

SEQ ID NO: 475 - protein, 123 aa, synthetic construct; identical to SEQ ID NO: 467
  REGION 1..123: note = VH

SEQ ID NO: 476 - protein, 110 aa, synthetic construct
  REGION 1..110: note = VL
NFMLTQPPSVSESPGKTVTISCTRSSGNIASFYVQWFQQRPGSSPTTVIYEDDQRPSGVPDRFSGSIDRSSNSASLTISGLTTDDEADYYCQSYDTNKVIFGGGTKLTVL

SEQ ID NO: 477 - protein, 123 aa, synthetic construct; identical to SEQ ID NO: 467
  REGION 1..123: note = VH

SEQ ID NO: 478 - protein, 110 aa, synthetic construct
  REGION 1..110: note = VL
NFMLTQPPSVSESPGKTVTISCTRSSGNIASFYVQWFQQRPGSSPTTVIYEDDQRPSGVPDRFSGSIDRSSNSASLTISGLTTDDEADYYCQSYDINKVIFGGGTKLTVL

SEQ ID NO: 479 - protein, 123 aa, synthetic construct; identical to SEQ ID NO: 467
  REGION 1..123: note = VH

SEQ ID NO: 480 - protein, 110 aa, synthetic construct
  REGION 1..110: note = VL
NFMLTQPPSVSESPGKTVTISCTRSSGNIASFYVQWFQQRPGSSPTTVIYDDDQRPSGVPDRFSGSIDRSSNSASLTISGLTTDDEADYYCQSYDINKVIFGGGTKLTVL

SEQ ID NO: 481 - protein, 120 aa, synthetic construct
  REGION 1..120: note = VH
QMQLVQSGAEVKKPGASVKVSCKASGYMPDGYYMHWVRQAPGQGLEWMGWINPRTGGTNYAQKFQGRVTITRDTSASTAYMELSSLRSEDTAVYYCAREGAAFRLELDAWGQGTLVTVSS

SEQ ID NO: 482 - protein, 106 aa, synthetic construct
  REGION 1..106: note = VL
SYELTQPPSVSVAPGKTARITCGGNNIGSKSVHWYQQKPGQAPVLVIYYDSDRPSGIPERFSGSNSGNTATLTISRVEAGDEADYYCQVWDSSSVVFGGGTQLTVL

SEQ ID NO: 483 - protein, 120 aa, synthetic construct
  REGION 1..120: note = VH
QMQLVQSGAEVKKPGASVKVSCKASGYSFTGYYMHWVRQAPGQGLEWMGWVNPKSGGTNYAQKFQGRVTITRDTSASTAYMELSSLRSEDTAVYYCAREGWARRIDLDEWGQGTLVTVSS

SEQ ID NO: 484 - protein, 106 aa, synthetic construct; identical to SEQ ID NO: 482
  REGION 1..106: note = VL

SEQ ID NO: 485 - protein, 117 aa, synthetic construct
  REGION 1..117: note = VH
QMQLVQSGAEVKKPGASVKVSCKASGYSFSGYYMHWVRQAPGQGLEWMGWVNPMSGGTNYAQKFQGRVTITRDTSASTAYMELSSLRSEDTAVYYCAREGMAMRLELDKWGQGTLVT

SEQ ID NO: 486 - protein, 106 aa, synthetic construct; identical to SEQ ID NO: 482
  REGION 1..106: note = VL

SEQ ID NO: 487 - protein, 120 aa, synthetic construct
  REGION 1..120: note = VH
QMQLVQSGAEVKKPGASVKVSCKASGYNFGGYYMHWVRQAPGQGLEWMGWVNPHSGGTNYAQKFQGRVTITRDTSASTAYMELSSLRSEDTAVYYCAREGEAWGLDLDLWGQGTLVTVSS

SEQ ID NO: 488 - protein, 106 aa, synthetic construct; identical to SEQ ID NO: 482
  REGION 1..106: note = VL

SEQ ID NO: 489 - protein, 120 aa, synthetic construct
  REGION 1..120: note = VH
QMQLVQSGAEVKKPGASVKVSCKASGYNWTGYYMHWVRQAPGQGLEWMGWINPMAGGTNYAQKFQGRVTITRDTSASTAYMELSSLRSEDTAVYYCAREGWARGVELDMWGQGTLVTVSS

SEQ ID NO: 490 - protein, 106 aa, synthetic construct; identical to SEQ ID NO: 482
  REGION 1..106: note = VL

SEQ ID NO: 491 - protein, 120 aa, synthetic construct
  REGION 1..120: note = VH
QMQLVQSGAEVKKPGASVKVSCKASGYNFGGYYMHWVRQAPGQGLEWMGWVNPKSGGTNYAQKFQGRVTITRDTSASTAYMELSSLRSEDTAVYYCAREGEAVGLDLDYWGQGTLVTVSS

SEQ ID NO: 492 - protein, 106 aa, synthetic construct; identical to SEQ ID NO: 482
  REGION 1..106: note = VL

SEQ ID NO: 493 - protein, 120 aa, synthetic construct
  REGION 1..120: note = VH
QMQLVQSGAEVKKPGASVKVSCKASGYSLEGYYMHWVRQAPGQGLEWMGWINPNSGGTNYAQKFQGRVTITRDTSASTAYMELSSLRSEDTAVYYCAREGEAVGLDLDYWGQGTLVTVSS

SEQ ID NO: 494 - protein, 106 aa, synthetic construct; identical to SEQ ID NO: 482
  REGION 1..106: note = VL

SEQ ID NO: 495 - protein, 120 aa, synthetic construct; identical to SEQ ID NO: 493
  REGION 1..120: note = VH

SEQ ID NO: 496 - protein, 106 aa, synthetic construct; identical to SEQ ID NO: 482
  REGION 1..106: note = VL

SEQ ID NO: 497 - protein, 120 aa, synthetic construct
  REGION 1..120: note = VH
QMQLVQSGAEVKKPGASVKVSCKASGYTFTGYYMHWVRQAPGQGLEWMGWINPNSGGTNYAQKFQGRVTITRDTSASTAYMELSSLRSEDTAVYYCAREGEAVGLDLDYWGQGTLVTVSS

SEQ ID NO: 498 - protein, 106 aa, synthetic construct; identical to SEQ ID NO: 482
  REGION 1..106: note = VL

SEQ ID NO: 499 - protein, 121 aa, synthetic construct
  REGION 1..121: note = VH
EVQLVQSGAEVKKPGSSVKVSCKASGYTFTSYYIYWVRQAPGQGLEWIGNIWPGNGGTFYGEKFMGRATFTADTSTSTAYMELSSLRSEDTAVYYCARRPDYSGDDYFDYWGQGTLVTVSS

SEQ ID NO: 500 - protein, 111 aa, synthetic construct
  REGION 1..111: note = VL
SVELTQPPSVSVSPGQTARITCKLNSGNIGSYYVHWYQQKPGQAPVTMIYRDDKRPDGIPERFSGSSDSSSNSAFLTISGVQAEDEADYYCHSYDSTITPVFGGGTKLTVL

SEQ ID NO: 501 - protein, 118 aa, synthetic construct
  REGION 1..118: note = VH
EVQLVQSGAEVKKPGSSVKVSCKASDYTFNDYWVSWVRQAPGQGLEWIGEIYPNSGATNFNGKFRGRATLTVDNSASTAYMELSSLRSEDTAVYYCTREYTRDWFAYWGQGTLVTVSS

SEQ ID NO: 502 - protein, 112 aa, synthetic construct
  REGION 1..112: note = VL
DVVLTQSPLSLPVTLGQPASISCRSSQSLLDSDGNTYLYWFQQRPGQSPRRLIYLVSNLGSGVPDRFSGSGSGTDFTLKISRVEAEDVGVYYCMQPTHAPYTFGQGTKLEIK

SEQ ID NO: 503 - protein, 118 aa, synthetic construct; identical to SEQ ID NO: 501
  REGION 1..118: note = VH

SEQ ID NO: 504 - protein, 112 aa, synthetic construct
  REGION 1..112: note = VL
DVQLTQSPSSLSASVGDRVTITCRSSQSLLDSDGNTYLYWFQQKPGKAPKRLIYLVSNLGSGVPSRFSGSGSGTDFTLTISSLQPEDFATYYCMQPTHAPYTFGQGTKVEIK

SEQ ID NO: 505 - protein, 117 aa, synthetic construct
  REGION 1..117: note = VH
EVQLLESGGGLVQPGGSLRLSCAASGFTFGYSYMSWVRQAPGKGLEVSSIGSGSSYTYYADSVKGRFTISRDNSKNTLYLQMNSLRAEDTAVYYCARVYSSPGIDYWGQGTLVTVSS

SEQ ID NO: 506 - protein, 107 aa, synthetic construct
  REGION 1..107: note = VL
DIQMTQSPSSLSASVGDRVTITCRASQSISSYLNWYQQKPGKAPKLLIYAASSLQSGVPSRFSGSGSGTDFTLTISSLQPEDFATYYCQQYYTWVPFTFGQGTKLEI

SEQ ID NO: 507 - protein, 120 aa, synthetic construct
  REGION 1..120: note = VH
VHEVQLLESGGGLVQPGGSLRLSCAASGFNFGYSYMSWVRQAPGKGLEWVSSIGSTSSHTYYADSVKGRFTISRDNSKNTLYLQMNSLRAEDTAVYYCARVYSSPGIDYWGQGTLVTVSS

SEQ ID NO: 508 - protein, 107 aa, synthetic construct
  REGION 1..107: note = VL
DIQMTQSPSSLSASVGDRVTITCRASQSIGSTLNWYQQKPGKAPKLLIYGASSLQSGVPSRFSGSGSGTDFTLTISSLQPEDFATYYCQQYYTWVPFFGQGTKLEIK

SEQ ID NO: 509 - protein, 118 aa, synthetic construct
  REGION 1..118: note = VH
QVQLQQSGAELVRPGVSVKISCKGSGYTFTDYAMHWVKQSHVKSLEWIGGISTYNGDASYNQKFKGKATMTVDKSSRTAYMELVRLTSEDSAIYYCAPSNYGYAMDYWGQGTSVTVSS

SEQ ID NO: 510 - protein, 113 aa, synthetic construct
  REGION 1..113: note = VL
DIVMSQSPSSLAVSVGEKVSVSCKSSQSLLYSSNQKNYLAWYQQKPGQSPKLLIYWASTRESGVPDRFTGSGSGTDFTLTISSVKAEDLAVYYCQQYLSYPWTFGGGTKLEIN

SEQ ID NO: 511 - protein, 118 aa, synthetic construct
  REGION 1..118: note = VH
QVQLQQSGAELARPGASVKMSCKASGYIFTTYAMHWVKQRPGQGLEWIGYINPTTVYTIYNQKFKDKATLTADKSSSTAYMQLSSLTSEDSAVYYCARLGGHWYFDVWGAGTTVTVSS

SEQ ID NO: 512 - protein, 111 aa, synthetic construct
  REGION 1..111: note = VL
DIVLTQSPASLAVSLGQRATISCRASESVDNYGISFMNWFQQKPGQPPKLLIYSASNQGSGVPARFSGSGSGTDFSLNIHPMEEDDTAMYFCQQSKEVPWTFGGGTKLEIK

SEQ ID NO: 513 - protein, 117 aa, synthetic construct
  REGION 1..117: note = VH
QVQLQQSGTELMKPGASVKLSCKASGYIFTGYWIEWVKQRPGHGLEWIGEILPGGGTVYYNEKFKGKATFTAETSSNTAYVQLSSLTTEDSAVYYCARWGRSWFTHWGQGTLVTVSA

SEQ ID NO: 514 - protein, 112 aa, synthetic construct
  REGION 1..112: note = VL
DVLMTQTPLSLPVSLGDQASISCRSSQSIVHSNGNTYLEWYLQKPGQSPKLLIYEVSNRFSGVPDRFSGSGSGTDFTLKISRVEAEDLGIYYCFQGSHVPRTFGGGTKLEIK

SEQ ID NO: 515 - protein, 121 aa, synthetic construct
  REGION 1..121: note = VH
QVQLQQPESELVRPGGSVKLSCKASGYTFTSYWMDWVKQRHGQGLEWIGNIYPDSGGTNYAEKFKSKATLTVDTSSSTAYMHLSSLTSEDSAVYYCTREEALGGYYELTYWGQGTLVTVSA

SEQ ID NO: 516 - protein, 111 aa, synthetic construct
  REGION 1..111: note = VL
DIVLTQSPASLPVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIE

SEQ ID NO: 517 - protein, 119 aa, synthetic construct
  REGION 1..119: note = VH
QVQLQQSGAELVRPGTSVKISCKASGYAFTNYWLGWVKQRPGHGLEWIGDIYPGNGNNYYSEKFKDKATLTADKSSSTVYIRLSSLTSEDSAVYFCARHGSFRSAMDYWGQGTSVTVSS

SEQ ID NO: 518 - protein, 107 aa, synthetic construct
  REGION 1..107: note = VL
DIQMTQSPASPSASVGETVTITCRASENIYSYLAWYQQKQGKSPQLLVYNAKTLADGVPSRFSGSGSGTQFSLKINSLQPEDFGSYYCQHHYGTPPTFGGGTKLEIK